(19) United States
(12) Patent Application Publication
(10) Pub. No.: US 2009/0281519 A1
(43) Pub. Date: Nov. 12, 2009

(54) AUTOMATED SYSTEM AND METHOD FOR DIABETES CONTROL

(76) Inventors: R. Harsha RAO, Pittsburgh, PA (US); Peter Perreiah, West Lafayette, IN (US); Candace Cunningham, Pittsburgh, PA (US)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 12/464,344

(22) Filed: May 12, 2009

Related U.S. Application Data
(60) Provisional application No. 61/071,672, filed on May 12, 2008.

(51) Int. Cl. A61M 5/172

(52) U.S. Cl. 604/504; 604/66

(57) ABSTRACT

An automated method and system of diabetes control. The method includes establishing a blood glucose target for an insulin user, measuring an existing blood glucose level for the insulin user, and inputting the existing blood glucose level into a computer processor formed to execute an algorithm designed to calculate a corrective amount of insulin to be administered intravenously in an integrated basal-bolus manner to the insulin user if the existing blood glucose level exceeds the blood glucose target. The algorithm is based on a plurality of factors that contribute to a non-linear glucose response. The method further includes automatically delivering the corrective amount of insulin to the insulin user, and further repeating the measuring, inputting, and delivering steps one or more times to maintain the insulin user within the blood glucose target range.

### CROSS-REFERENCE TO RELATED APPLICATION(S)

This application claims the benefit of provisional patent application U.S. Ser. No. 61/071,672, filed May 12, 2008, which is expressly incorporated herein by reference.

### BACKGROUND OF INVENTION

a. Field of Invention

The invention relates generally to diabetes treatment and control, and more particularly to an automated system and method for diabetes treatment and control for patients admitted to the hospital or at non-hospital facilities.

b. Description of Related Art

Known protocols and algorithms have been developed and are in use for calculating IV insulin administration rate, or “insulin drip rate” for critically ill patients with diabetes. Such known protocols and algorithms all achieve varying degrees of success in reaching the goal of normalizing blood glucose levels, and generally share two core features/concepts: (1) they are two-dimensional (e.g., either linear or curvilinear), and (2) they incorporate quantum threshold and/or step functions. The operational application of these core concepts may vary from protocol to protocol, but the fundamental decision-making processes are very similar, and are based on the premise that the relationship between the insulin drip rate (the dependent variable) and ambient plasma glucose concentrations (the independent variable) can be fitted to a relatively simple natural function, such as a straight line or a curve (e.g., exponential or polynomial function).

The role of insulin sensitivity or resistance as a glycemic response determinant is mathematically reflected by the slope of the line in a linear model or a first order determinant in a curvilinear model. The degree of insulin sensitivity may be adjusted in individual patients in one of two ways, depending on the observed glycemic response: (1) in “formula-driven protocols”, a designated “insulin sensitivity factor” is adjusted up or down to change the slope of the linear model; or (2) in the so-called “dose-driven protocols”, a preset threshold function triggers a step-change to a different preset slope (in a linear model) or first-order determinant (in a curvilinear model).

Insulin bolus doses are used to supplement or augment the glycemic response to the drip, using a separate preset threshold function to trigger a bolus dose calculation, either when the ambient blood glucose exceeds a preset threshold level, or when the observed response fails to meet a preset threshold change. Insulin bolus doses are calculated from a two-dimensional linear or curvilinear model, which is separate from and independent of the insulin drip rate calculation. Insulin bolus dose calculations are recalibrated, based on the glycemic response, through a step-wise change in the slope of the line or first derivative of the curve, according to either or both of the following: (1) a quantum threshold response (achieving a preset fall in plasma glucose); and/or, (2) a quantum threshold level (achieving a preset plasma glucose level). A hypoglycemic intervention protocol must be instituted when blood glucose falls below the target range (i.e., the management of low blood glucose levels is not integrated seamlessly into the main protocol).

Known glucose monitoring software exist. However, such known glucose monitoring software erroneously assume a linear relationship between glucose levels and insulin administration.

The frequency of life-threatening hypoglycemia (BG (blood glucose) <40 mg/dl) in patients receiving these aggressive intervention protocols is unacceptably high (averaging approximately 8% in Surgical ICU patients, and 13% in Medical ICU patients), and may be responsible for the fact that existing protocols have failed to demonstrate a significant mortality benefit from aggressive insulin management to achieve tight glycemic control in hospital.

Accordingly, there remains a need for an automated system and method for diabetes control that provides advantages over known systems and methods.

### SUMMARY OF THE INVENTION

In an embodiment of the invention, there is provided an automated method for diabetes control. The method includes the step of establishing a blood glucose target for an insulin user. The method further includes the step of measuring an existing blood glucose level for the insulin user. The method further includes the step of inputting the existing blood glucose level into at least one computer processor. The at least one computer processor is formed to execute at least one algorithm designed to calculate a corrective amount of insulin to be administered intravenously in an integrated basal-bolus manner to the insulin user if the existing blood glucose level exceeds the blood glucose target. The at least one algorithm is based on a plurality of factors that contribute to a non-linear glucose response. The method further includes the step of automatically delivering the corrective amount of insulin to the insulin user. The method further includes the step of repeating the measuring, inputting, and delivering steps one or more times to maintain the insulin user within the blood glucose target range.

For the method described above, the blood glucose target may be 80-110 mg/dl (milligrams per deciliter), or alternatively, 90-140 mg/dl (milligrams per deciliter). The plurality of factors include a magnitude of the existing blood glucose level, the preceding blood glucose level, a time between the existing blood glucose level and a preceding blood glucose level, a calculated rate of change in the blood glucose, a total insulin dose, and a fractional change in total insulin dose. The method further includes the step of progressively reducing the corrective amount of insulin to the insulin user as insulin sensitivity improves as the existing blood glucose level reaches the blood glucose target. The method also includes providing a safety warning in an integrated user interface to recheck the existing blood glucose level at a variable predetermined time, and is designed for use in a hospital intensive care unit.

In another embodiment of the invention, there is provided an automated method for diabetes control. The method includes the step of establishing a blood glucose target for an insulin user. The method further includes the step of establishing a carbohydrate target for the insulin user. The method further includes the step of measuring an existing blood glucose level for the insulin user. The method further includes the step of measuring an existing amount of carbohydrate consumed by the insulin user. The method further includes the step of inputting the existing blood glucose level into at least one computer processor. The at least one computer processor is formed to execute at least one algorithm designed to calculate a corrective amount of insulin to be administered subcutaneously in an integrated basal-bolus manner to the insulin user if the existing blood glucose level exceeds the blood glucose target in relation to the existing amount of carbohydrate consumed. The method further includes the step of automatically delivering the corrective amount of insulin to the insulin user after the insulin user eats a meal. The method further includes the step of repeating the measuring, inputting, and delivering steps one or more times to maintain the insulin user within the blood glucose target range.

For the method described above, the blood glucose target may be 90-140 mg/dl (milligrams per deciliter), and the carbohydrate target may be 75 gm (grams) of carbohydrate per meal. The corrective amount of insulin to be administered subcutaneously may include a bolus dose of a fast-acting analog insulin and a basal dose of a 24-hour insulin. The bolus dose of the fast-acting analog insulin may be selected from the group comprising aspart insulin, lispro insulin and glulisine insulin. The basal dose of the 24-hour insulin may include glargine. The method may be designed for use in a hospital non-intensive care unit setting.

In another embodiment of the invention, there is provided an automated system for diabetes control. The system includes a database for obtaining a blood glucose target for an insulin user. The system further includes a glucose measuring device for measuring an existing blood glucose level for the insulin user. The system further includes an insulin delivery unit. The system further includes a computer having a computer processor that receives blood glucose level measurements from the glucose measuring device, executes a software program that calculates a corrective amount of insulin to be administered intravenously in an integrated basal-bolus manner to the insulin user if the existing blood glucose level exceeds the blood glucose target, wherein the software program is based on a plurality of factors that contribute to a non-linear glucose response, and further wherein the software program has a communication element that communicates to the insulin delivery unit the corrective amount of insulin to be administered to the insulin user.

For the system described above, the blood glucose target may be 80-110 mg/dl (milligrams per deciliter), or alternatively, 90-140 mg/dl (milligrams per deciliter). The plurality of factors may include a magnitude of the existing blood glucose level, a magnitude of the preceding blood glucose level, a calculated rate of change in the blood glucose, a time between the existing blood glucose level and a preceding blood glucose level, a total insulin dose, and a fractional change in total insulin dose.

In another embodiment of the invention, there is provided an automated system for diabetes control. The system includes a database for obtaining a blood glucose target for an insulin user. The system further includes a database for obtaining a carbohydrate target for the insulin user. The system further includes a glucose measuring device for measuring an existing blood glucose level for the insulin user. The system further includes a carbohydrate measuring device for measuring an existing amount of carbohydrate consumed by the insulin user. The system further includes an insulin delivery unit. The system further includes a computer having a computer processor that receives blood glucose level measurements from the glucose measuring device and receives carbohydrate consumed measurements from the carbohydrate measuring device, and executes a software program that calculates a corrective amount of insulin to be administered subcutaneously in an integrated basal-bolus manner to the insulin user if the existing blood glucose level exceeds the blood glucose target in relation to the existing amount of carbohydrate consumed, wherein the software program has a communication element that communicates to the insulin delivery unit the corrective amount of insulin to be administered to the insulin user after the insulin user eats a meal.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1A and FIG. 1B are schematic representations of the concepts embodied in the ICU GENIE programs (Cardiac Surgery and Critical Care GENIE), according to the present invention;

FIG. 2A and FIG. 2B show the performance of the ICU GENIE programs;

FIG. 3A and FIG. 3B are charts showing glycemic performance comparisons between GENIE and different published protocols, showing that the ICU GENIE programs (i) are superior in achieving their stated target, (ii) maintain much better stability in blood glucose levels (as reflected in the significantly lower standard deviation); and (iii) do not provoke life-threatening hypoglycemia;

FIG. 4A and FIG. 4B are screen shots of the respective User Interfaces of the two ICU GENIE components in the program according to the present invention (the Cardiac Surgery and Critical Care GENIEs);

FIG. 5A and FIG. 5B are printouts of the respective charts of the Drip Grid Arrays of the two ICU GENIE components in the program according to the present invention (the Cardiac Surgery and Critical Care GENIEs);

FIG. 6 is a printout of the decision surface of the ICU GENIEs;

FIGS. 7A-7C are printouts of the Insulin Control Tables for the Initial Glycemic Management Strategy in Cardiac Surgery GENIE according to the present invention;

FIGS. 8A-8F are printouts of the Insulin Control Tables for the Subsequent Glycemic Management Strategy in Cardiac Surgery GENIE according to the present invention;

FIGS. 9A-9C are printouts of the Insulin Control Tables for the Initial Glycemic Management Strategy in Critical Care GENIE according to the present invention;

FIGS. 10A-10E are printouts of the Insulin Control Tables for the Subsequent Glycemic Management Strategy in Critical Care GENIE according to the present invention;

FIG. 11A and FIG. 11B are schematic representations of the concepts embodied in the Subcutaneous Insulin GENIE programs, according to the present invention;

FIG. 12 is a schematic representation of the decision making strategy for determining basal insulin dosing in the Subcutaneous Insulin GENIE programs, according to the present invention;

FIG. 13A and FIG. 13B are screenshots showing the Start-up screen for initiating the Subcutaneous Insulin GENIE program (FIG. 13A), and the Discharge Instruction prior to termination of the program (FIG. 13B), according to the present invention.

### DETAILED DESCRIPTION OF PREFERRED EMBODIMENTS

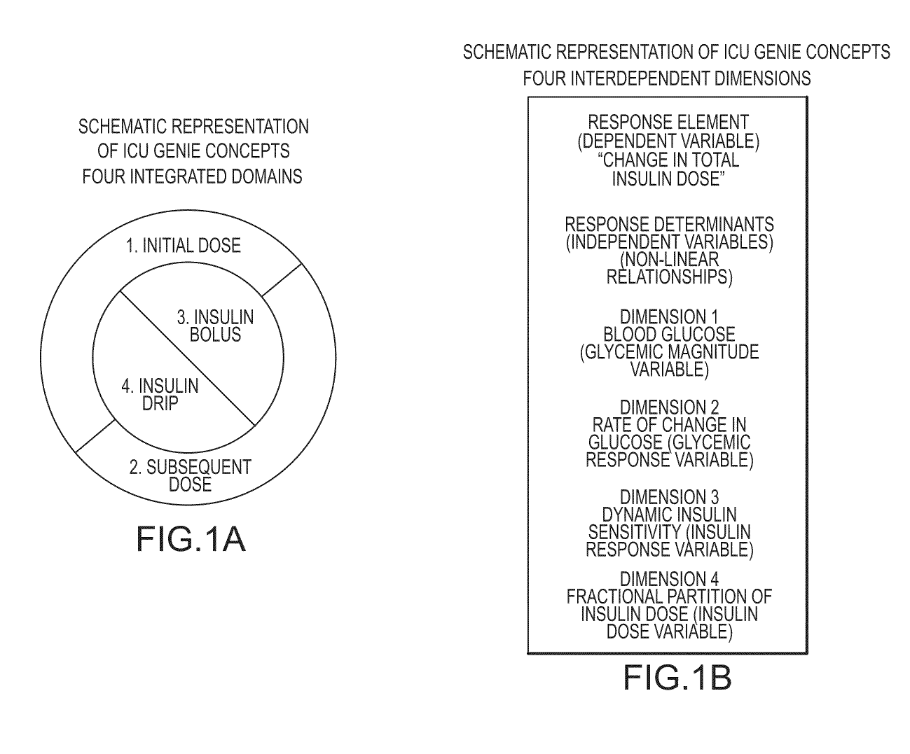

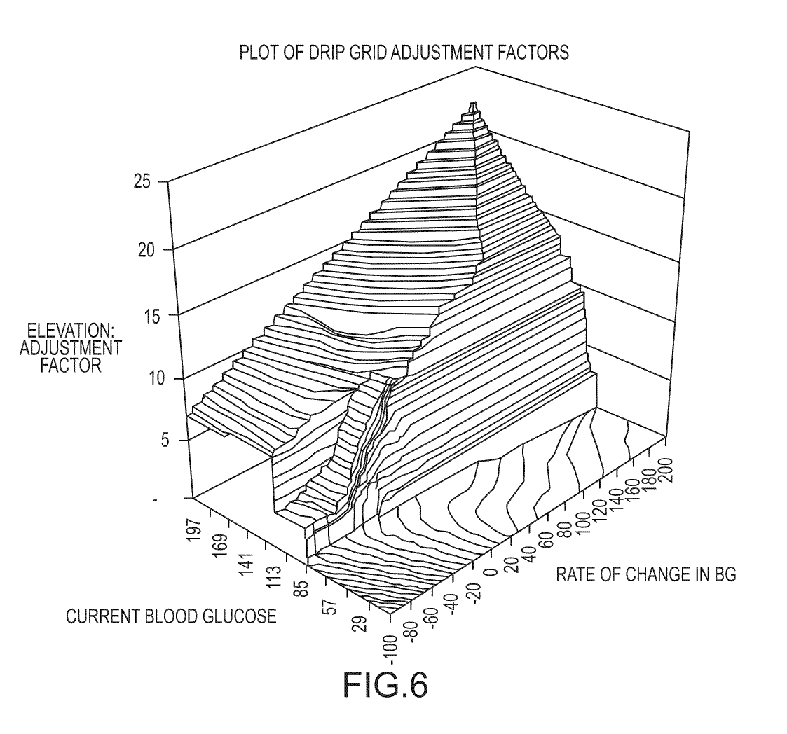

Referring now to the drawings wherein like reference numerals designate corresponding parts throughout the several views, FIGS. 1-13B illustrate various diagrams and screen print-outs for the GENIE program according to the present invention.

Referring to FIGS. 1-13B, as discussed below, the GENIE program is a single software package intended for use in a hospital by nurses, without direct physician supervision, for automated application of basal-bolus insulin regimens in adult patients. For pediatric care or other patients, the GENIE program may be readily modified to include a weight based dosing algorithm. The GENIE program thus enables precise blood sugar management for hospitalized diabetic patients in the ICU, as well as post-ICU transition.

The GENIE program includes four components, respectively named “Cardiac Surgery GENIE”, “Critical Care GENIE”, “DKA GENIE”, and “Subcutaneous Insulin GENIE”.

The Cardiac Surgery GENIE is an automated decision making tool for intensive glycemic management using intravenous basal-bolus insulin therapy in patients admitted to the intensive care unit after undergoing, for example, open heart surgery, with a target blood glucose of approximately 80-110 mg/dl.

The Critical Care GENIE is an automated decision making tool for intensive glycemic management using intravenous basal-bolus insulin therapy in patients admitted, for example, to the intensive care unit, with a target blood glucose of approximately 90-140 mg/dl.

The DKA GENIE is an automated decision making tool for intensive glycemic management using intravenous basal-bolus insulin therapy in patients with Type 1 diabetes admitted in Diabetic Keto-acidosis (DKA), with a target blood glucose of approximately 90-140 mg/dl.

The Subcutaneous Insulin GENIE, is an automated decision making tool for managing subcutaneous basal-bolus insulin therapy to maintain a target blood glucose of approximately 90-180 mg/dl in hospitalized patients outside the ICU setting.

The Cardiac Surgery GENIE and Critical Care GENIE, which are indicated for use in the intensive care units (ICUs), share a unique common platform that calculates the amount of insulin to be administered intravenously in an integrated basal-bolus manner, differing only in the targeted goal for maintenance of blood glucose.

The Subcutaneous Insulin GENIE is based on a different platform, which takes a unique approach to calculating subcutaneous insulin doses and administering those in a basal-bolus insulin regimen.

According to the present invention, the underlying premise of the Cardiac Surgery and Critical Care GENIE programs (collectively identified hereinafter as the “ICU GENIE programs”, because of their common platform) differs from previously published protocols in recognizing, for example, that the relationship between the dose of insulin to be administered intravenously and the ambient glucose concentration is far more complex than hitherto envisioned.

ICU GENIE (Cardiac Surgery and Critical Care GENIE)

A first unique concept embodied in the common platform of the two “ICU GENIE programs”, schematically represented in FIG. 1A, envisions the decision-making strategy in four domains: (1) the initial dose and (2) the subsequent dose, which are in turn partitioned into (3) the bolus dose and (4) the drip rate.

The second unique concept embodied in the common platform of the two “ICU GENIE programs”, described in FIG. 1B, is the relationship at the core of the model, consisting of the “change in total insulin dose” as the dependent variable and four “independent variables” that are used to calculate the dependent variable.

The ICU GENIE programs according to the present invention apply the common principles that govern the decision-making strategy differently in different ranges of the blood glucose spectrum. As a result, they are modeled in several parts, depending on the BG (blood glucose), that differ significantly from each other: “Below Target”, “At Target”, “Above Target” and “Well Above Target” (e.g. >approximately 200). In Cardiac Surgery GENIE only, because of its relatively narrow Target Range (approximately 80-115 mg/dl compared to approximately 90-140 for Critical Care GENIE), an additional “Just Above Target” part is included.

The multi-part model approach used in the ICU GENIE programs is based on the recognition that rapidly declining glucose toxicity is a major determinant of the glycemic response above BG approximately 200, whereas, the closer that BG approaches the target range, the greater the importance of safety factors to prevent overshoot into hypoglycemia, such as the allocation of insulin dosing to drip versus bolus administration, and the use of 5% dextrose infusions.

Below BG approximately 200 mg/dl, the ICU GENIE programs model the relationship between the dependent and the independent variables as a “continuous, multidimensional decision surface area” that can be readily adjusted if the supervising endocrinologist wants to change the surface settings.

The ICU GENIE programs provide separate recommendations depending on whether the inputs dictate an “initial management strategy” (i.e. “Starting” or “Restarting” dose calculations for insulin drip and bolus), or a “subsequent management strategy” (“Continuous” dose calculations for insulin drip and bolus).

Above BG approximately 200 mg/dl, the ICU GENIE programs use a complex equation that mathematically incorporates four different inputs (independent variables) into a coherent decision-making strategy, namely, (a) BG level, (b) the preceding BG level, (c) time between previous and current BG level and (d) the current IV insulin drip rate.

The ICU GENIE programs use a concept of “excess glucose” to represent a combination of independent variables that are the inputs for the multidimensional decision surface area. Below BG approximately 200, the model considers the amount that the excess glucose needs to be reduced, taking into account safety factors such as “distance to target” and preceding glycemic response. Above BG approximately 200, excess BG is applied directly in the formula and drives the insulin calculation.

The ICU GENIE programs apply a concept of “total insulin dose” to the dependent variable, which is the total amount of insulin to be administered before the next plasma glucose estimation, both as drip and as bolus. A concept of “fractional change in total insulin dose” is used to estimate the change in the dependent variable from the previous calculation, so that insulin dose to be administered is determined as a function of the previously administered dose.

The ICU GENIE programs calculate the “fractional change in insulin dose” in relation to both the observed glycemic response and the prevailing (ambient) glucose level, and the desired glycemic response, and partition the “fractional change in total insulin dose” into two elements, the “drip rate” and the “bolus dose”, making them inter-dependent, not independent.

The ICU GENIE programs apply the concept of “interdependence in the fractional changes in drip rate and bolus dose” to improve precision of the estimated overall glycemic response. This recognizes the immediate impact on insulin sensitivity of the bolus component, and accordingly sets the drip component to take into account the increasing insulin sensitivity, thereby creating an appropriate “total insulin dose”.

Above BG approximately 200, the ICU GENIE programs use a concept of “dynamically changing insulin sensitivity” to reflect the fact that insulin sensitivity is not only an independent glycemic response variable, but also a “dependent glycemic response variable” that is affected by both the level of ambient glucose, and the observed glycemic response.

The ICU GENIE programs apply the concept of “dynamically changing insulin sensitivity” to an “ongoing recalibration of the model” based on both the observed glycemic response and the “total insulin dose” administered since the previous glucose estimation. A concept of a “weaning effect” is incorporated to progressively reduce the insulin dosing as insulin sensitivity improves as the BG enters the high end of the BG target range. This prevents BG levels from dropping below the target euglycemic range and further into the hypoglycemic range.

The ICU GENIE programs use a “twin dial” strategy to improve precision in control at both ends of the spectrum of glycemia. In the hyperglycemic range, the “twin dial strategy” is applied through the concept of “proportional fractionation of the total insulin dose into insulin drip rate and insulin bolus dose” for improved precision in accelerating the correction of hyperglycemia without undershooting or overshooting the target. In the euglycemic range, the “twin dial strategy” is applied through the concept of “glycemic modulation” whereby the addition of a variable 5% dextrose infusion improves precision in decelerating the correction of hyperglycemia as the target is approached. Stable euglycemia is achieved through the increased level of external control of the glucose level afforded by the “twin-dial strategy” of starting a dextrose infusion while continuing to administer insulin, rather than simply discontinuing the insulin drip.

Recommendations for insulin administration are provided via both drip and bolus, using a unique algorithm based on a range of parameters, including the magnitude and rate of change in the patient's blood sugar, the existing and preceding blood glucose (as an index of prior insulin responsiveness), and the existing insulin drip rate (as an index of prevailing insulin sensitivity).

As discussed earlier, existing glycemic management software erroneously assume a linear relationship between glucose levels and insulin administration. Factors that contribute to non-linear glucose response are accounted for, thus providing an insulin administration protocol that is more accurate, and tailored to the patient's individual glucose metabolism. The ICU GENIE programs thus not only provide a better insulin regimen, but they do so faster than other tools currently in use.

A hypoglycemia intervention protocol for the management of low blood glucose levels is integrated seamlessly into the ICU GENIE programs. The hypoglycemic intervention is based on the level of blood glucose, the direction of blood glucose change, and the amplitude of the change, in contrast to other protocols, which base intervention on the level of blood glucose alone.

The ICU GENIEs are uniquely able to account for changes in BG that are ascribable to clearly identifiable external inputs by incorporating into the decision making strategy specific interventions for glycemic management in special circumstances. Recommendations are provided for glycemic management in anticipation of hyperglycemia as a result of Steroid administration. Recommendations are provided for glycemic management in anticipation of hyperglycemia as a result of Hespan administration. Recommendations are also provided for glycemic management in anticipation of hyperglycemia as a result of Food intake, allowing for the continuation of the IV insulin drip, if required, even when the patient is able to eat.

The ICU GENIE programs create a dependent variable “Recheck time” based on the blood glucose level and rate of change in the blood glucose. This recheck time determines the safe period in minutes before blood glucose must be checked again and insulin dosing appropriately modified. This “Recheck Time” is presented in an integrated user interface along with the requirements for settings to the insulin drip rate, insulin bolus dose and safety warnings.

The ICU GENIE programs may present safety warnings in an integrated user interface to recheck blood glucose levels at 30 minutes from the initiation or termination of a vasopressor drip. For example, when insulin dosing reaches high levels, verbal alerts may be presented in an integrated user interface to ensure potential errors have not been entered as inputs to the calculations. A verbal alert may be presented to terminate the dextrose infusion and maintain normal saline solution infusion when blood glucose levels are in the euglycemic range.

The ICU GENIE programs achieve near-normoglycemia within approximately 240 minutes for approximately 95% of ICU patients. Patient outcomes are improved with much better control by reducing BOTH level and duration of glycemic exposure, while maintaining sustained near-normoglycemia.

The ICU GENIE programs maintain safe glucose levels in ICU patients better than traditional methods, and better than existing software tools. Thus the risk of serious hypoglycemia is reduced, and the duration of need for vasopressor therapy in patients developing hypotension is also reduced.

The ICU GENIE programs are Microsoft Excel and Visual Basic based, and compatible with all windows based platforms and services, and are easy to use, allowing for a seamless transition to the ward-based protocol (Subcutaneous Insulin GENIE).

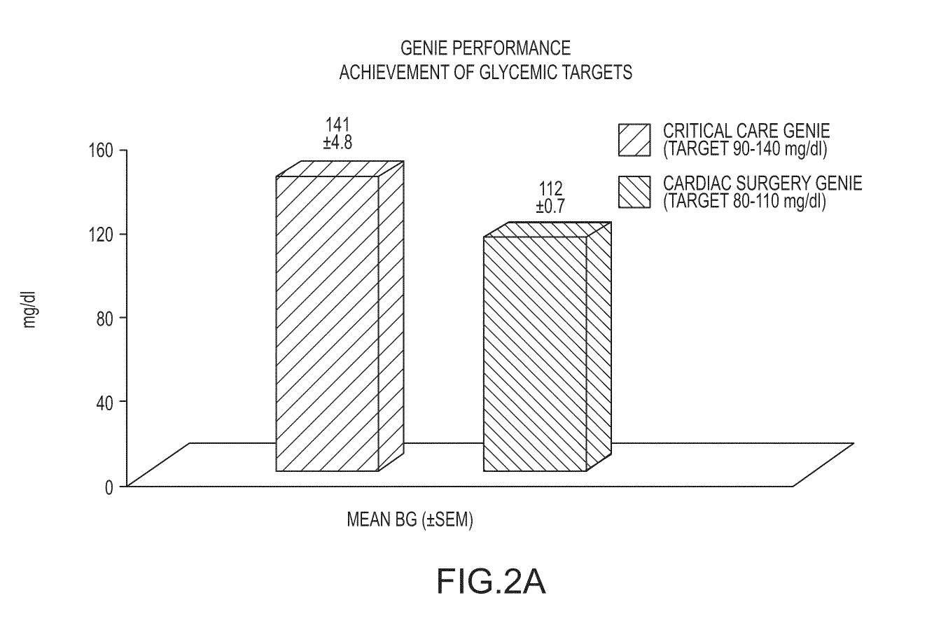

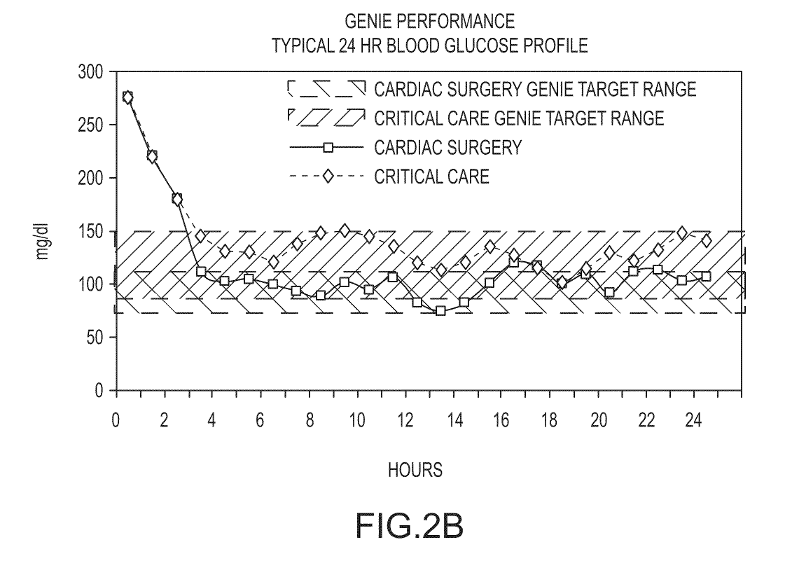

FIGS. 2A and 2B show the analysis of the performance of the two ICU GENIEs (Cardiac Surgery and Critical Care GENIE). FIG. 2A depicts the mean blood glucose attained in 363 patients treated with the final version of Cardiac Surgery GENIE and 32 patients treated with the final version of the more recently developed Critical Care GENIE. These show that both ICU GENIEs achieve their target range without significant variability.

FIG. 2B shows the pattern of blood glucose levels attained in an individual patient treated with either Cardiac Surgery GENIE or Critical Care GENIE. The patterns seen are typical of the majority of patients treated with one or the other of the ICU GENIEs.

Thus, for instance, in patients treated with the Cardiac Surgery GENIE, target blood glucose (80-110 mg/dl) is achieved in 90% of patients within 4 hours, and blood glucose exceeds approximately 150 mg/dl only 17% of the total time (including the time taken to reach target). In the “typical” profile for Cardiac Surgery GENIE shown in FIG. 2B, the BG can be seen to decline into the target range at 3 hours and stay within range for most of the time.

Similarly, in patients treated with the Critical Care GENIE, target blood glucose (approximately 90-150 mg/dl) is achieved within 4 hours in 90% of patients and blood glucose exceeds approximately 200 mg/dl only 3.3% of the total time (including the time taken to reach target). In the “typical” profile for Critical Care GENIE shown in FIG. 2B, the BG can be seen to decline into the target range at 3 hours and stay within range for most of the time.

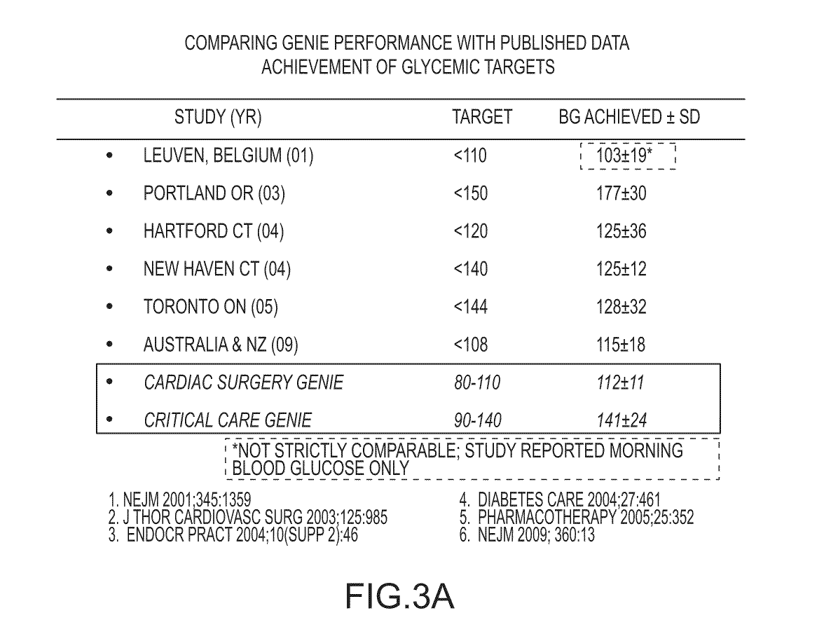

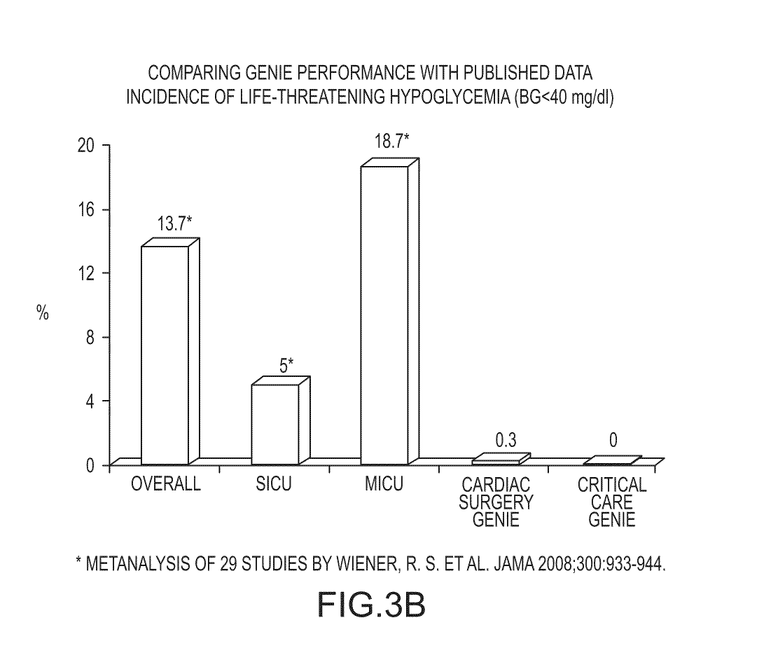

FIGS. 3A and B show a comparison between GENIE performance and the performance of several published protocols. As can be seen in FIG. 3A, both ICU GENIEs achieve their respective targets with significantly lower variability than the published protocols. An even more striking advantage of the two ICU GENIEs over published protocols for tight glycemic controls is evident in FIG. 3B. A meta-analysis of all randomized controlled trials using tight glycemic control protocols shows that all of these are associated with an unacceptably high incidence of life-threatening hypoglycemia, with an average of 5% in the Surgical ICU (compared to 0.3% with the Cardiac Surgery GENIE) and an average of almost 13% in the Medical ICU (compared to which, there was not a single such occurrence in the 32 patients treated with the Critical Care GENIE).

Rules for Determining ICU GENIE Recommendations

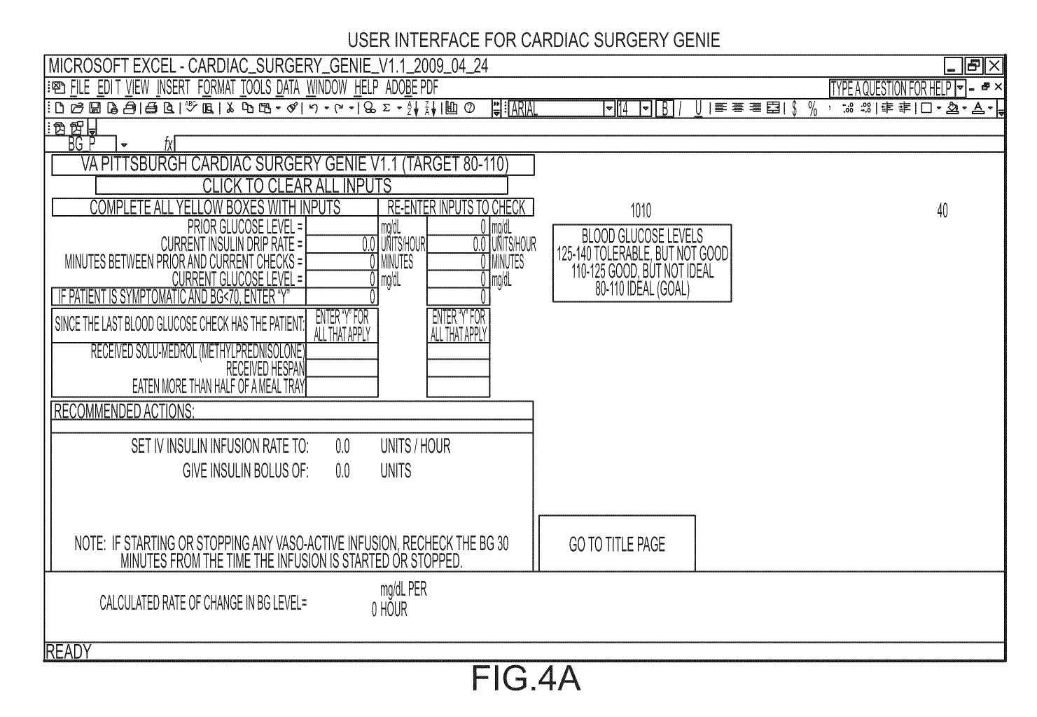

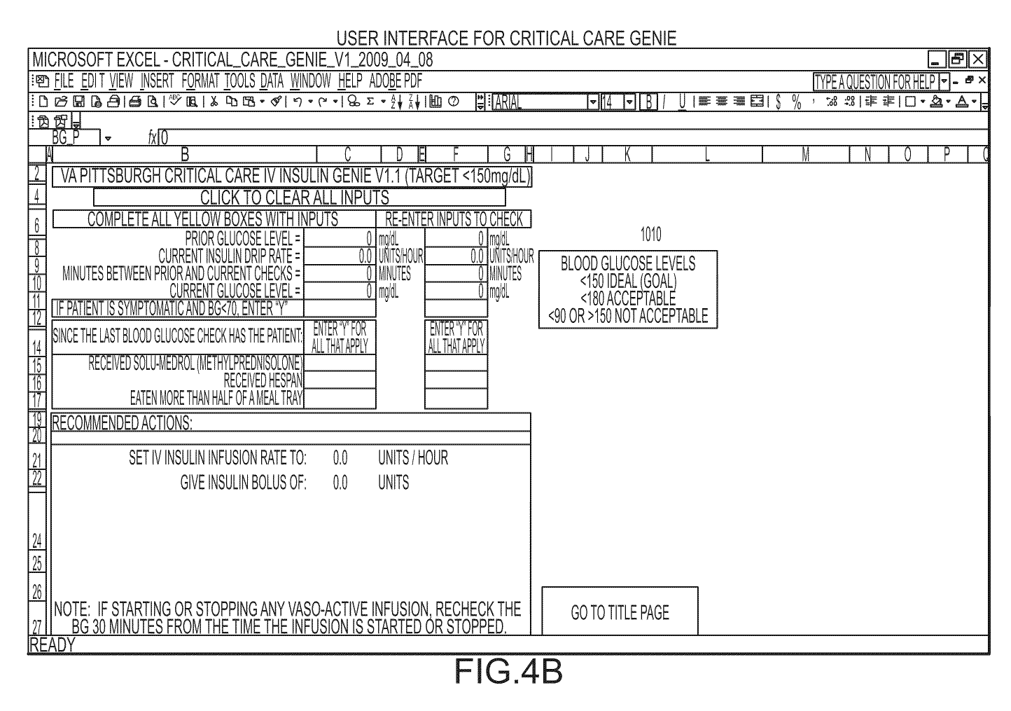

The User-interfaces for the Cardiac Surgery and Critical Care GENIES™ are shown in FIGS. 4A and 4B. These interfaces, which are uniquely user-friendly and intuitively easy to follow for data entry, provide therapy recommendation for IV Insulin administration, consisting of four components:

1. IV insulin drip rate;

2. IV insulin bolus dose;

3. Verbal instructions; and

4. A time interval for the next blood glucose check.

These four therapy components are specific to a unique patient ‘case.’ A ‘case’ is defined by the user inputs of the patient's current blood glucose measurement, the patient's prior blood glucose, the time (minutes) since the prior blood glucose, the current IV insulin drip rate and, if the patient is hypoglycemic, whether the patient is symptomatic. A case thus provides three sources of information on the patient for use in evaluating therapy changes:

1. Glycemic Magnitude—the current blood glucose;

2. Glycemic Response—the rate of change in blood glucose in mg/dl/hour and

3. Insulin Response—the current insulin drip rate, which impounds information on patient's insulin sensitivity/resistance, given the cumulative therapy changes.

A fourth independent variable described in the concepts outlined above (fractional partitioning of insulin dose) is also factored into the recommendations, as described below.

The approach to determining dosing recommendations (total insulin dose and fractional partition into drip or bolus insulin) is similar to both the Cardiac Surgery and Critical Care GENIEs. The shared approach will be described in Steps 1-4, after which the specific application of these Steps in the two ICU GENIEs will be described, as those relate to their different target ranges.

A. Steps for Determining Insulin Dosing Recommendations that are Common to both ICU Genies

STEP 1: Creating a Case Code for Formula Reference

Each of the four user inputs is categorized and assigned a reference index. If no entry or ‘zero’ is input for the prior blood glucose, then the patient is assumed to be starting or restarting on a drip (Initial dose Calculation). A six-digit ‘case code’ is created by concatenating the four indices. The case code is then used by formulas in the user interface to return from the Insulin Control Table the four components of the therapy recommendation.

STEP 2: Calculating the IV insulin drip rate

1. The User Interface returns the value for a new drip rate from the Insulin Control Table that is specific to each of the two GENIEs. These two Tables are described below in detail individually.

2. The GENIE program performs two tests before making a recommendation on the insulin dosing, as follows:
a. The first check is to see if the patient has eaten more than one half of a meal tray.
b. The second check is to determine the change in blood glucose since the previous value.

3. Based on these two tests, the User interface returns recommendations as follows:
a. If the patient has eaten more than one half of a meal tray, and the blood glucose has increased approximately 100 or more points, then the current insulin rate is increased by one unit for every approximately 50 points of increase.
b. If the patient has eaten and the increase is less than 100 points, then the current insulin rate is left unchanged.
c. If the patient has not eaten but the new IV insulin drip rate returned from the Insulin Control Table is less than approximately 0.5 units, then the IV insulin drip is turned off (normal saline at approximately 20 cc/hour is kept running to keep the line clear).

4. In all other circumstances, the IV insulin drip rate is calculated as shown below from the Insulin Control Table for the specific patient case and displayed to the user.

5. All IV insulin drip rates are rounded to the nearest approximately 0.2 units. As a safety precaution, the maximum drip rate allowed is twice the current drip rate plus ten units. A new IV insulin drip rate (I_{new}) is calculated one of three ways depending on the range of the blood glucose:
a. For blood glucoses approximately 70-79 where the blood glucose has decreased at a rate greater than approximately 40 mg/dL/hour, and all blood glucose less than approximately 70 mg/dL: I_{new}=0. That is, the drip is turned off to avoid pushing the patient into hypoglycemia.
b. For blood glucose approximately 70-199 mg/dL: A drip rate modifying factor is looked up in the Drip Grid array and multiplied by the current drip rate (see detailed discussion on the Drip Grid, below). Again the formula rounds the new drip rate to the nearest approximately 0.2 units/hour.
c. For blood glucose above approximately 200 mg/dL: the following formula is used to calculate a new IV insulin drip rate:

I_{new}=I_{current}+{[[(BG_{current}−BGₚᵣᵢₒᵣ)/((T_{current}−Tₚᵣᵢₒᵣ)/60))]−[(BG_{current}−80)*(0.14*ln(BG_{current}−80)−1)]]*[(1/ISF_{standard})*(BG_{current}/100)]}

Where:

| Compnent | Definition |
|---|---|
| I_{new} | New IV insulin drip rate (to be |
| | calculated) |
| I_{current} | Current IV insulin drip rate |
| BG_{current} | Current Blood Glucose in mg/dL |
| BGₚᵣᵢₒᵣ | Prior Blood Glucose in mg/dL |
| T_{current} | Current Time |
| Tₚᵣᵢₒᵣ | Prior Time |
| ISF_{standard} | Standard Insulin Sensitivity |
| | Factor(constant): mg/dL change in BG |
| | per unit of insulin (Set to |
| | approximately 40 mg/dL/unit) |
| [(BG_{current} − BGₚᵣᵢₒᵣ)/((T_{current} − | Current rate of change in blood glucose |
| Tₚᵣᵢₒᵣ)/60))] | per 60 minutes |
| [(BG_{current} − 80) * (0.14 * | Target rate of change in blood glucose |
| ln(BG_{current} − 80) − 1)] | per 60 minutes |
| (1/ISF_{standard}) | Multiplying the difference between the |
| | current and target rates of change in |
| | blood glucose by this inverse of the |
| | insulin sensitivity factor gives the |
| | amount of insulin per hour the current |
| | drip rate needs to be changed (un- |
| | adjusted for insulin resistance). |
| (BG_{current}/100) | Insulin Resistance Factor used to adjust |
| | the standard insulin sensitivity |
| | (ISF_{standard}) to account for increasing |
| | insulin resistance with increasing blood |
| | glucose. |

STEP 3: Calculating the IV Insulin Bolus

IV insulin bolus dose recommendations are case-specific. A formula on the User Interface references the case-specific bolus dose and returns its value. If a bolus dose is recommended, it will have a value no less than approximately 2 units and no more than approximately 12 units for safety reasons. However, minimum and maximum bolus values may be higher and lower, respectively, depending on the case (see the GENIE specific Insulin Control Table listings described below for Cardiac Surgery GENIE and Critical Care GENIE). Since the GENIE™ algorithm uses variable recheck times, it is likely that a patient with very high blood glucose may get multiple boluses within an hour depending on the measured response to therapy.

Bolus dose recommendations are of three types:

1. No bolus (zero units).

2. A specified constant: for patients who are starting or restarting a drip with hyperglycemia; or for patients on a drip, up to a maximum approximately 12 units.

3. A fraction of the new drip rate recommendation: approximately 25%, 50% or 100% of the new drip rate rounded up to the next whole number of units. All these variable IV insulin bolus recommendations have minimum and maximum units allowed for a bolus.

STEP 4: Referencing an IV Insulin Drip Rate Modifying Factor

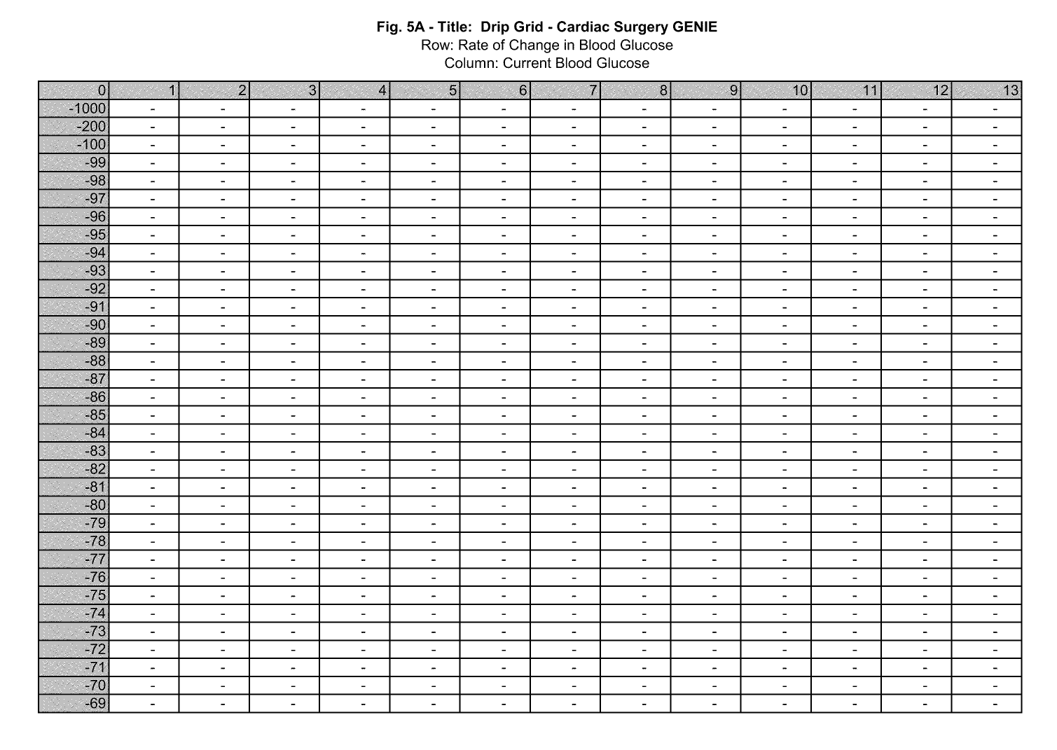

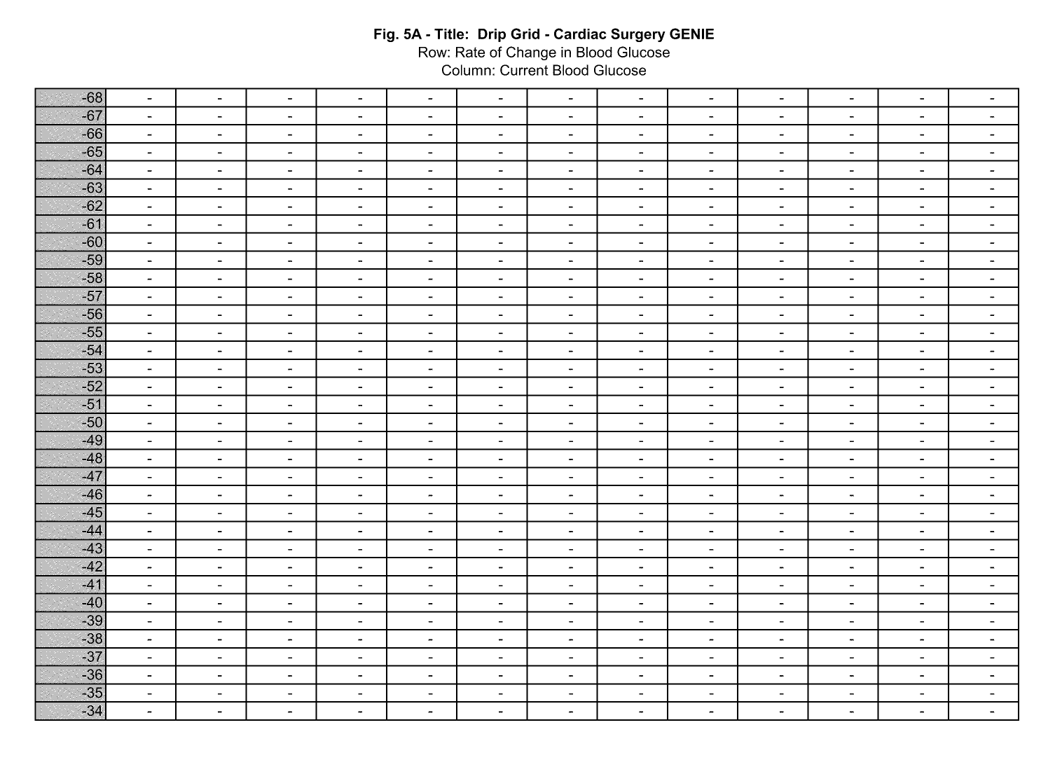

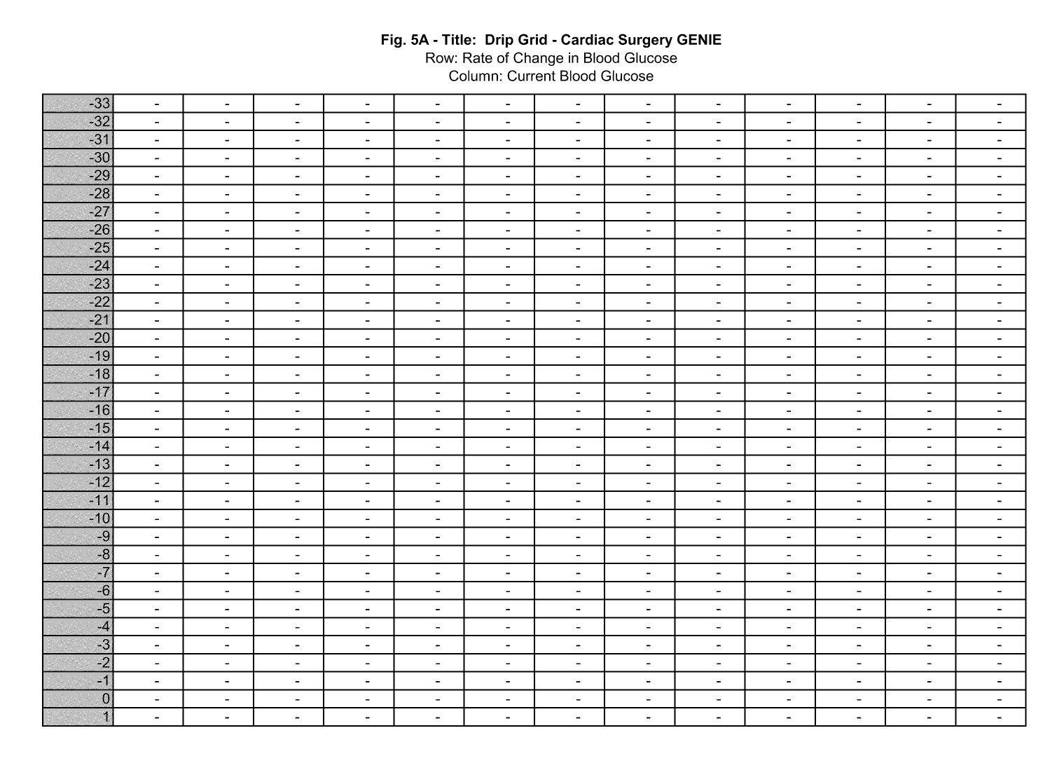

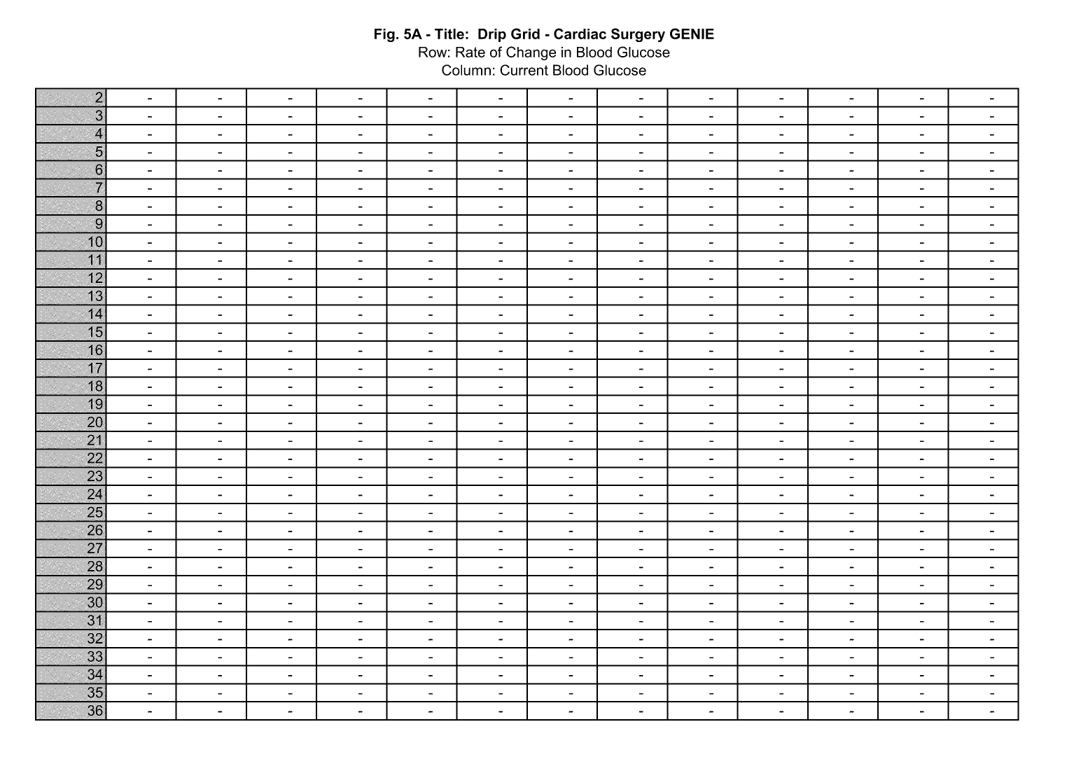

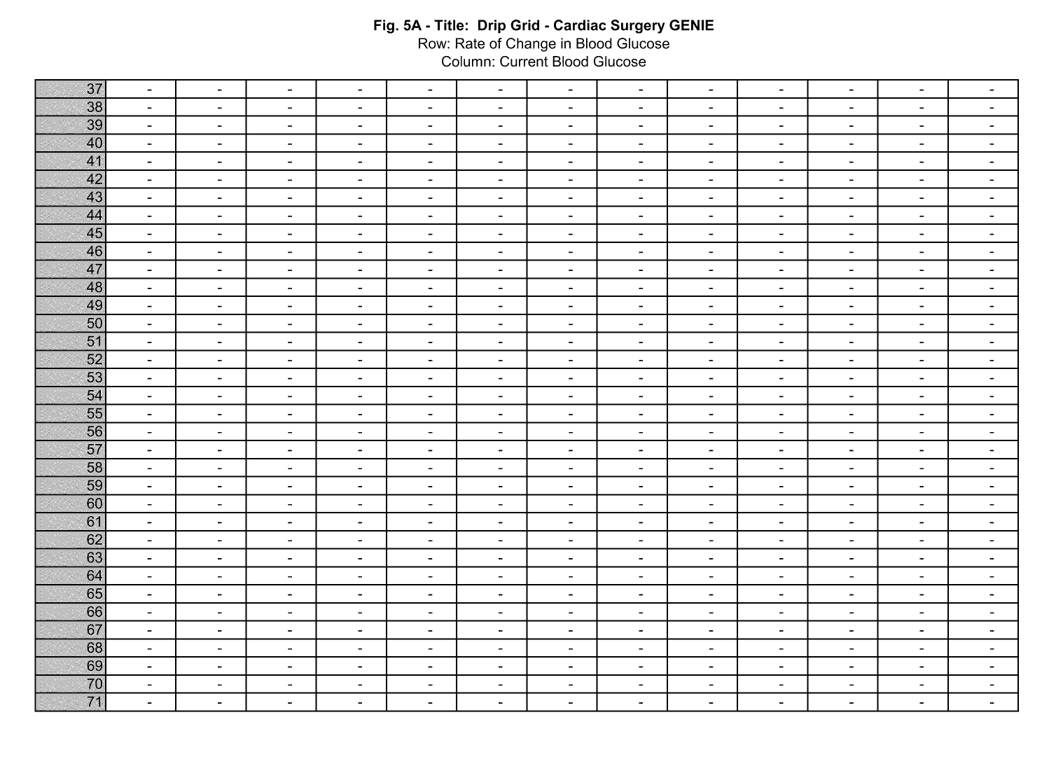

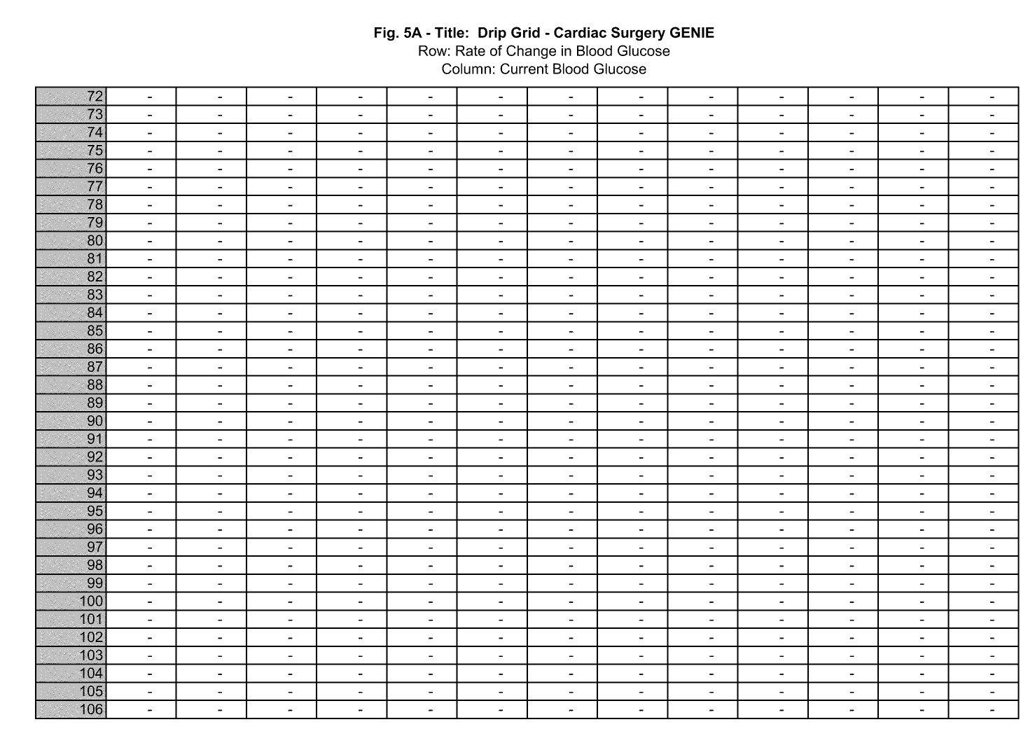

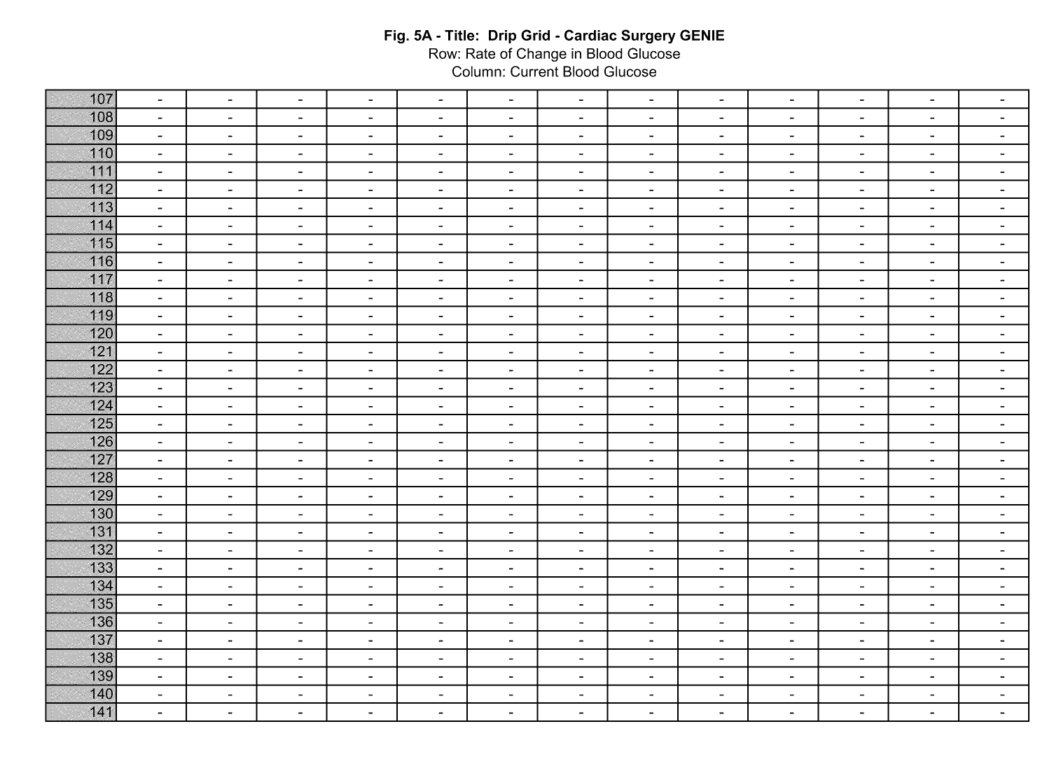

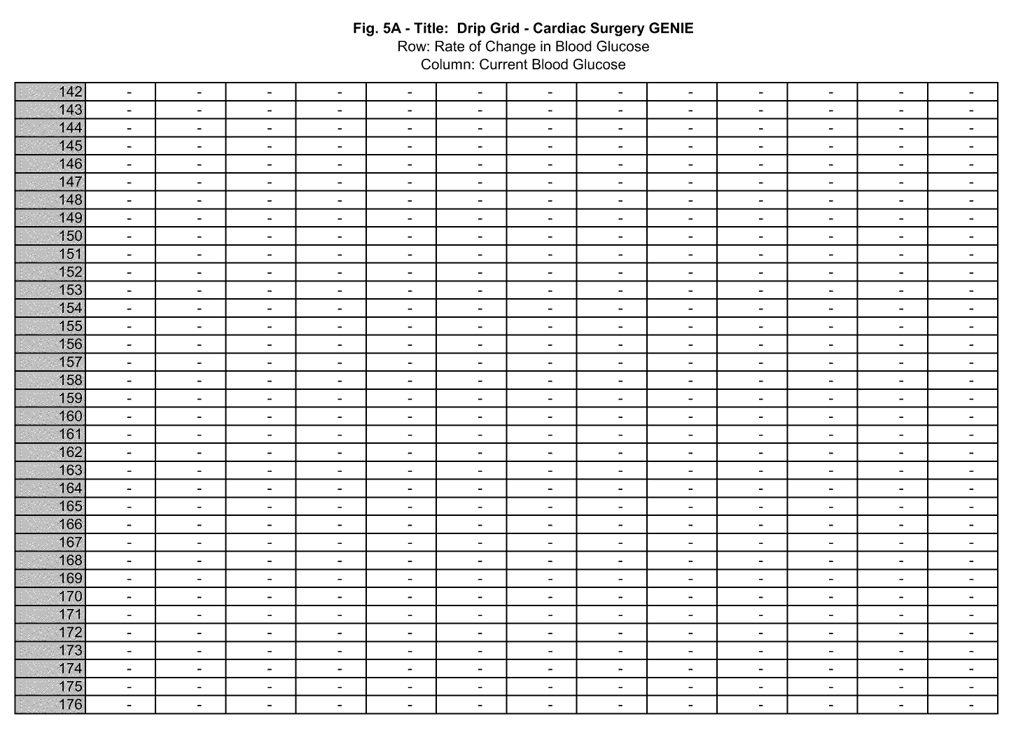

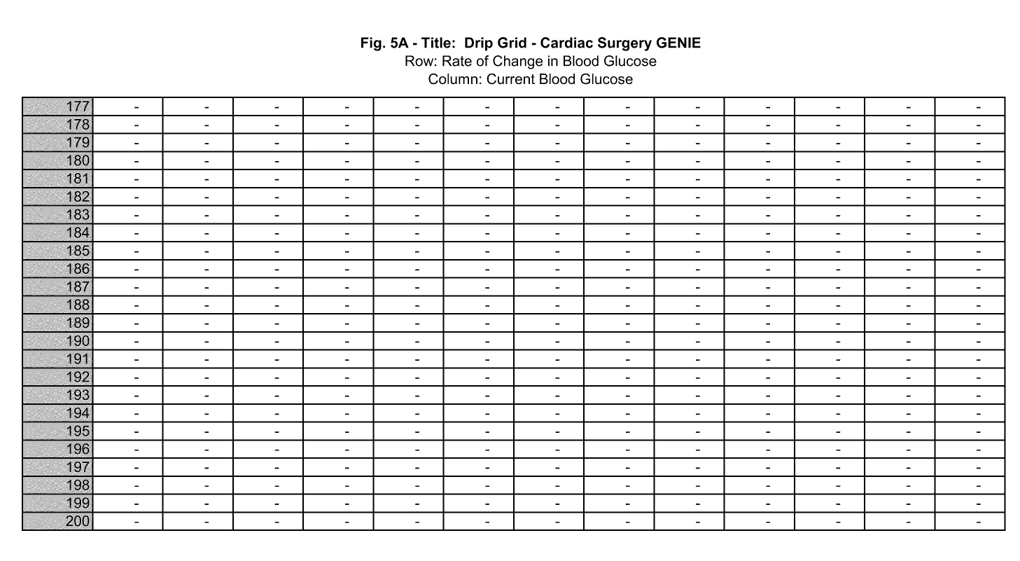

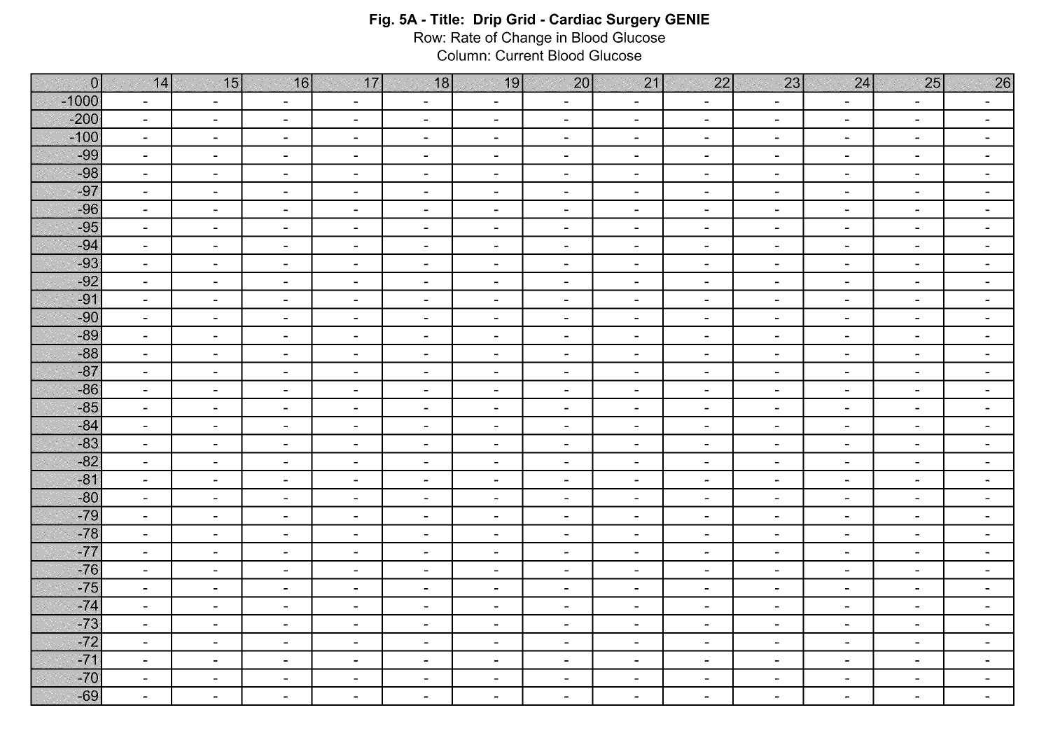

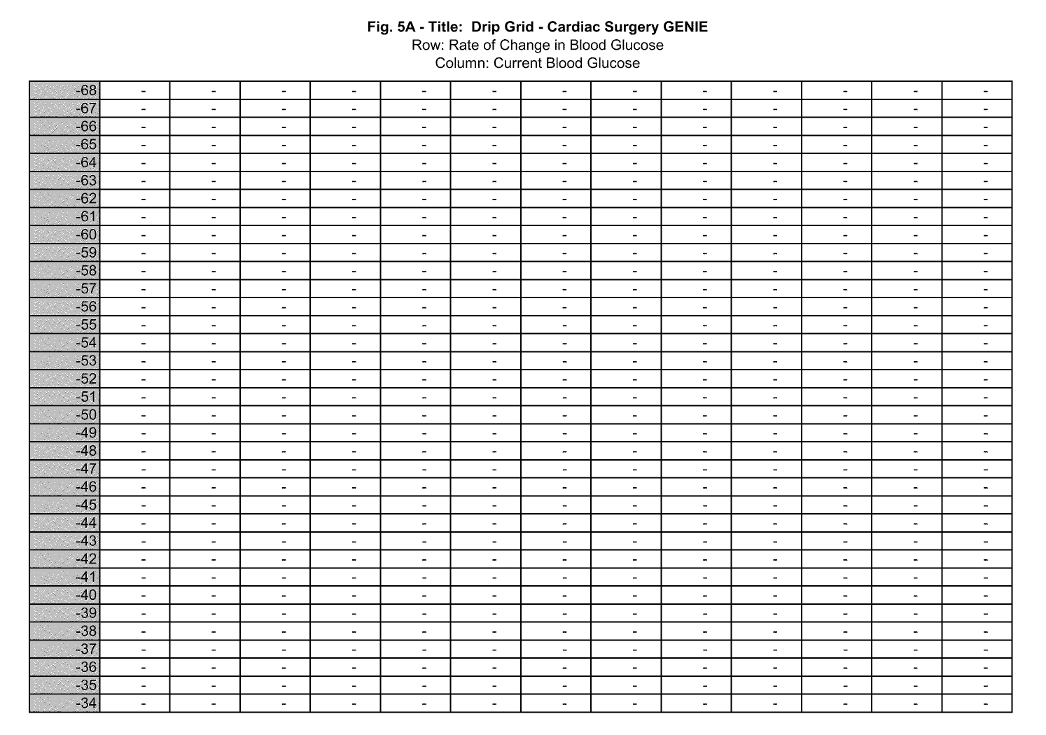

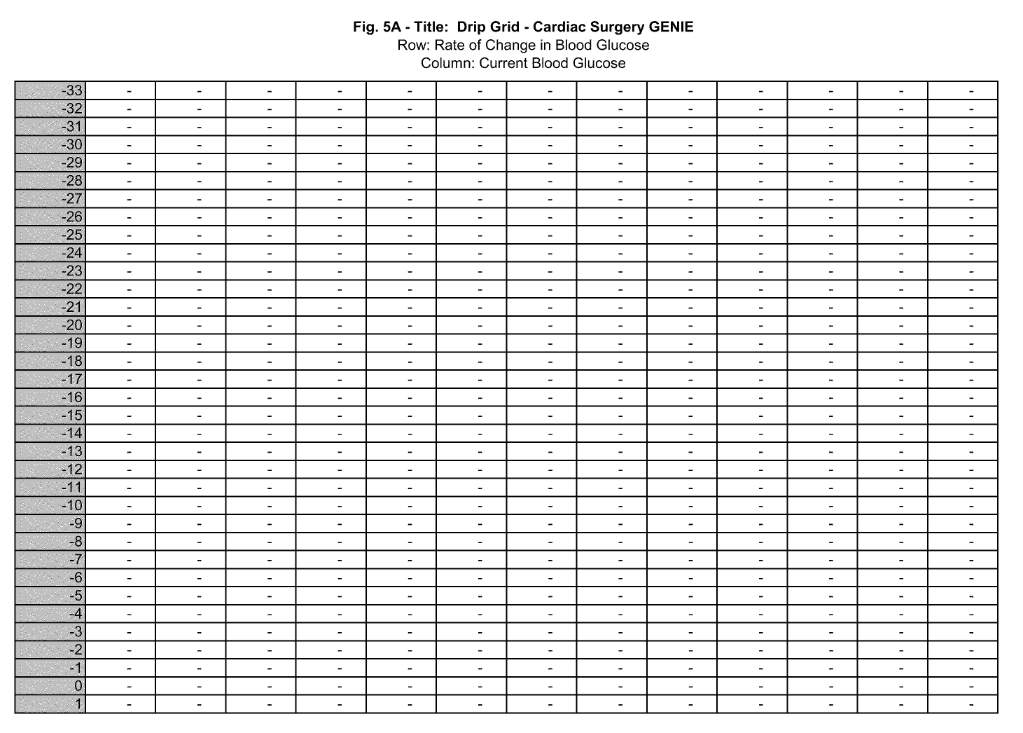

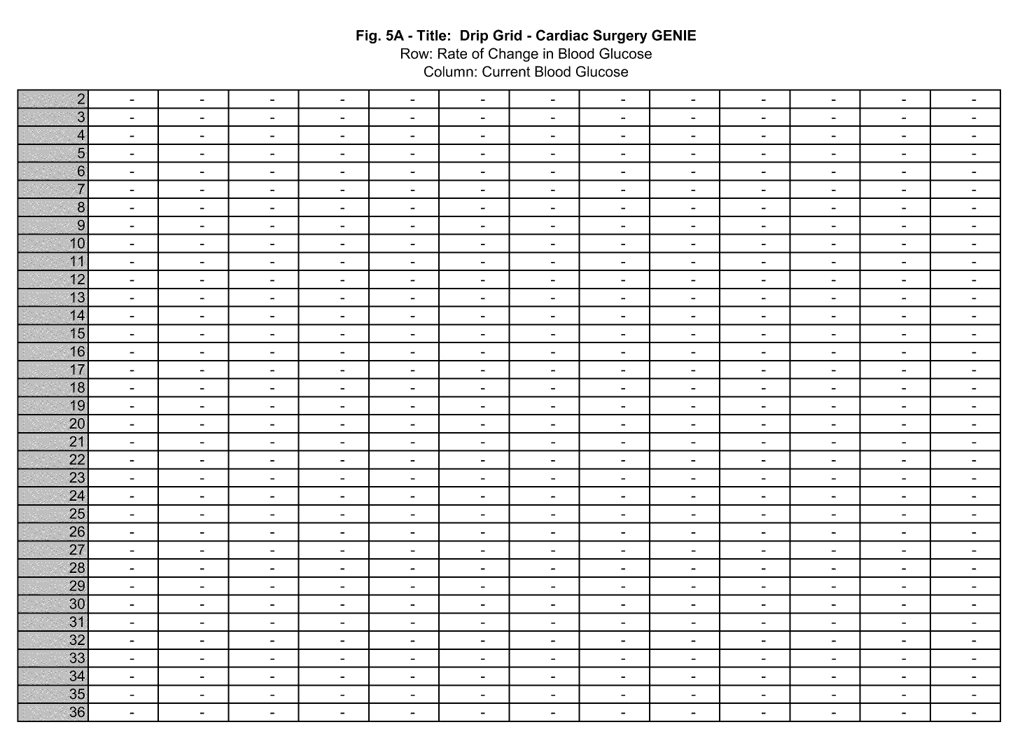

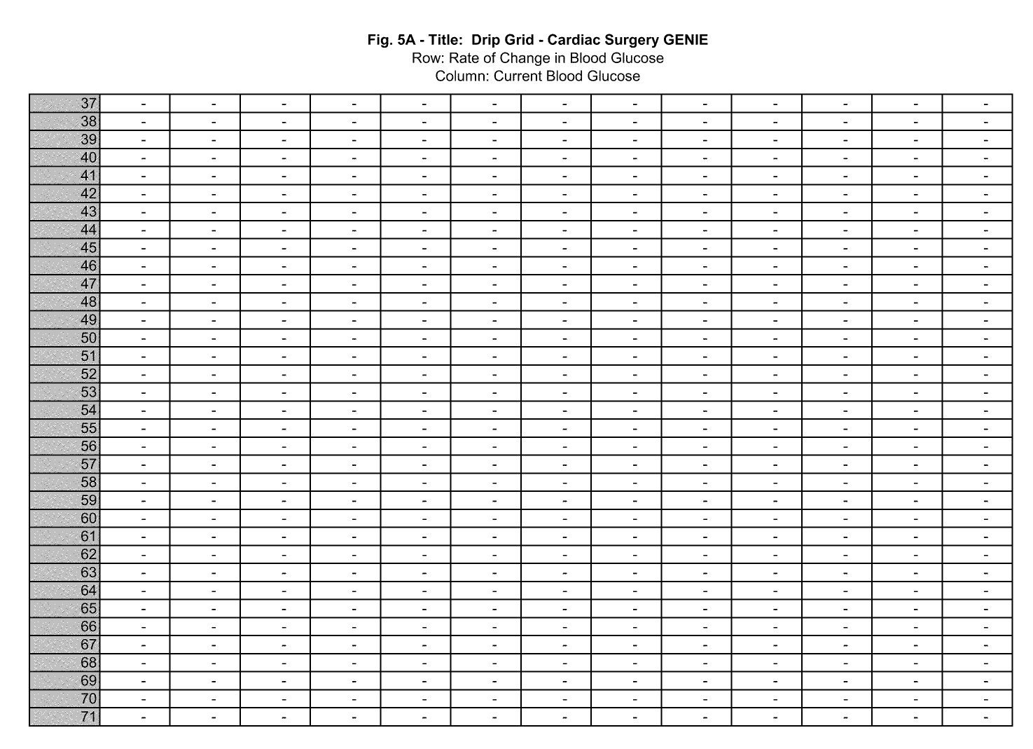

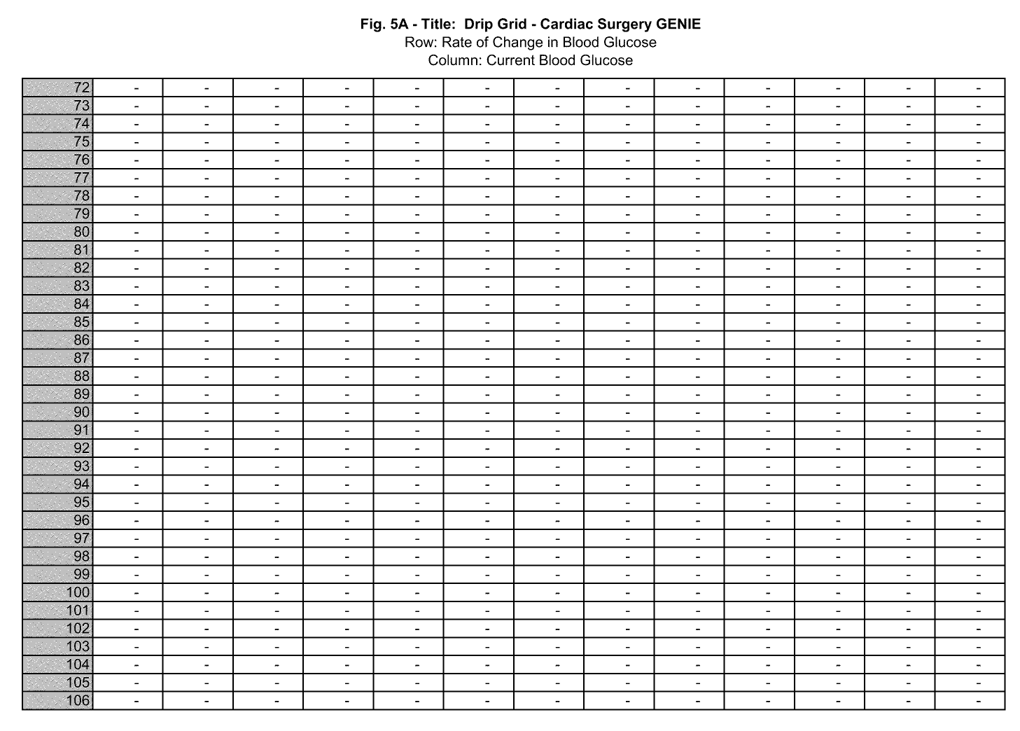

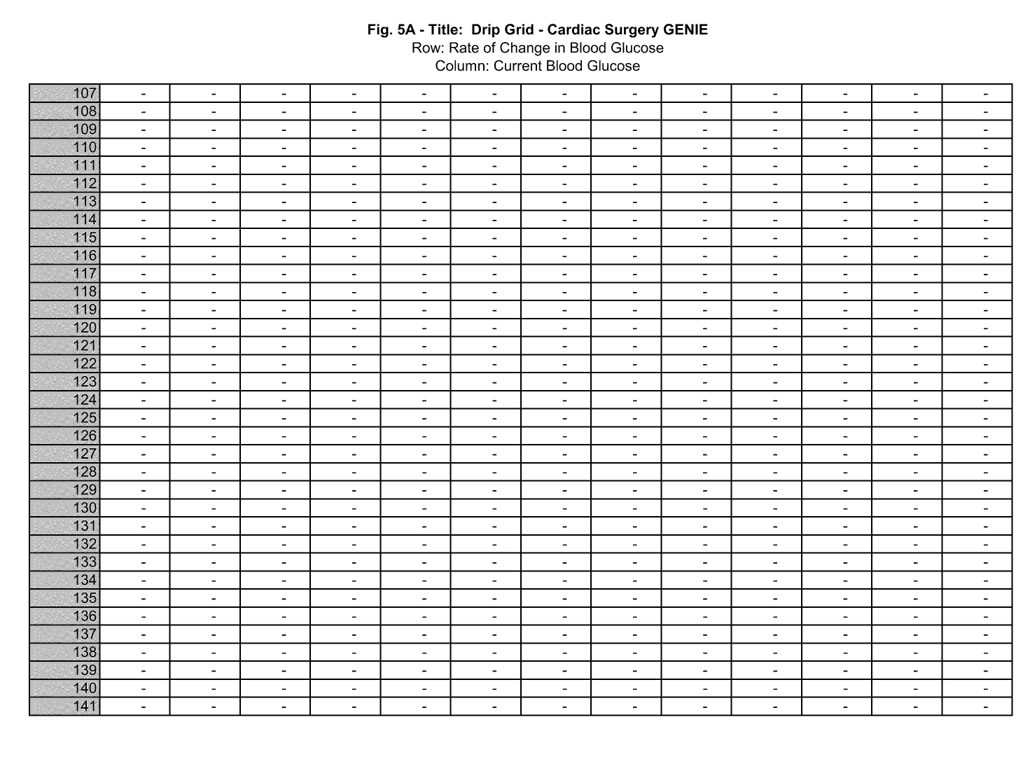

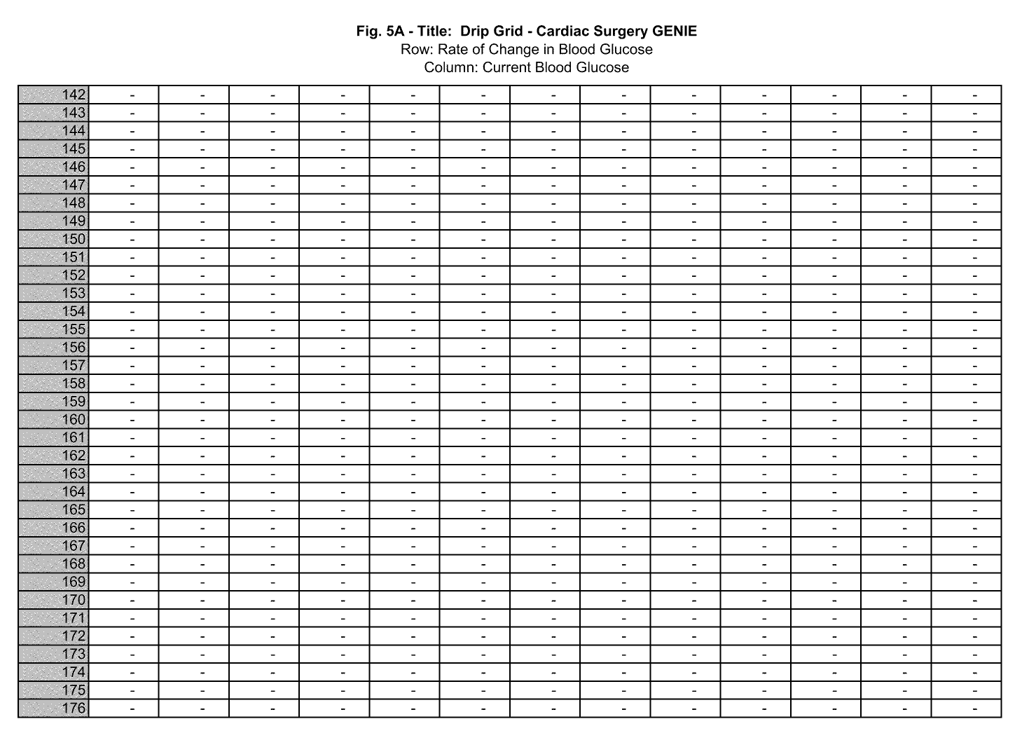

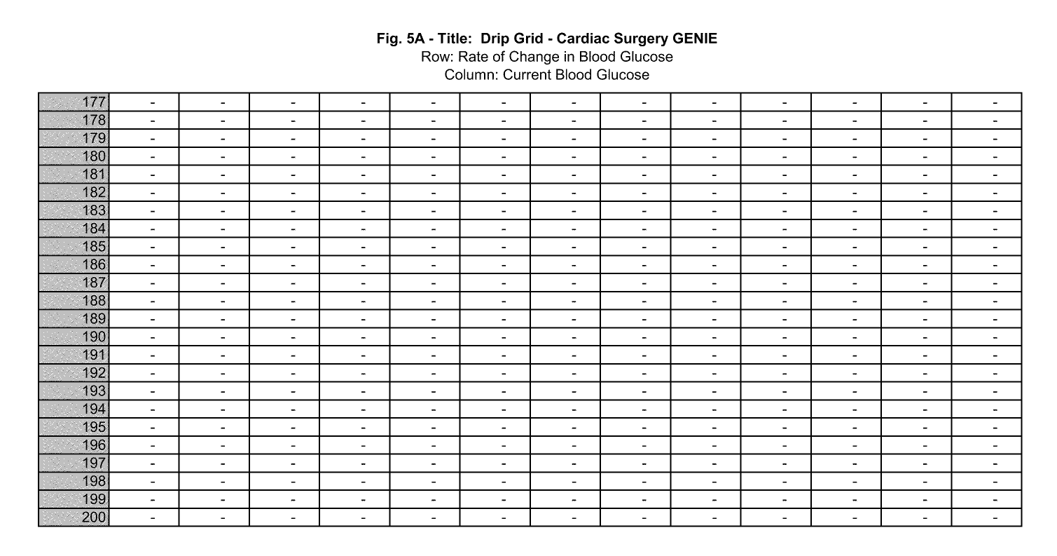

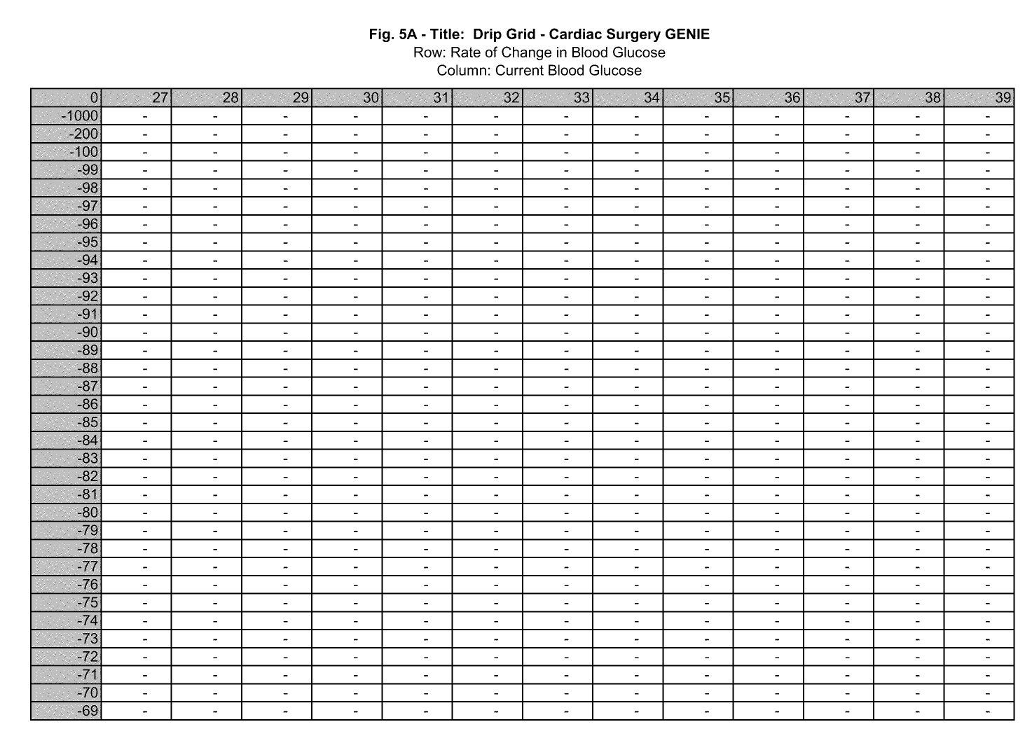

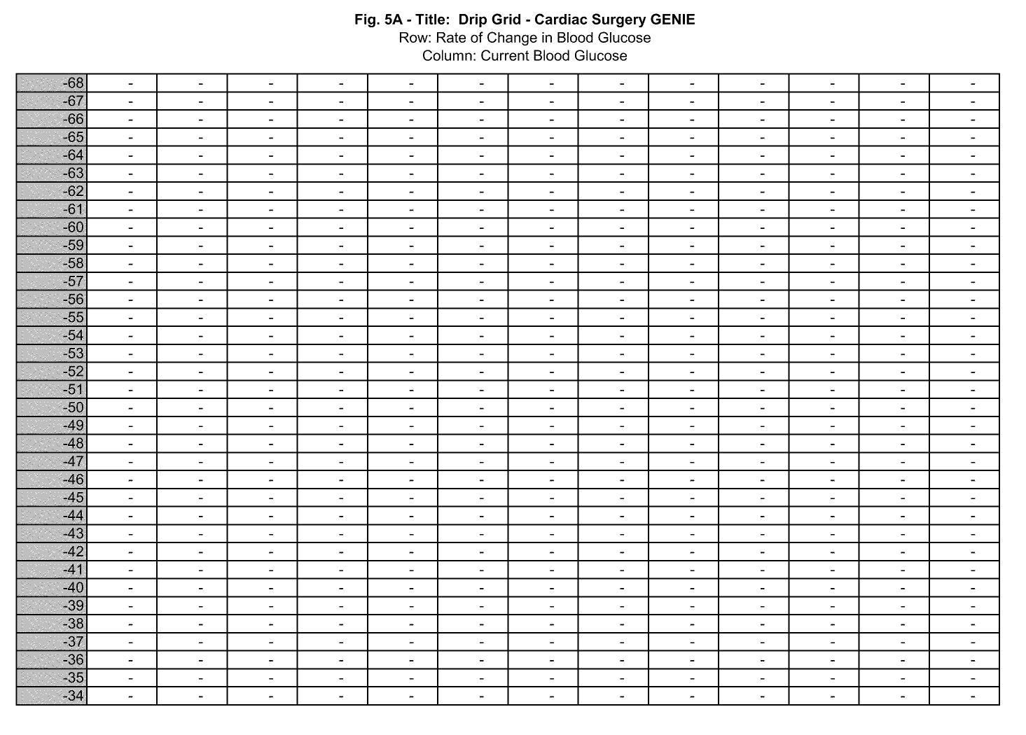

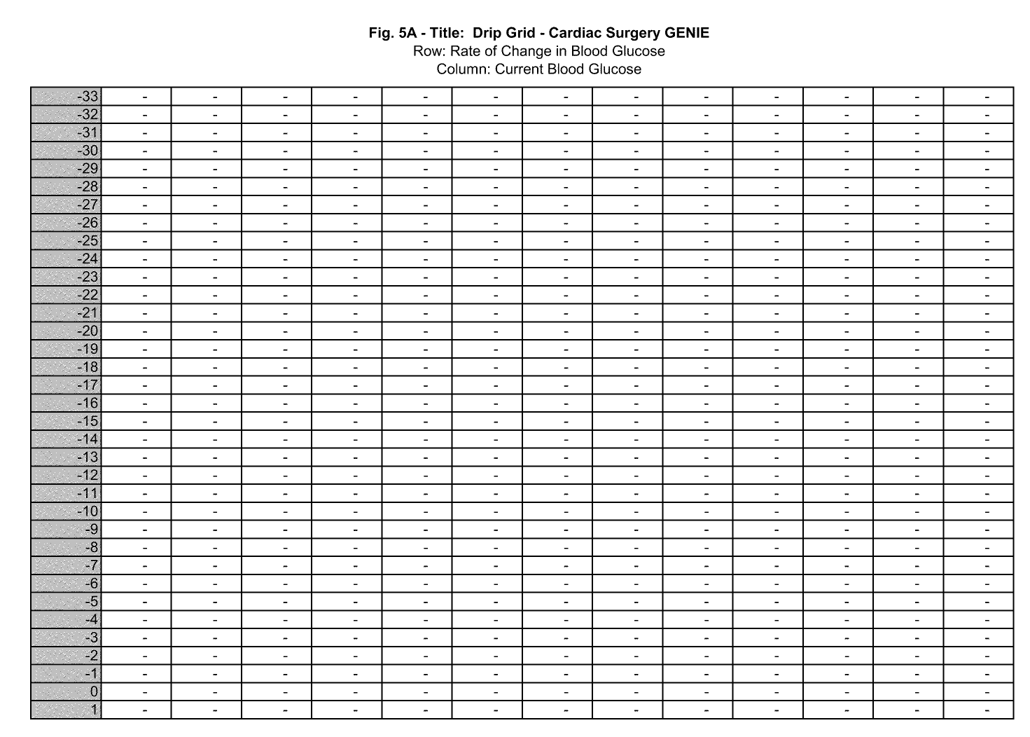

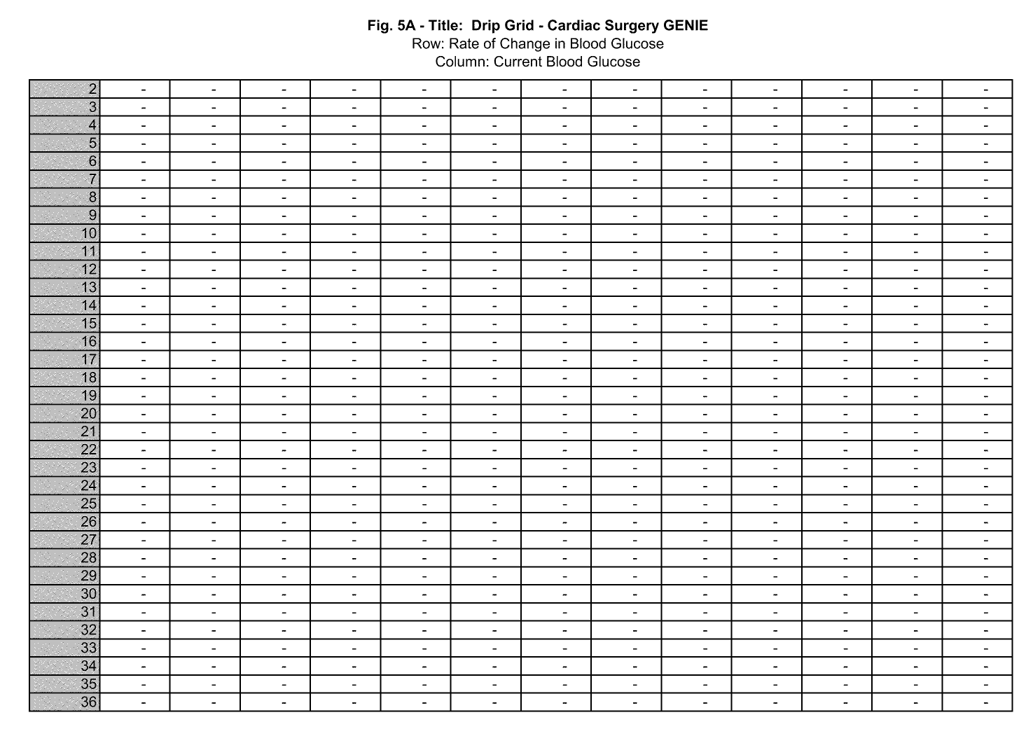

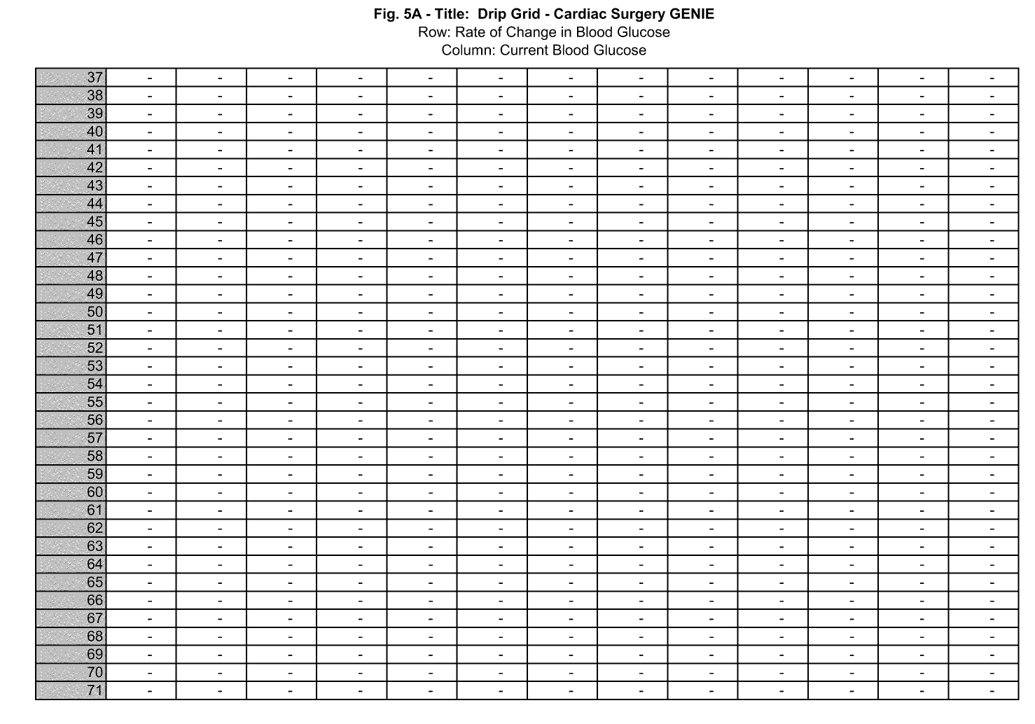

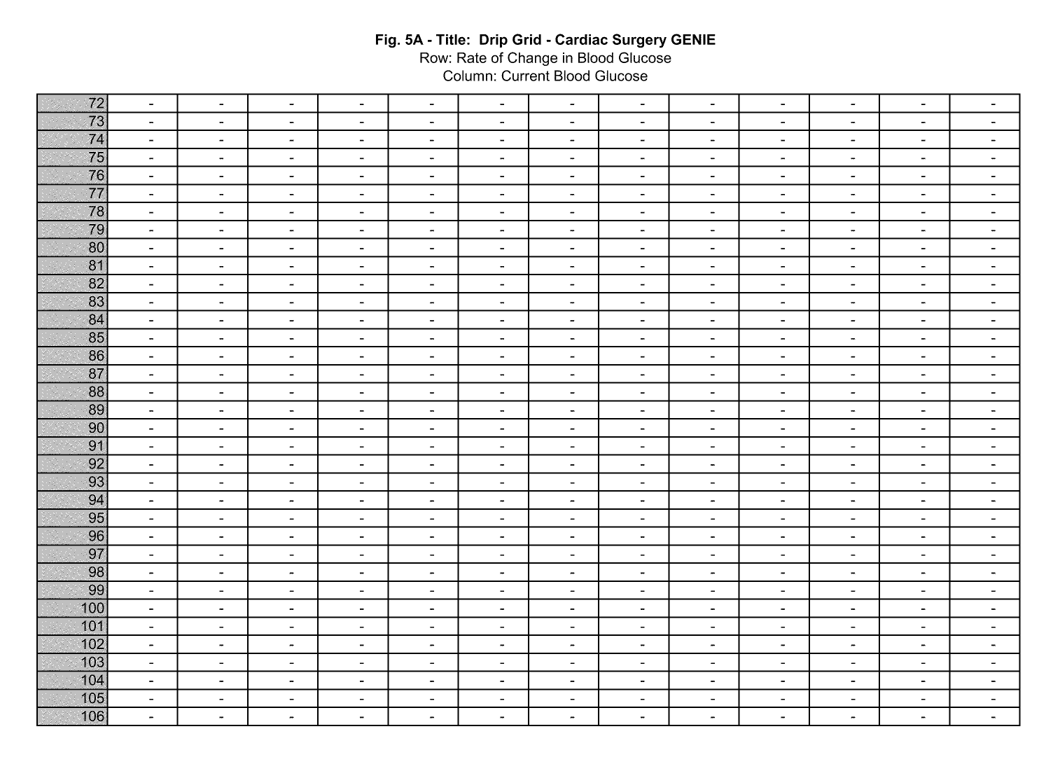

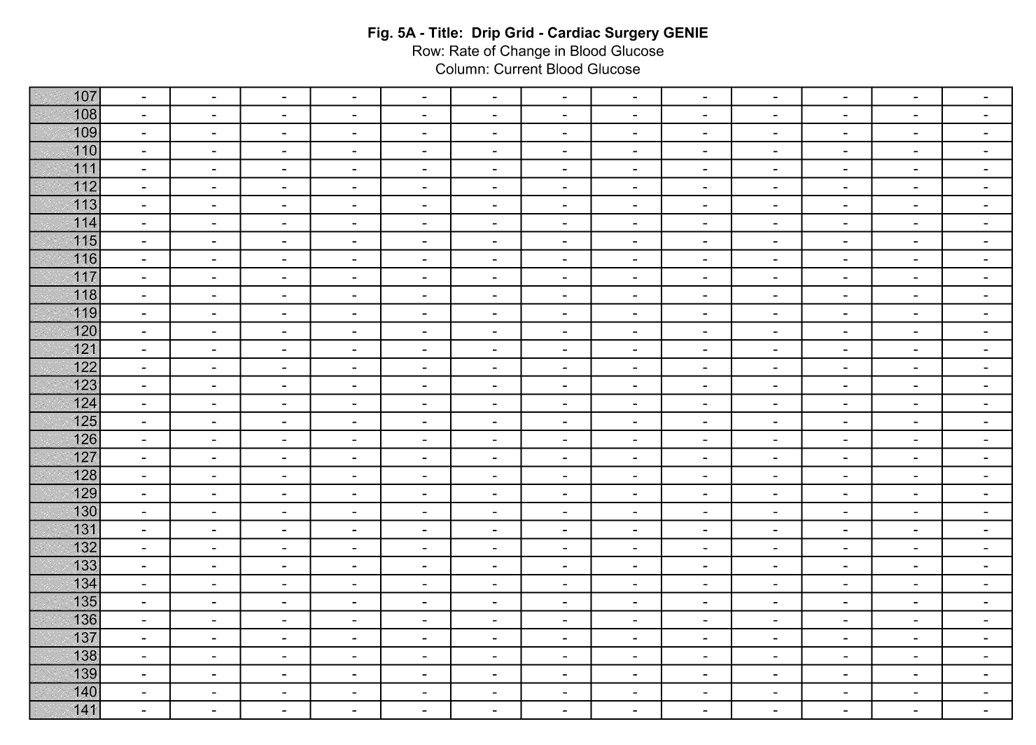

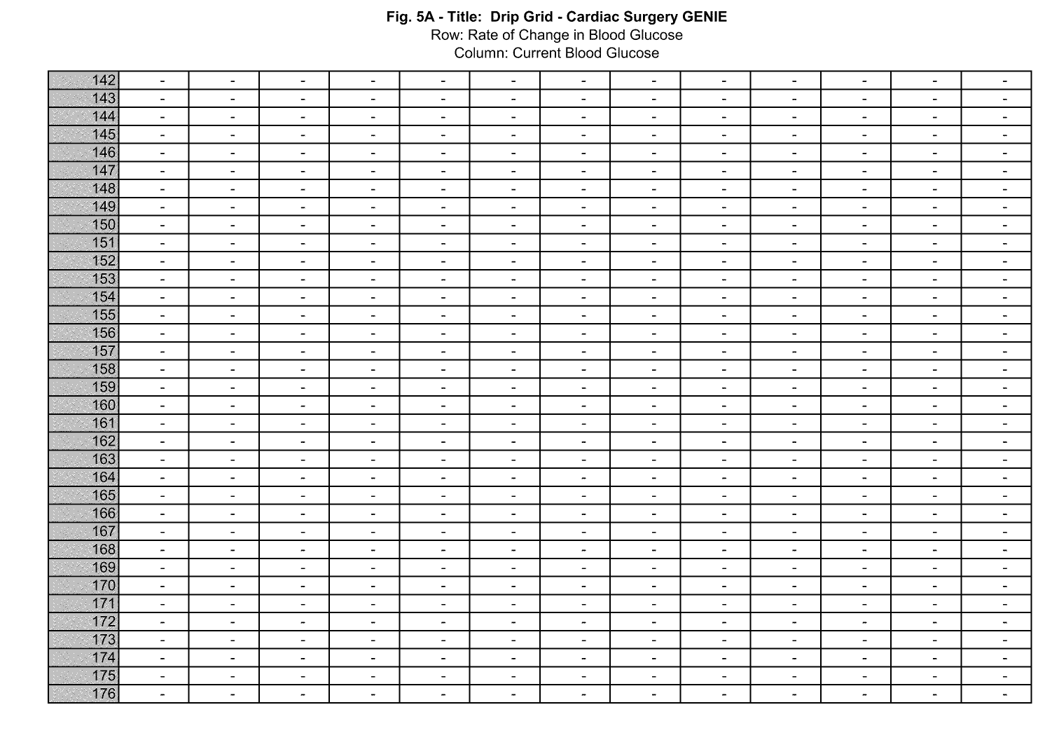

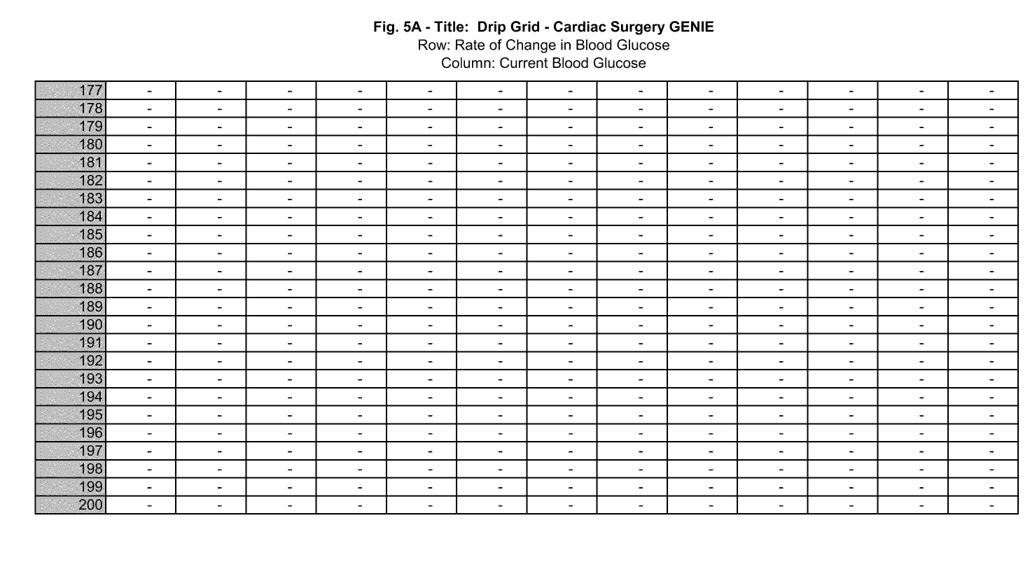

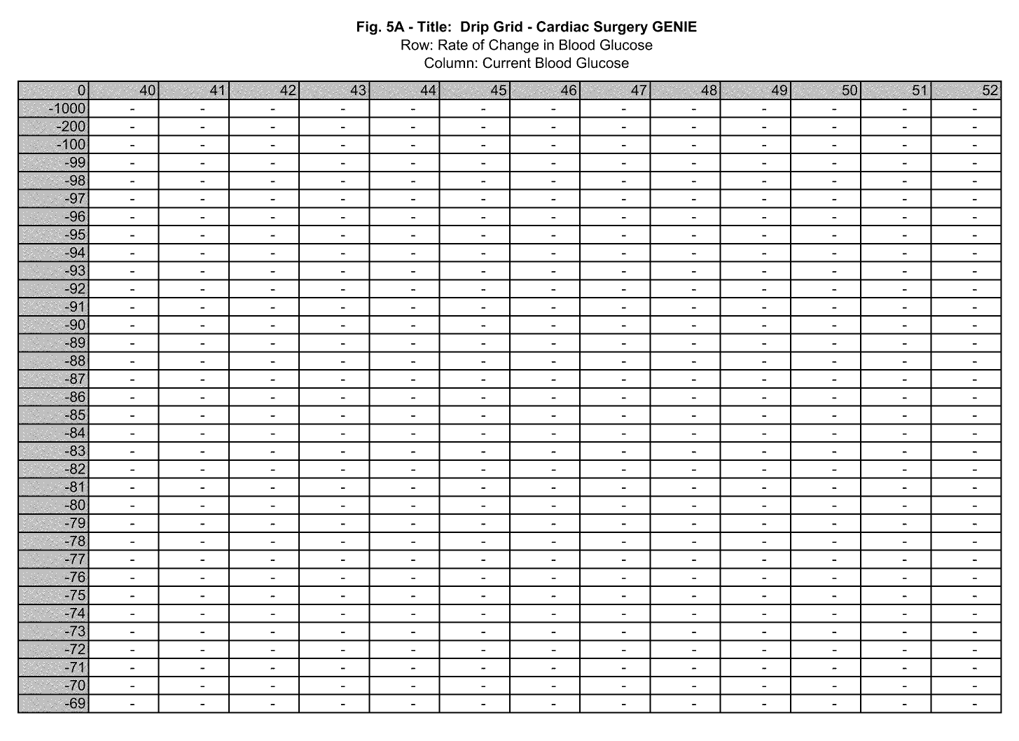

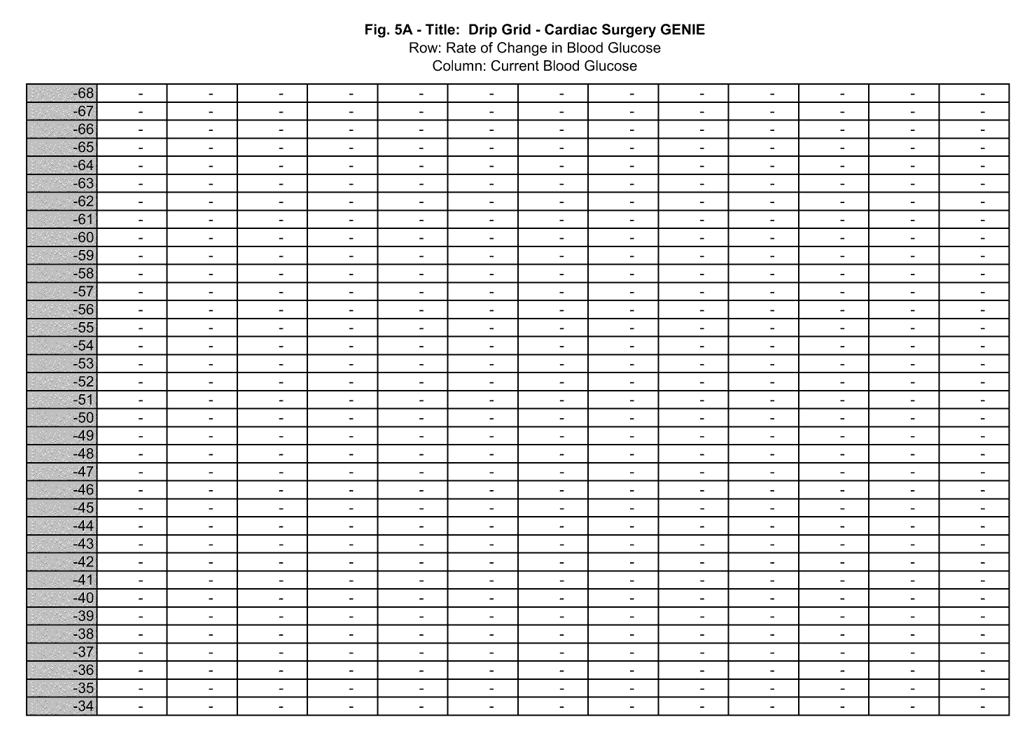

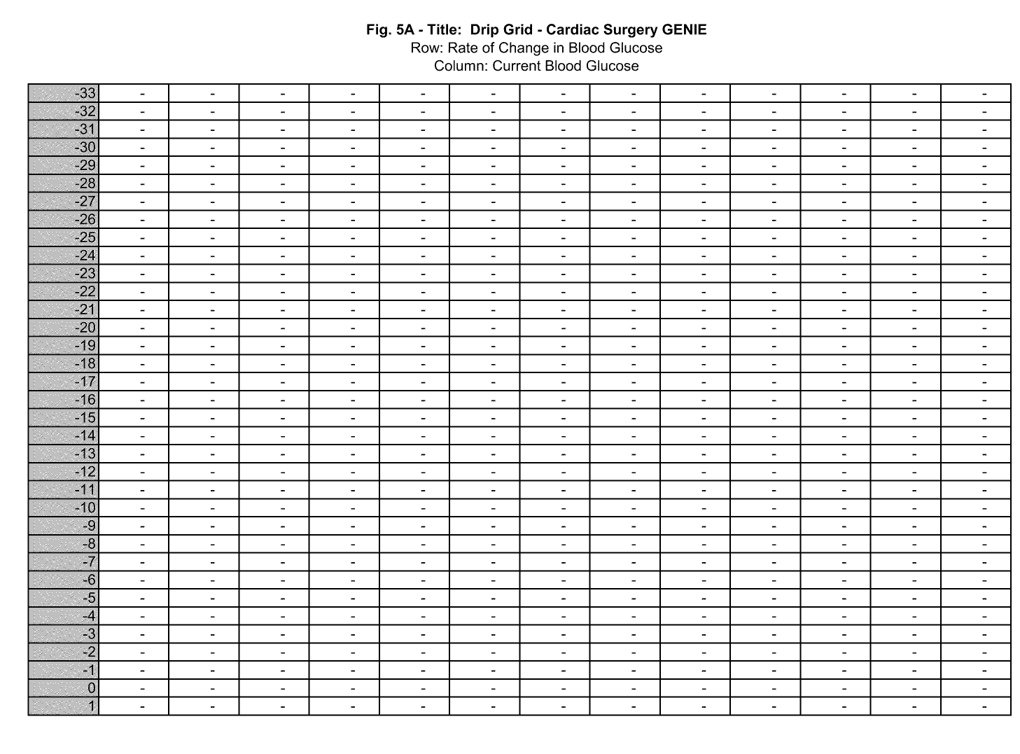

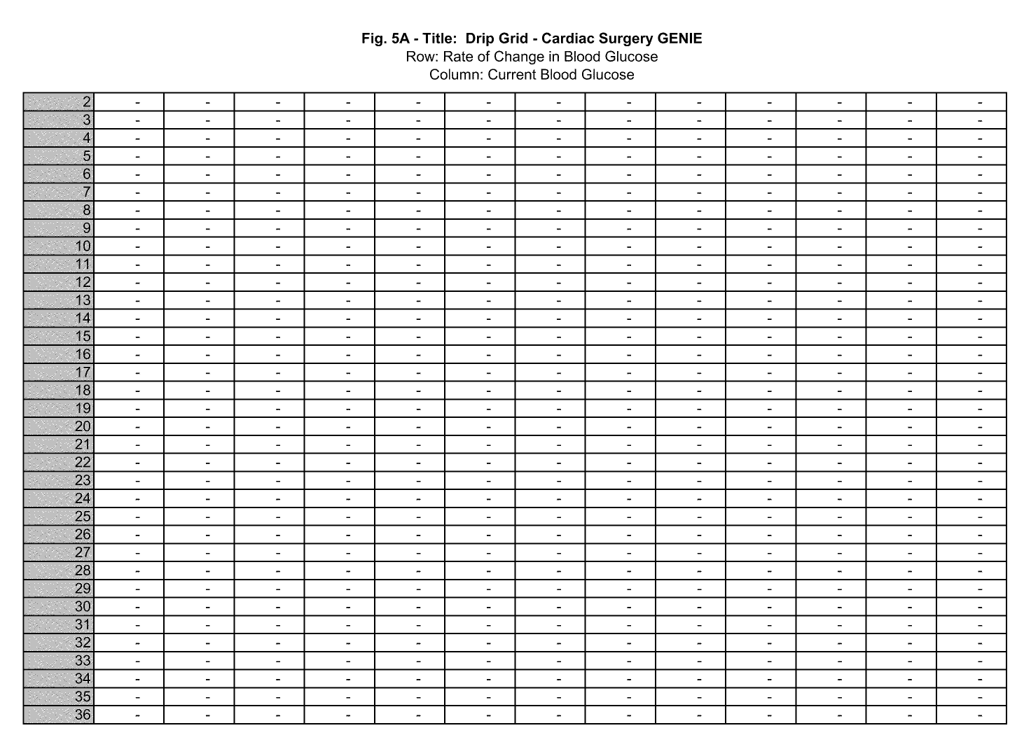

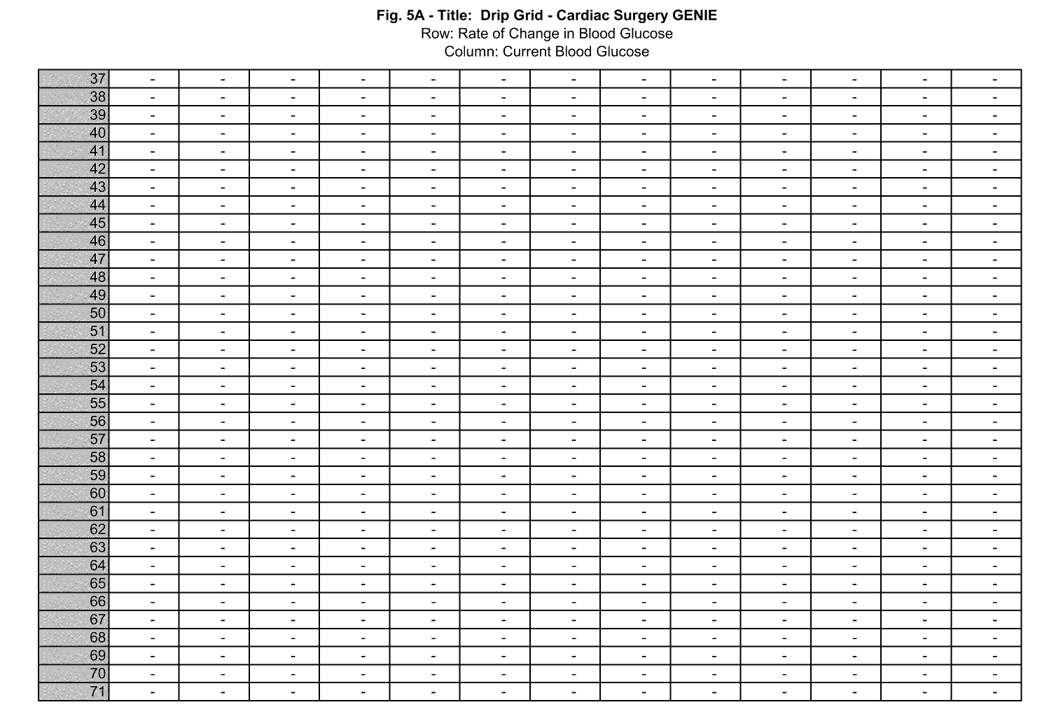

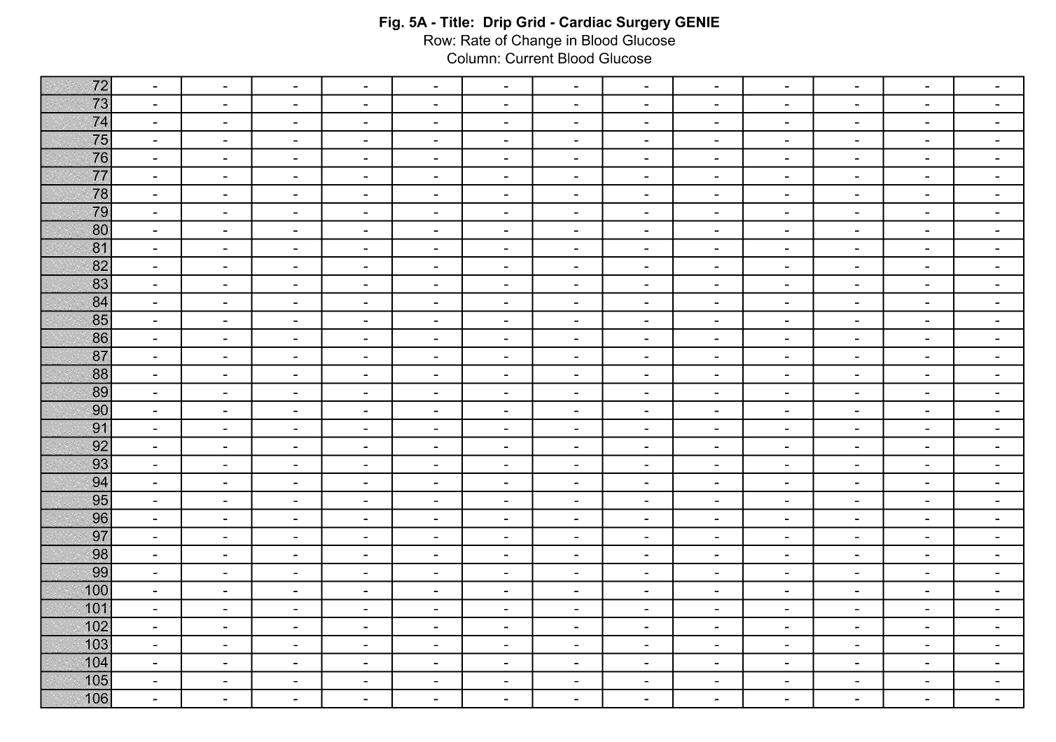

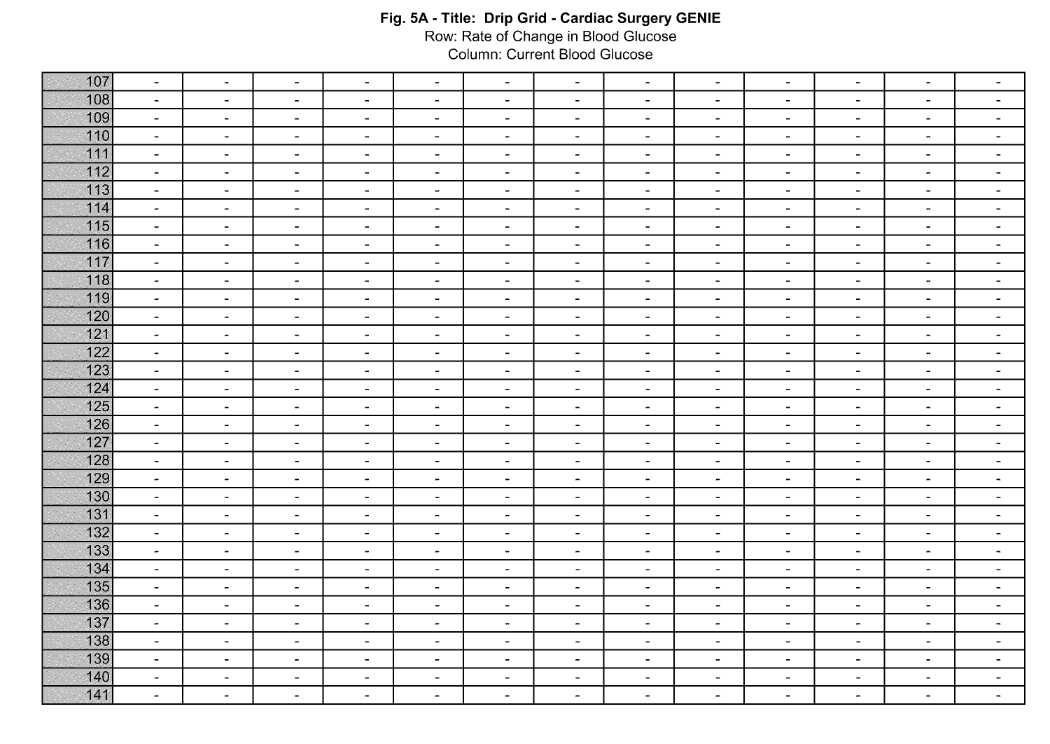

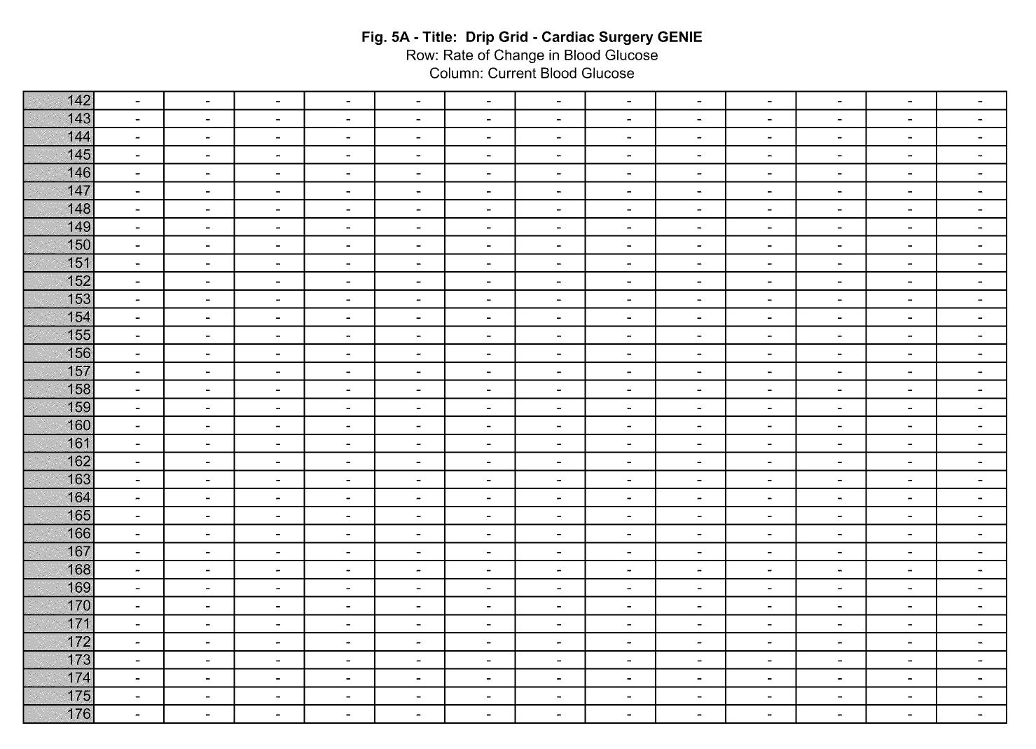

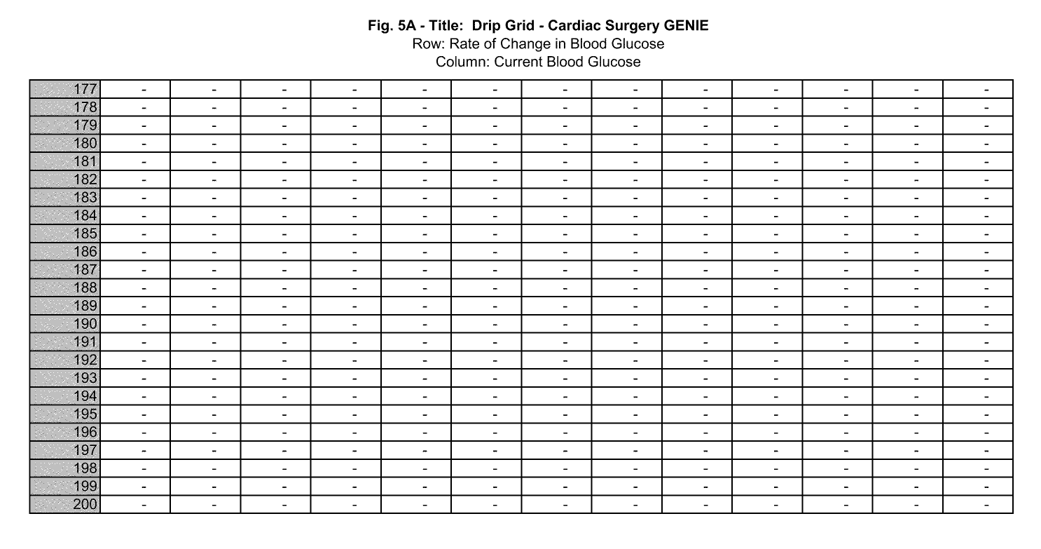

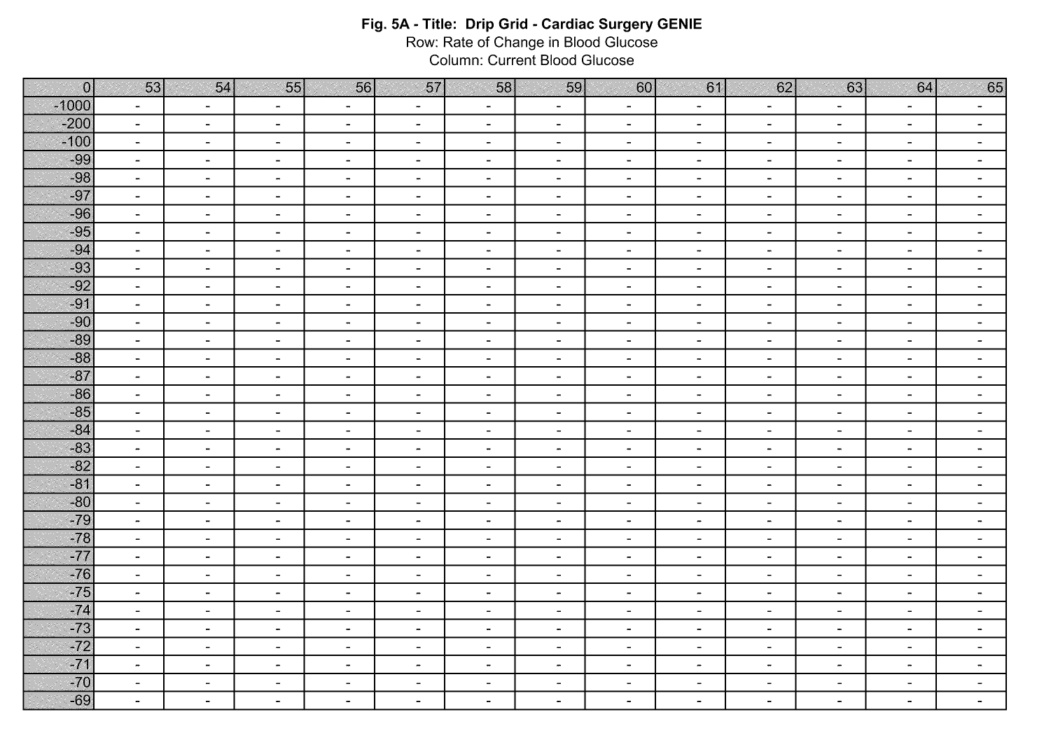

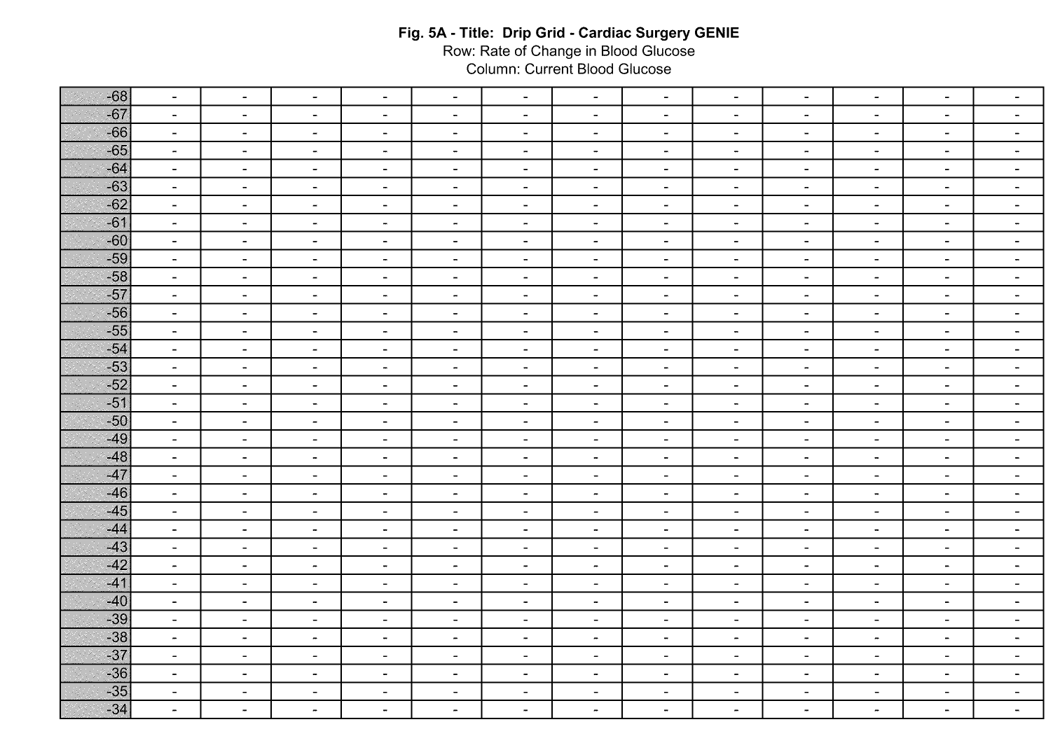

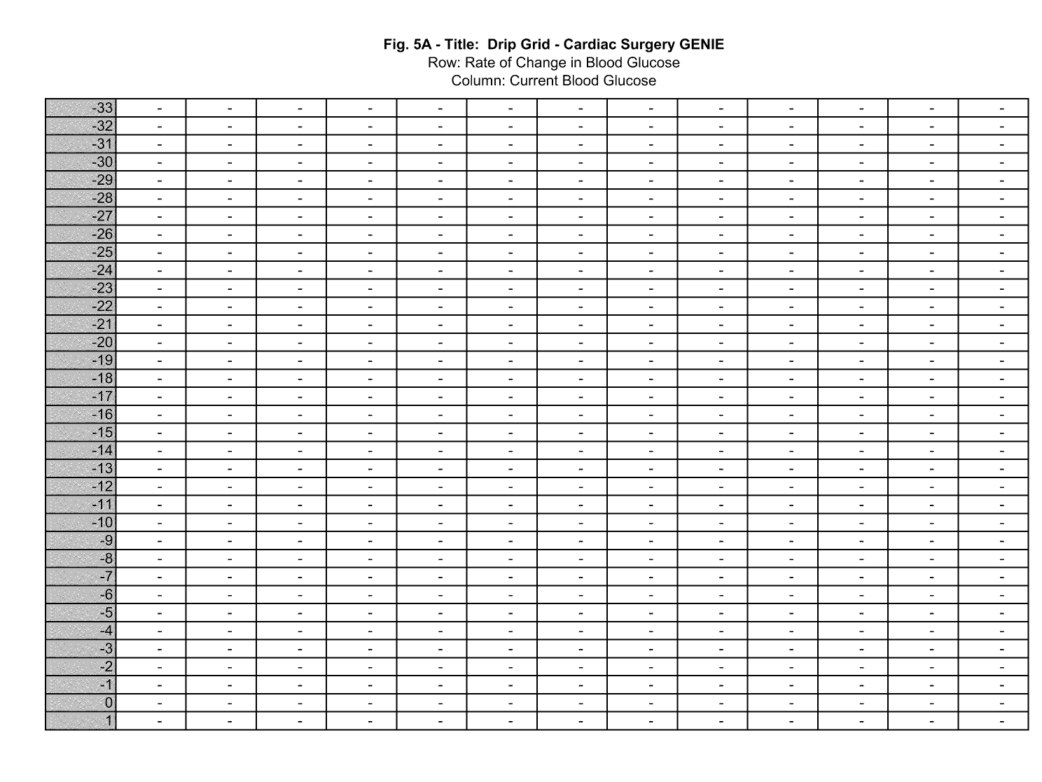

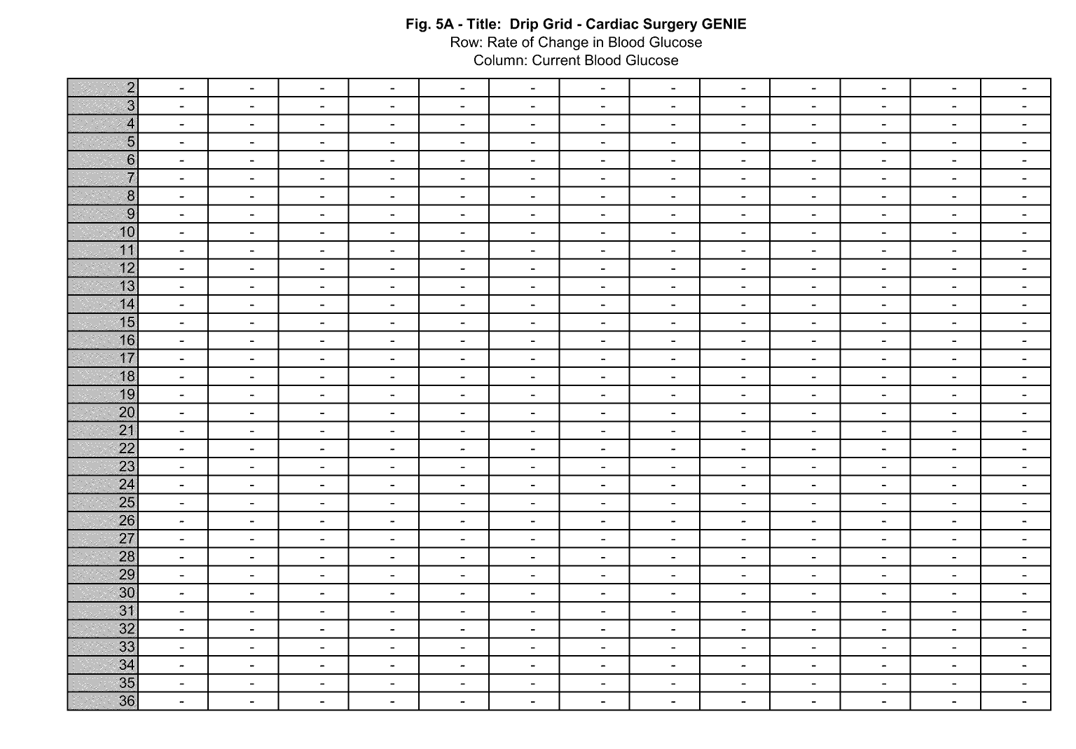

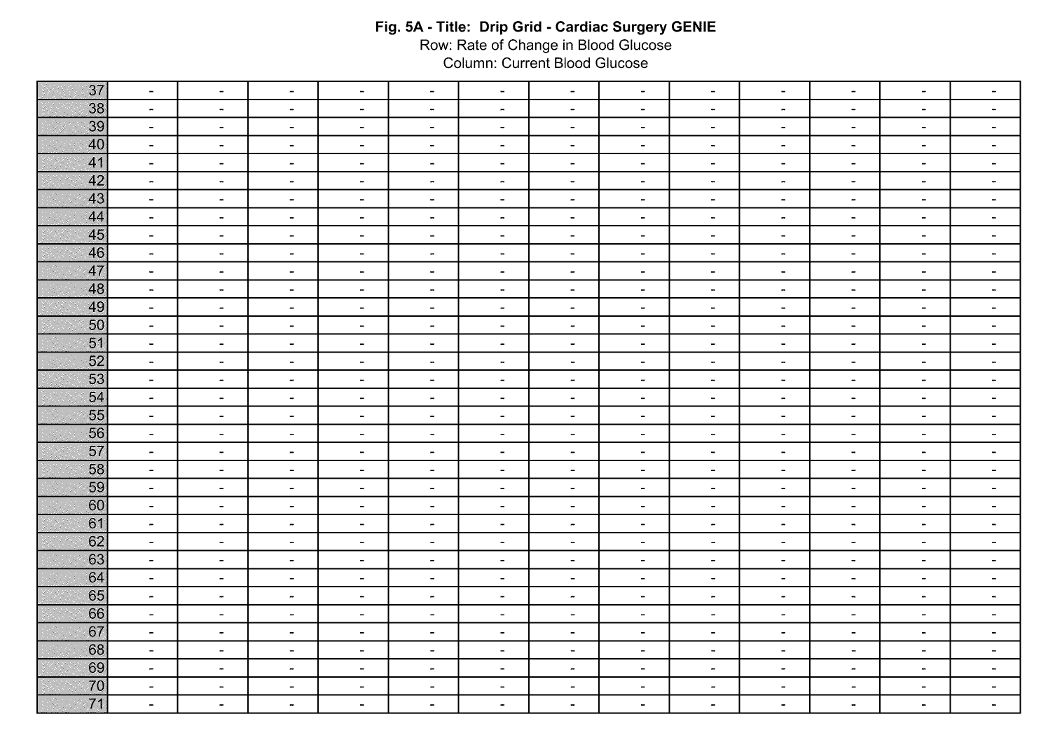

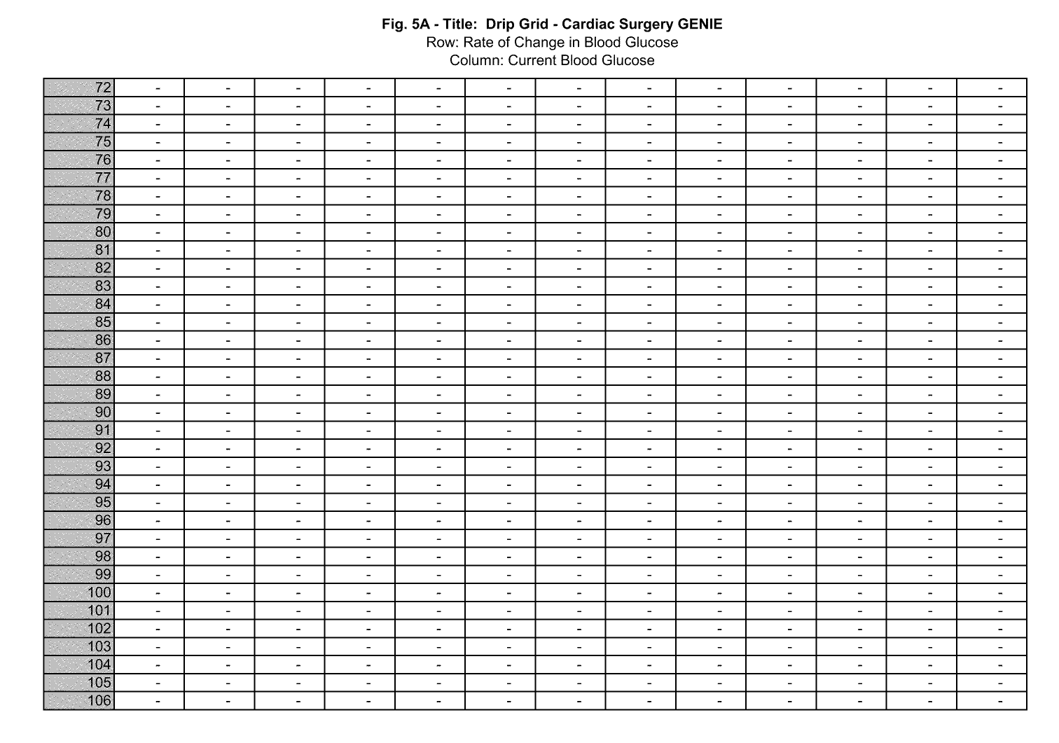

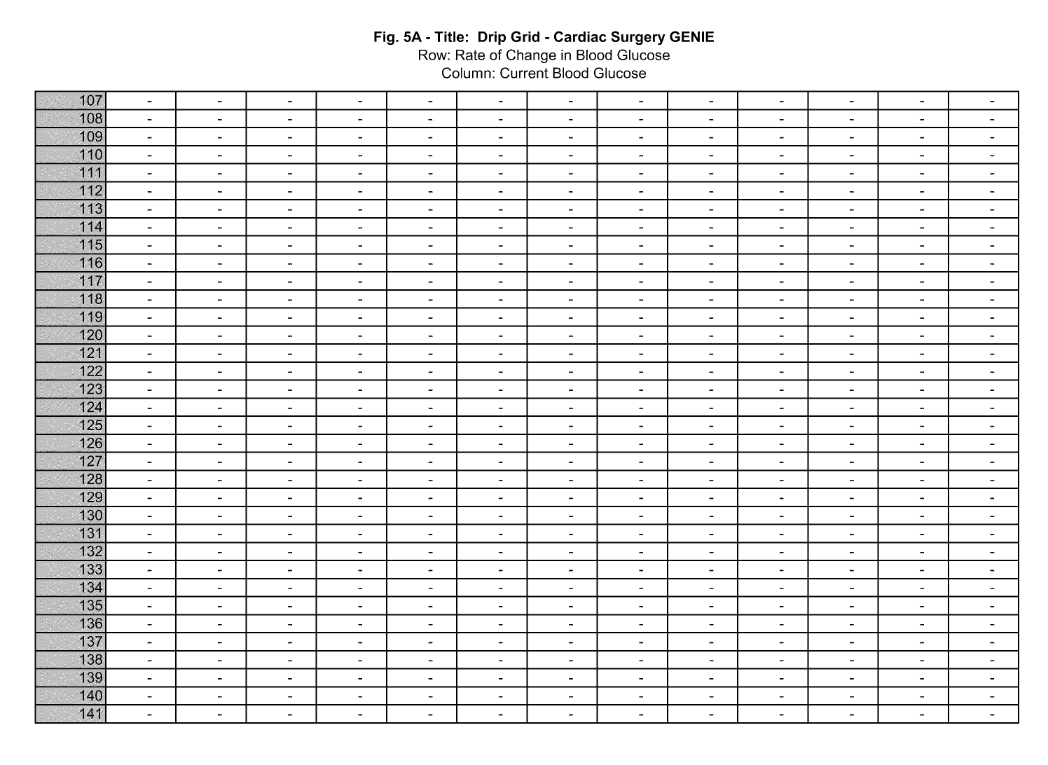

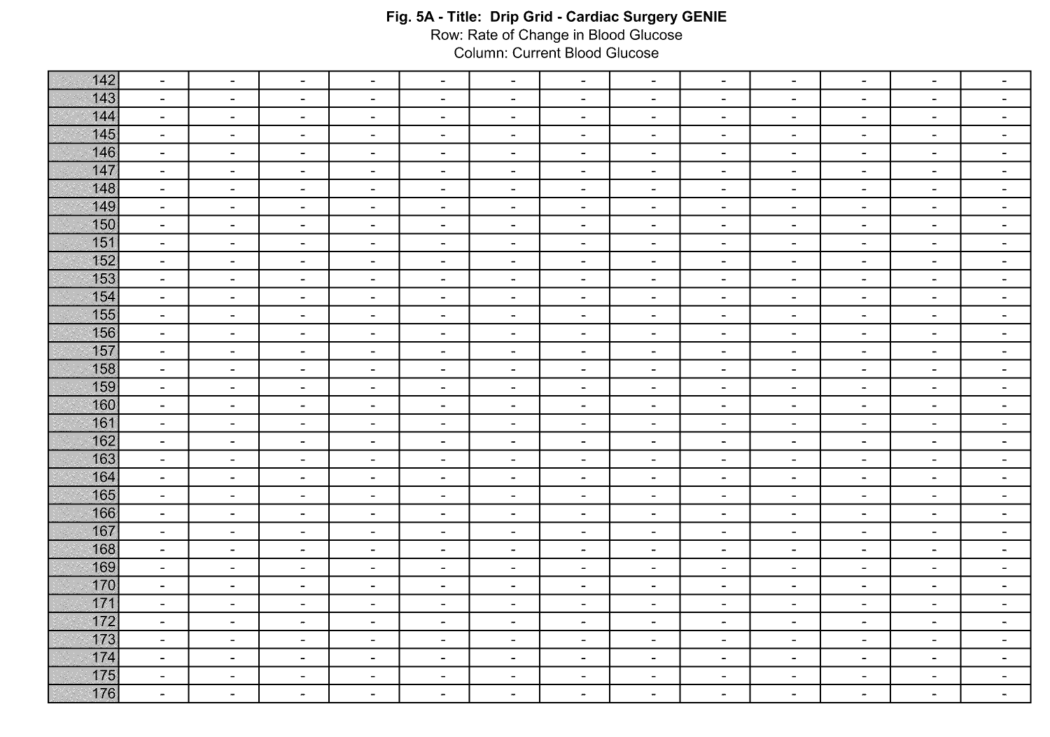

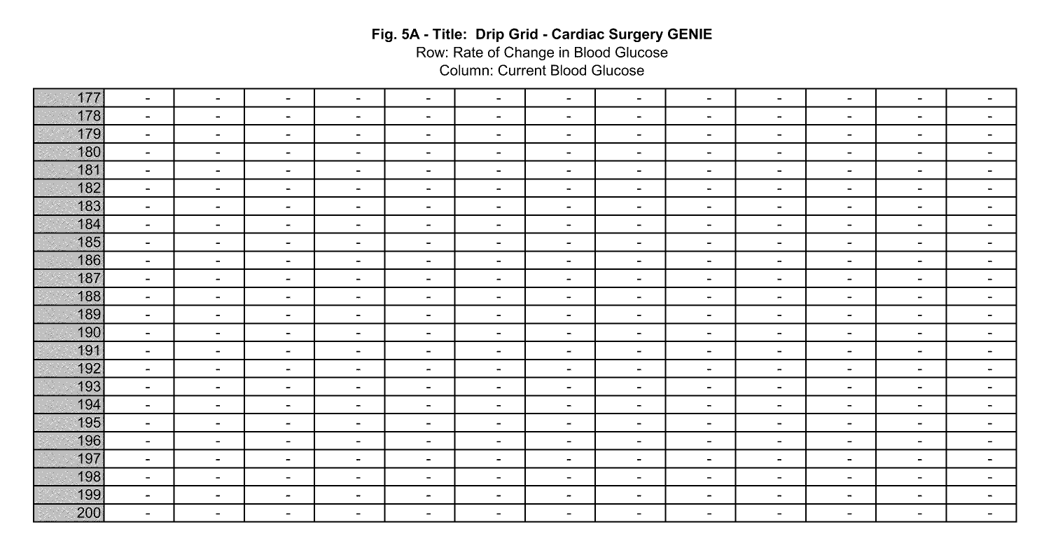

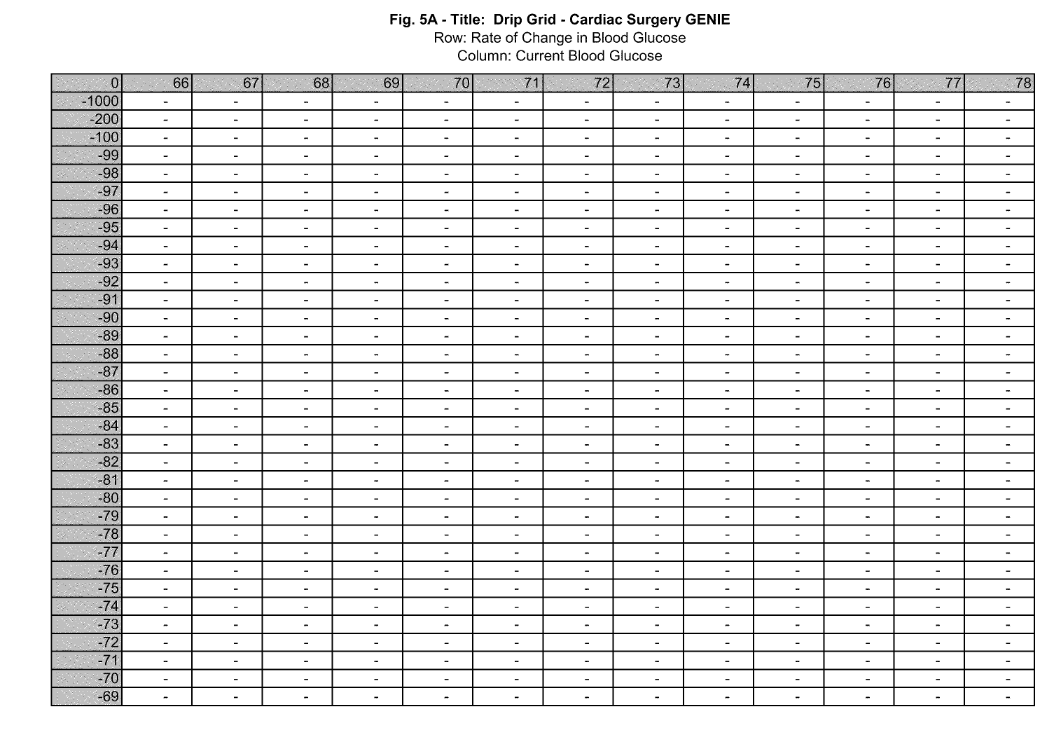

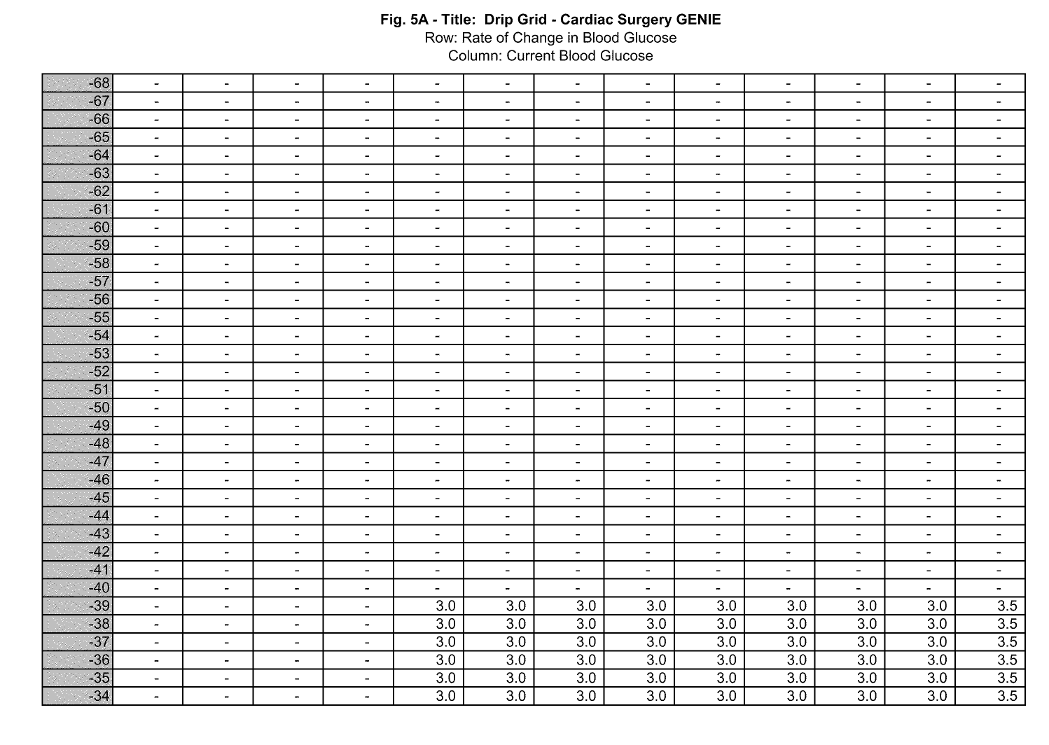

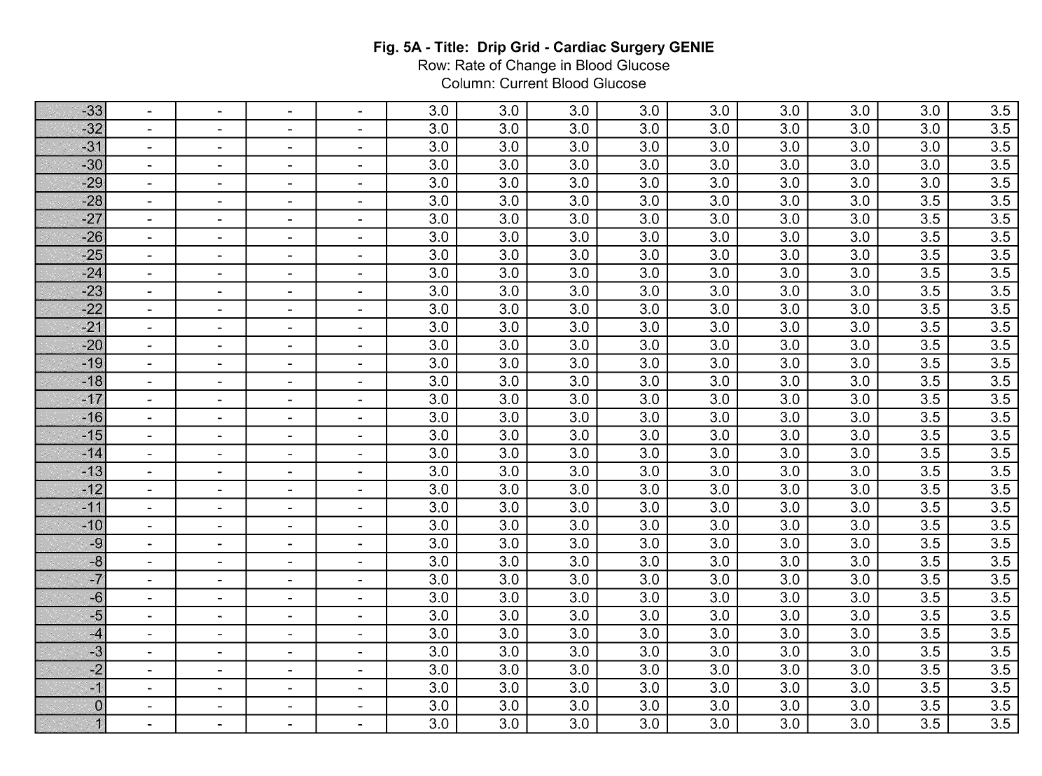

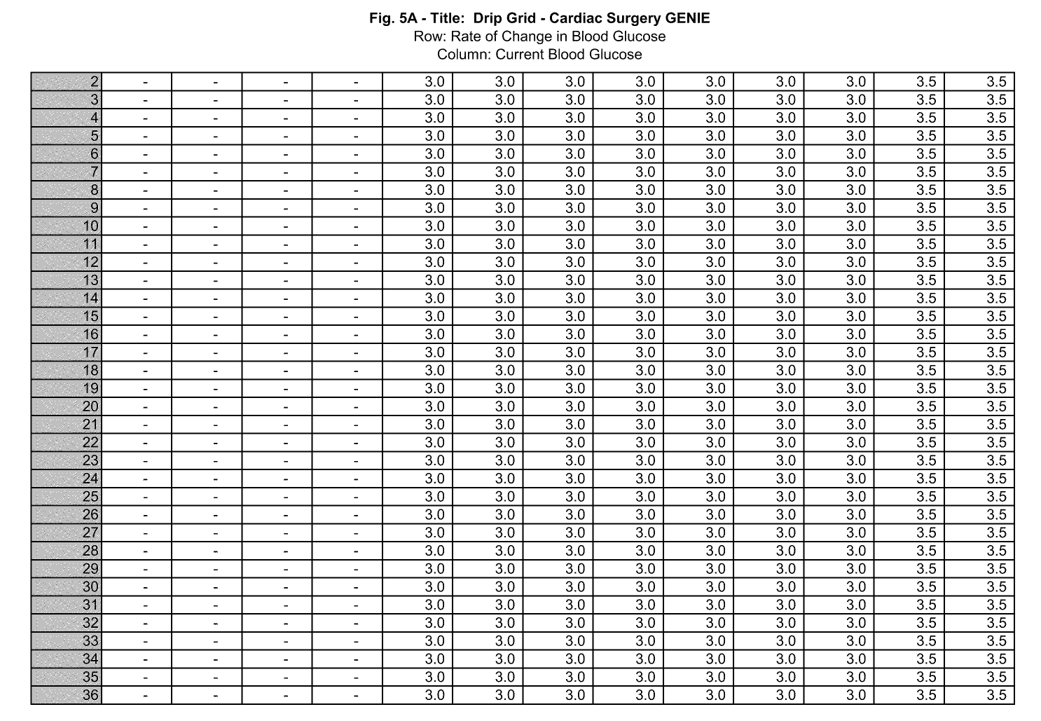

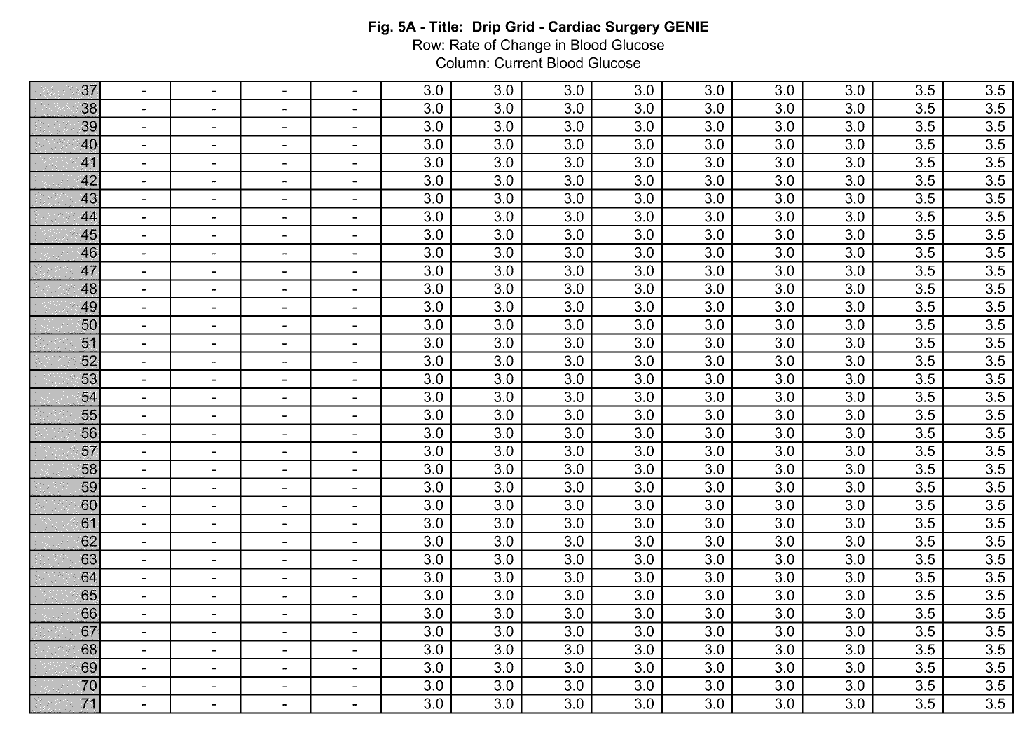

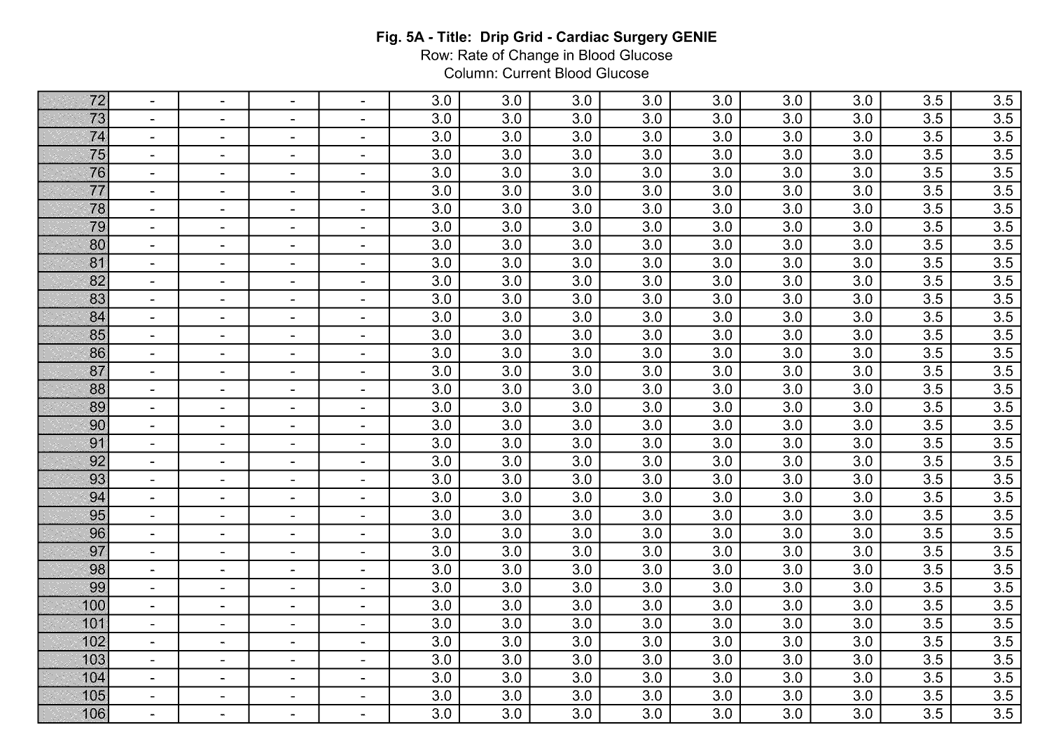

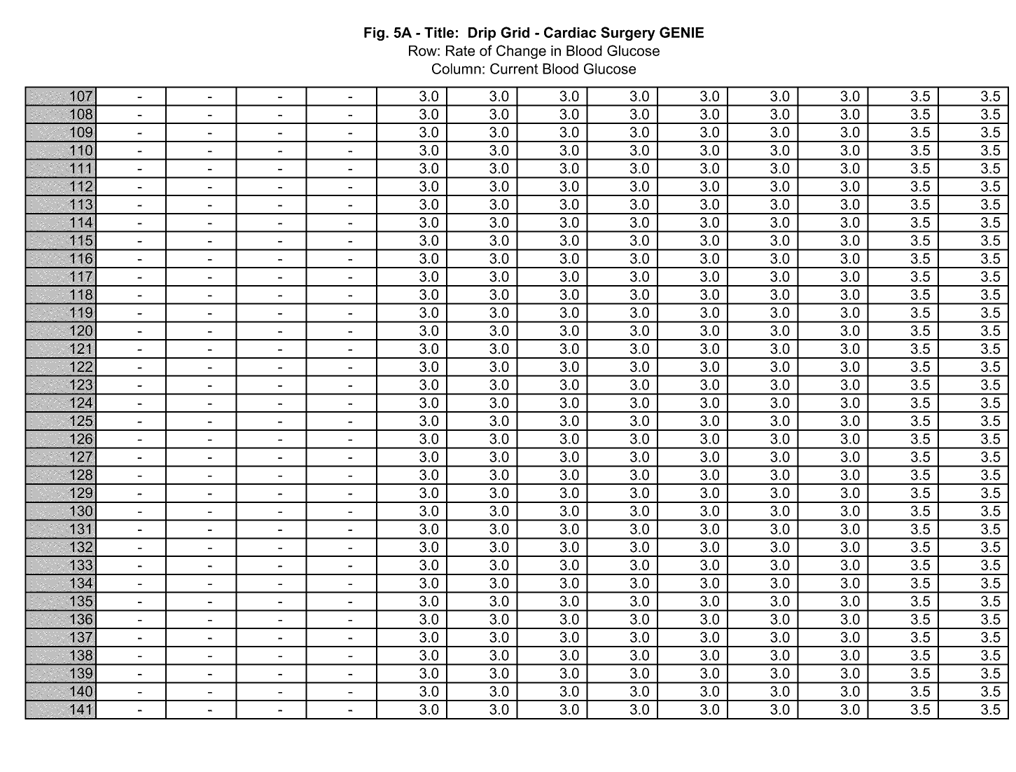

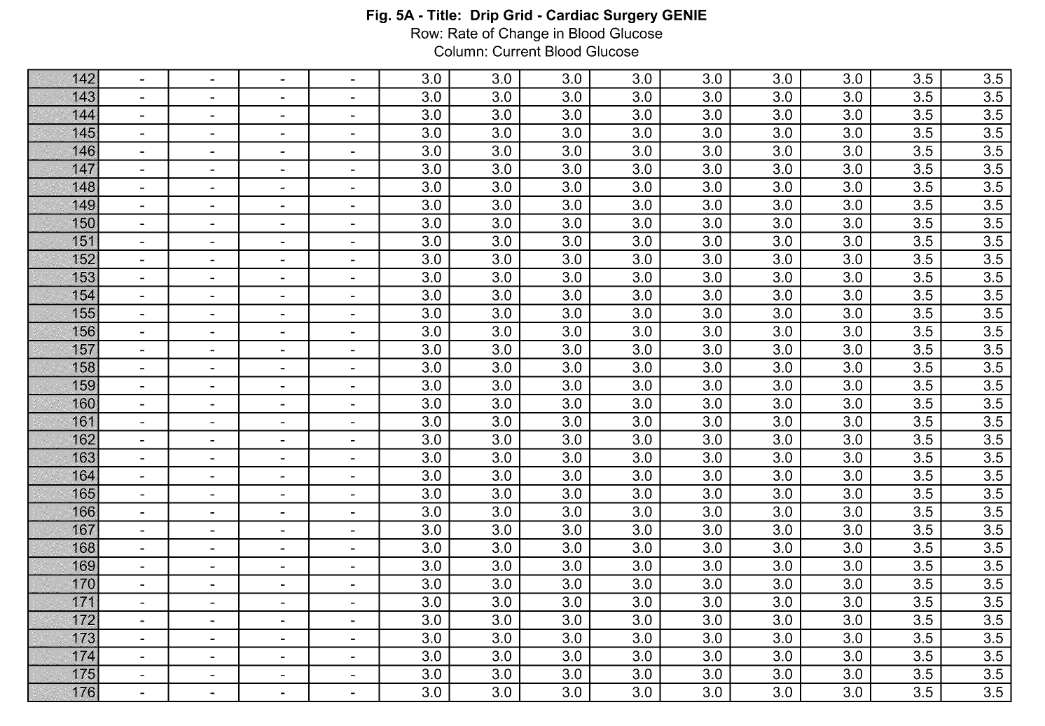

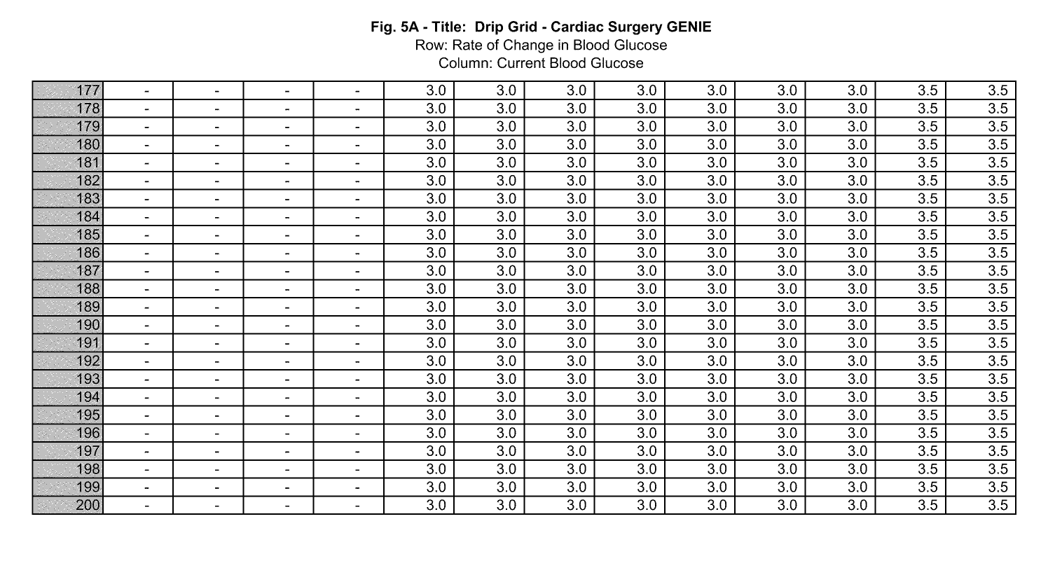

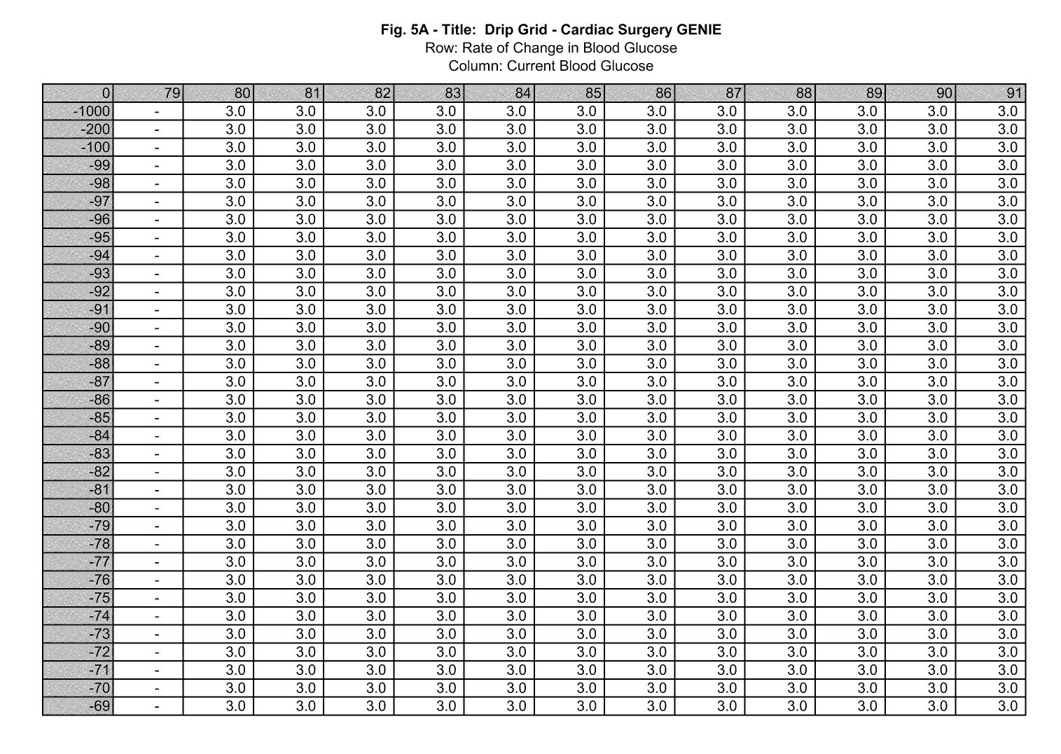

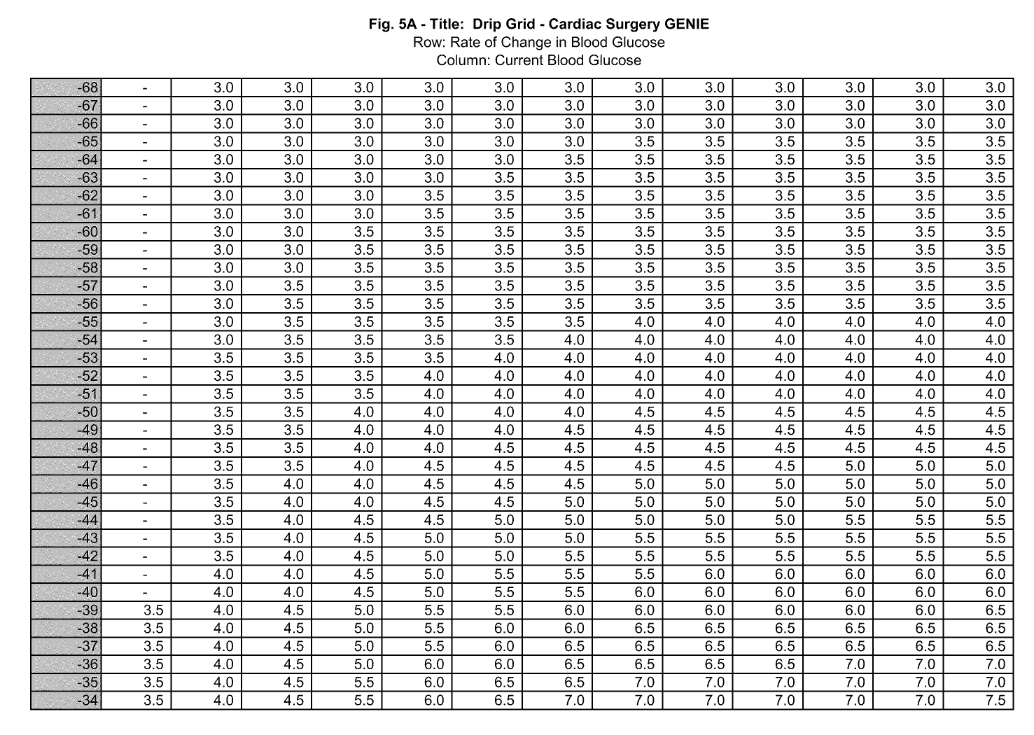

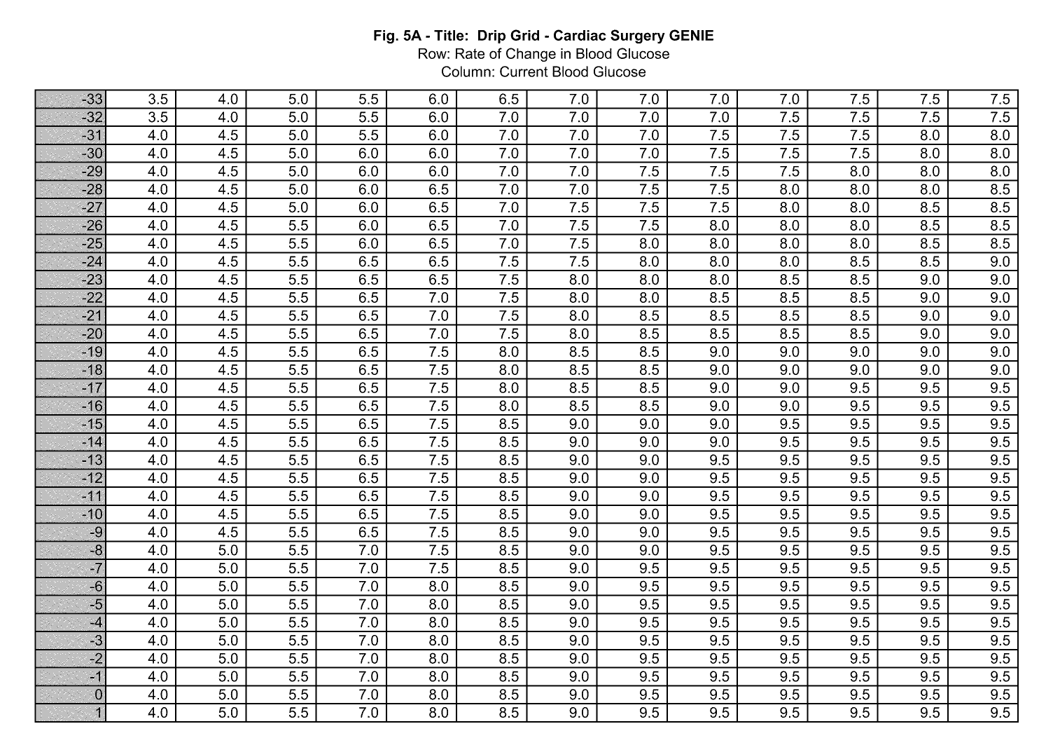

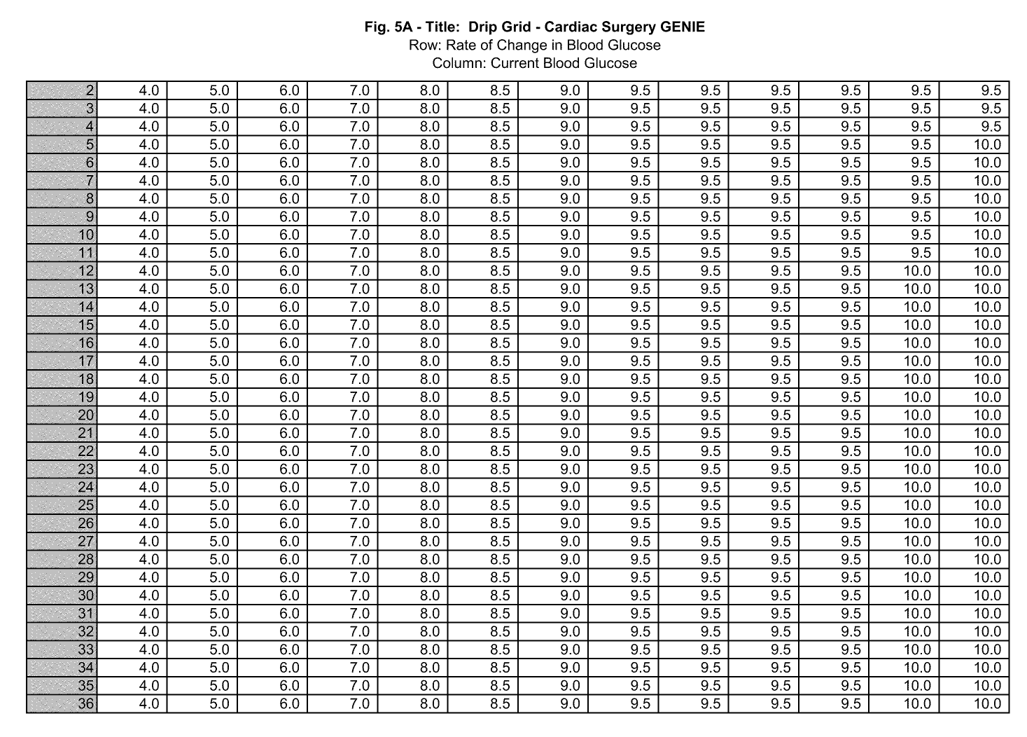

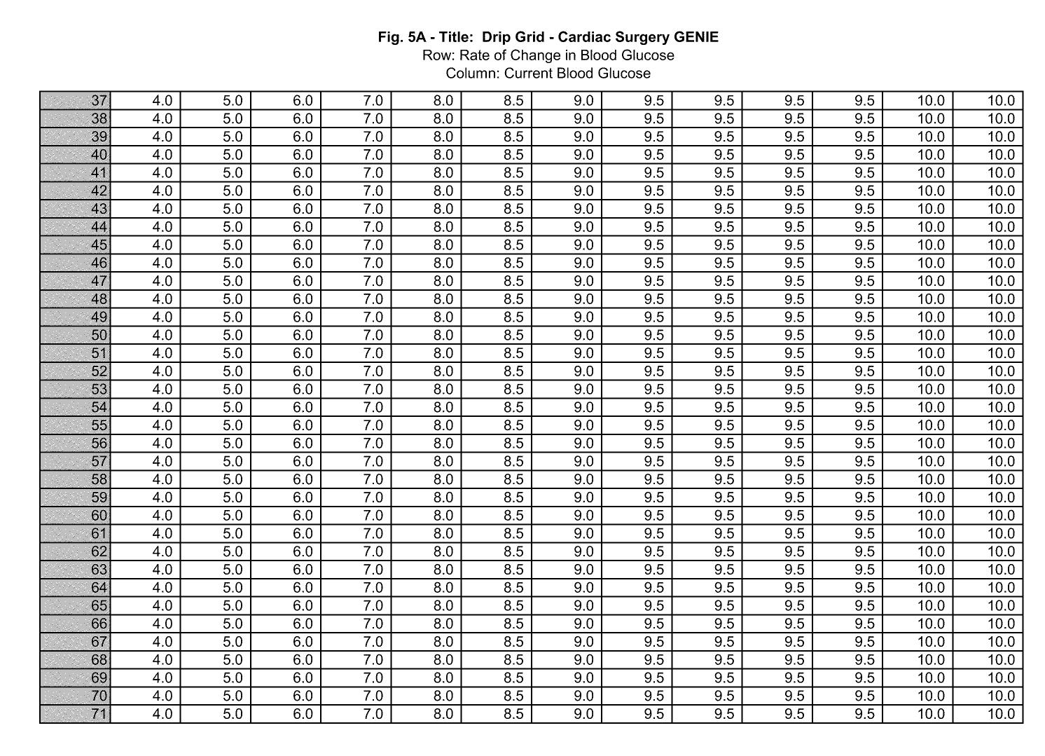

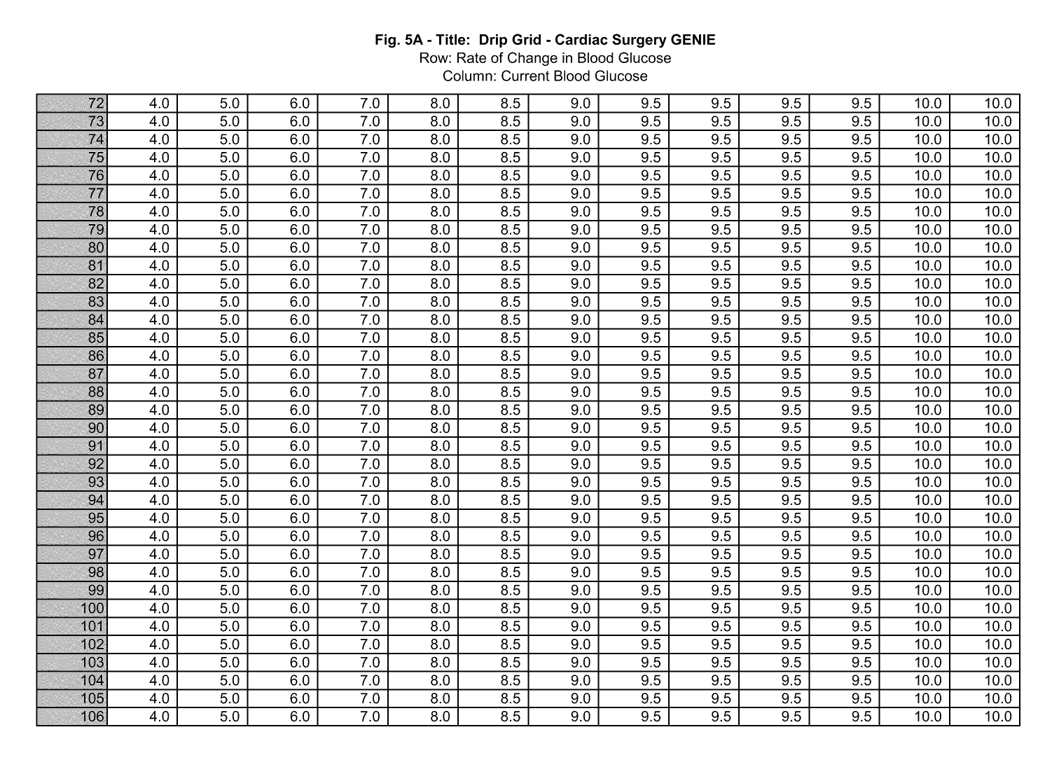

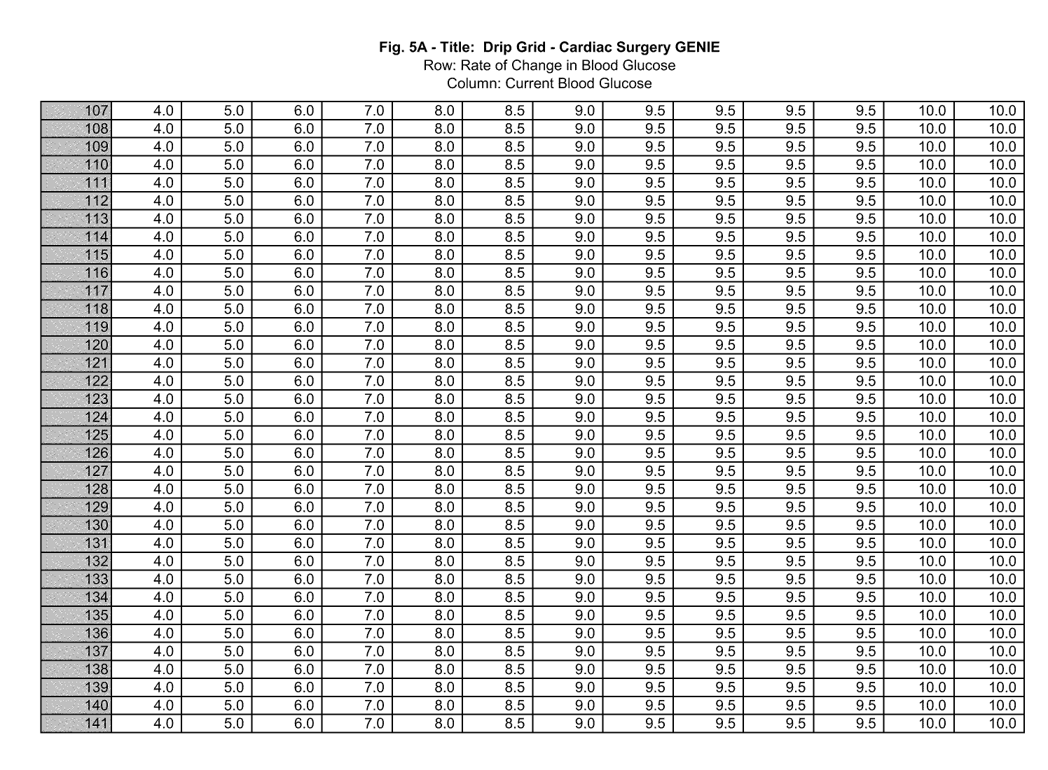

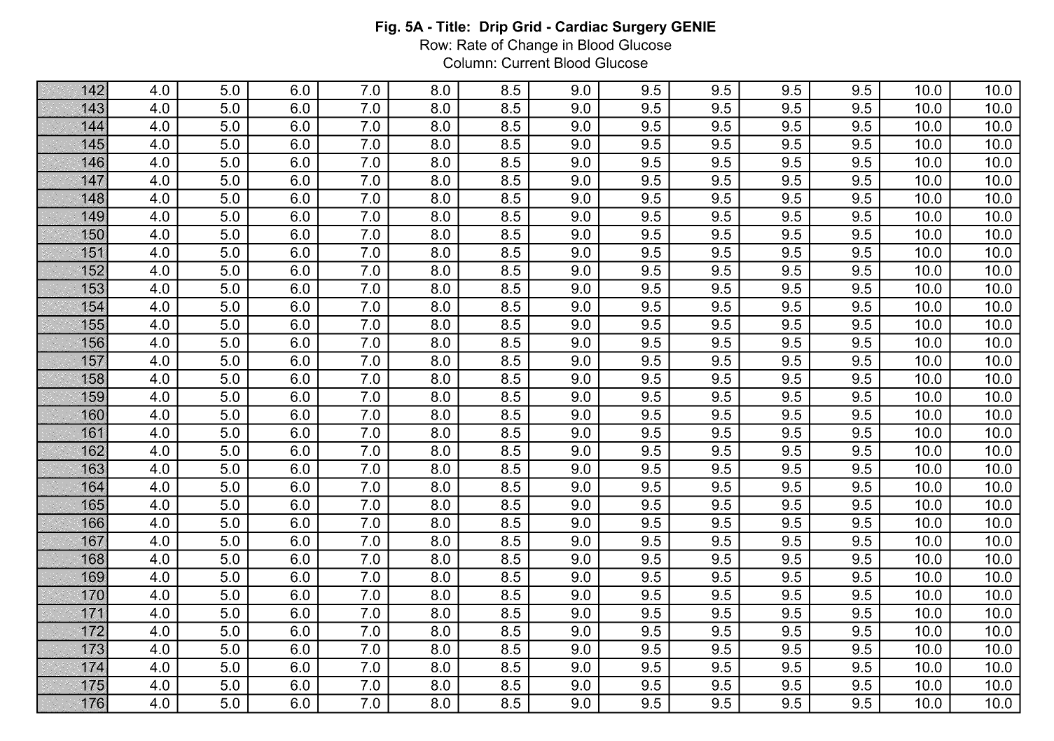

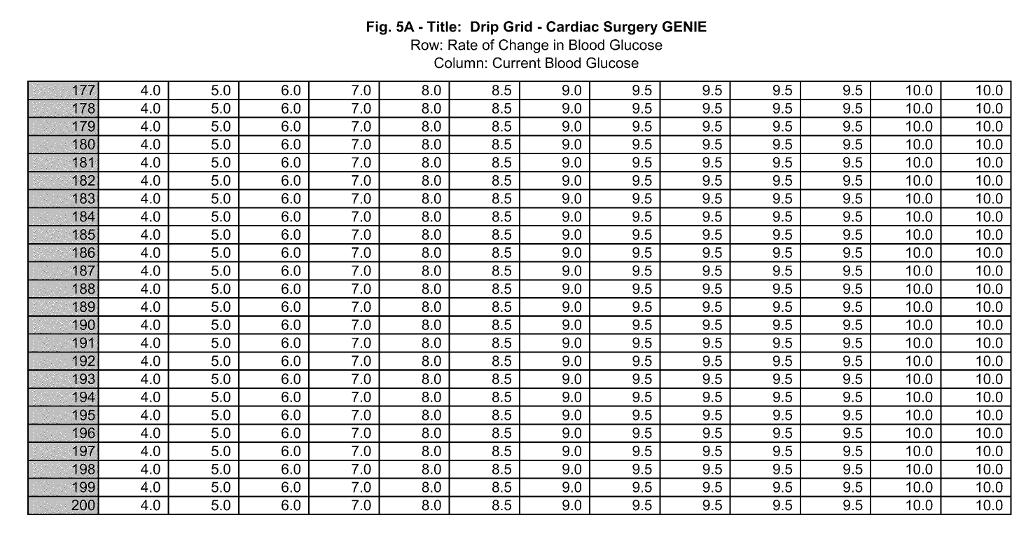

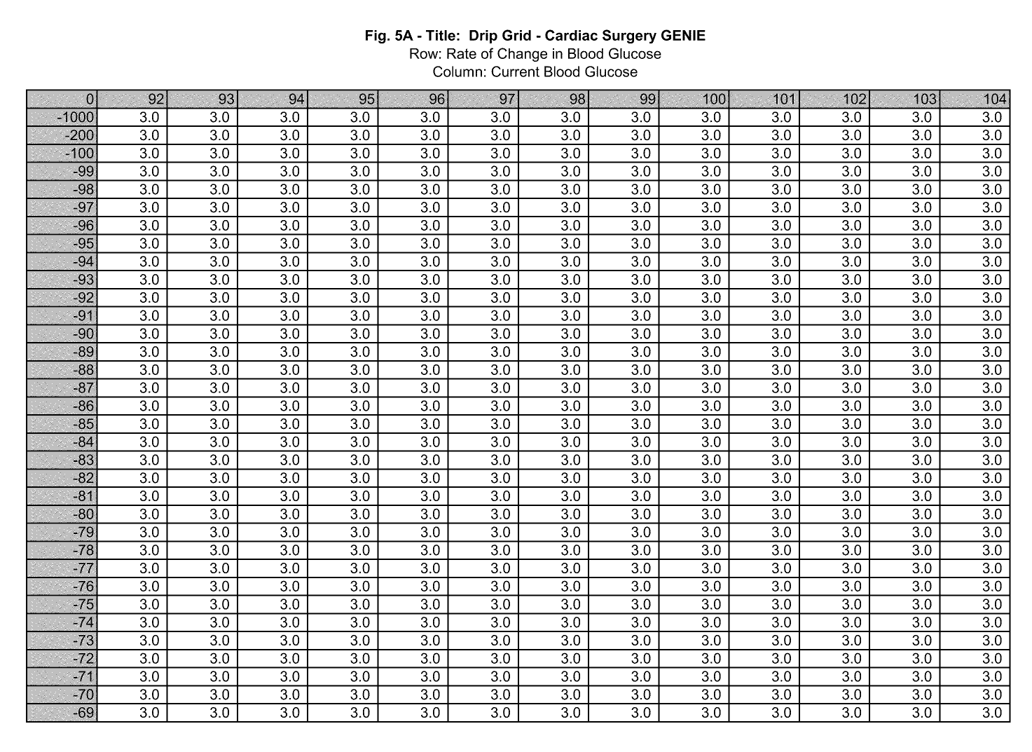

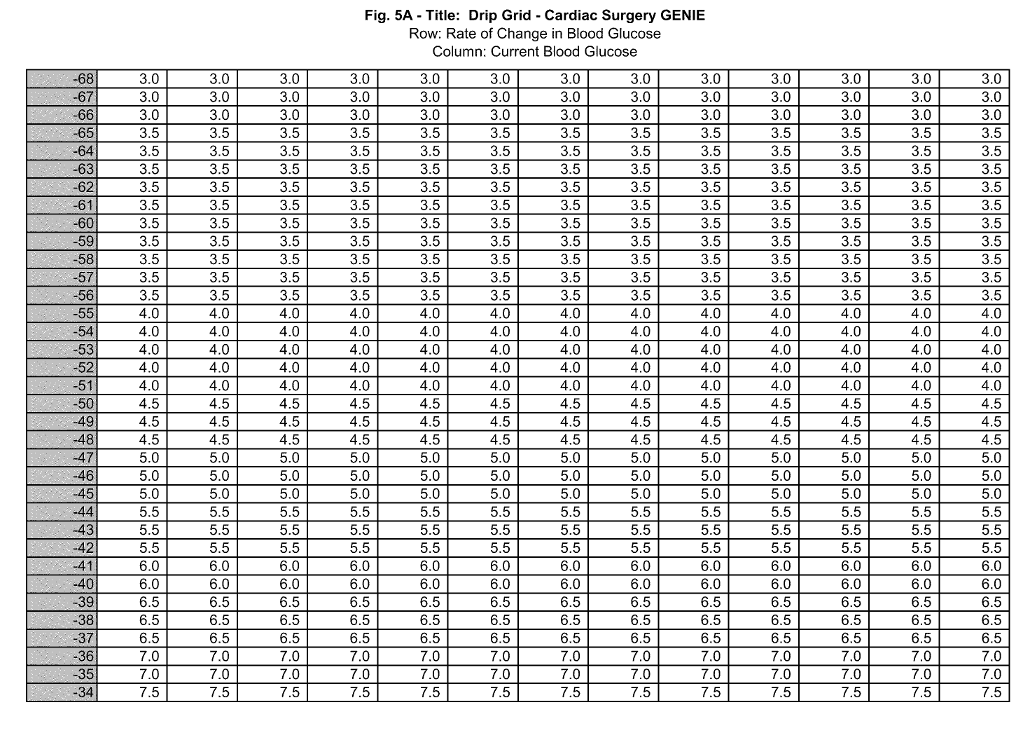

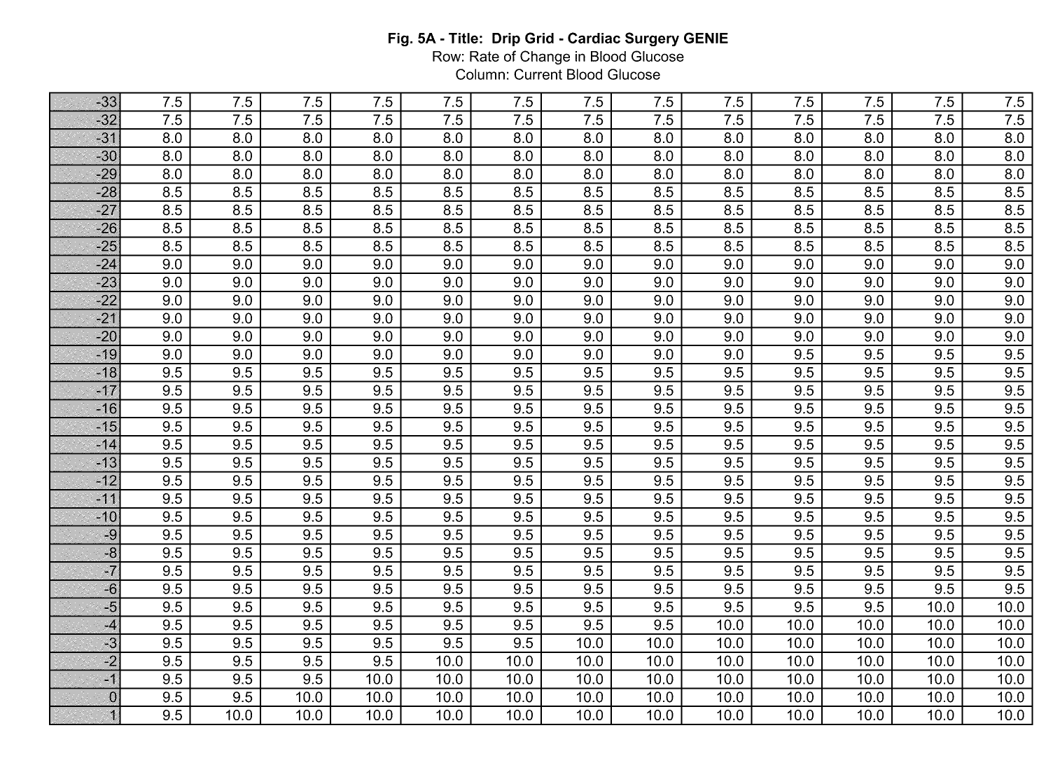

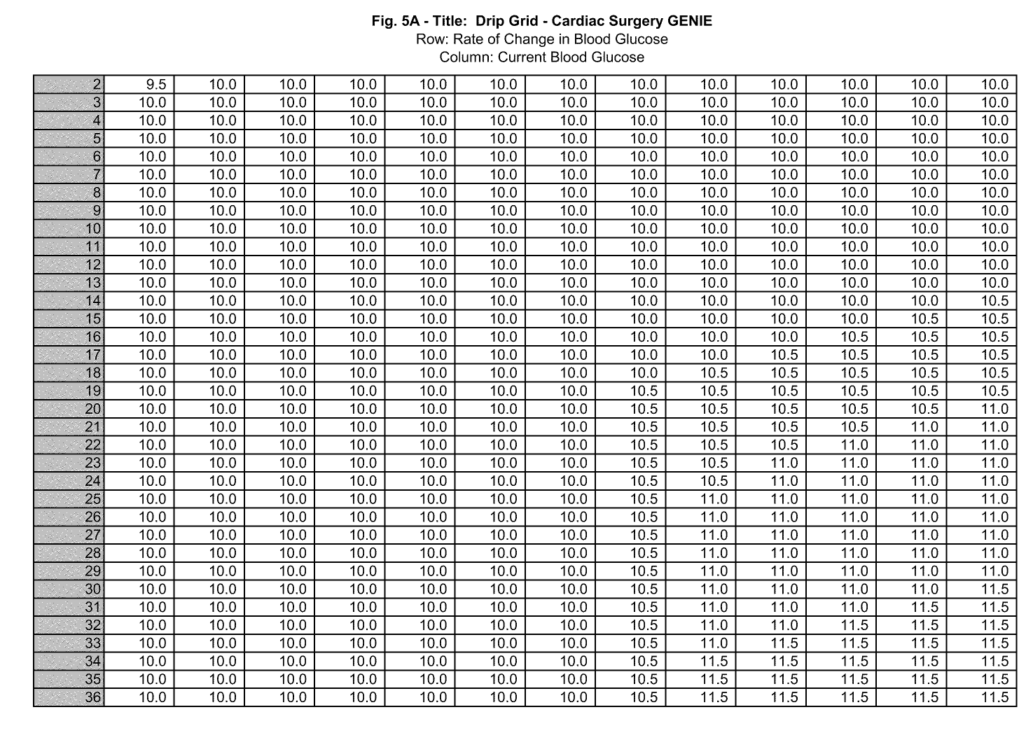

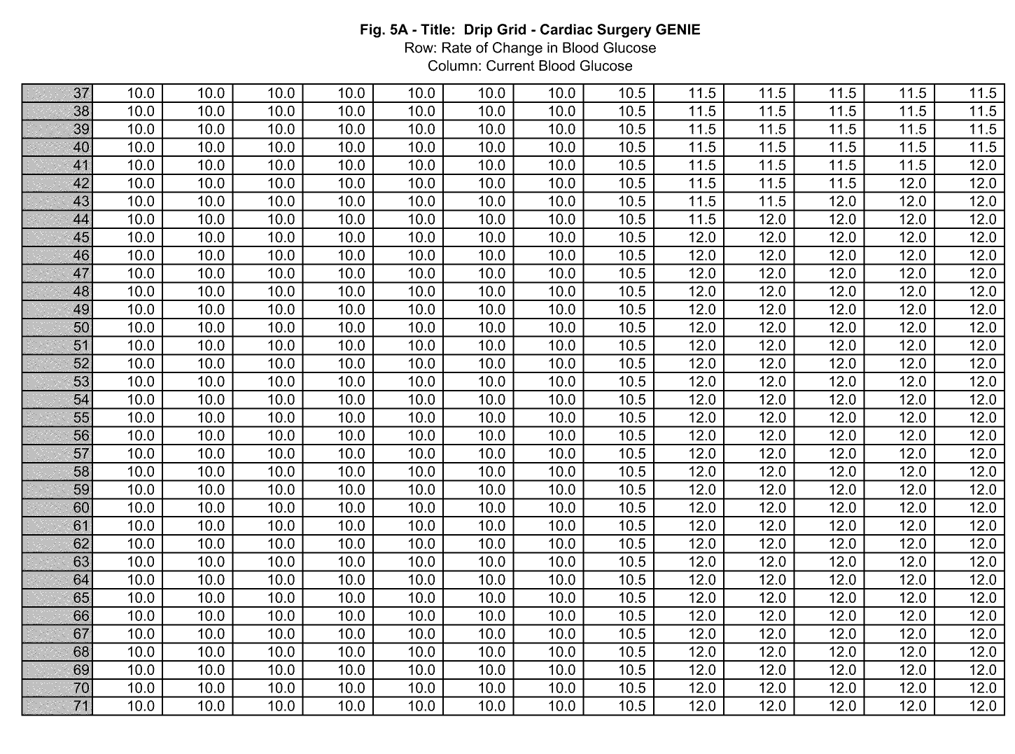

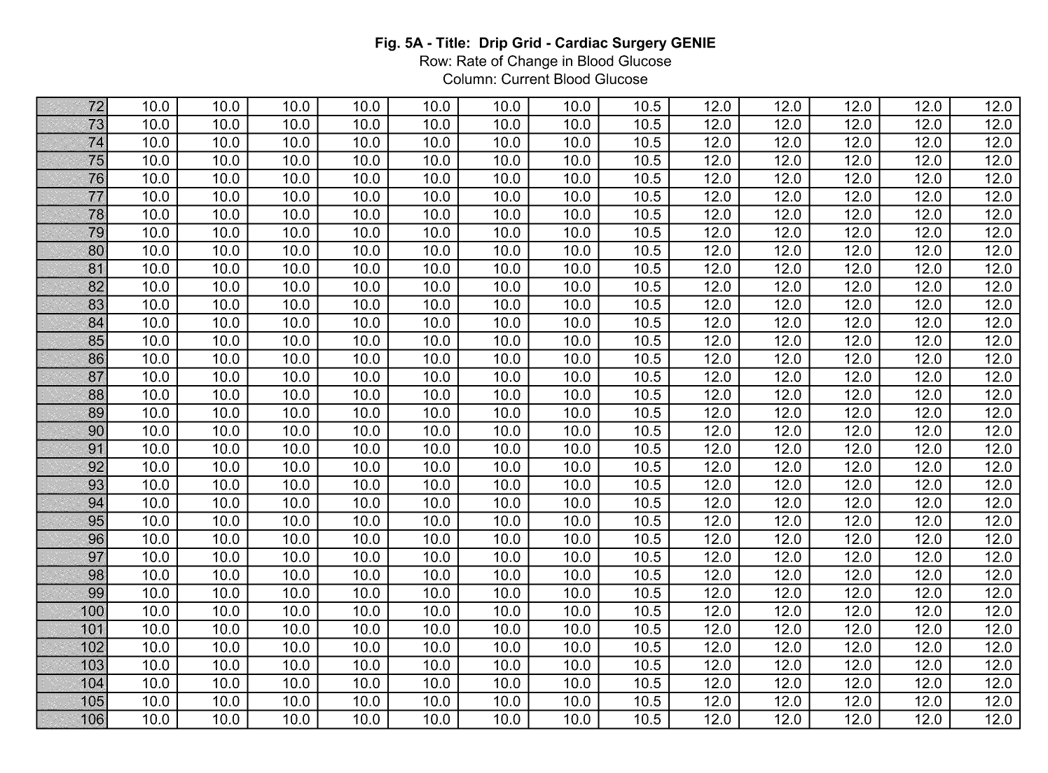

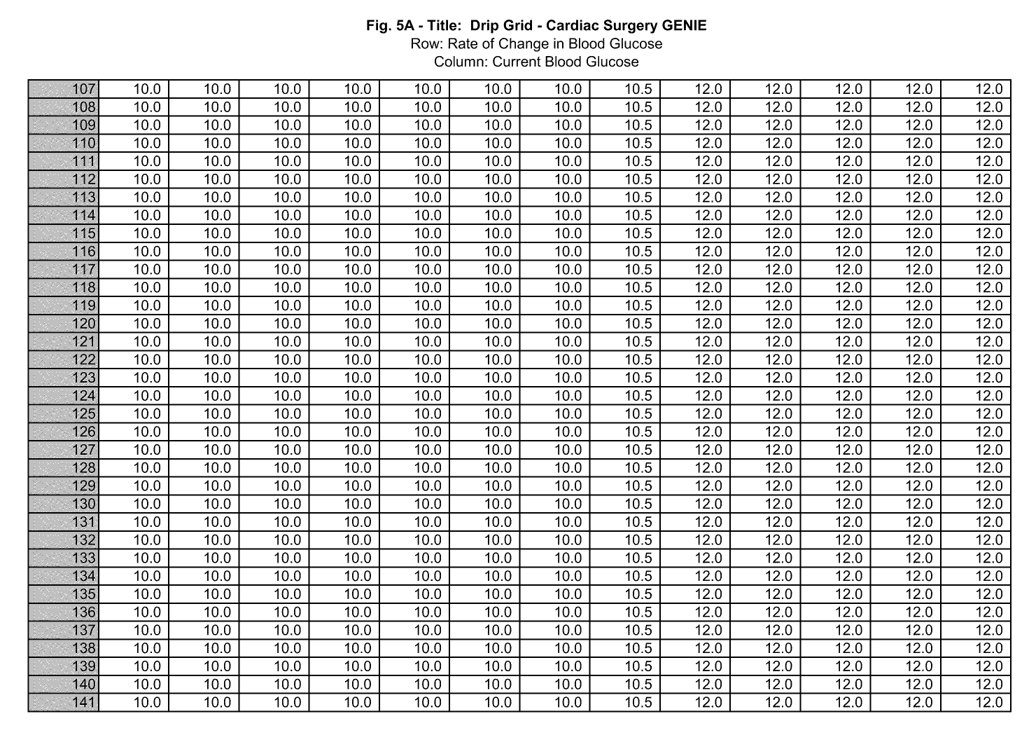

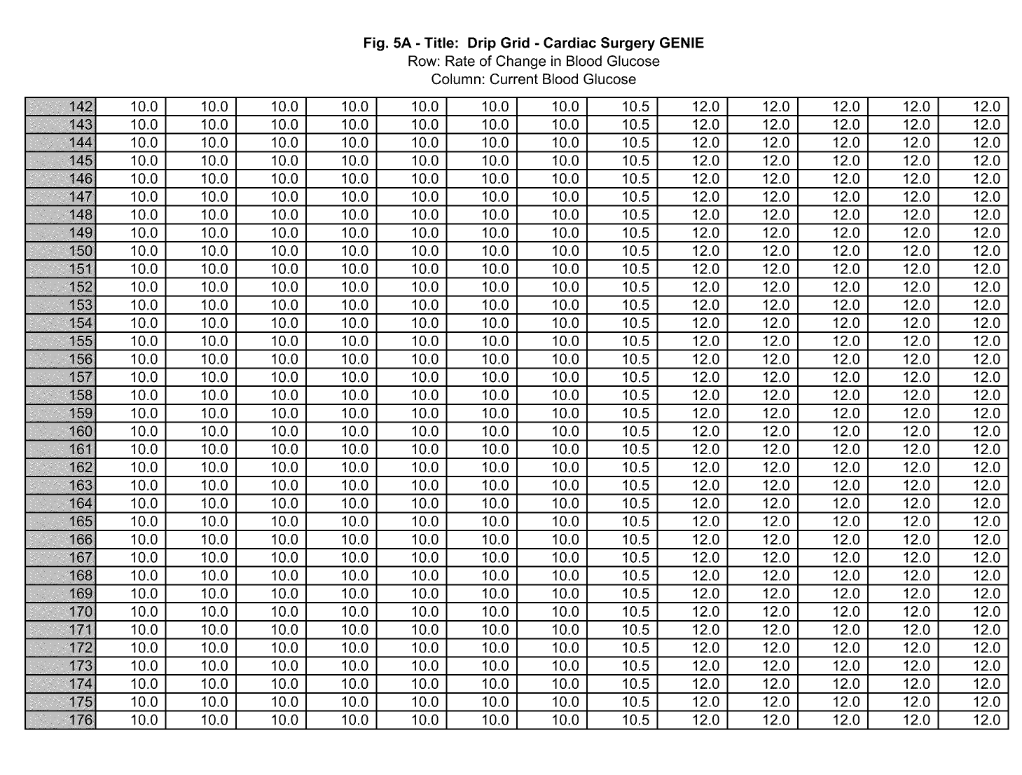

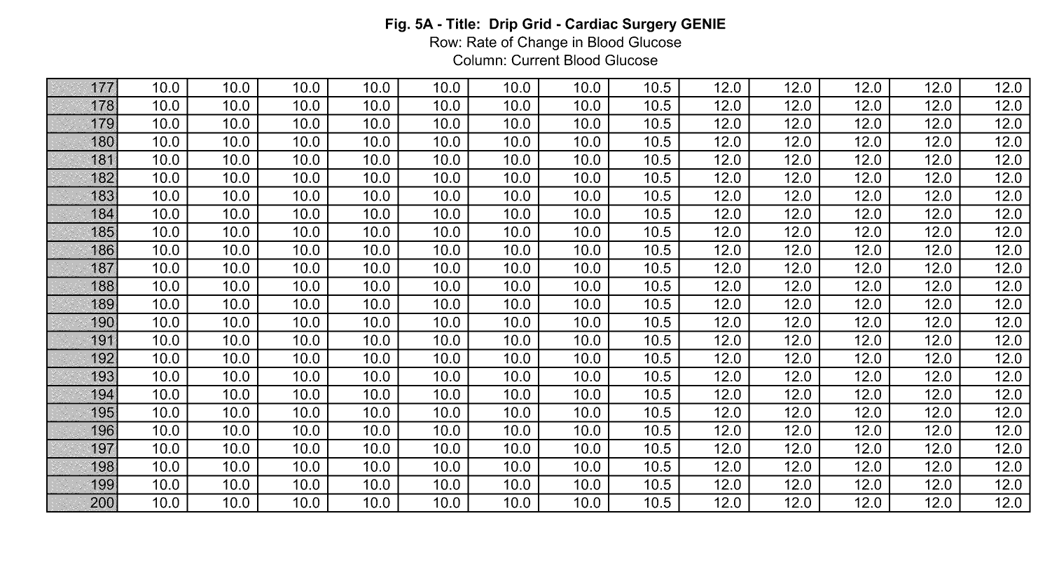

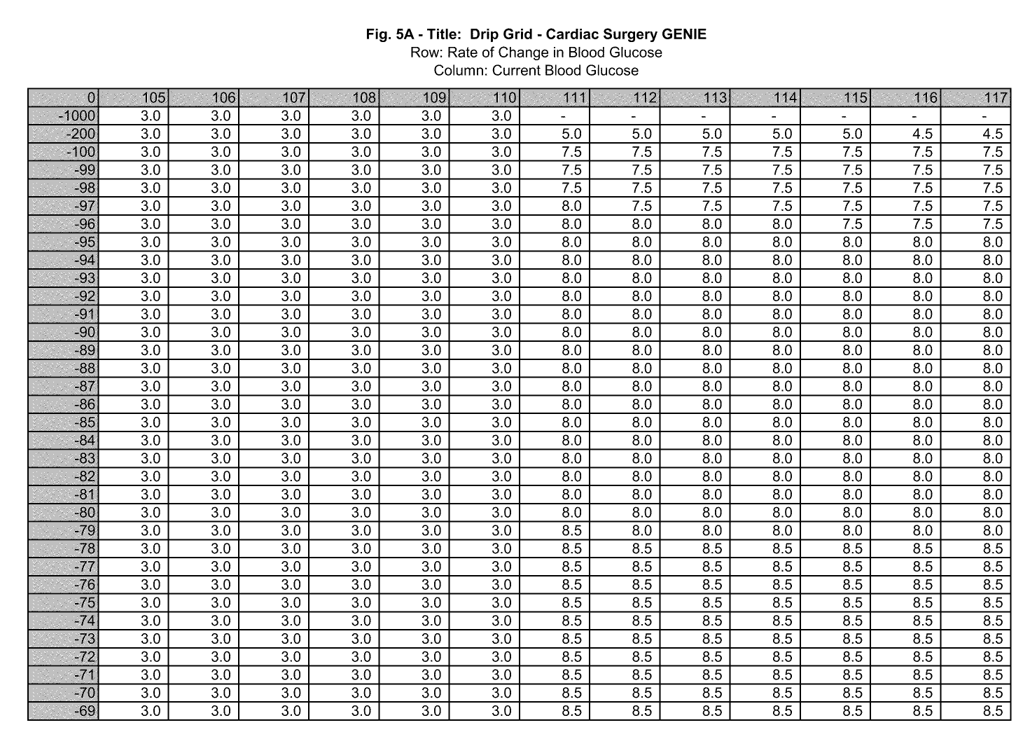

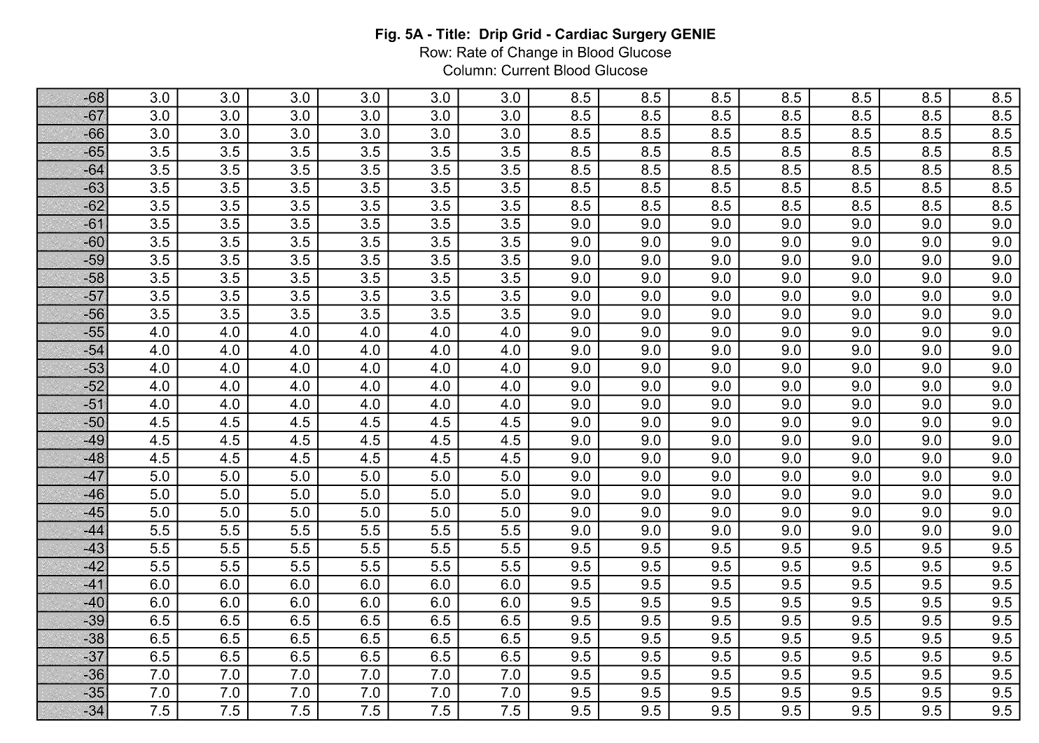

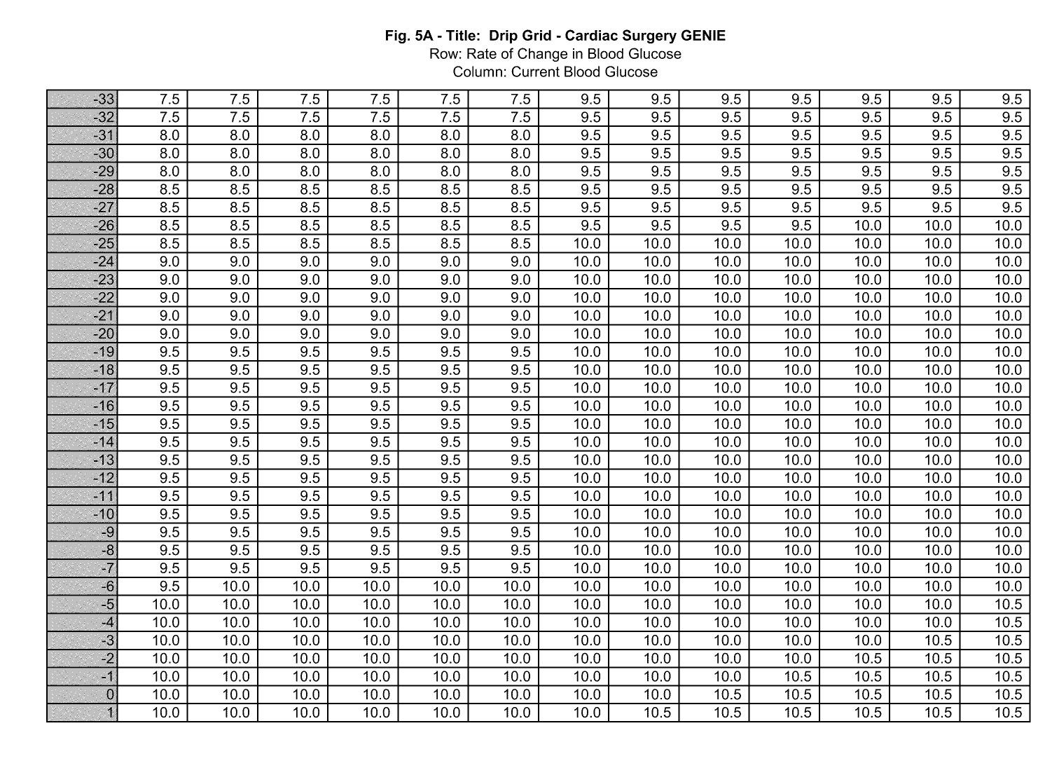

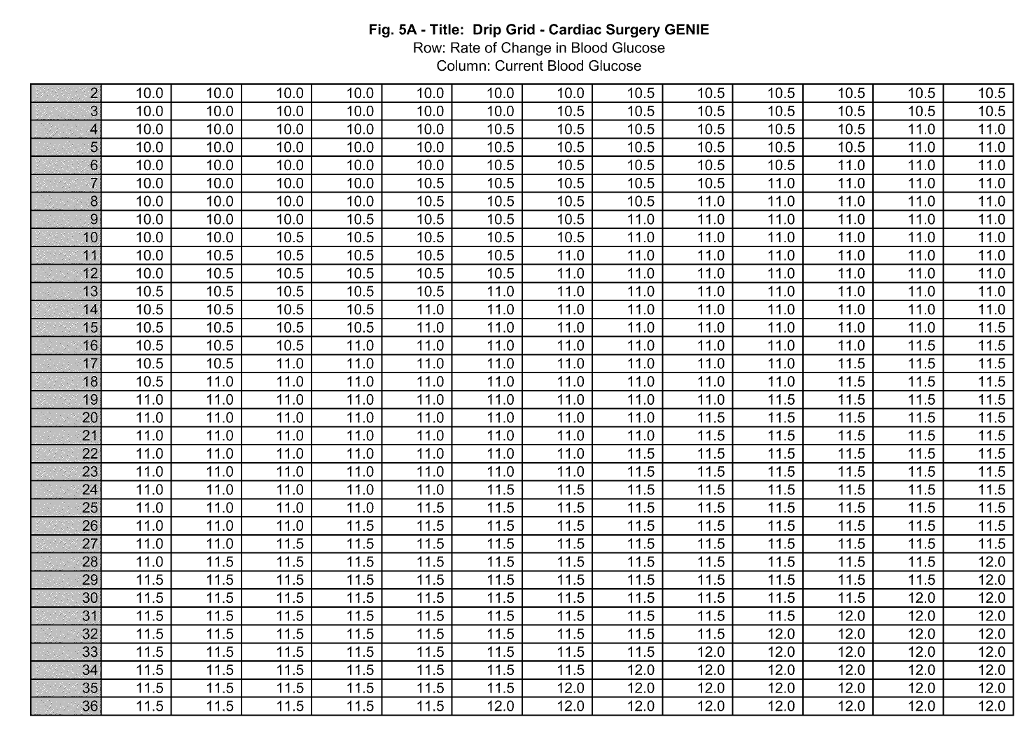

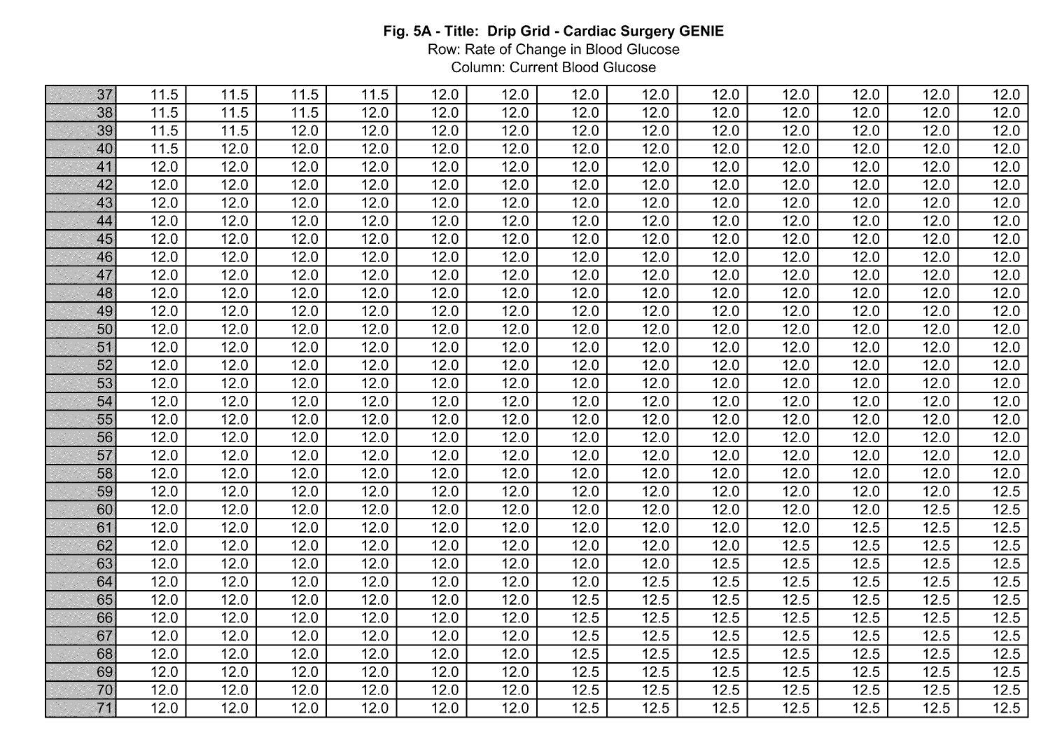

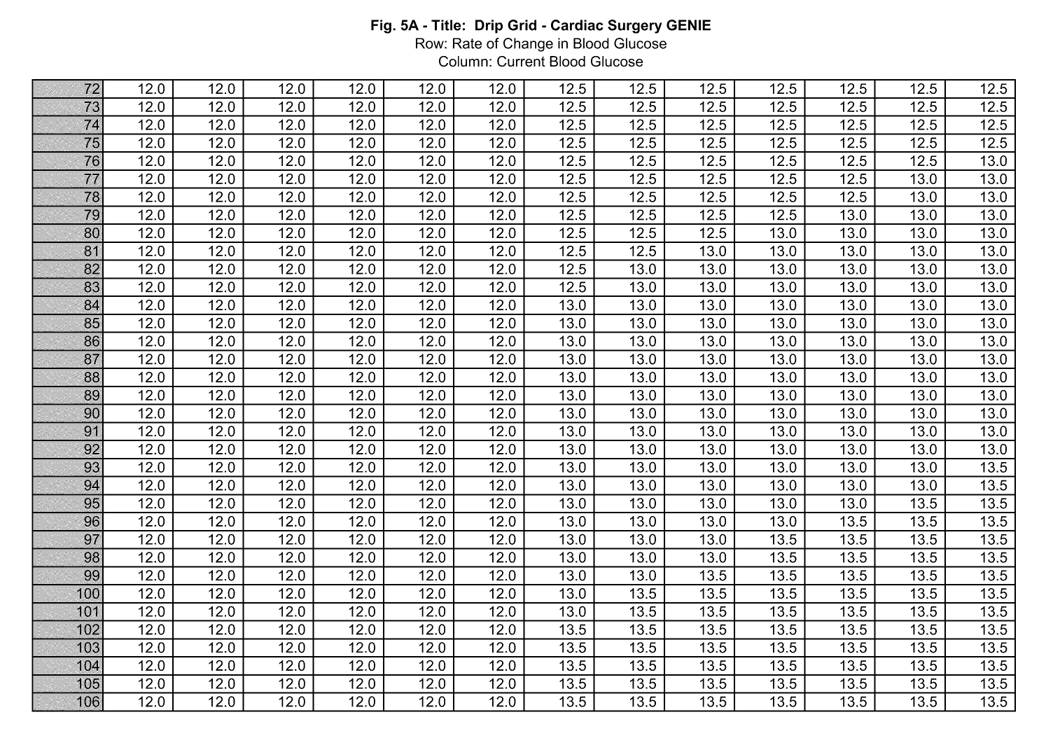

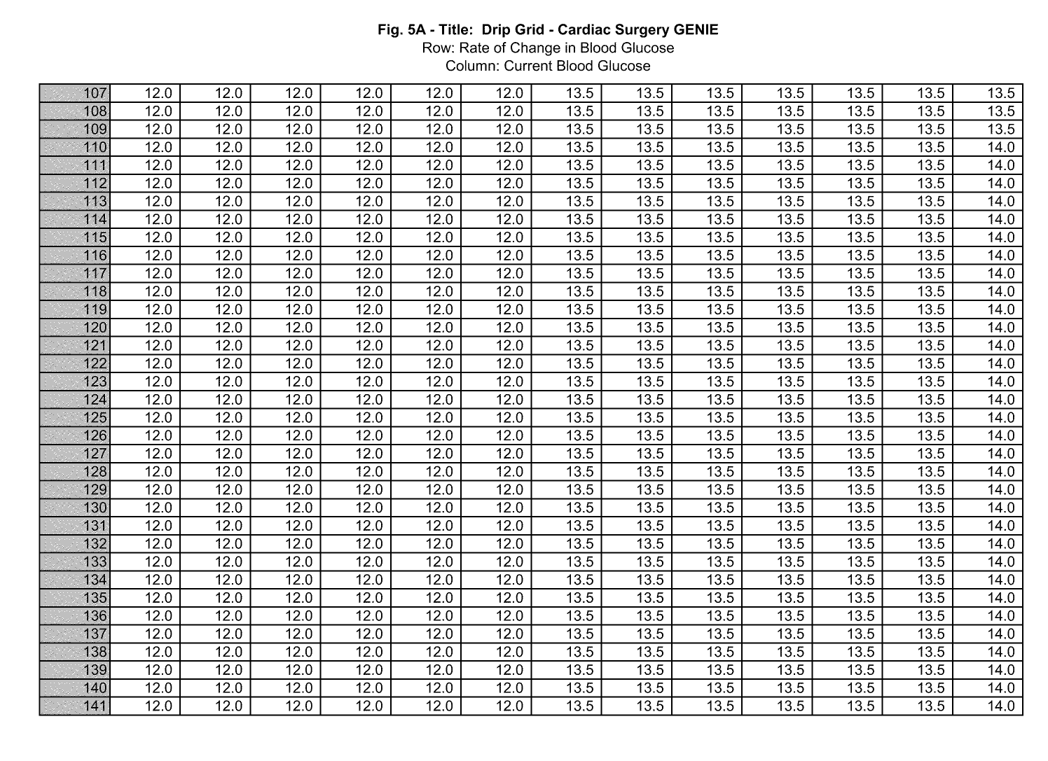

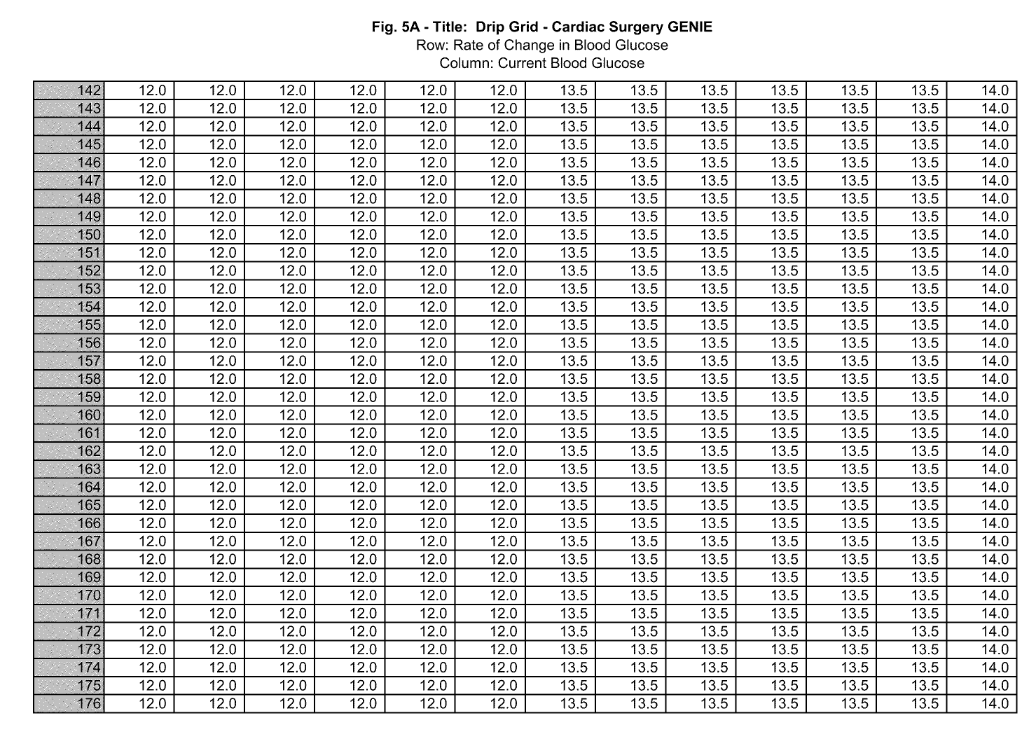

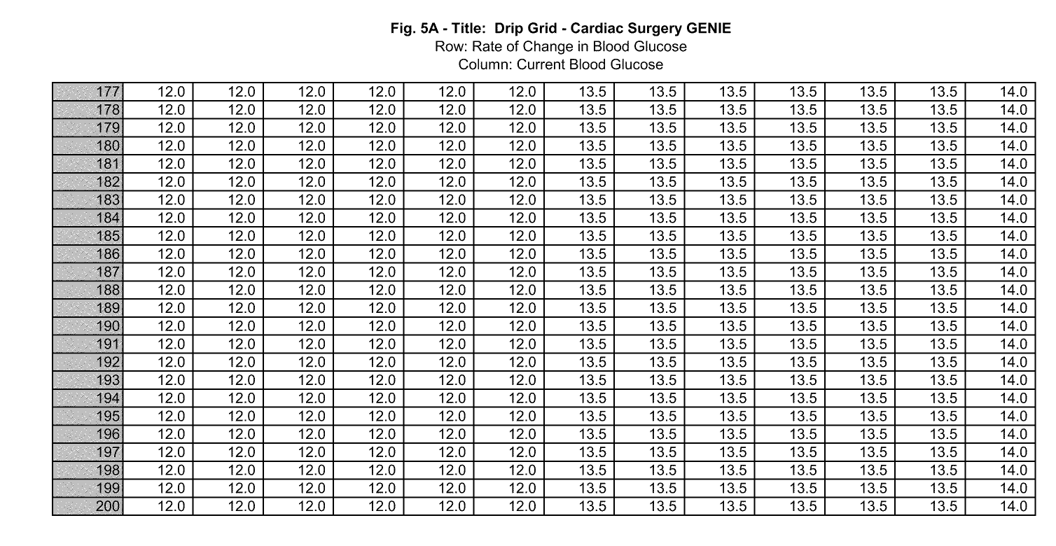

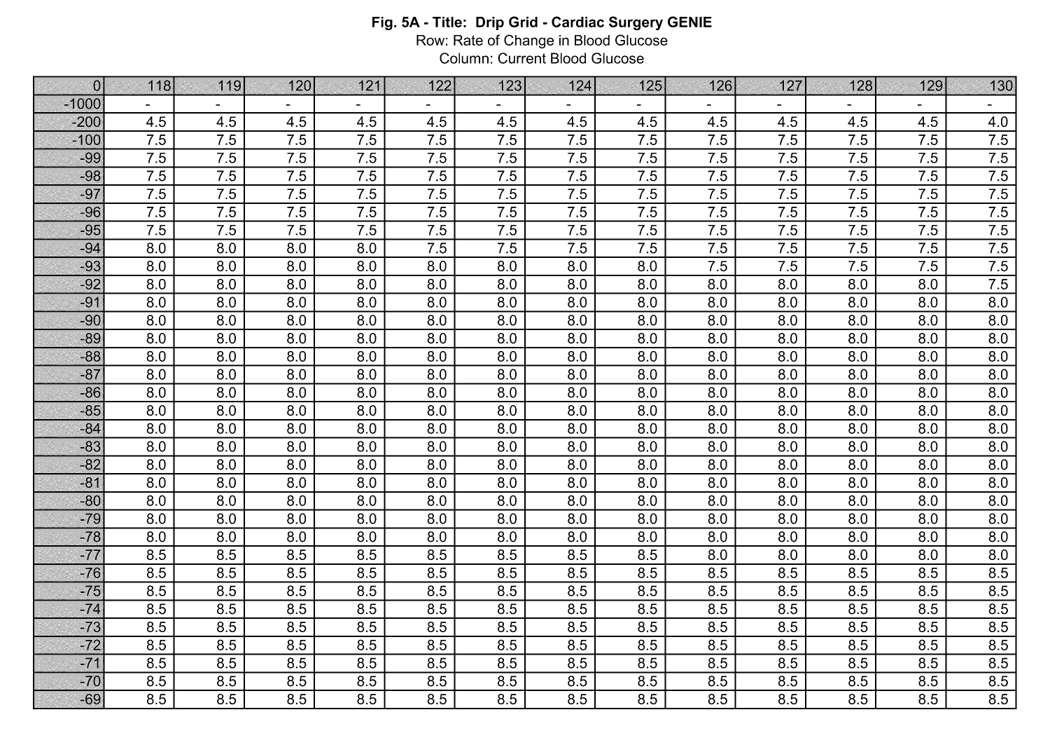

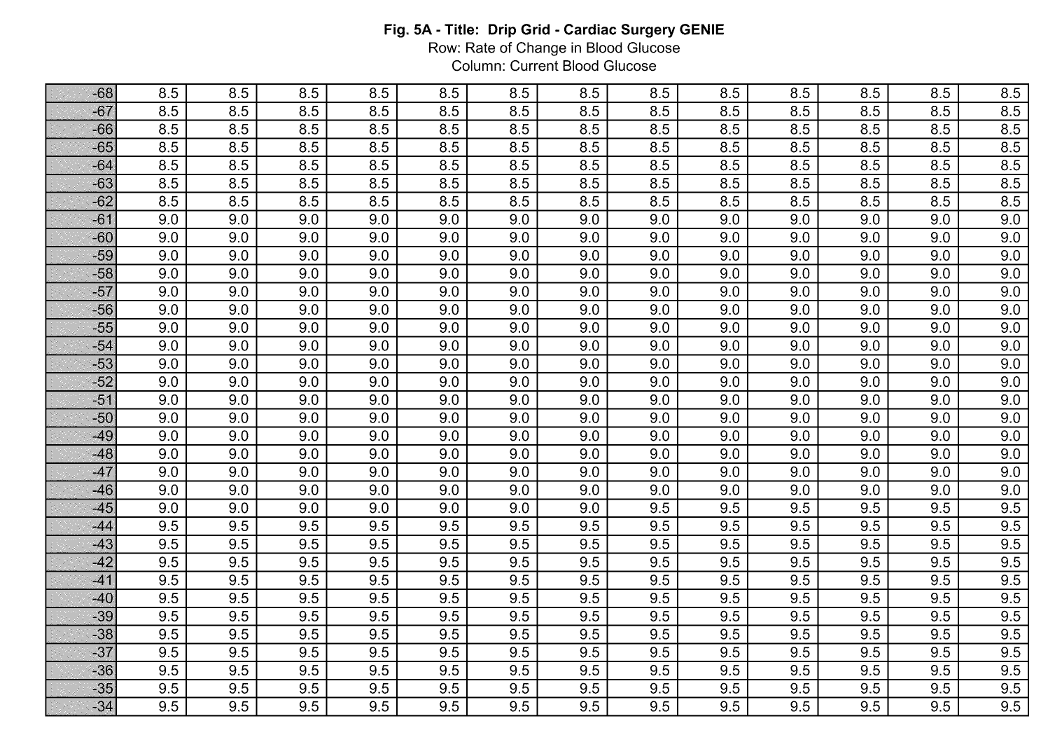

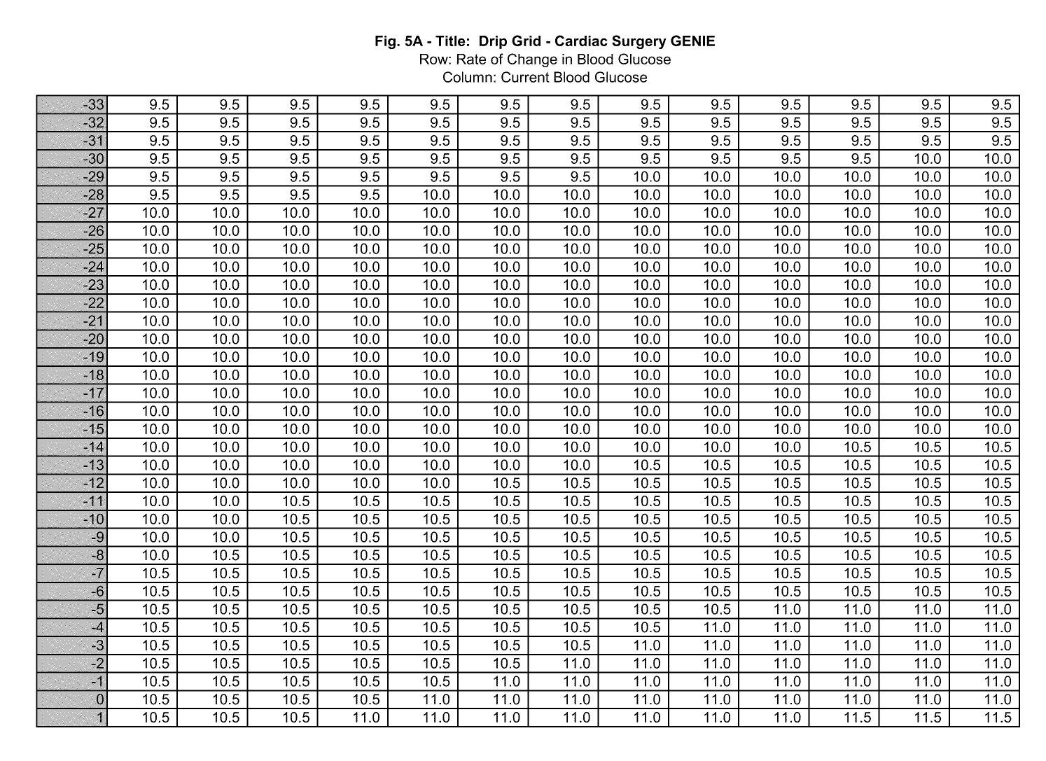

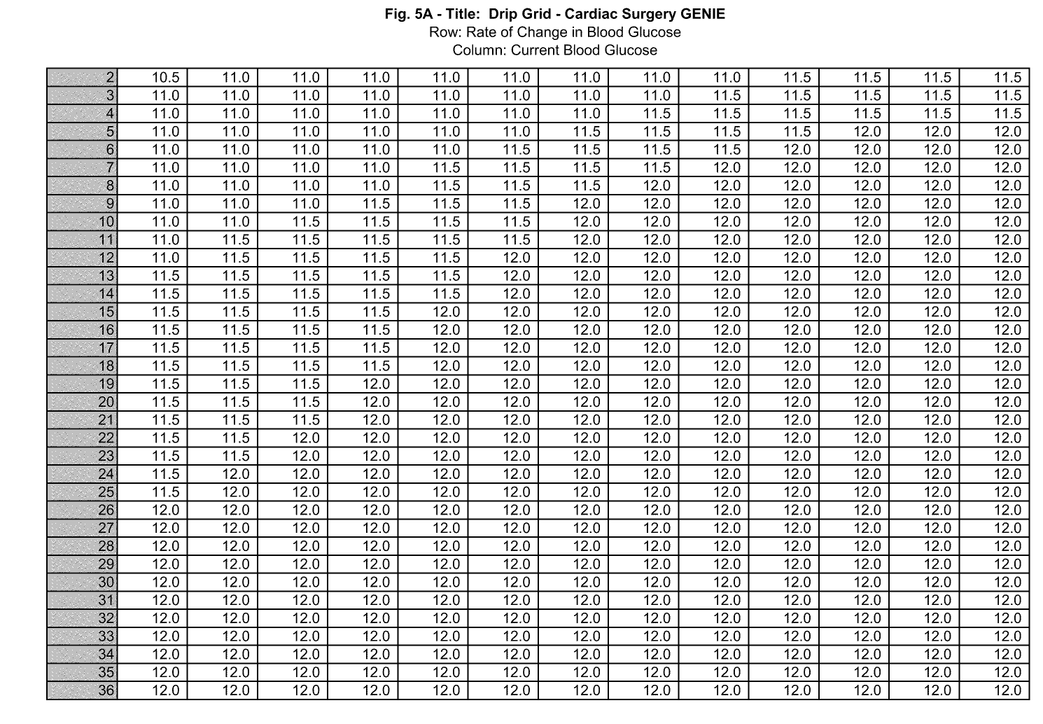

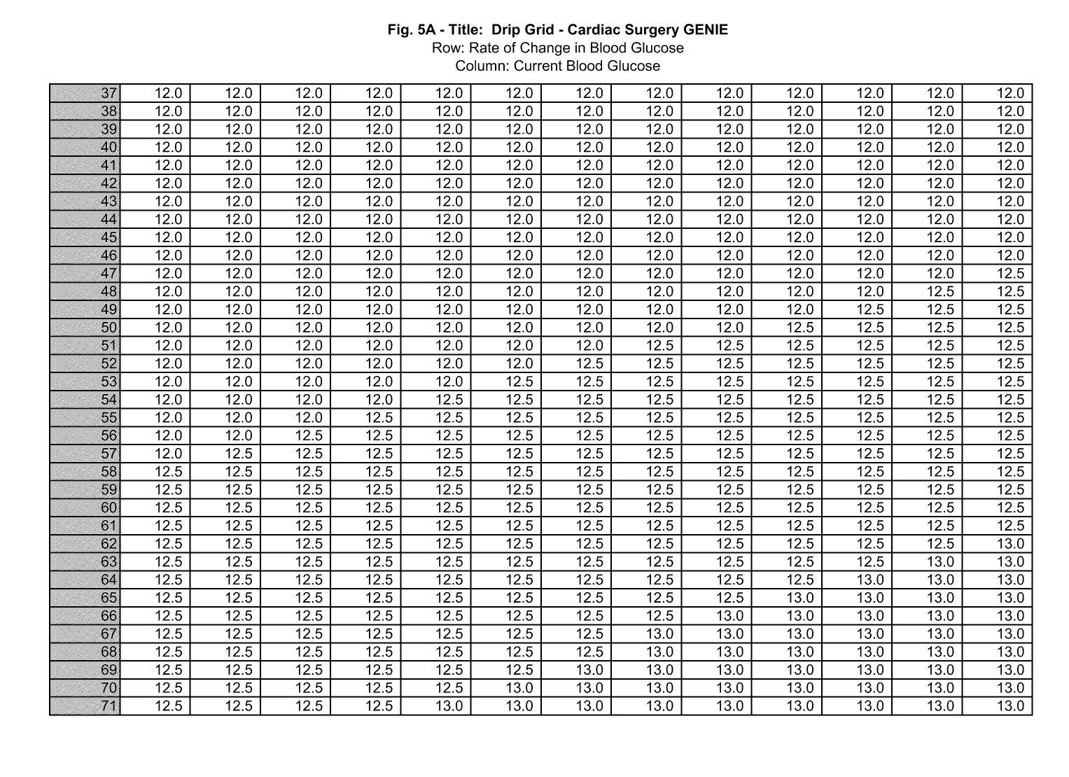

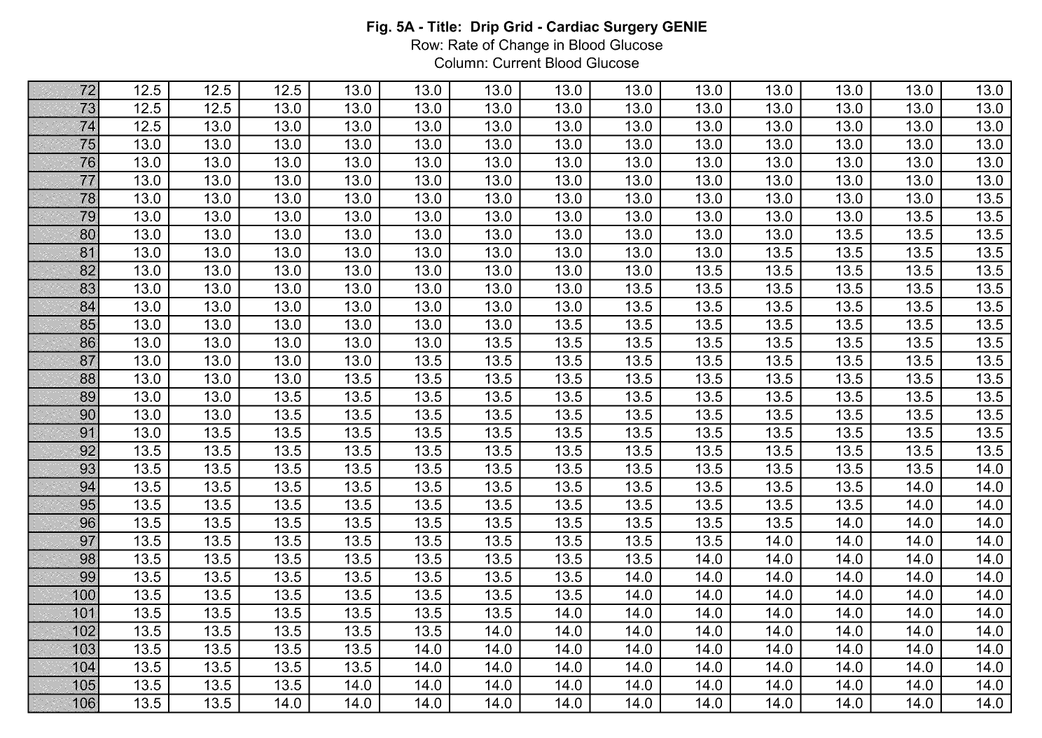

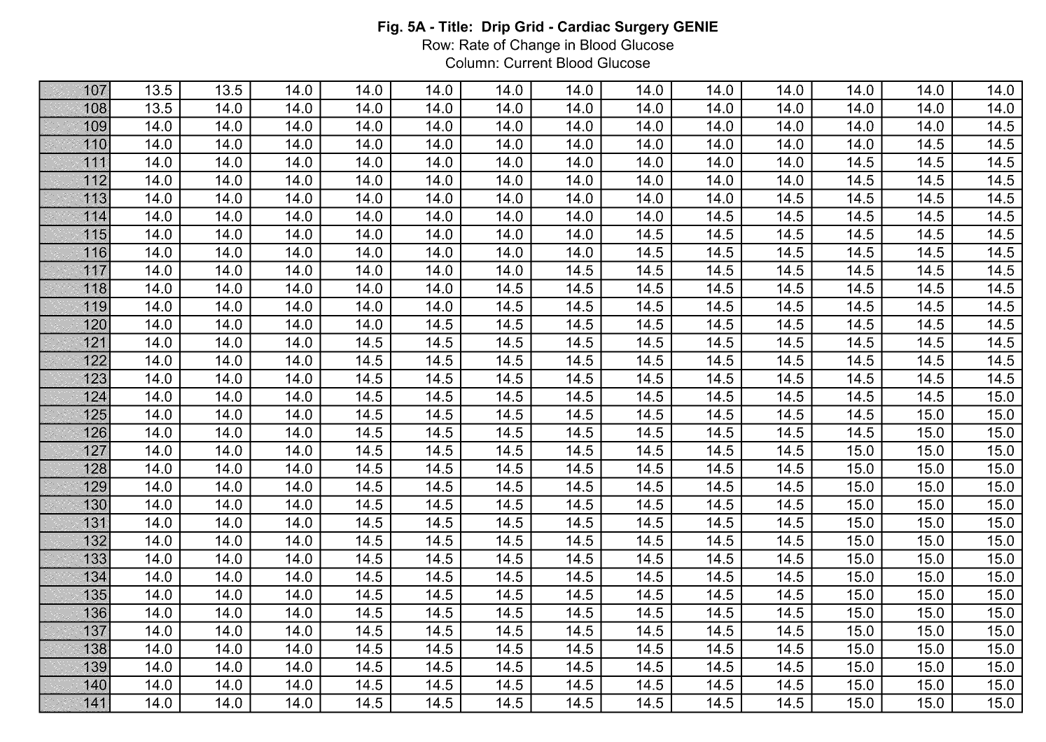

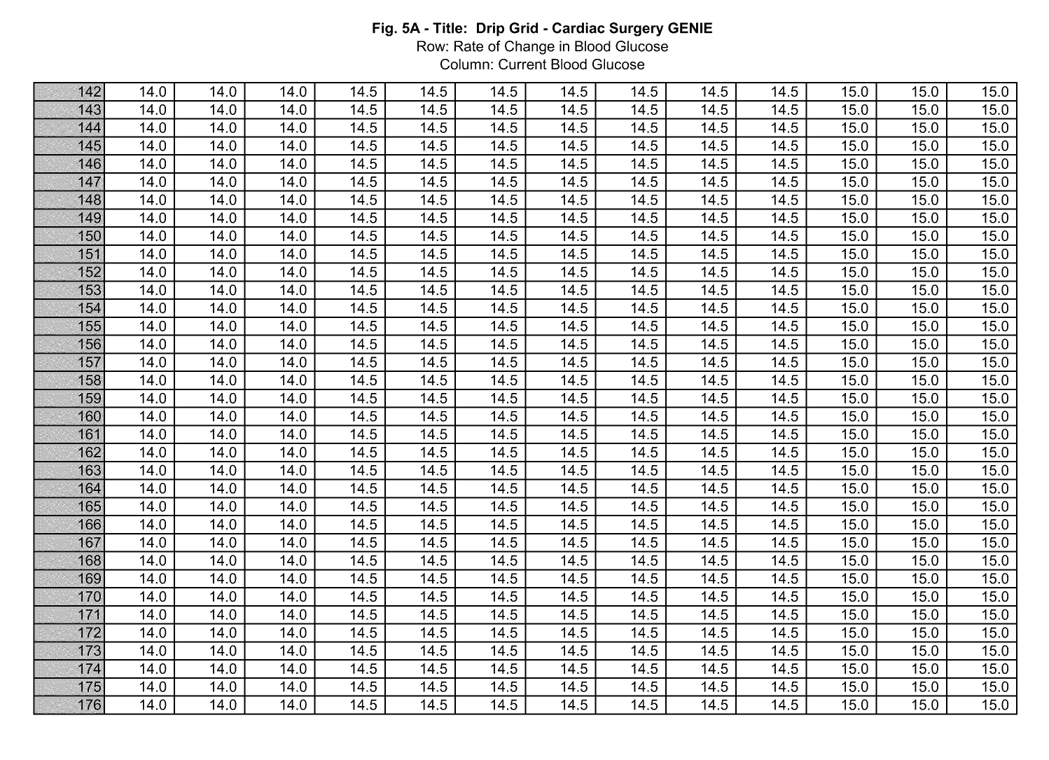

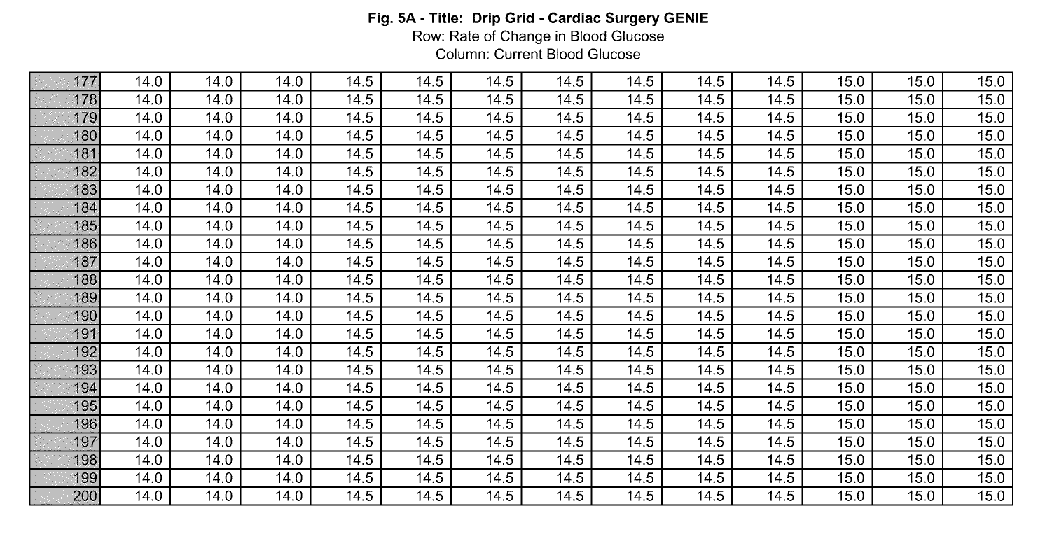

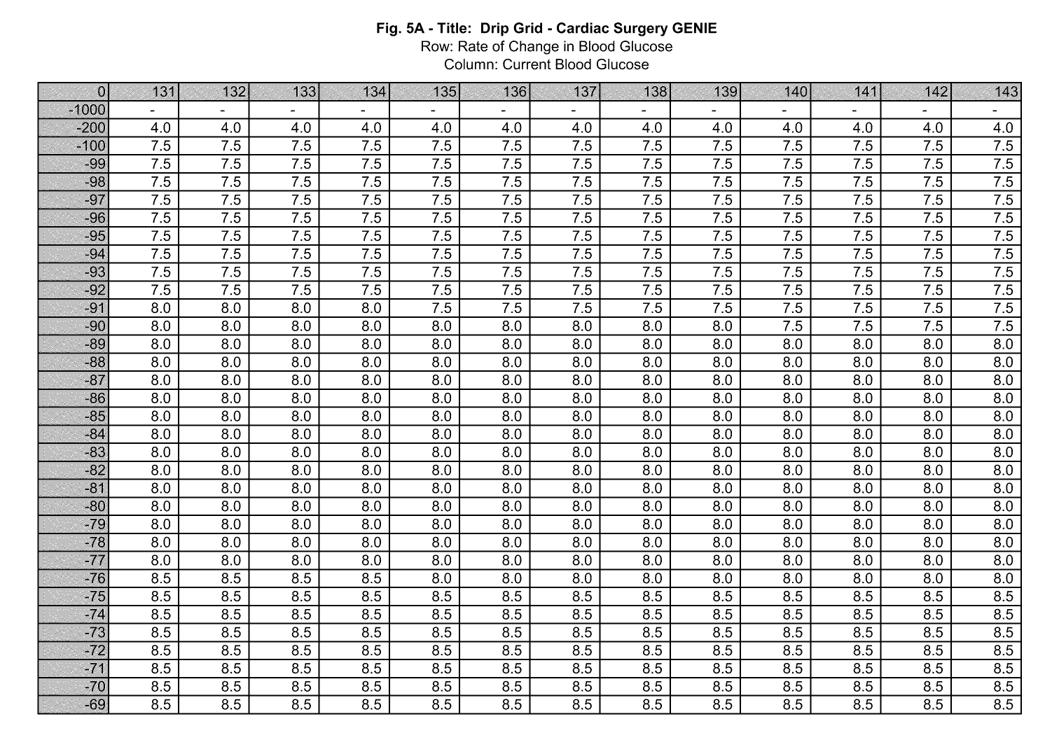

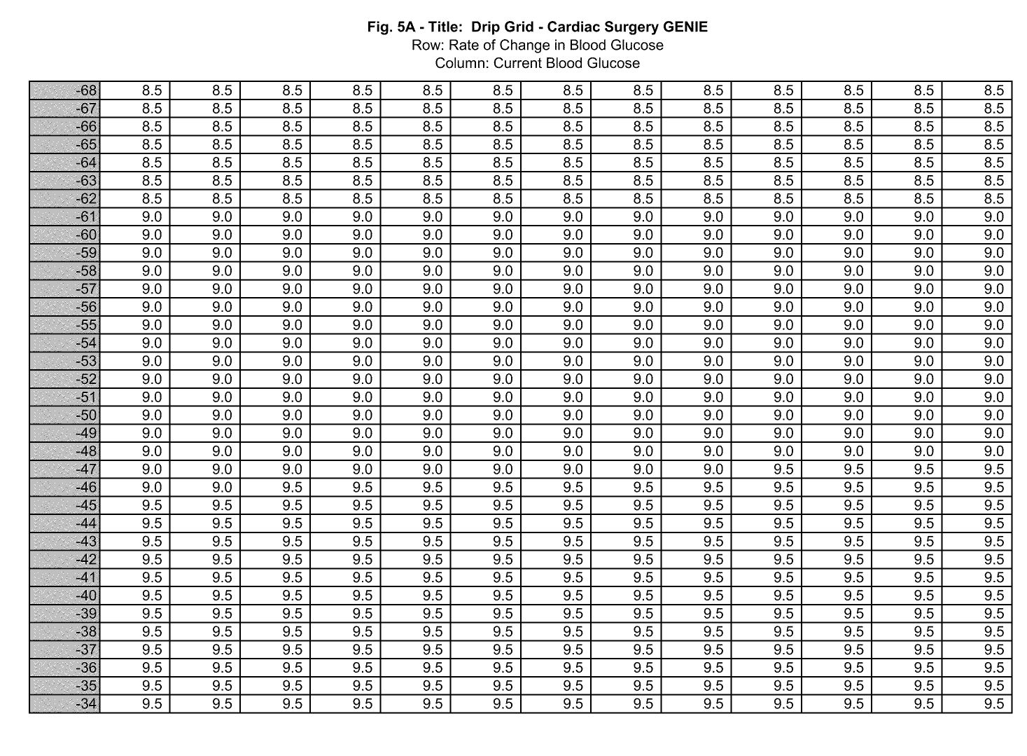

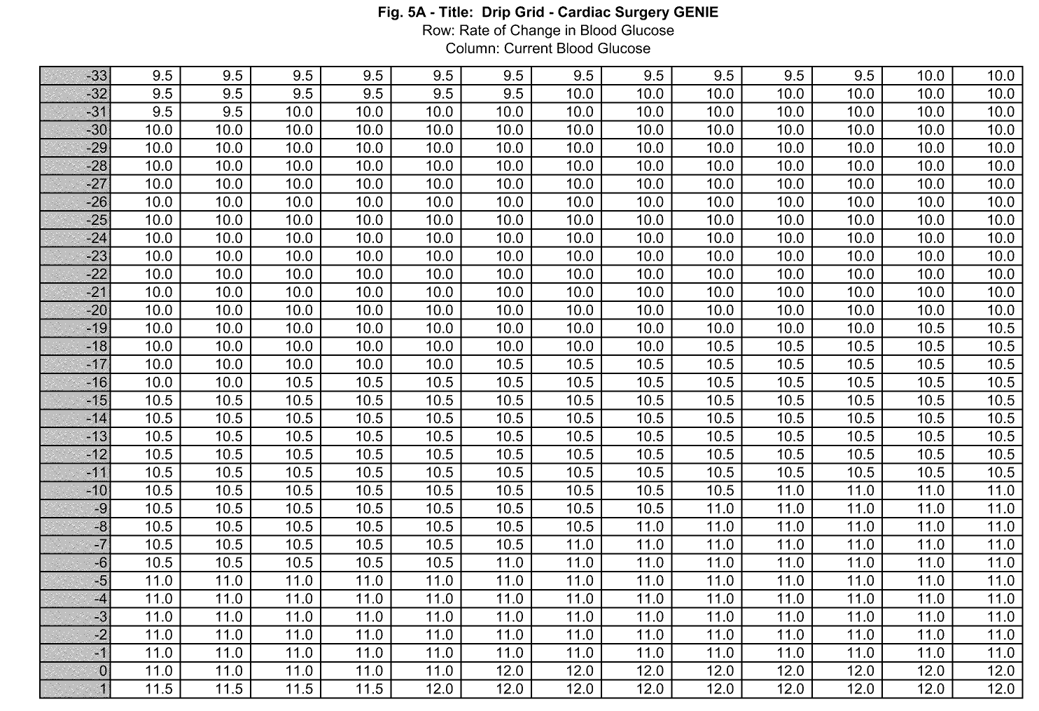

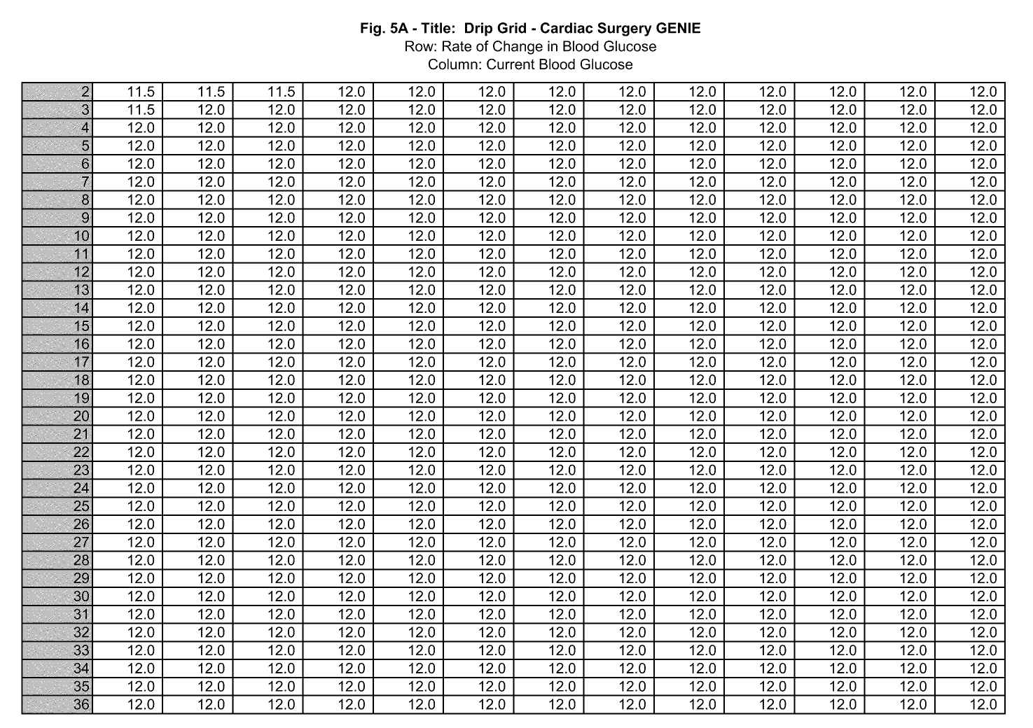

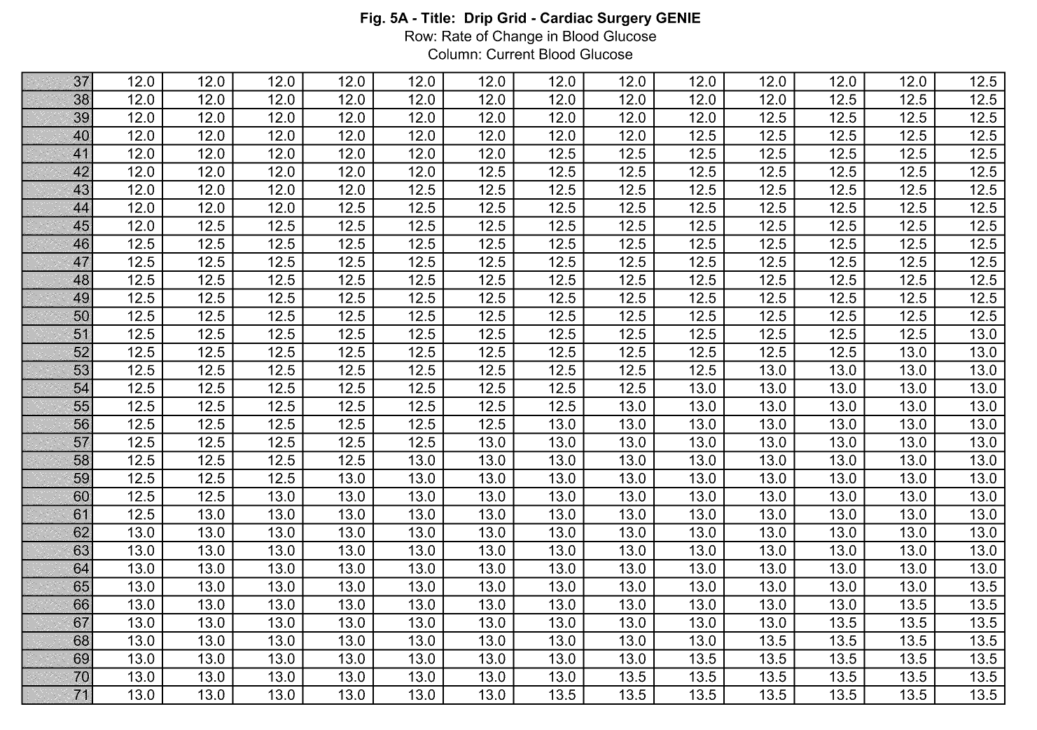

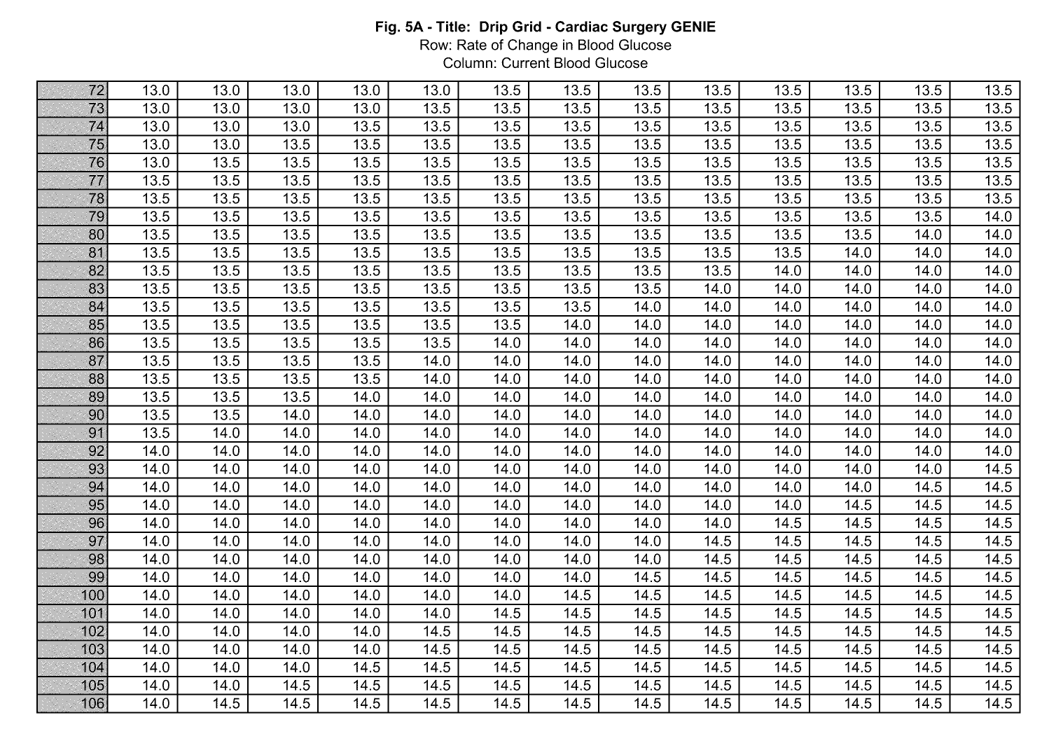

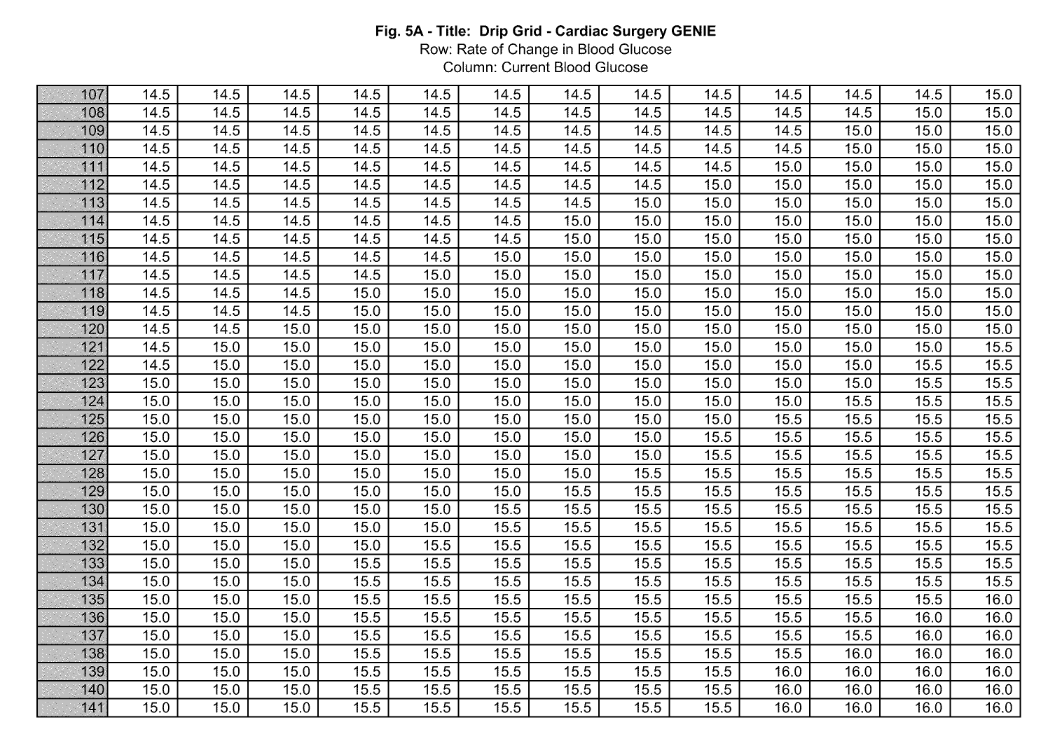

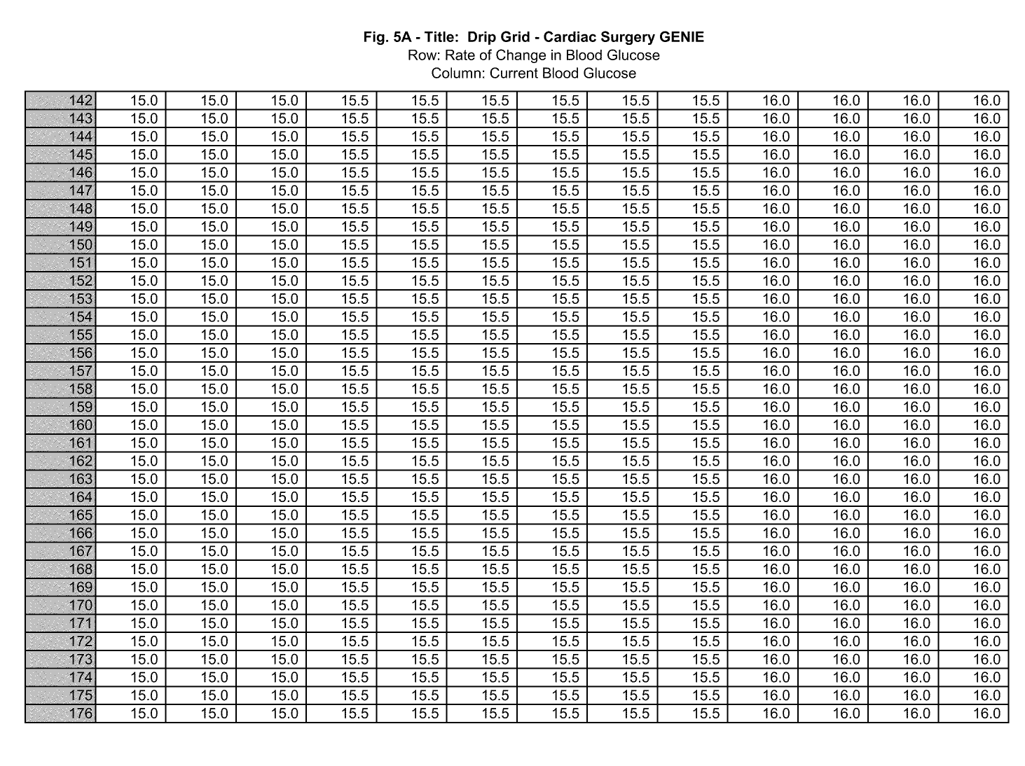

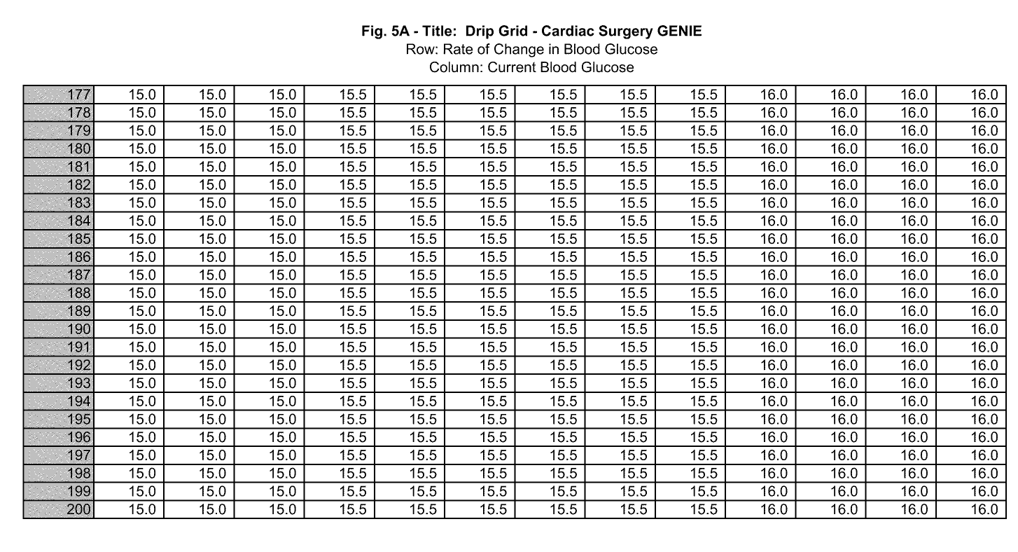

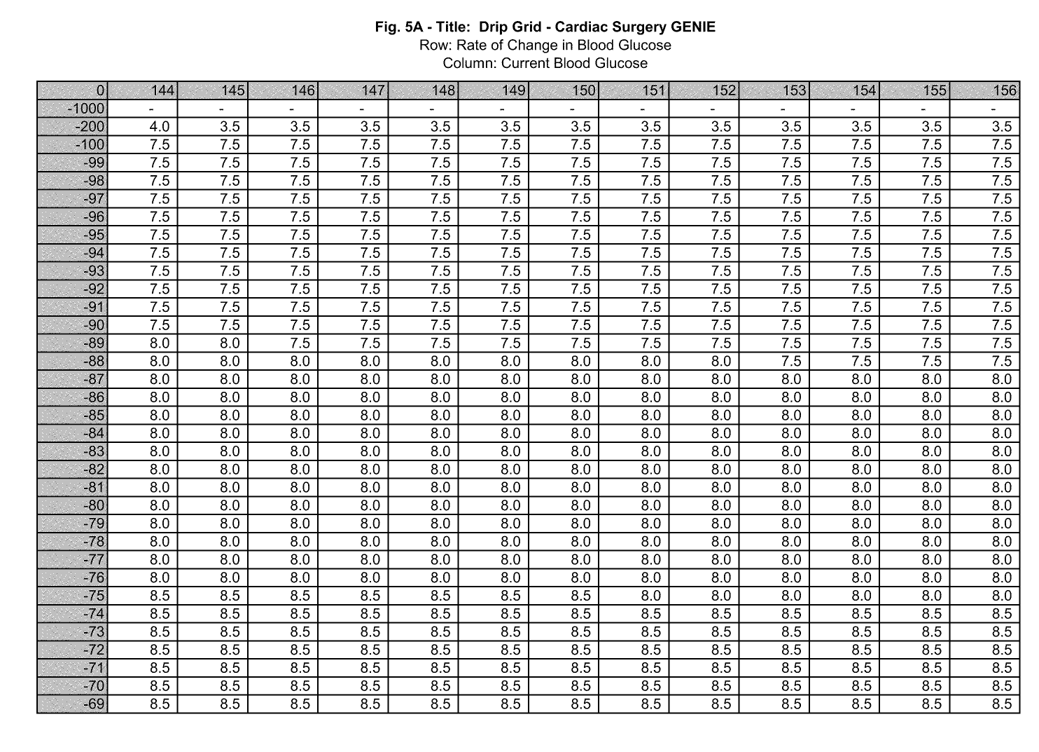

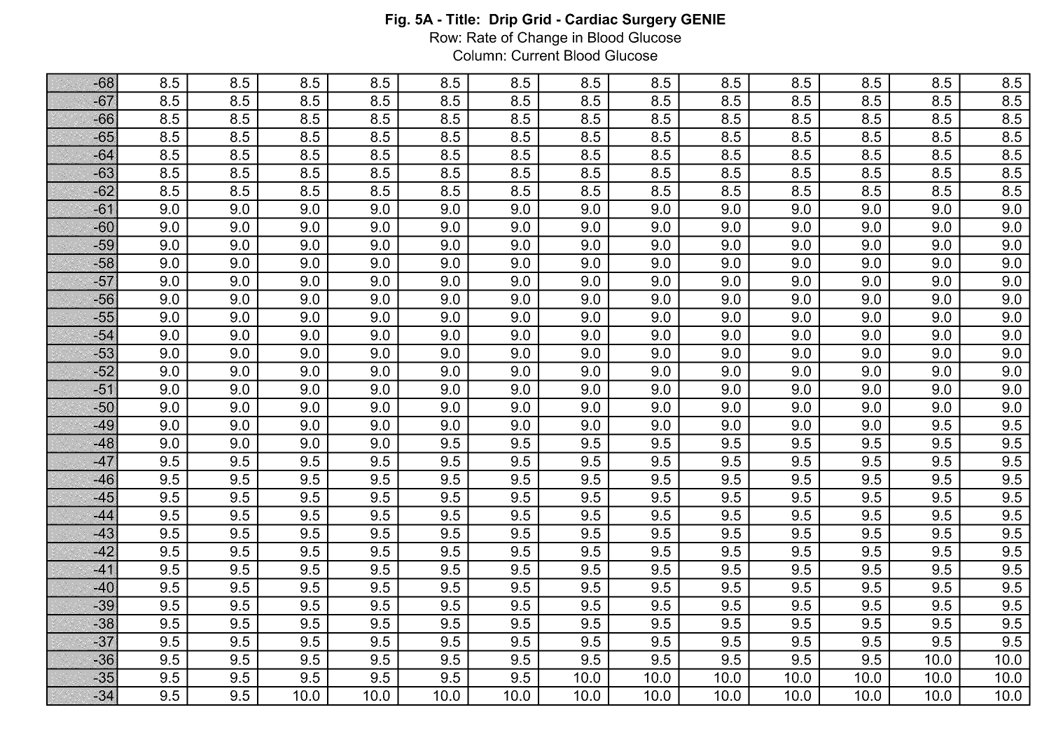

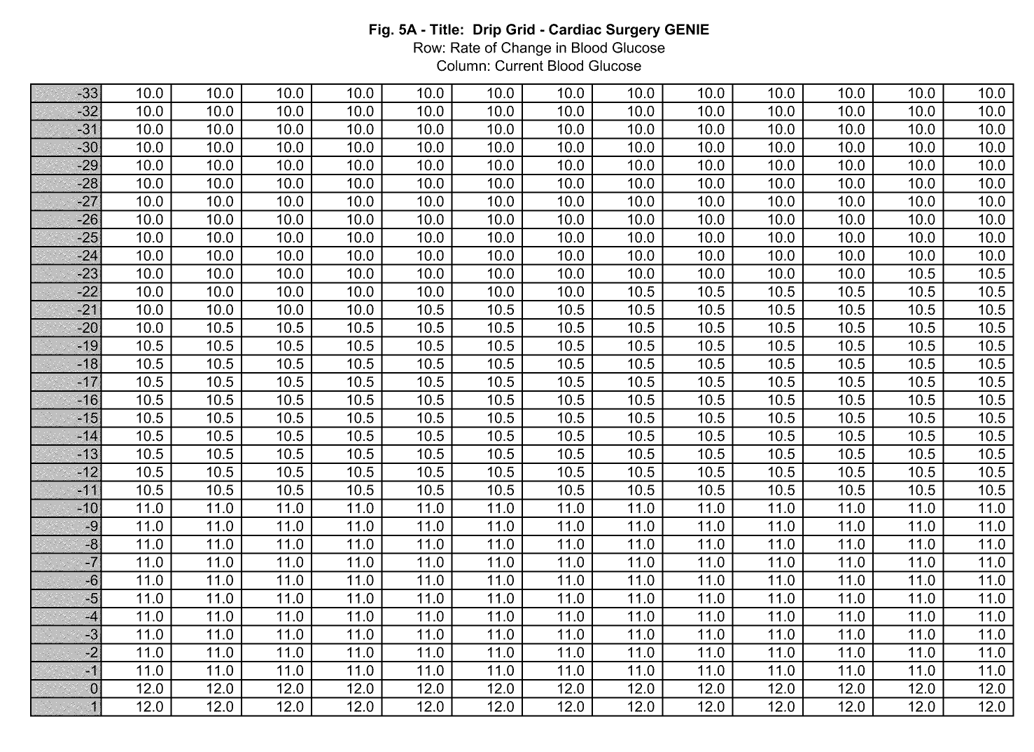

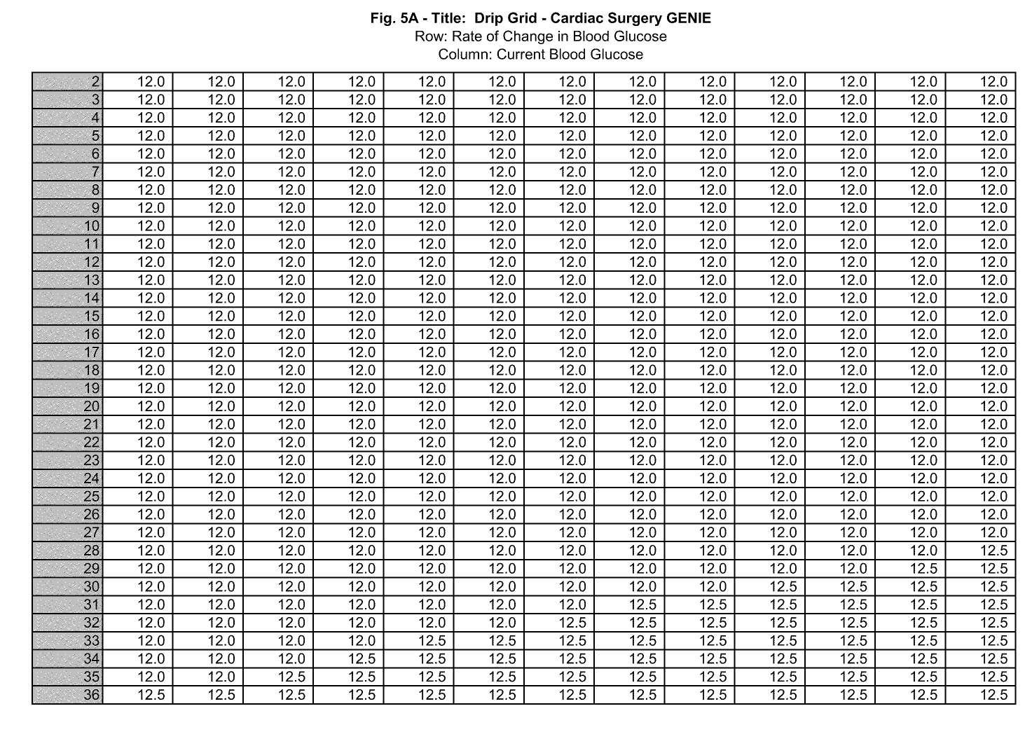

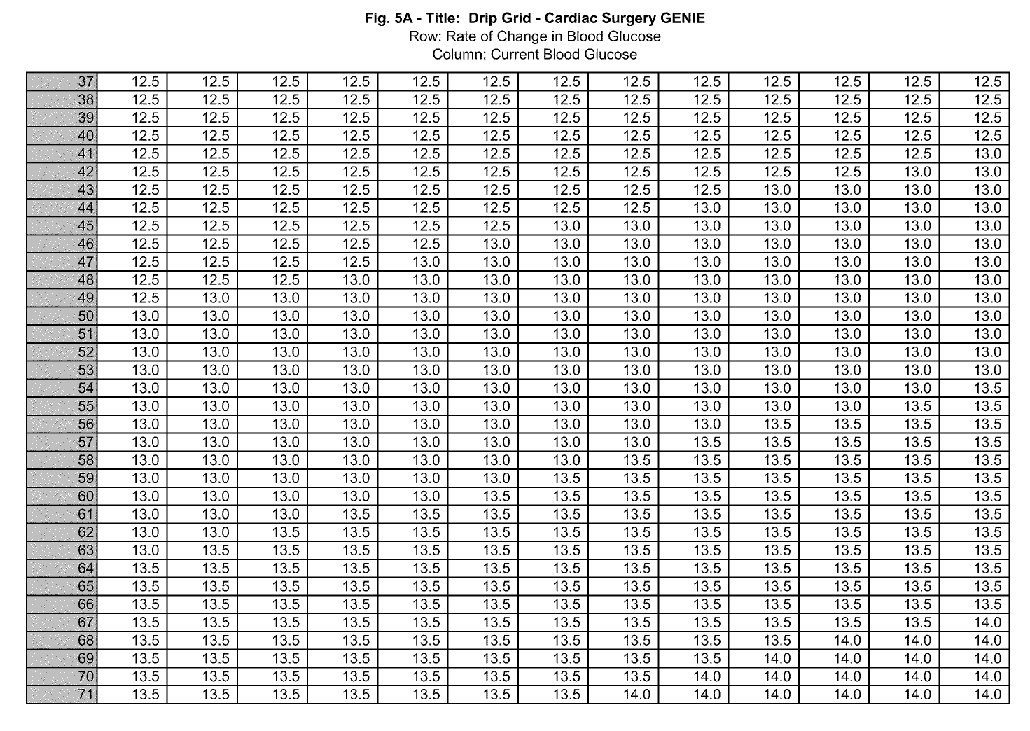

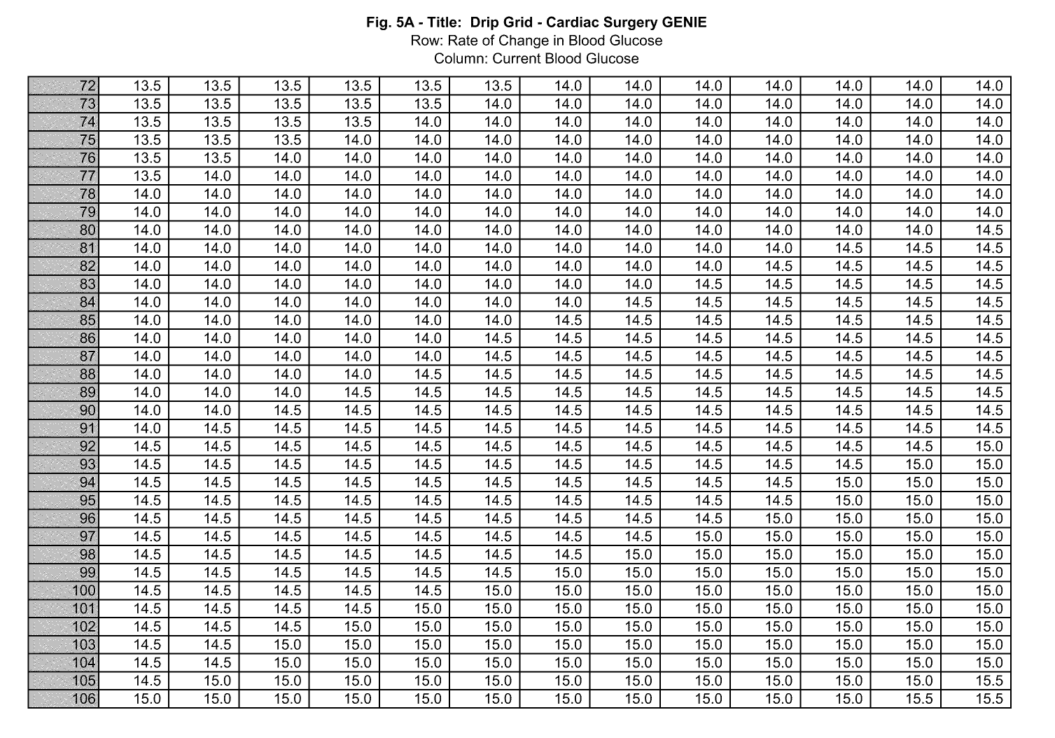

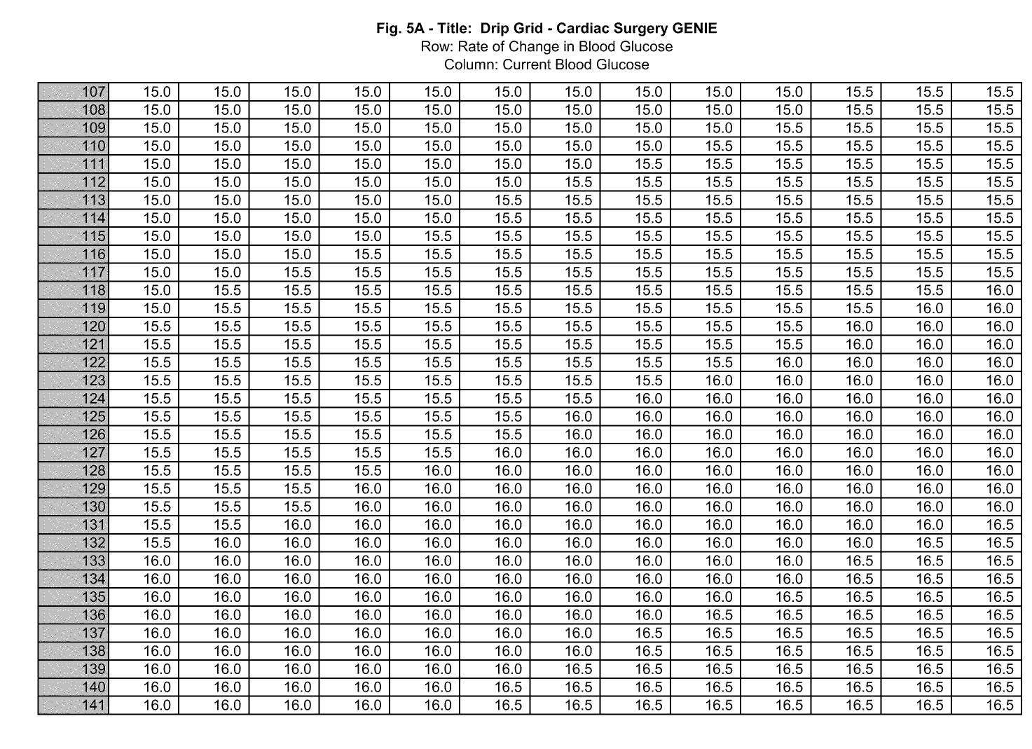

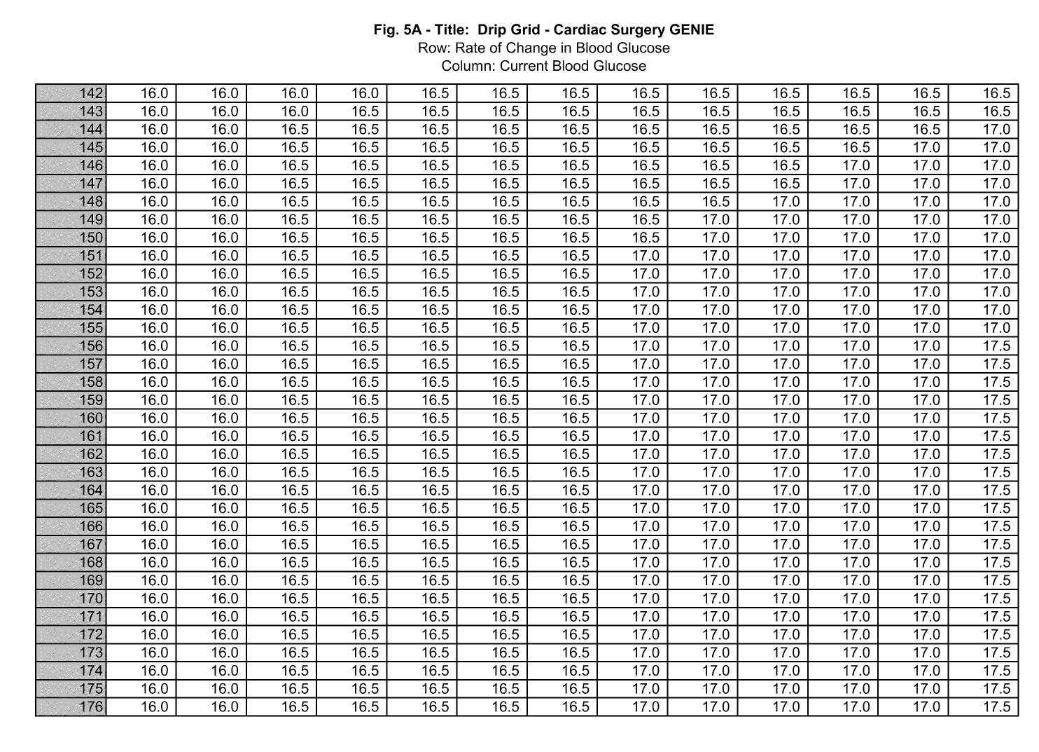

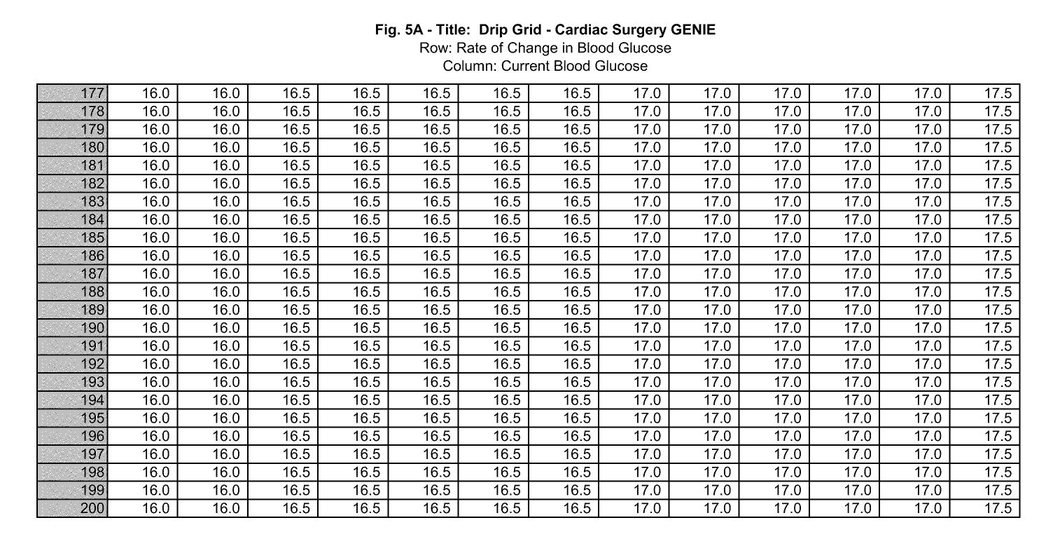

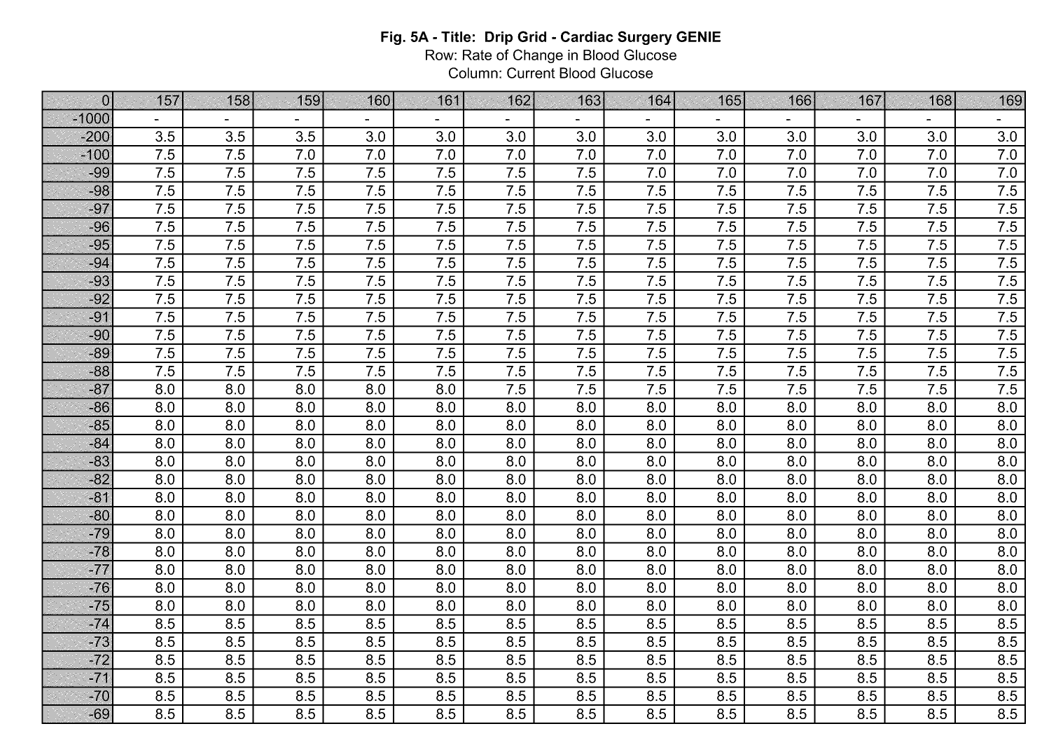

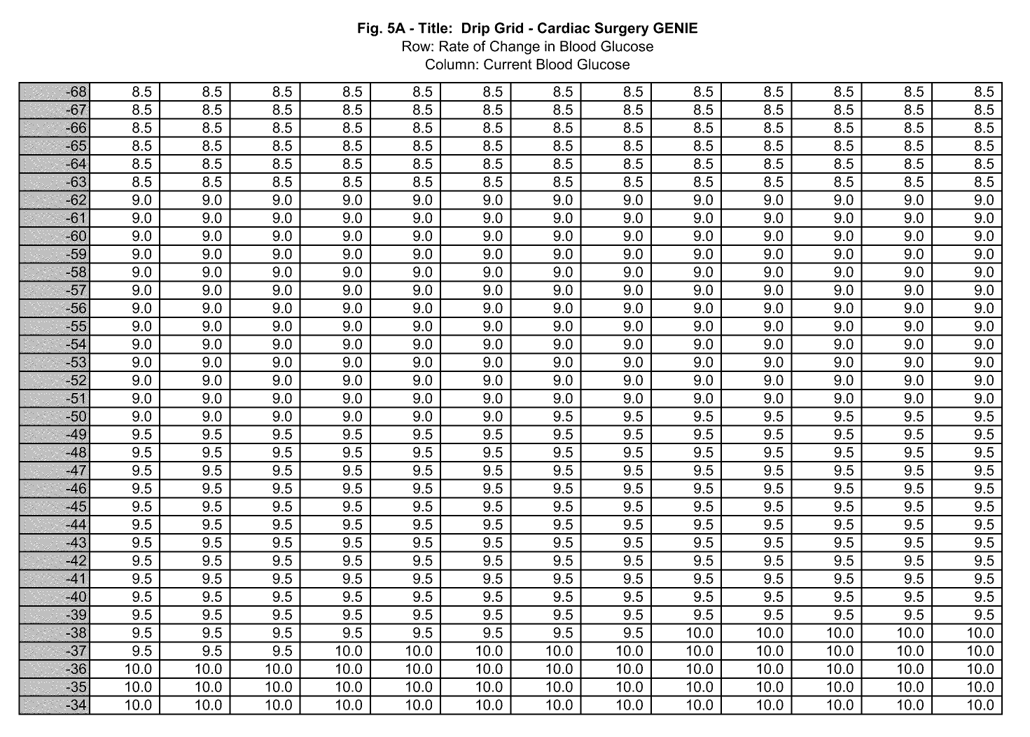

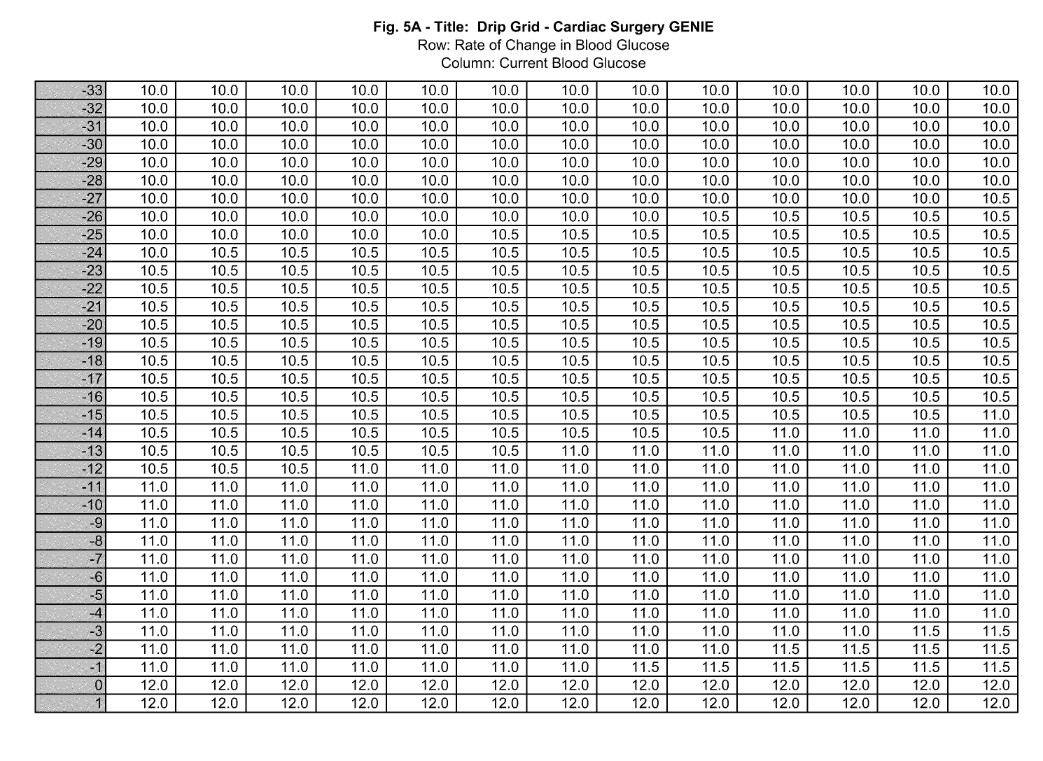

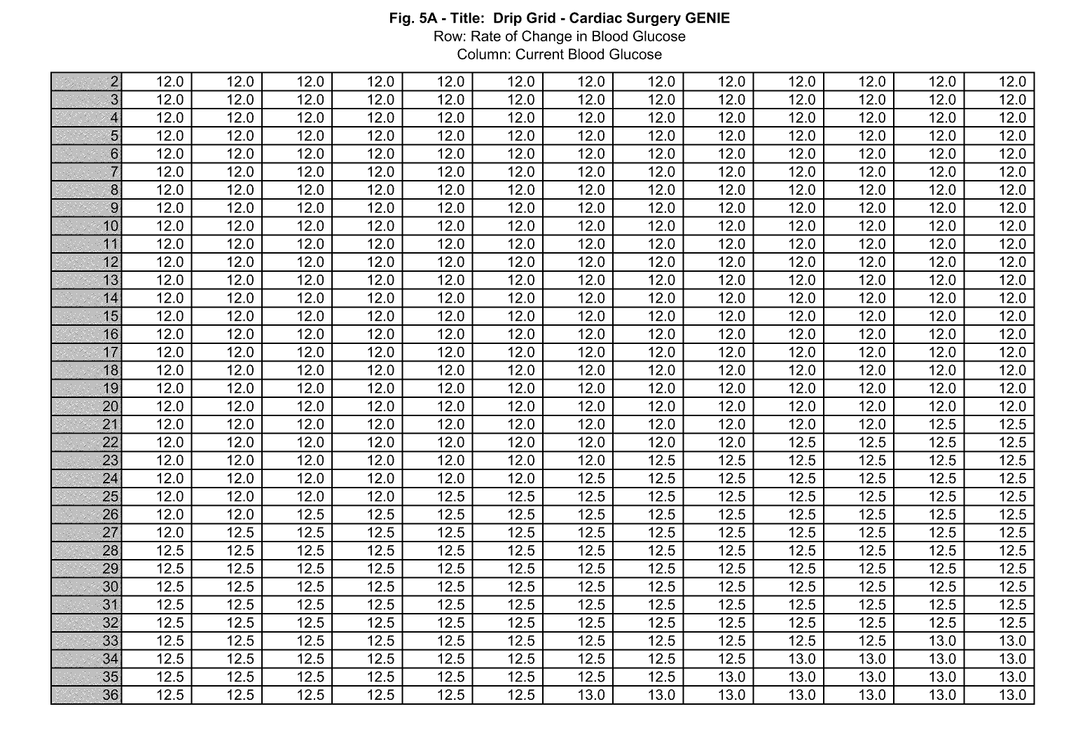

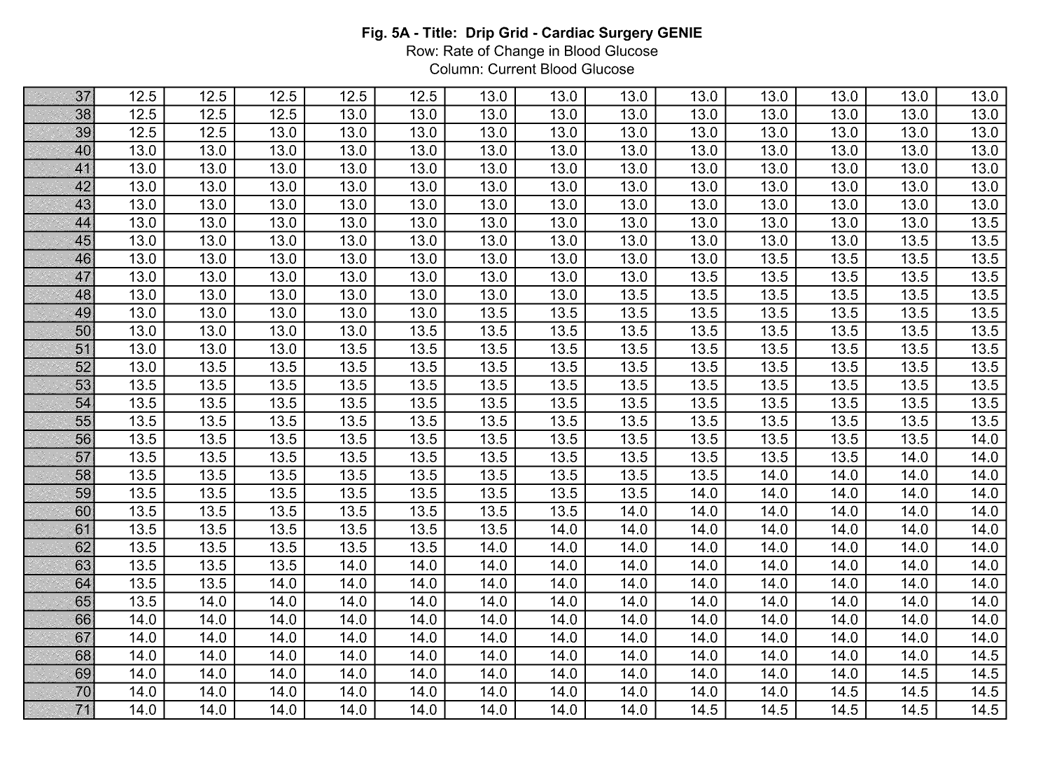

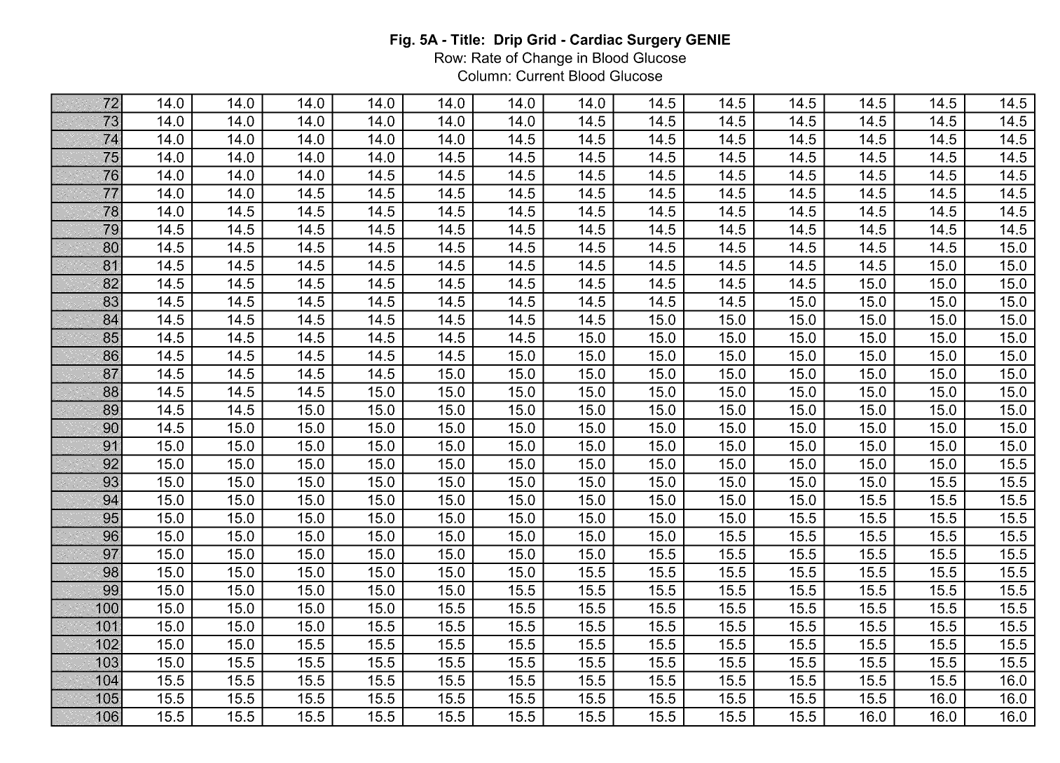

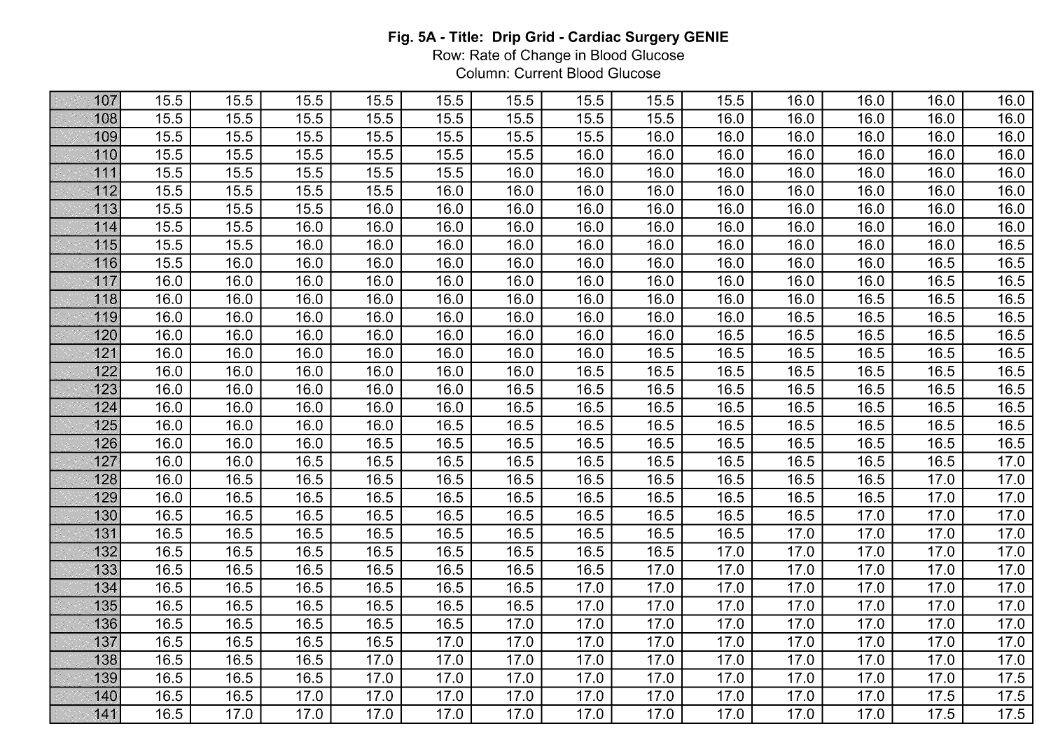

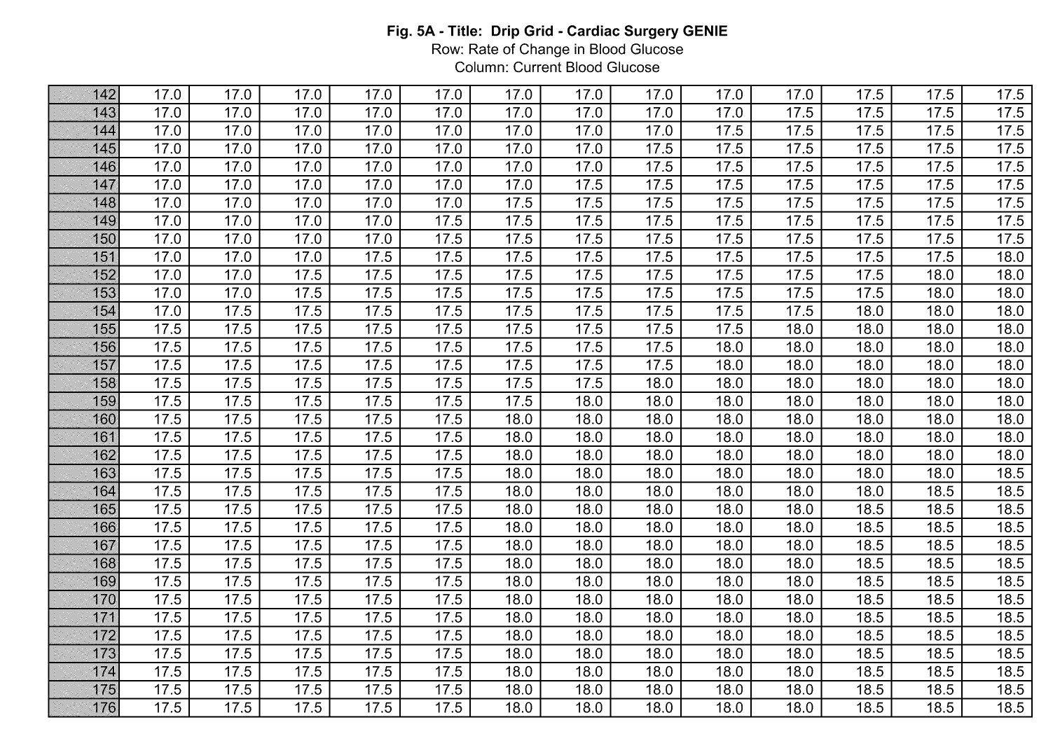

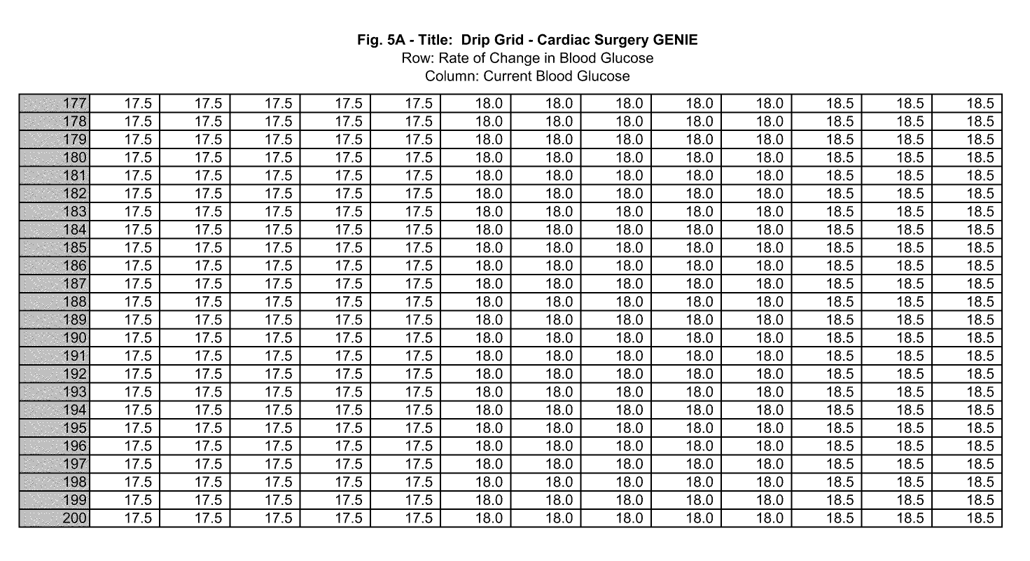

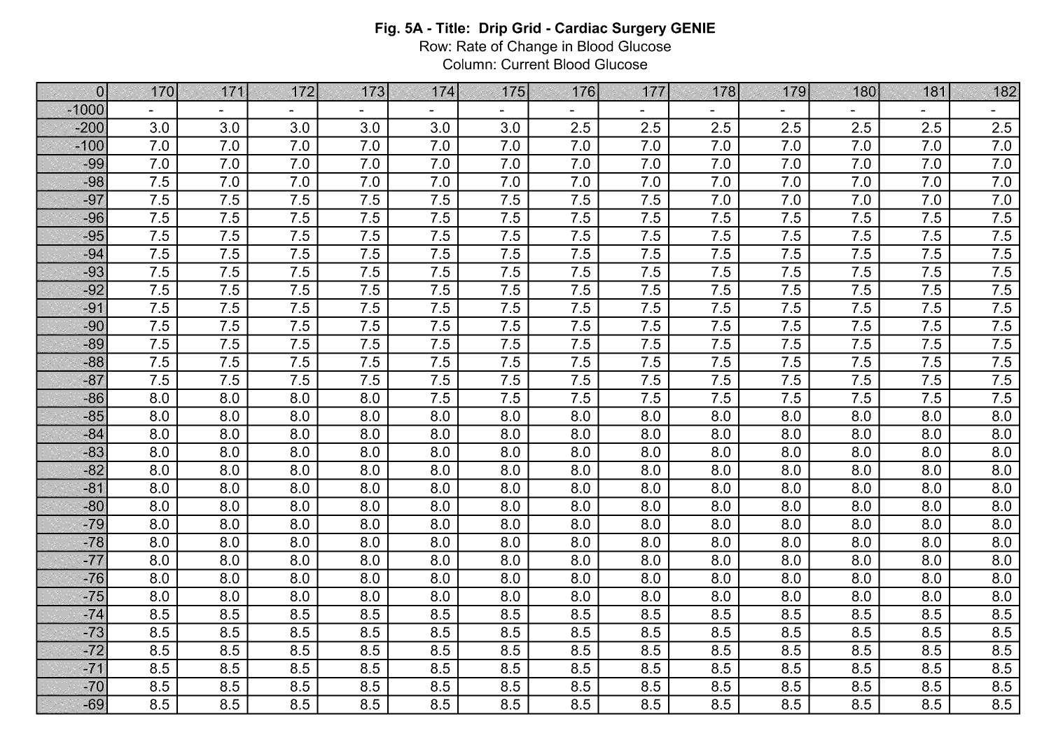

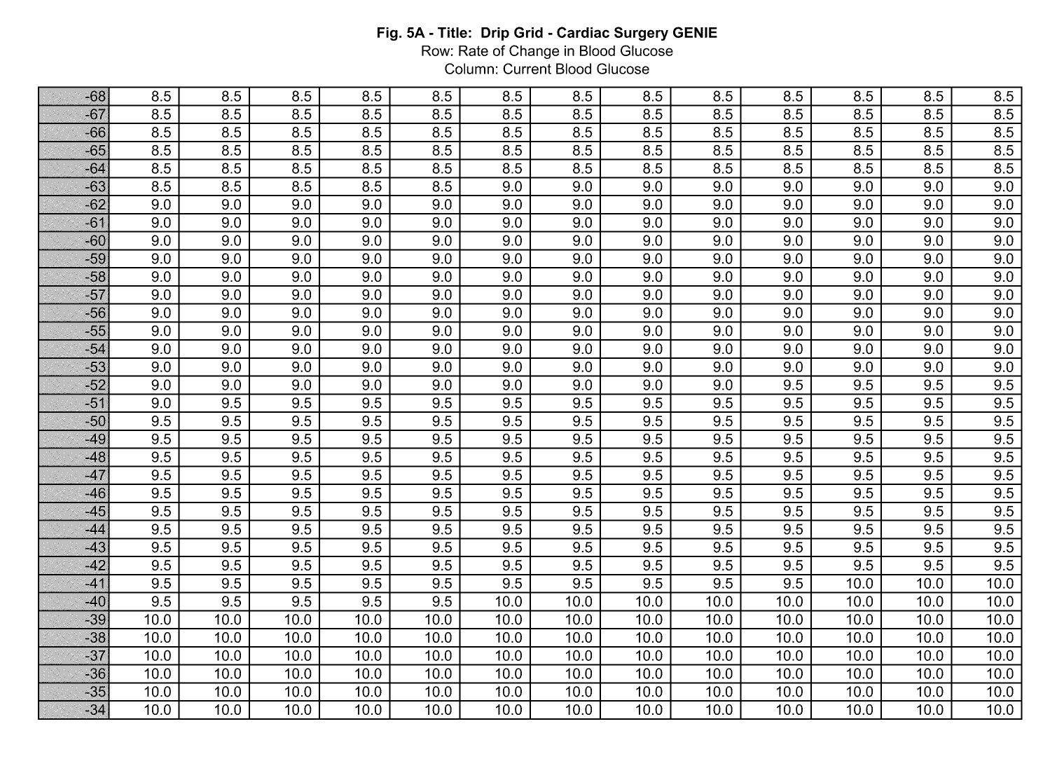

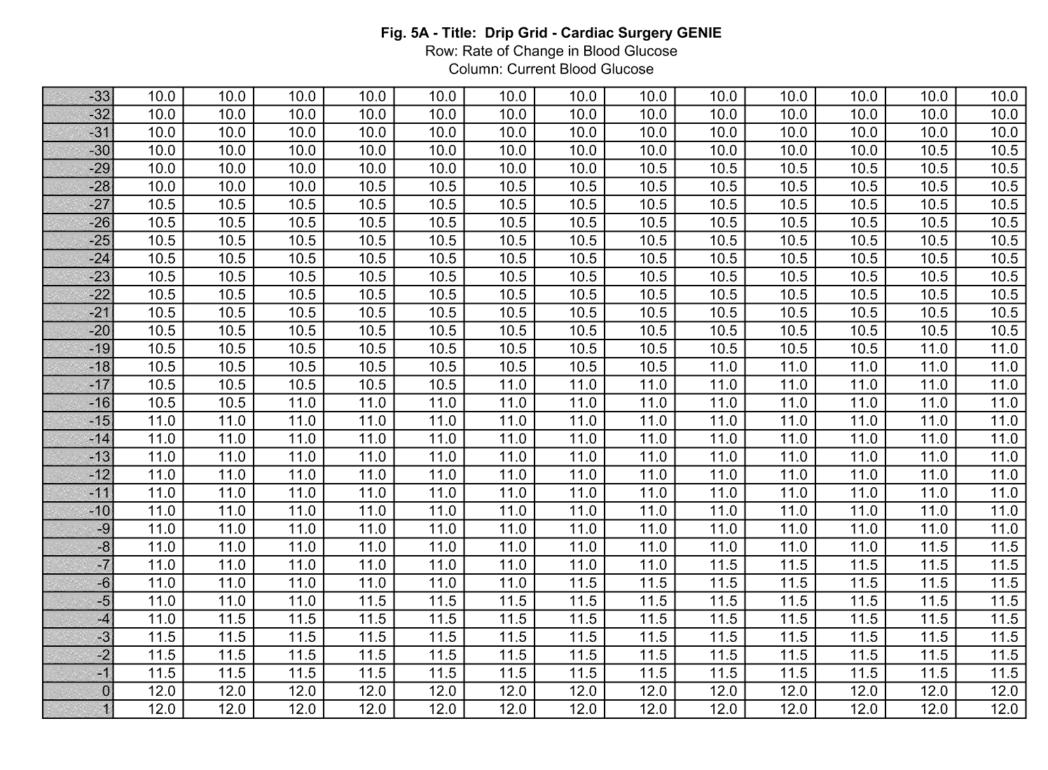

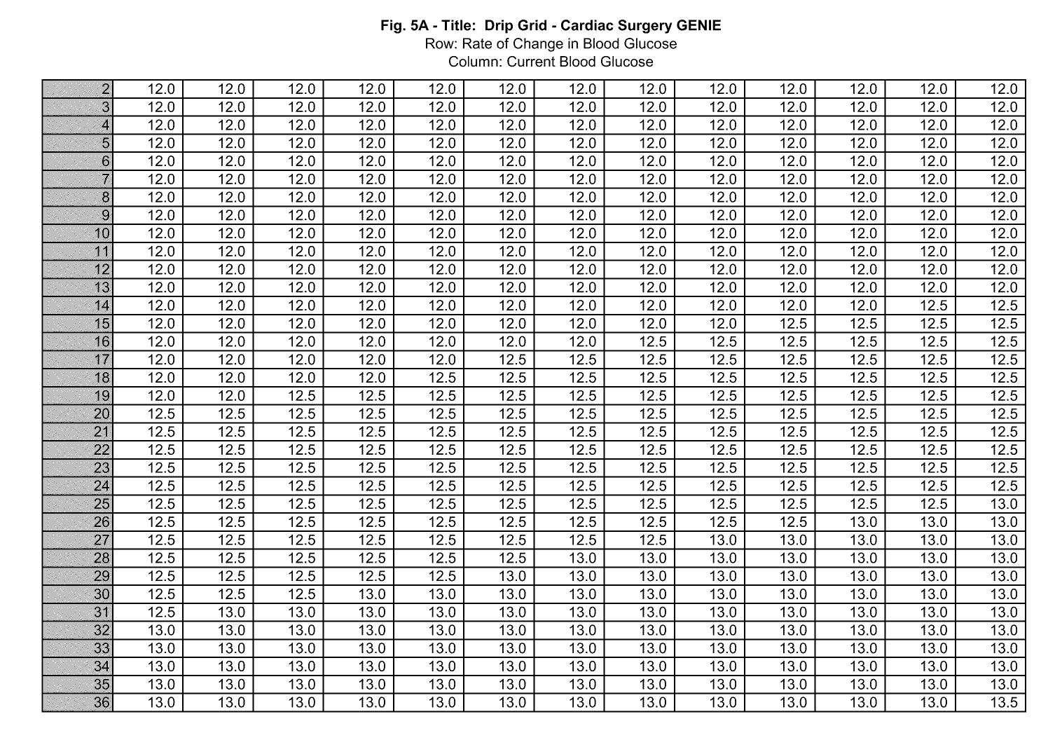

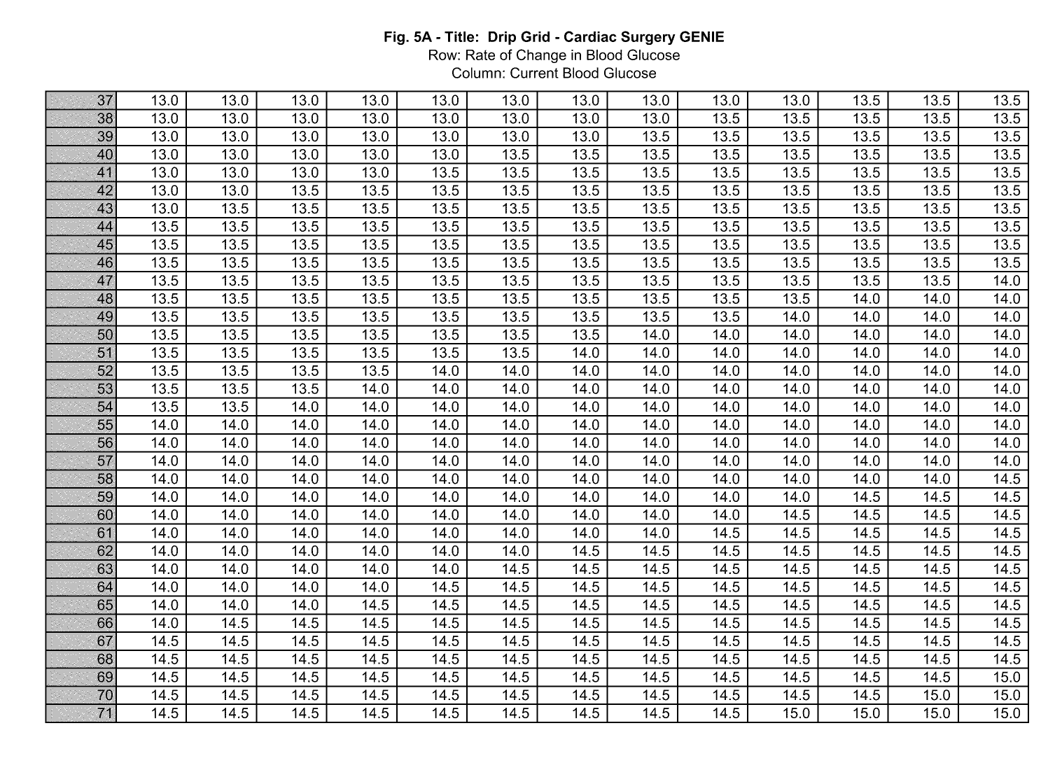

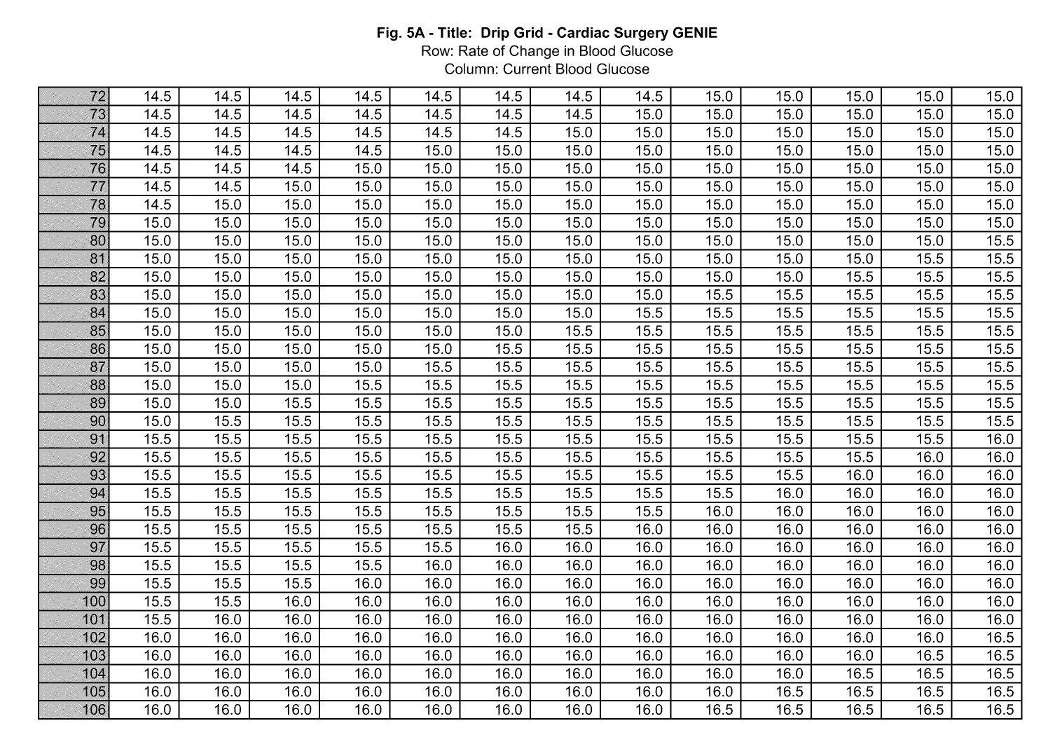

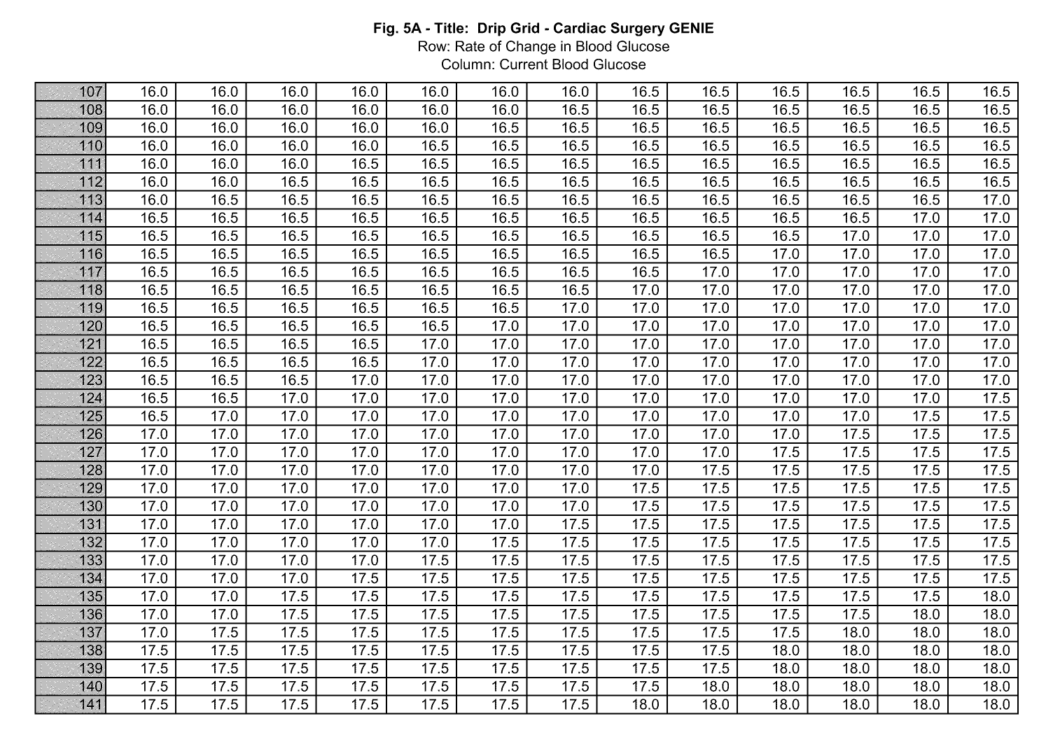

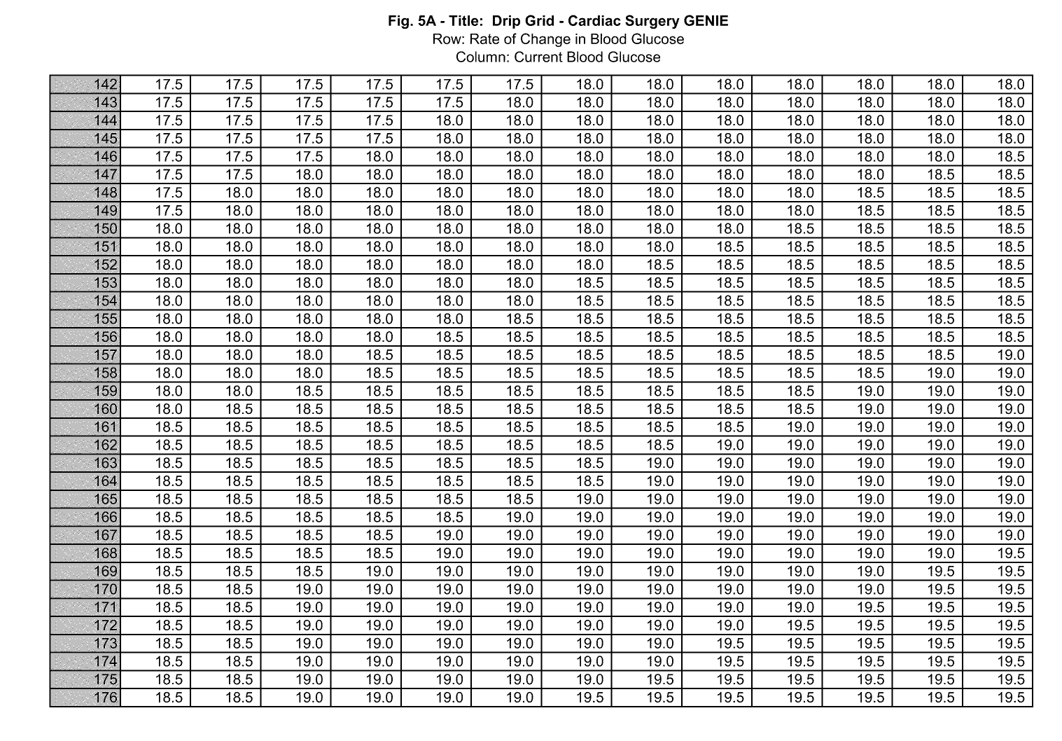

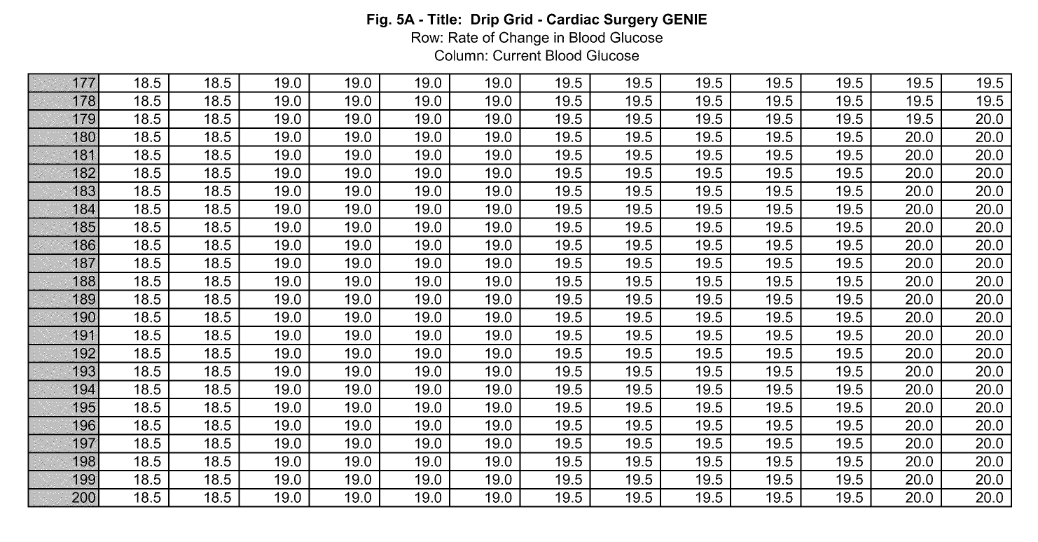

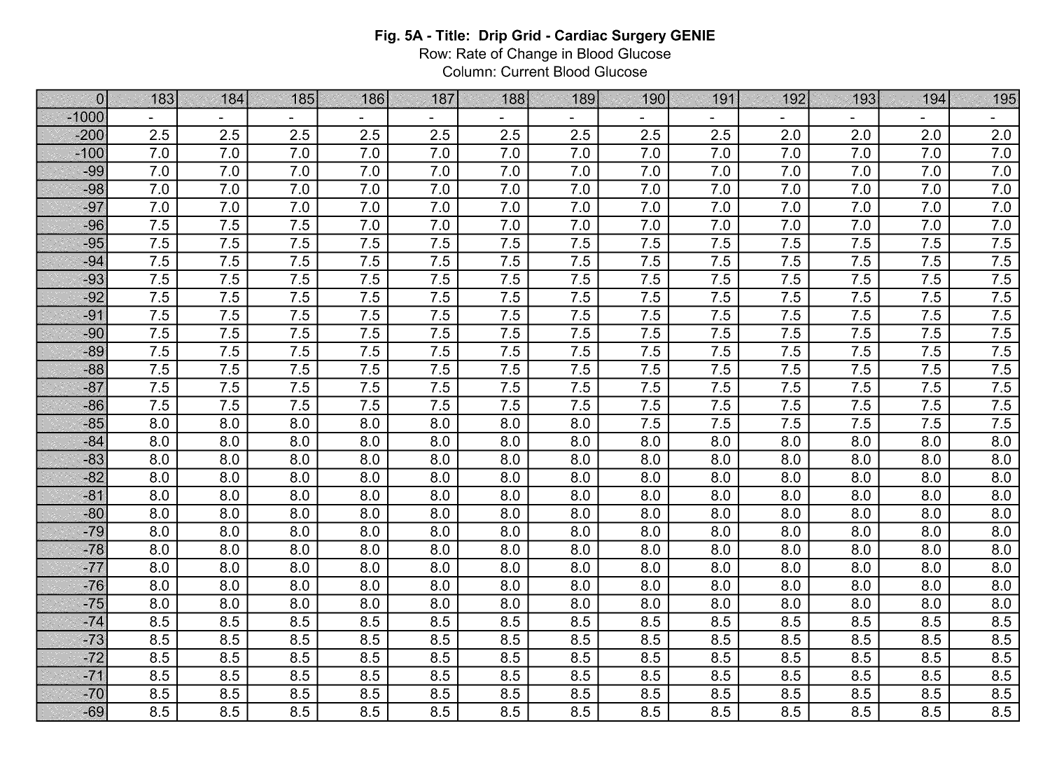

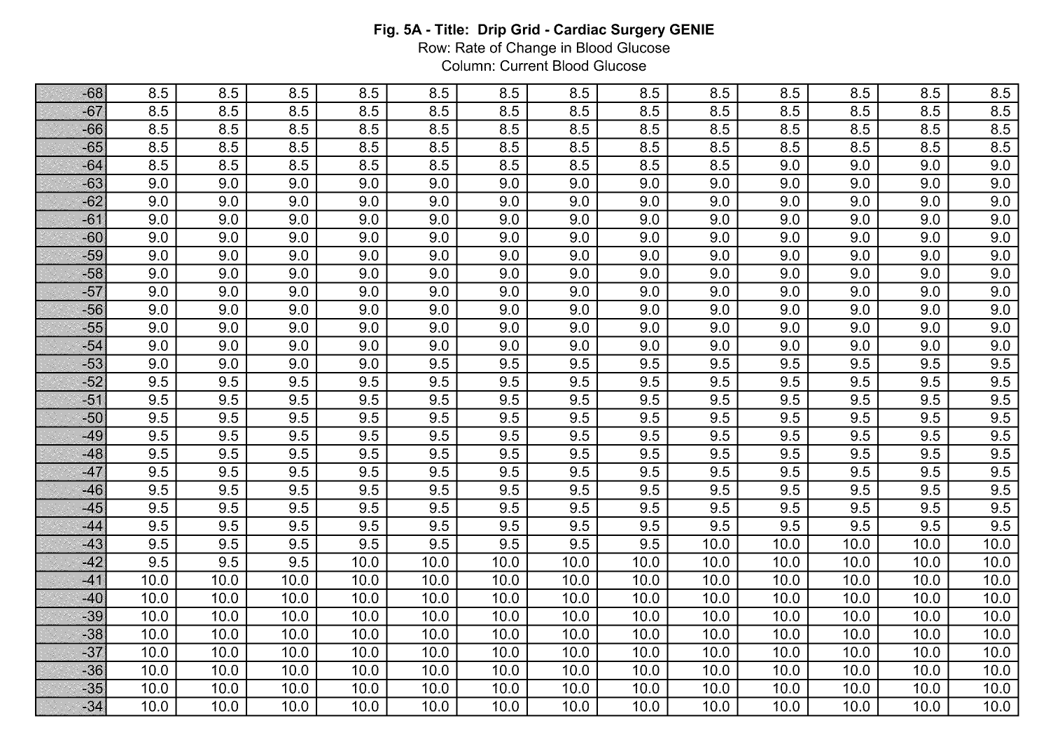

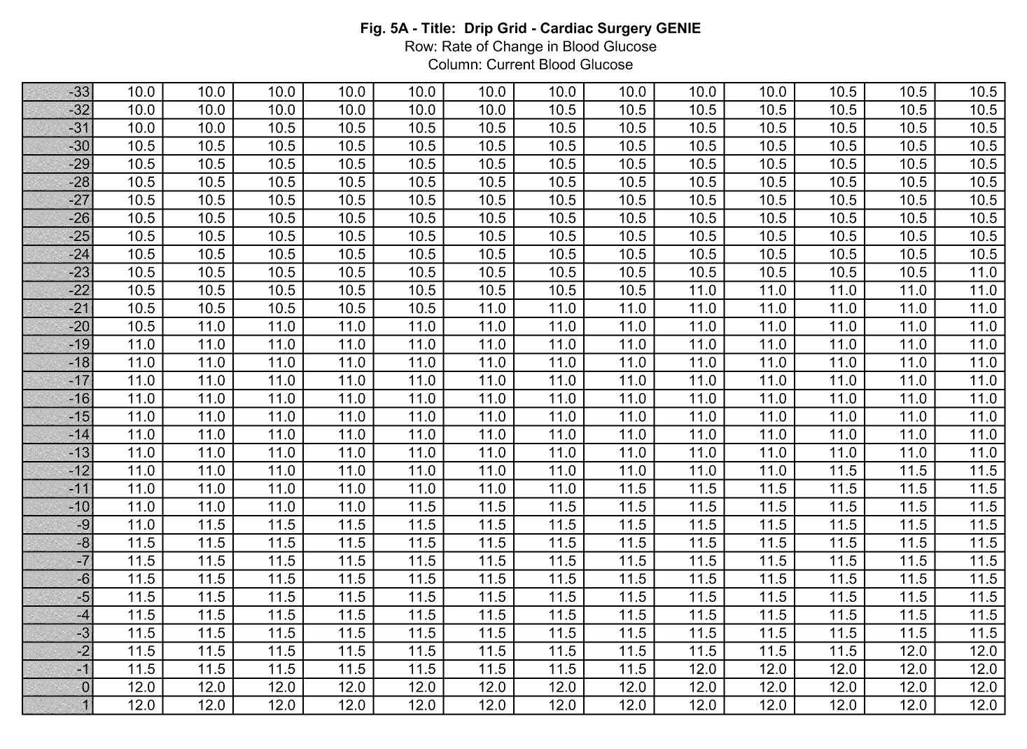

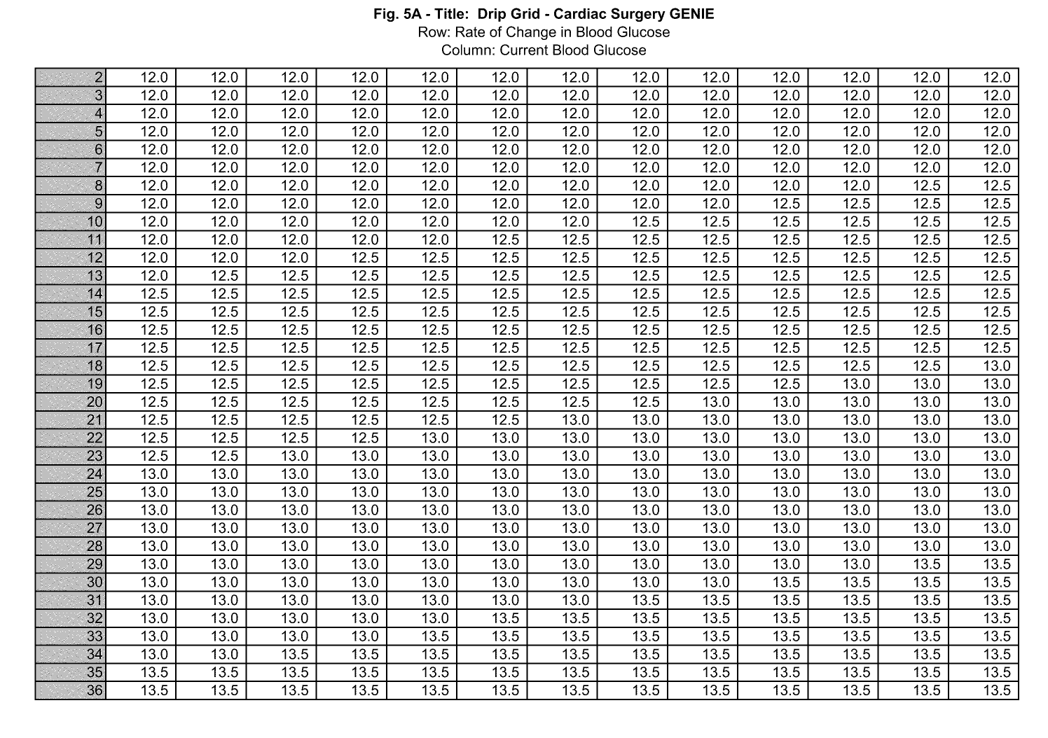

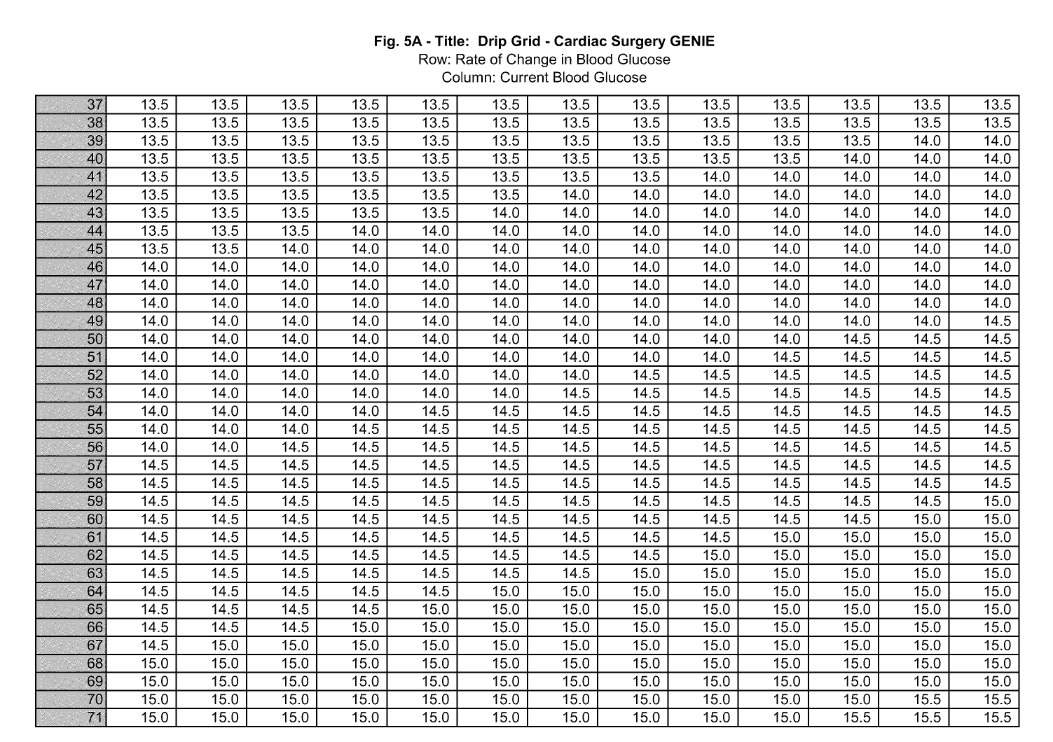

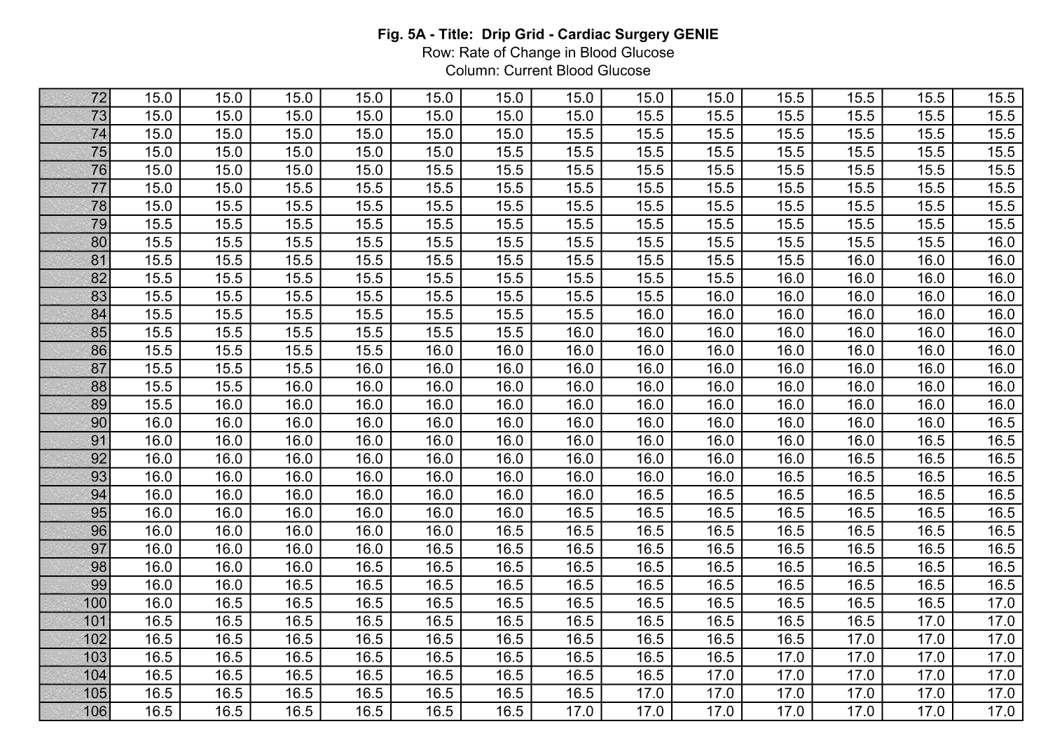

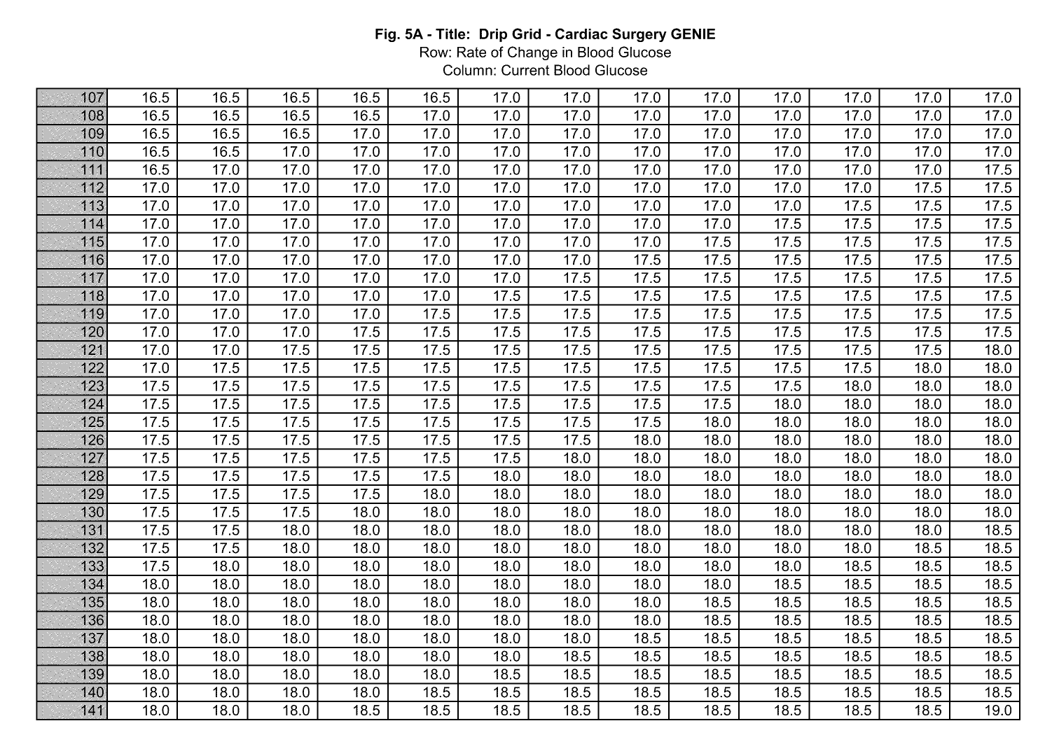

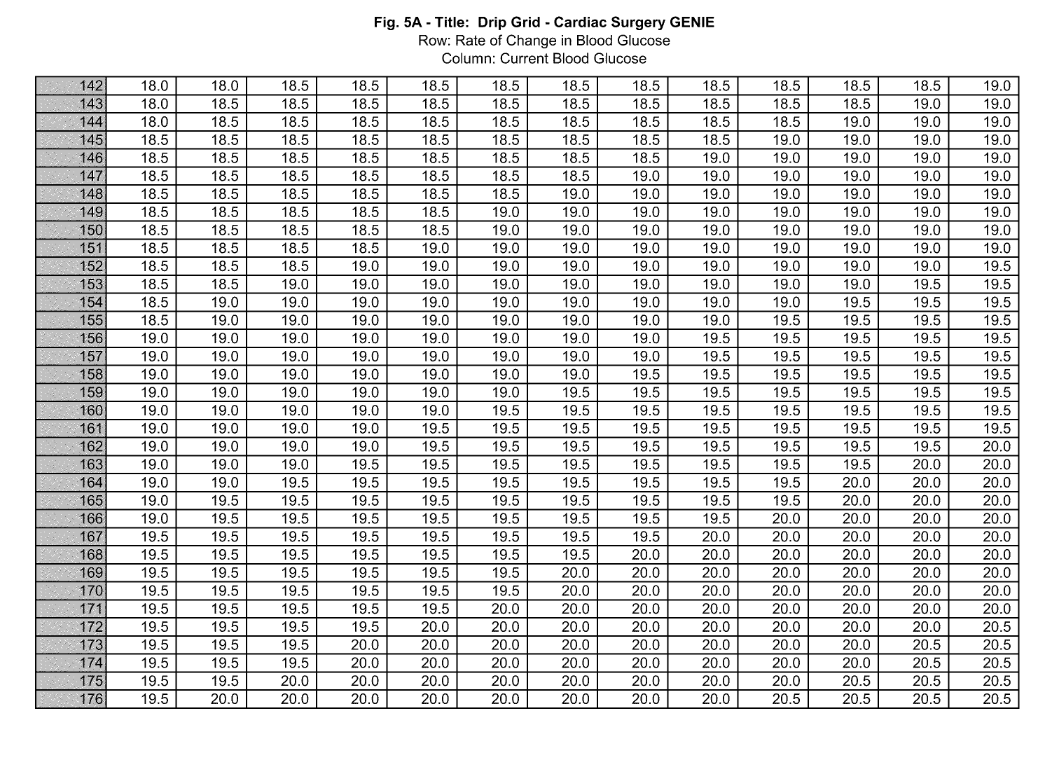

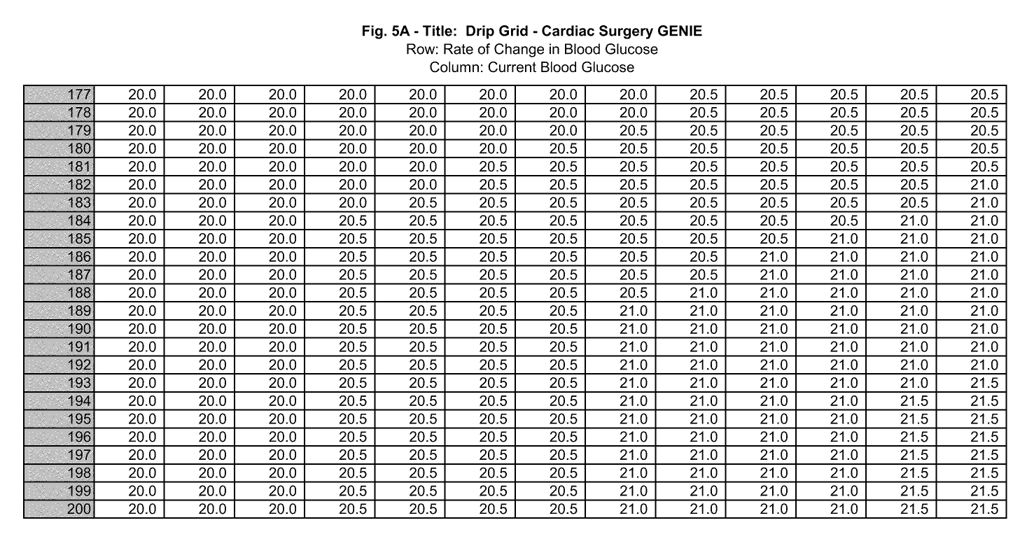

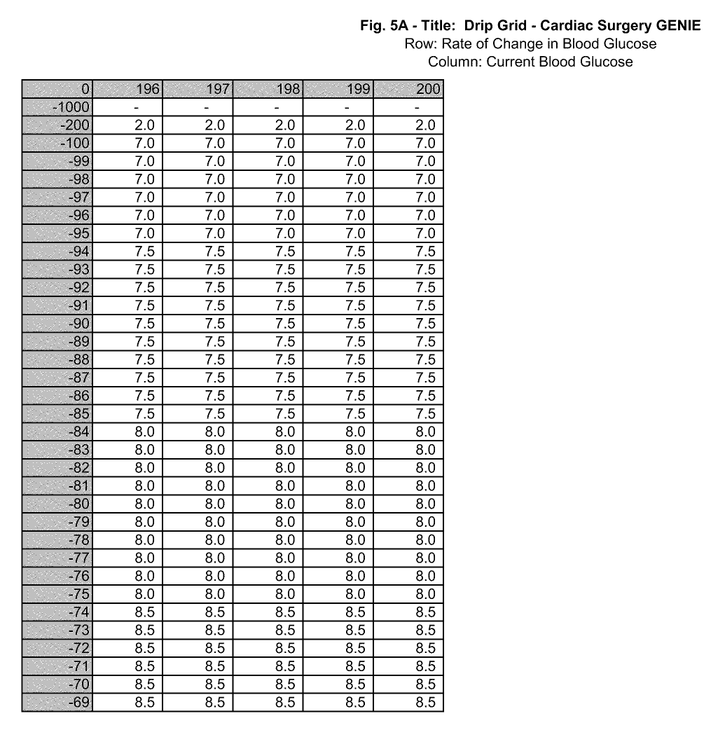

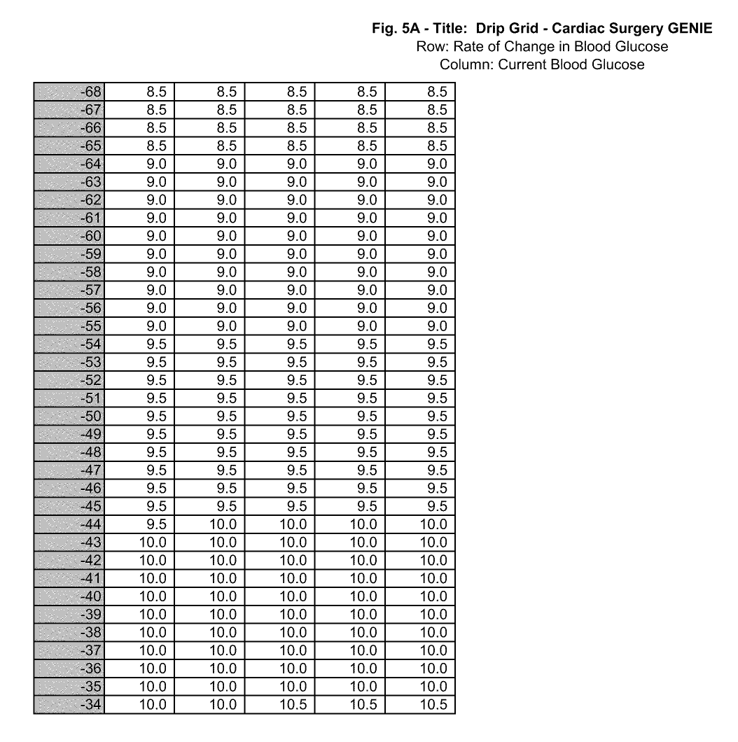

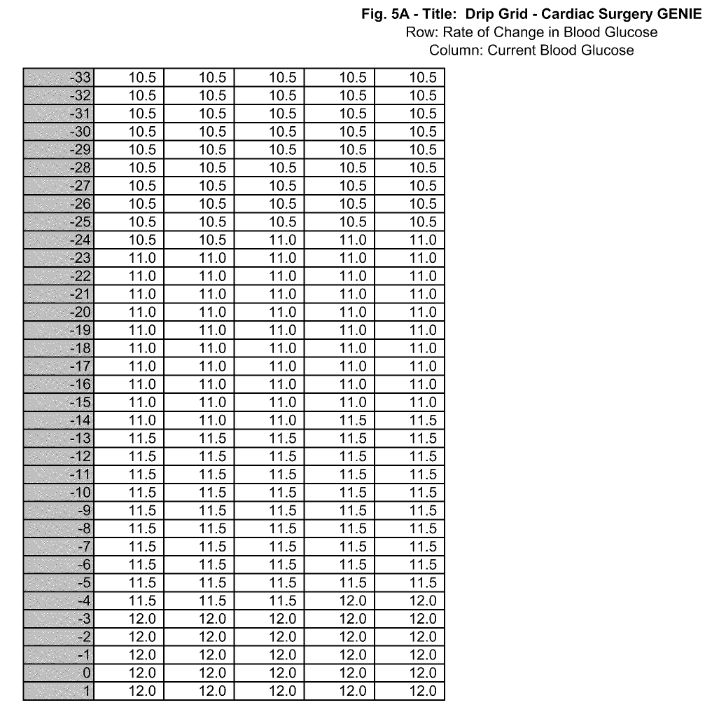

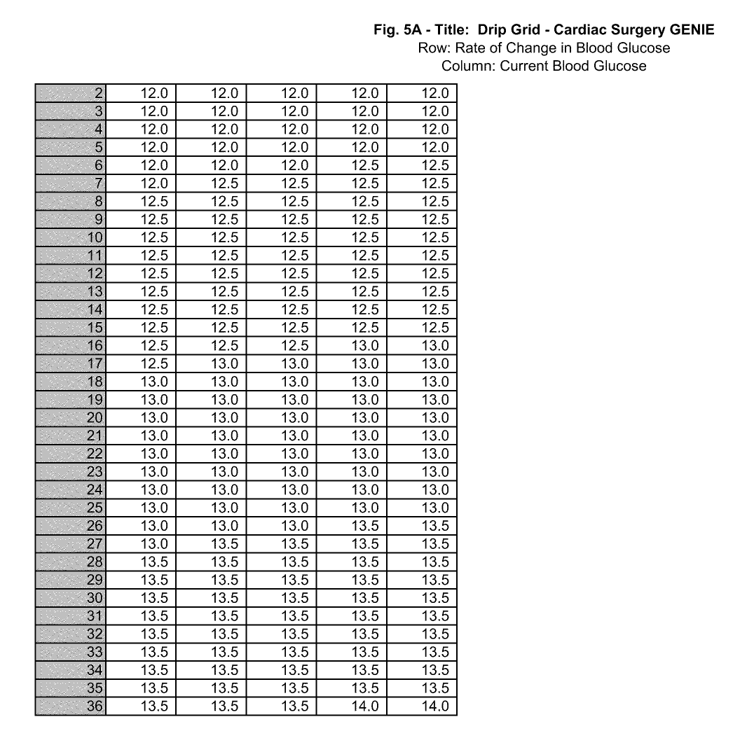

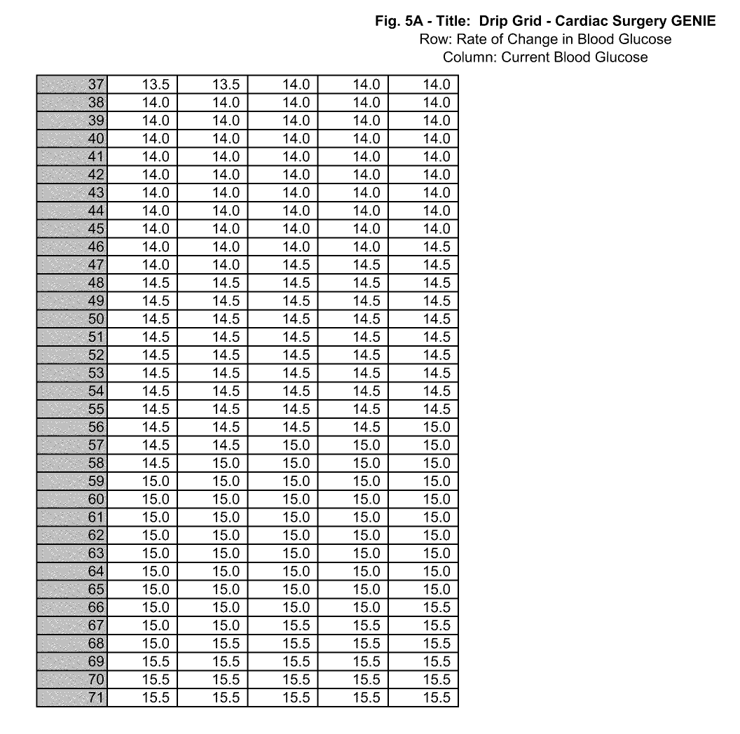

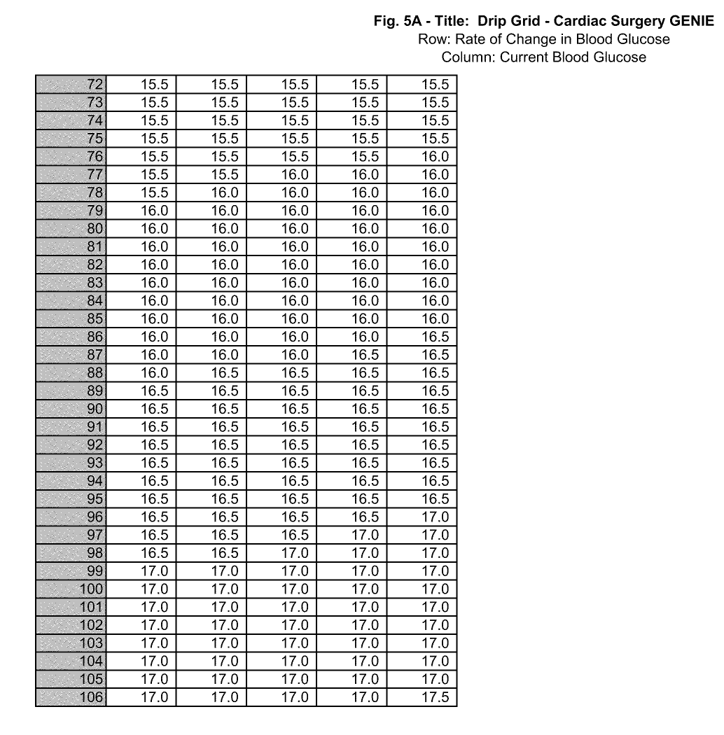

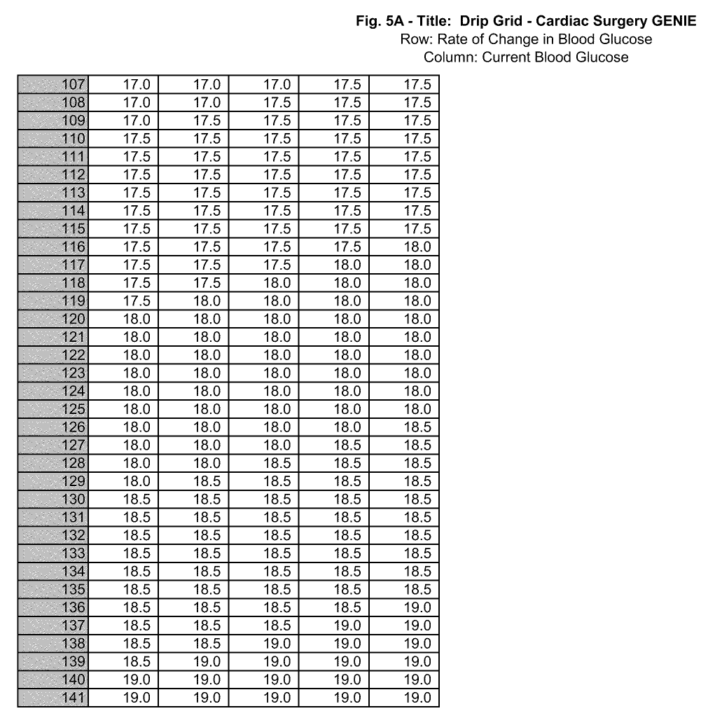

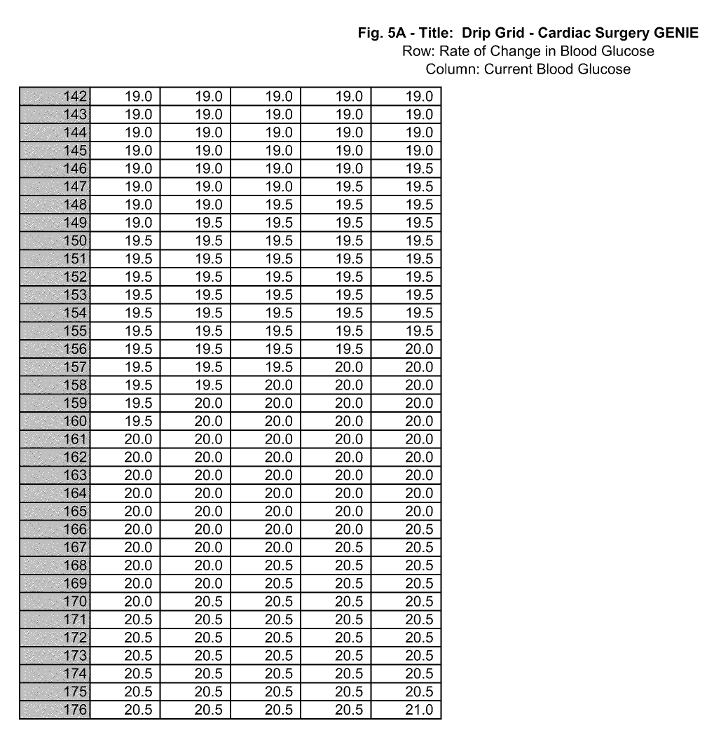

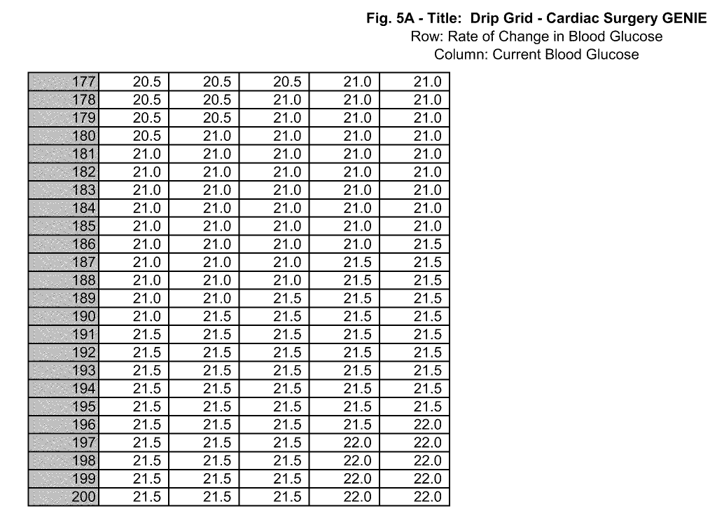

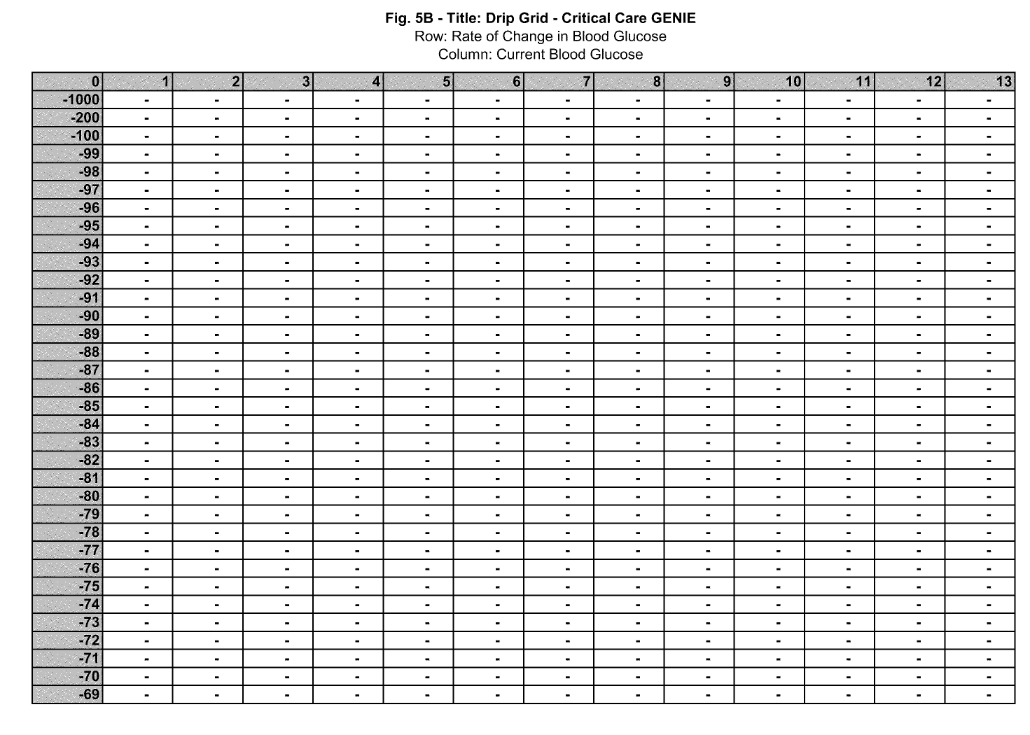

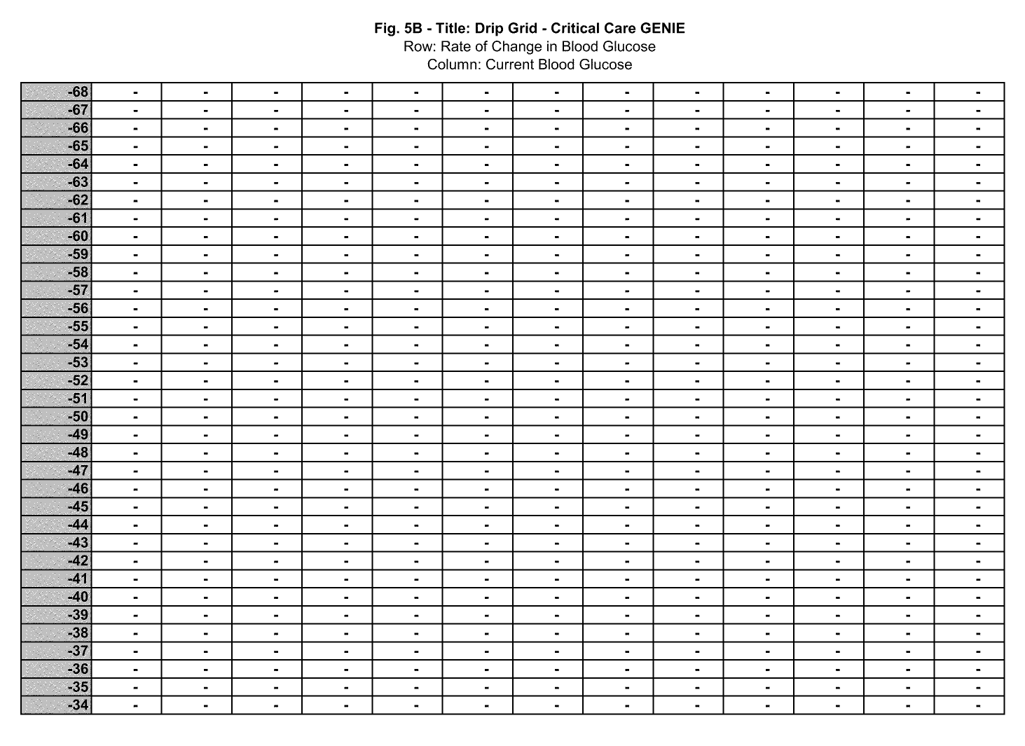

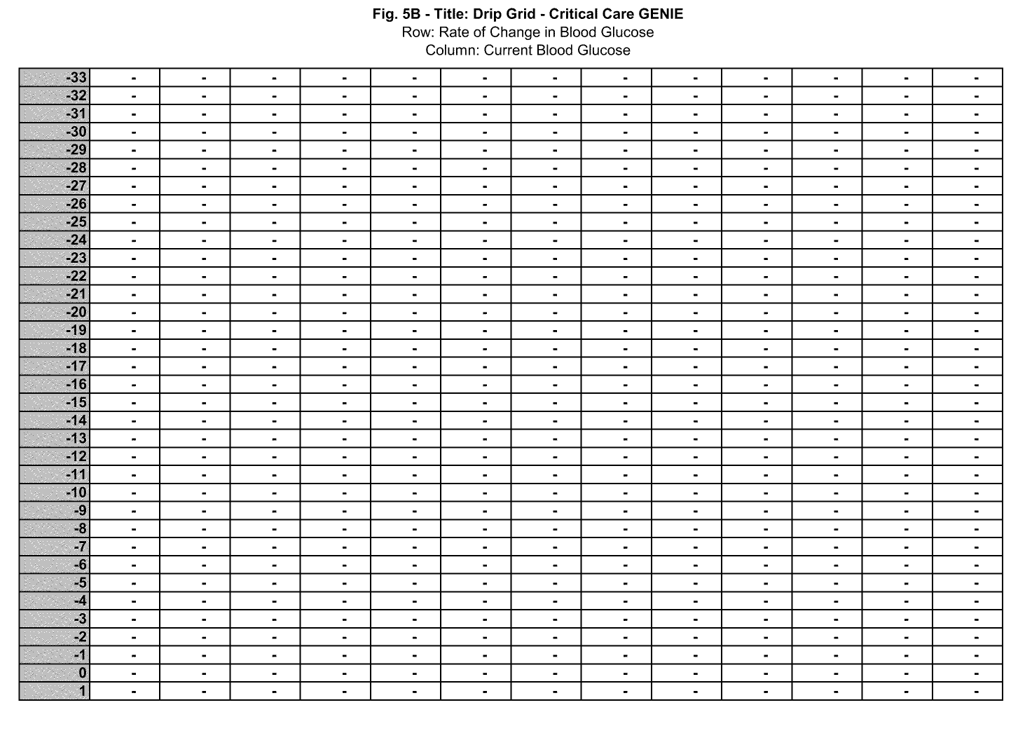

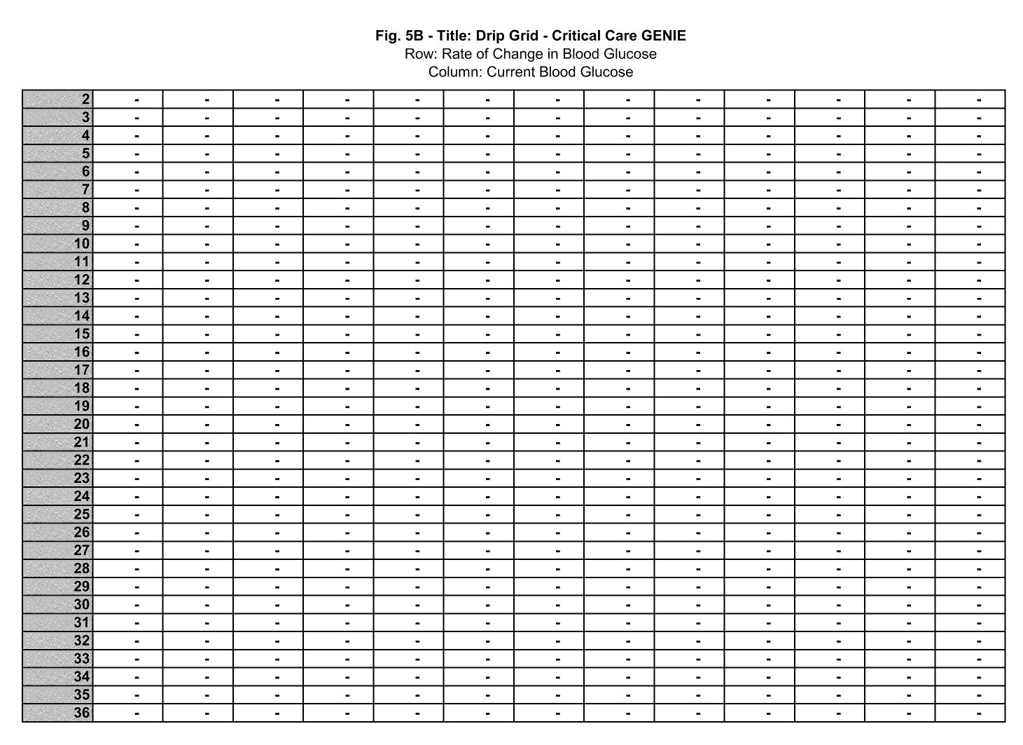

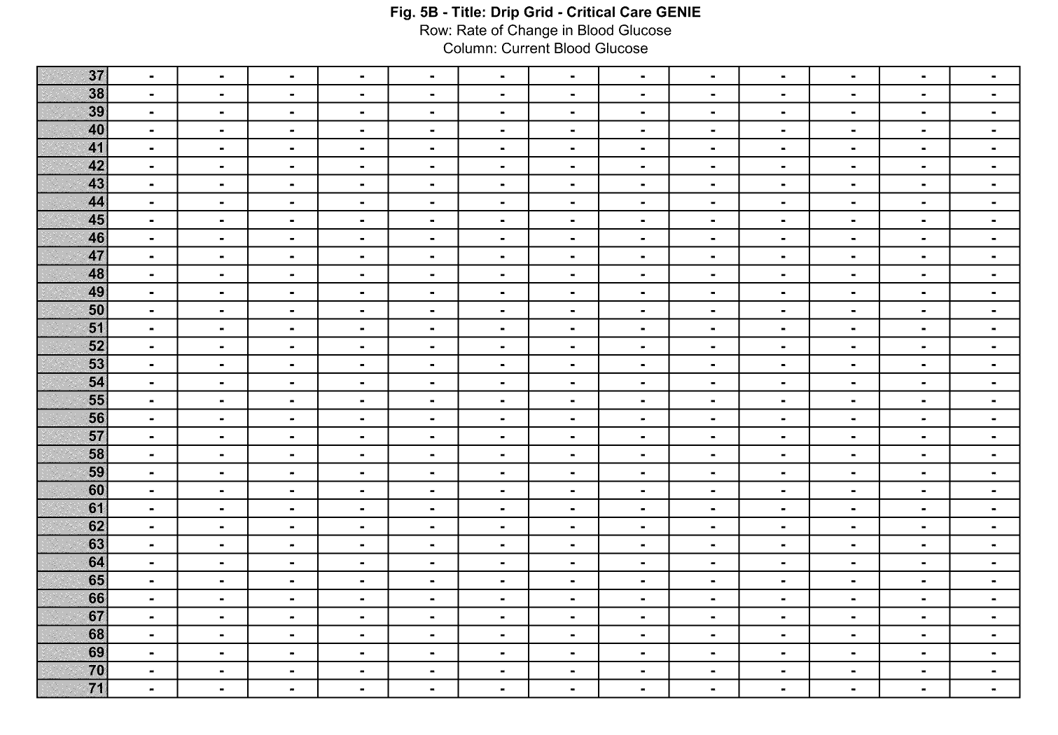

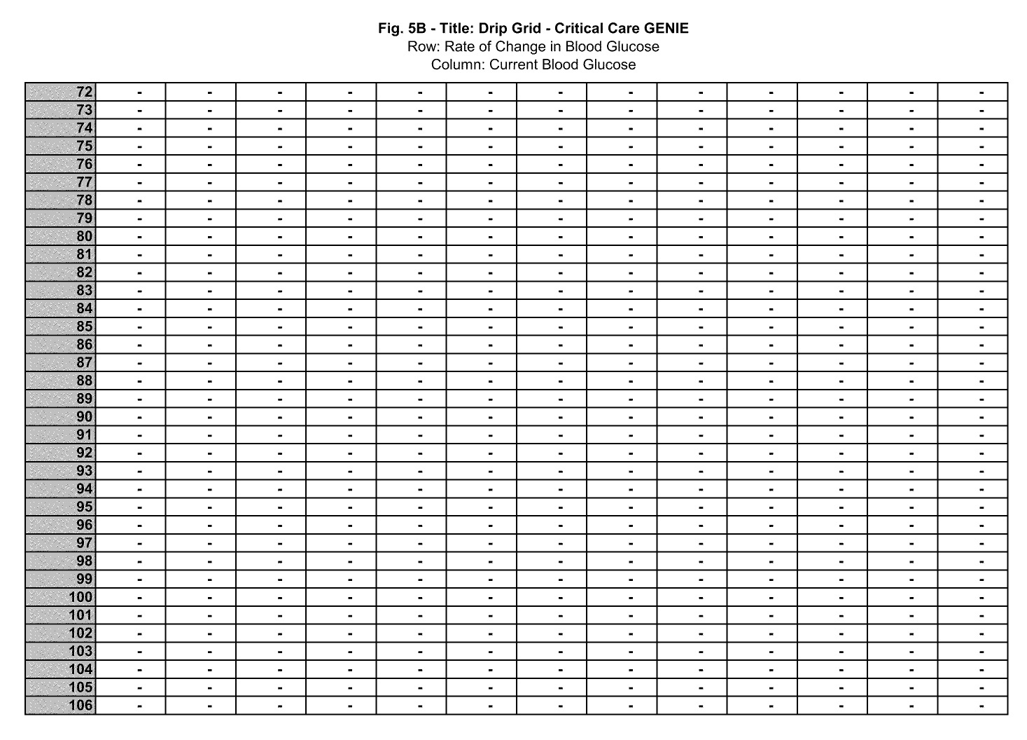

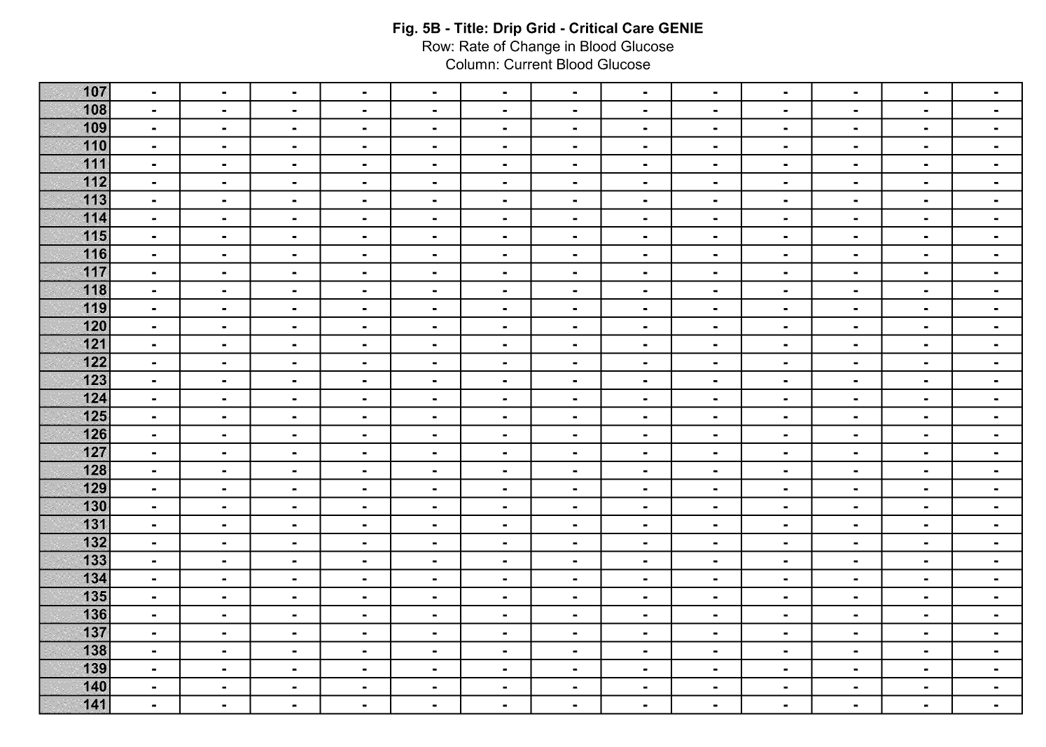

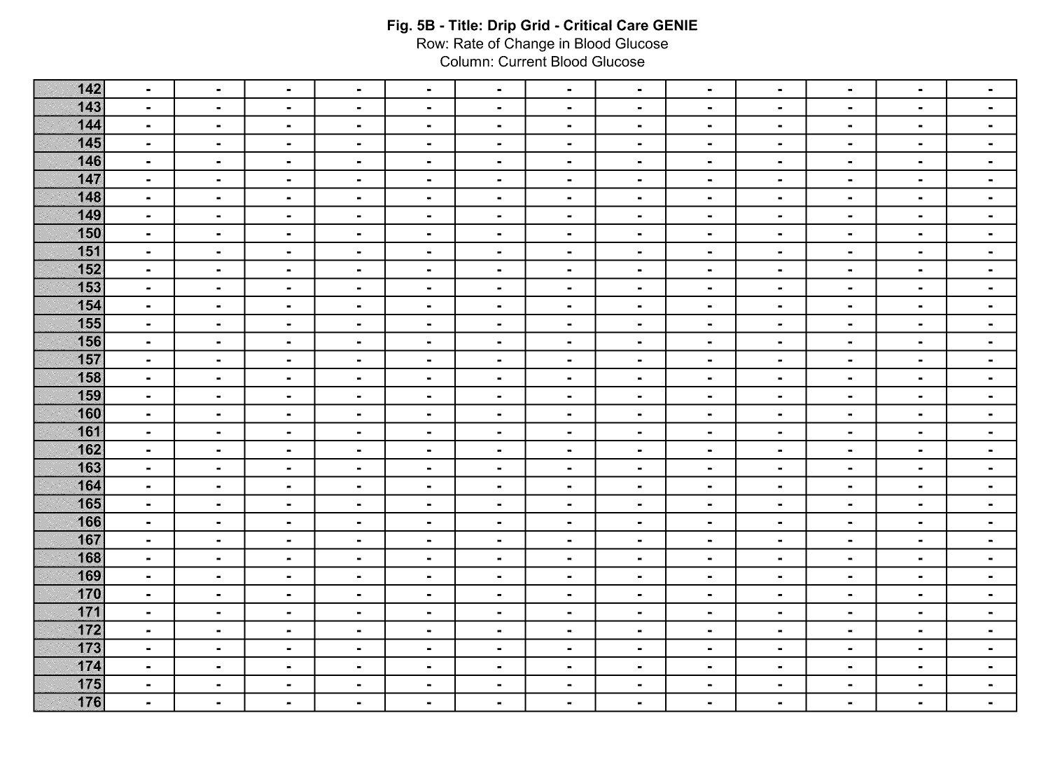

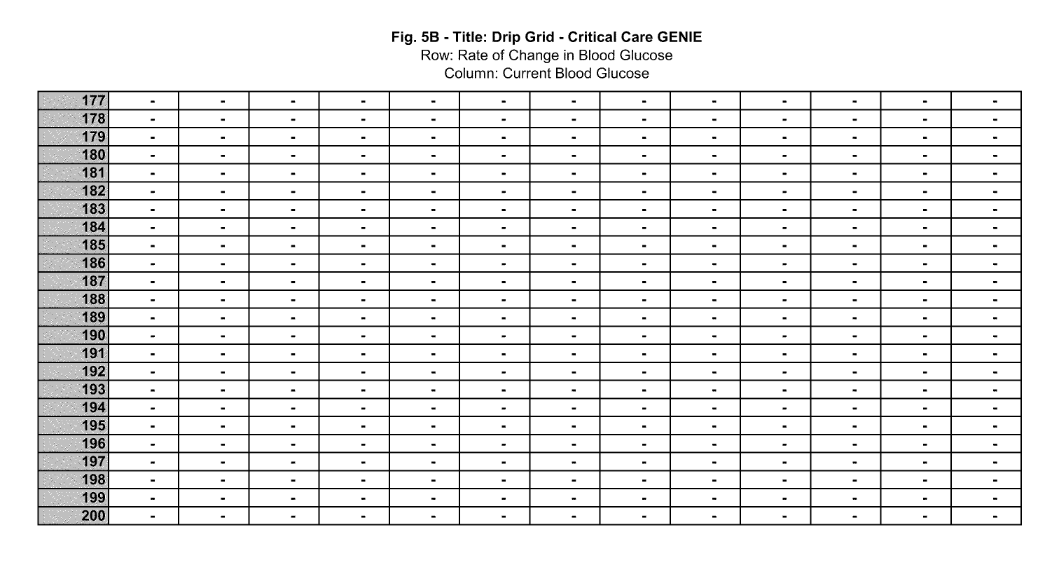

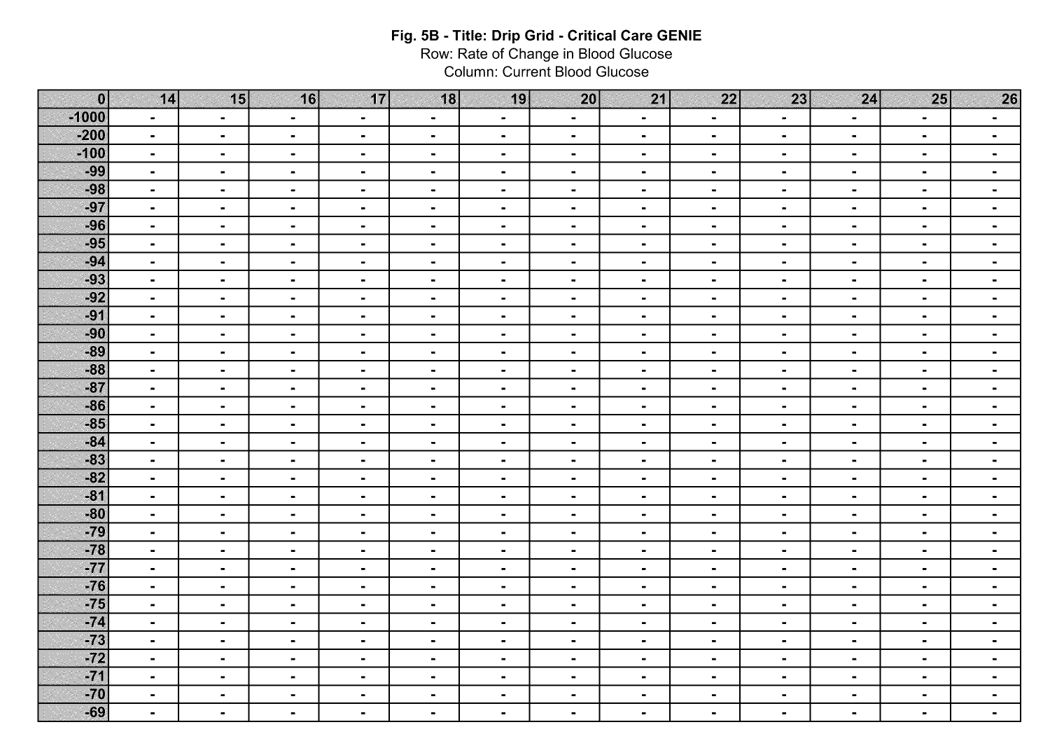

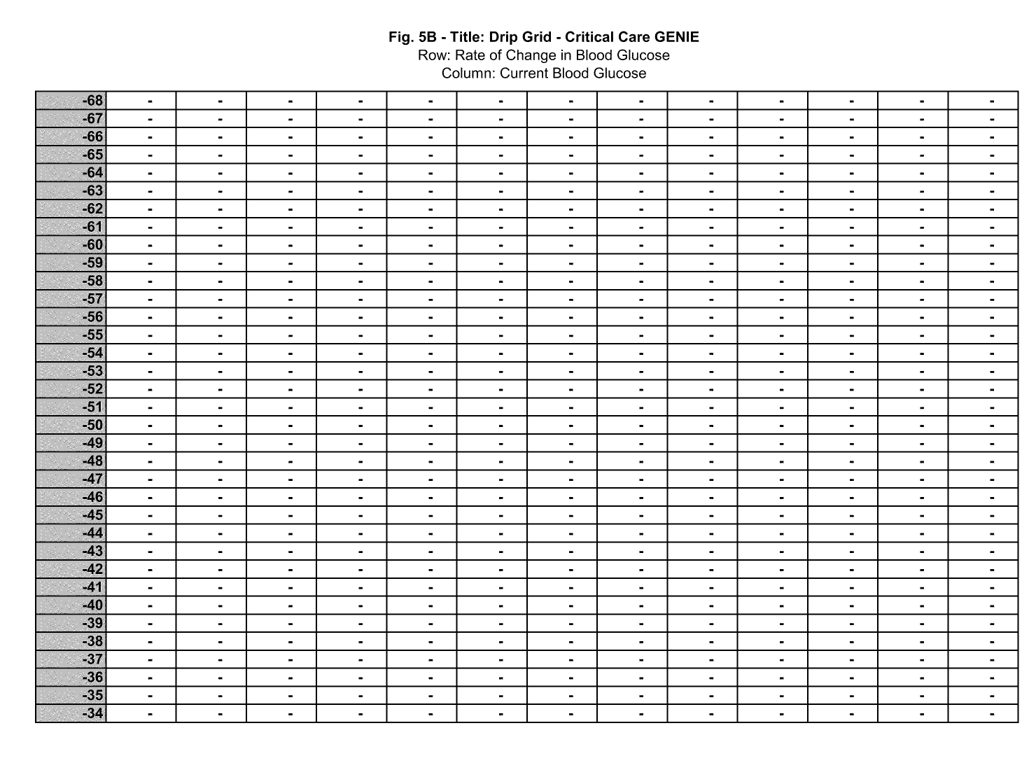

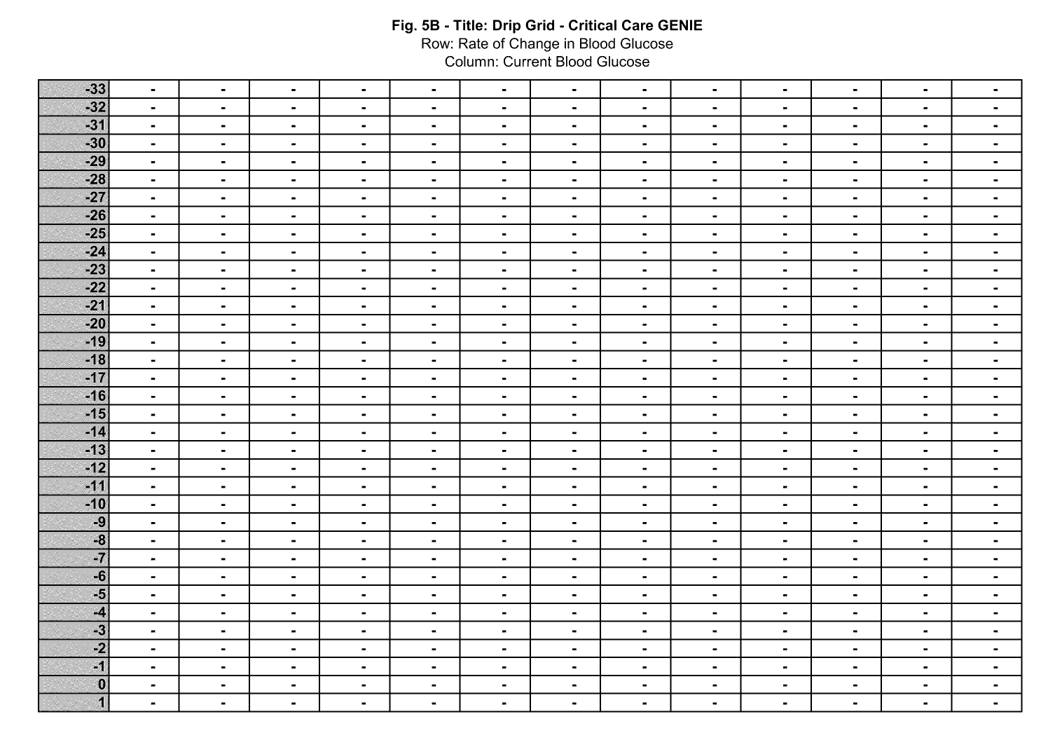

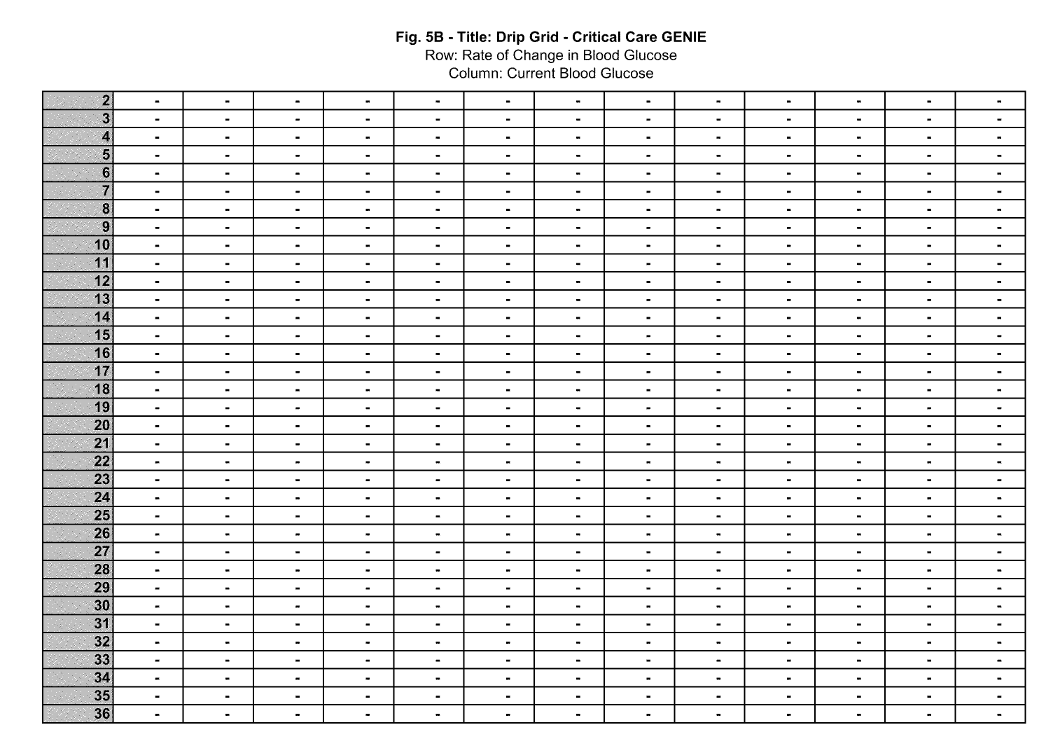

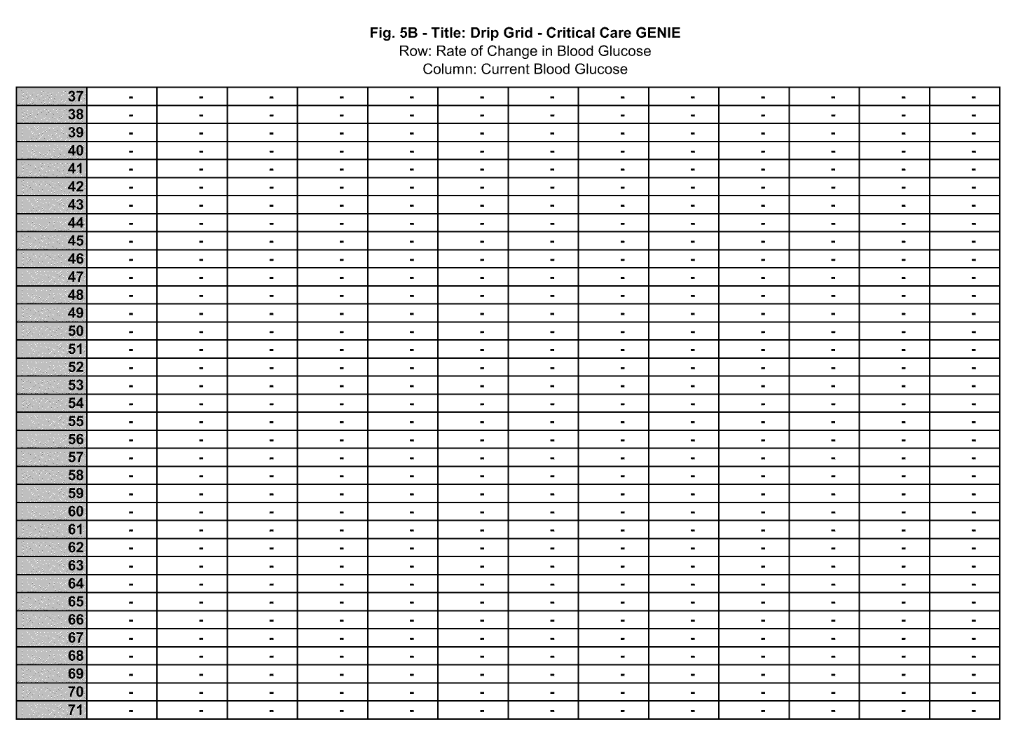

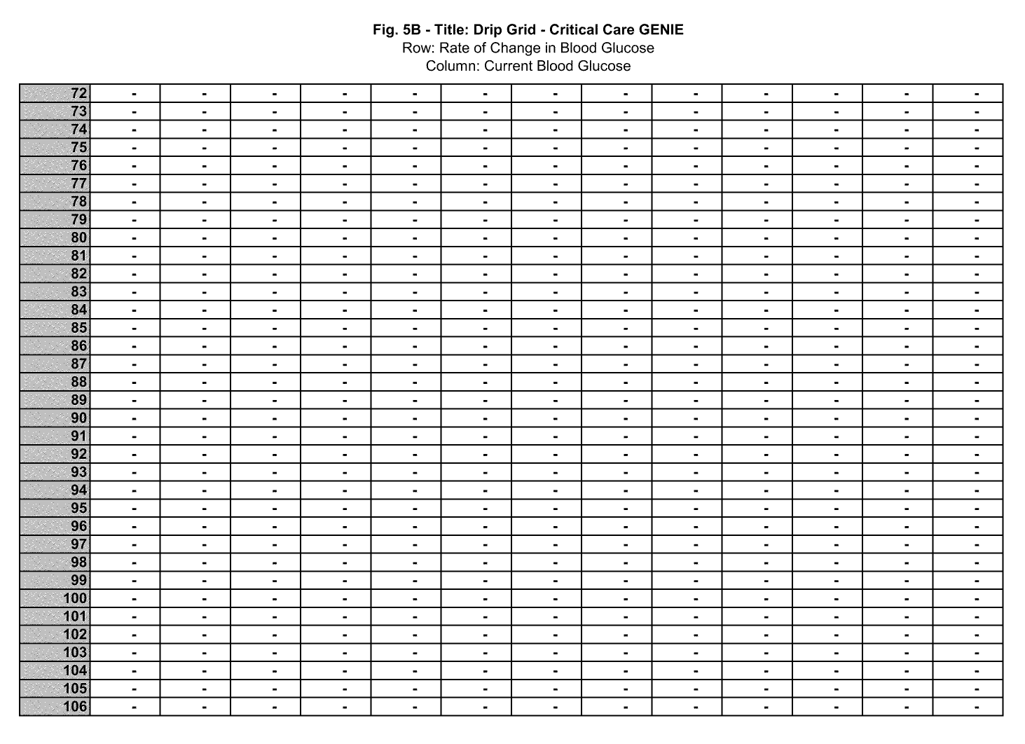

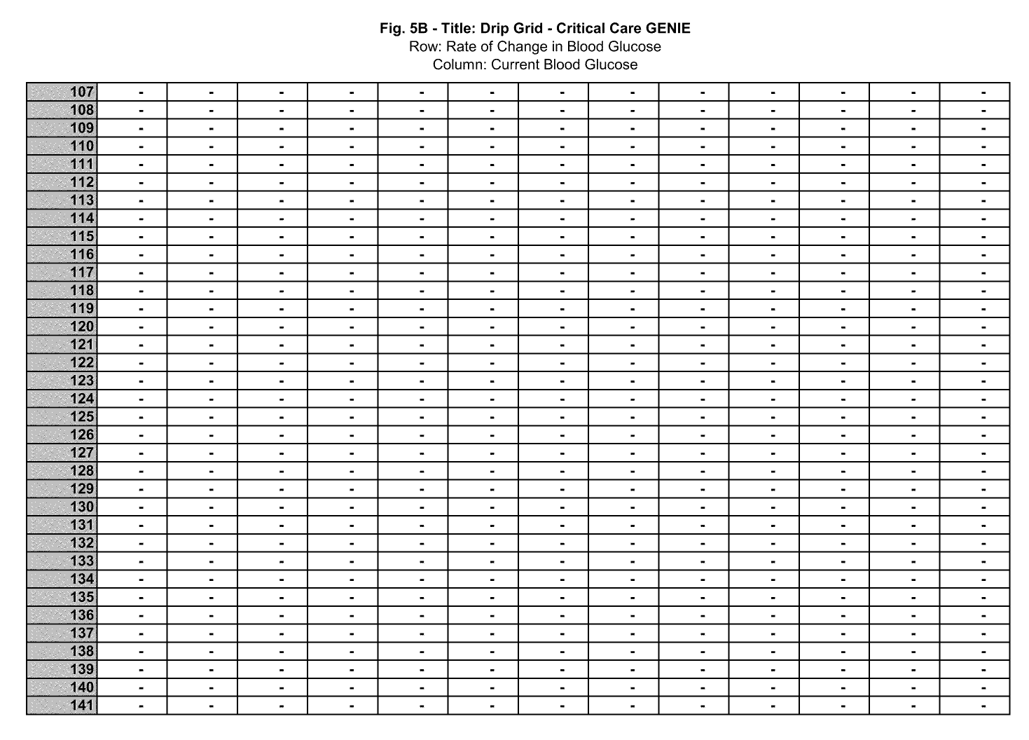

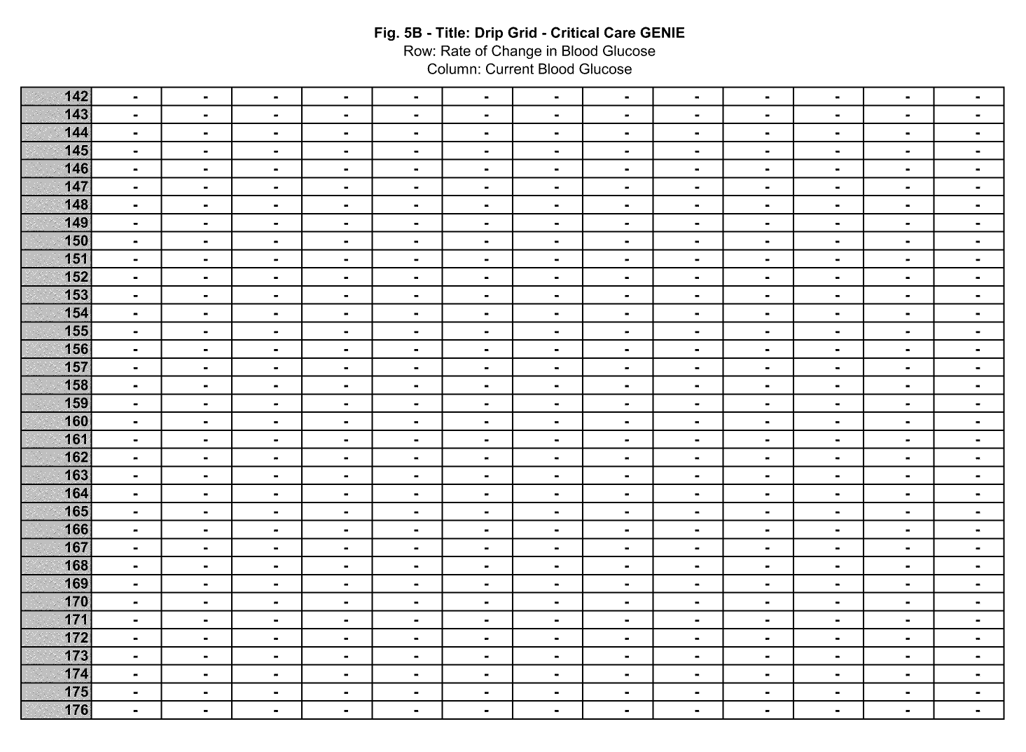

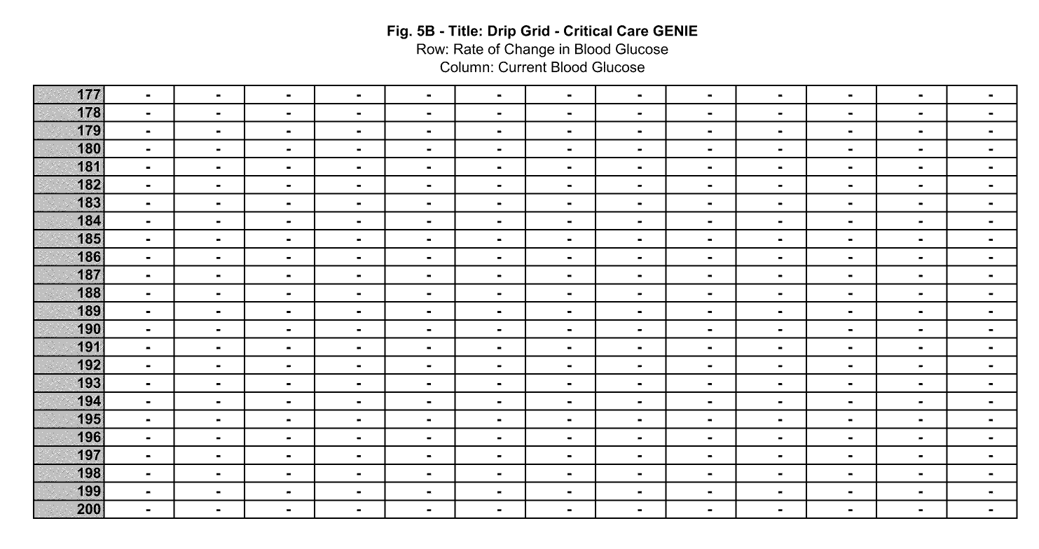

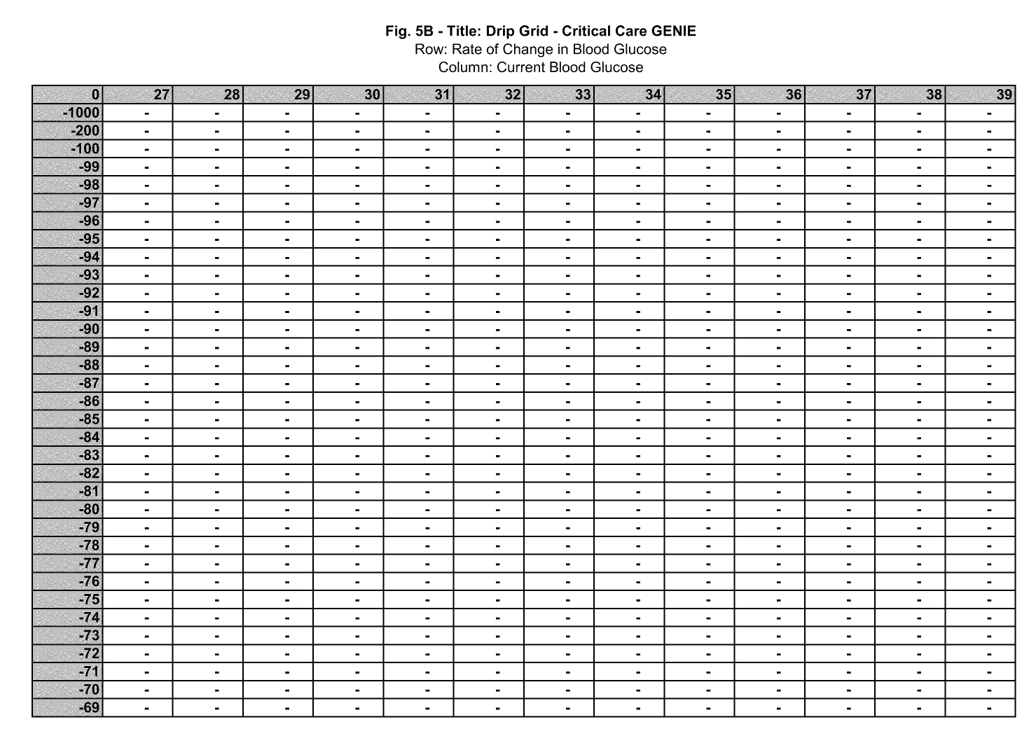

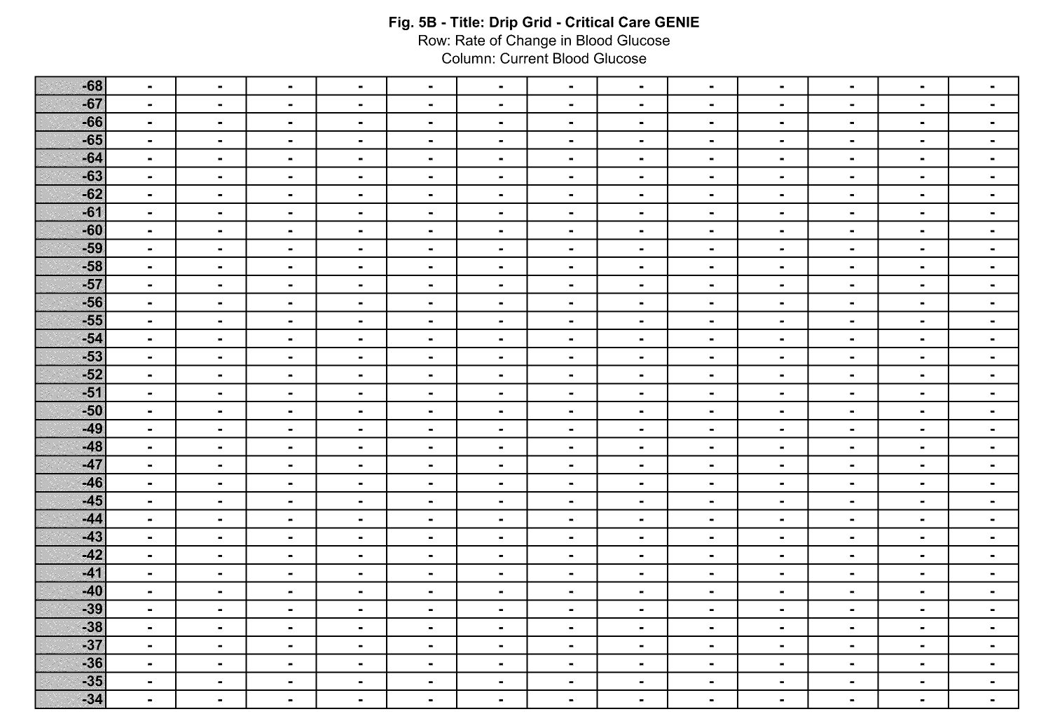

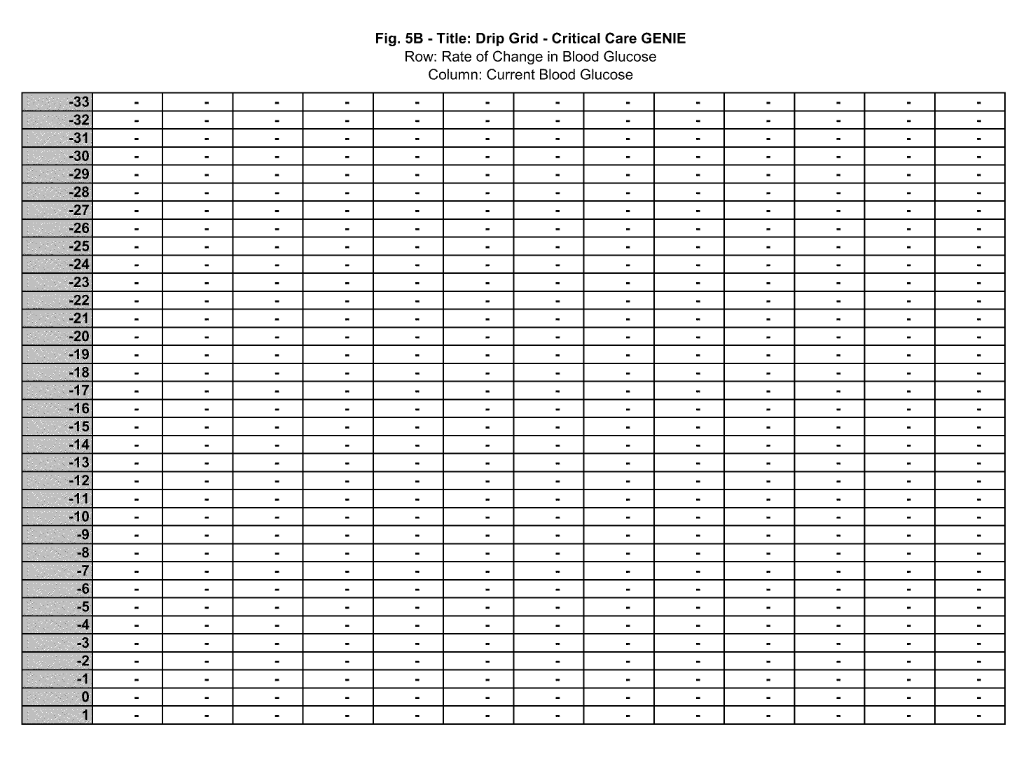

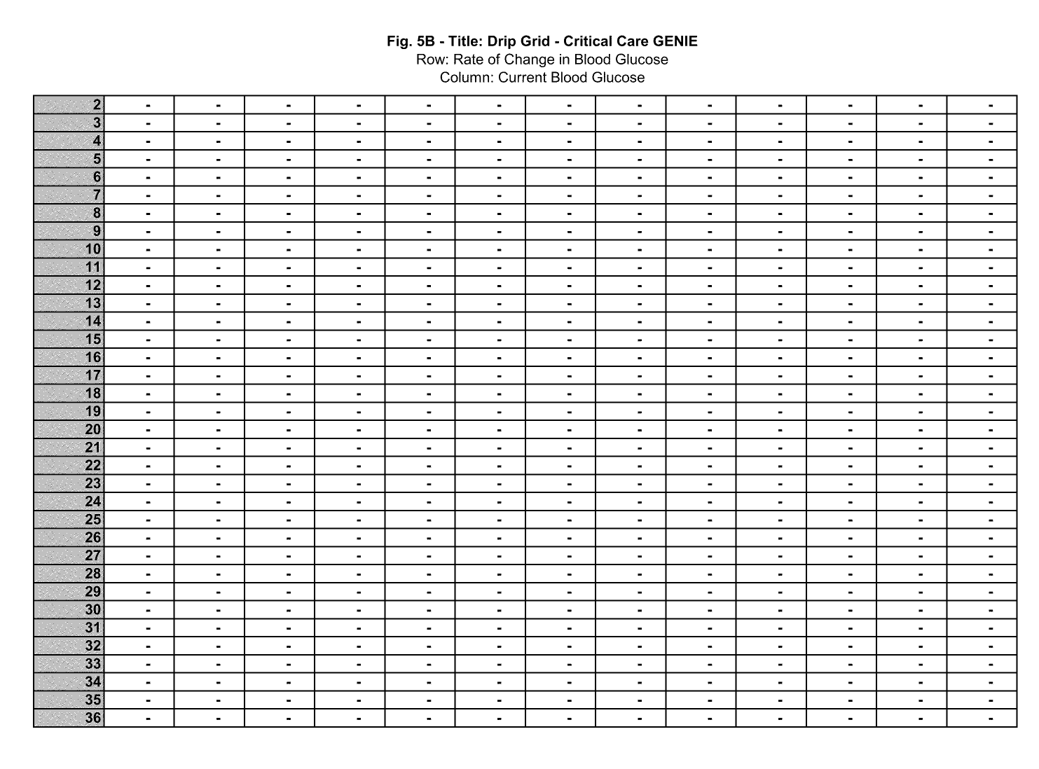

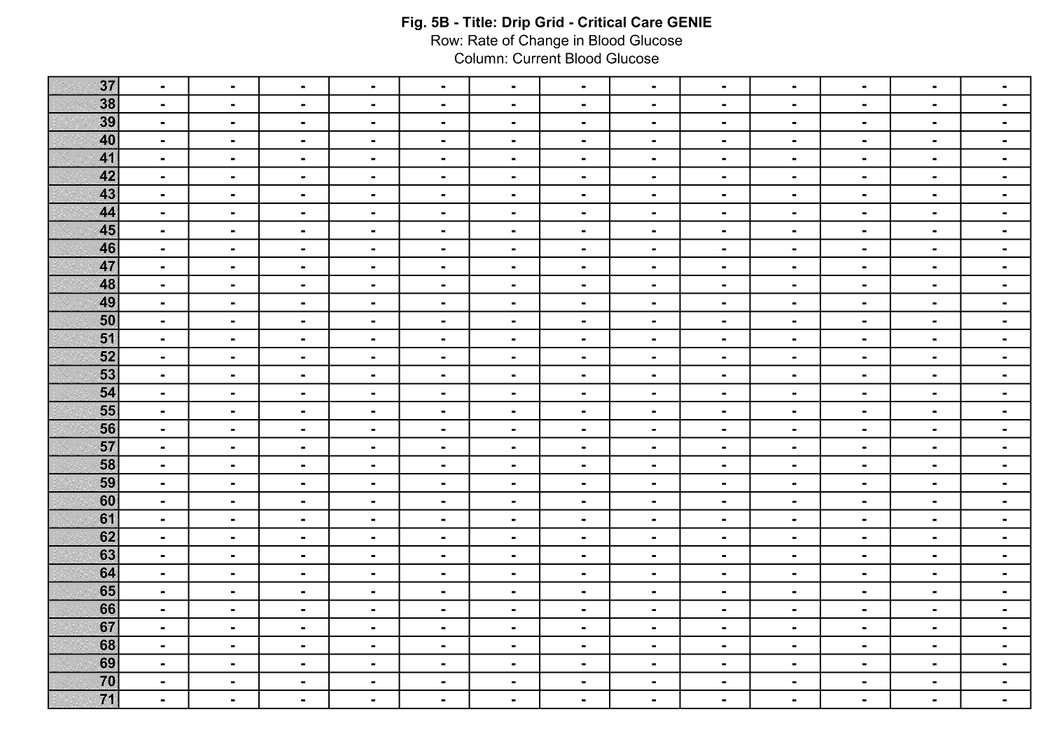

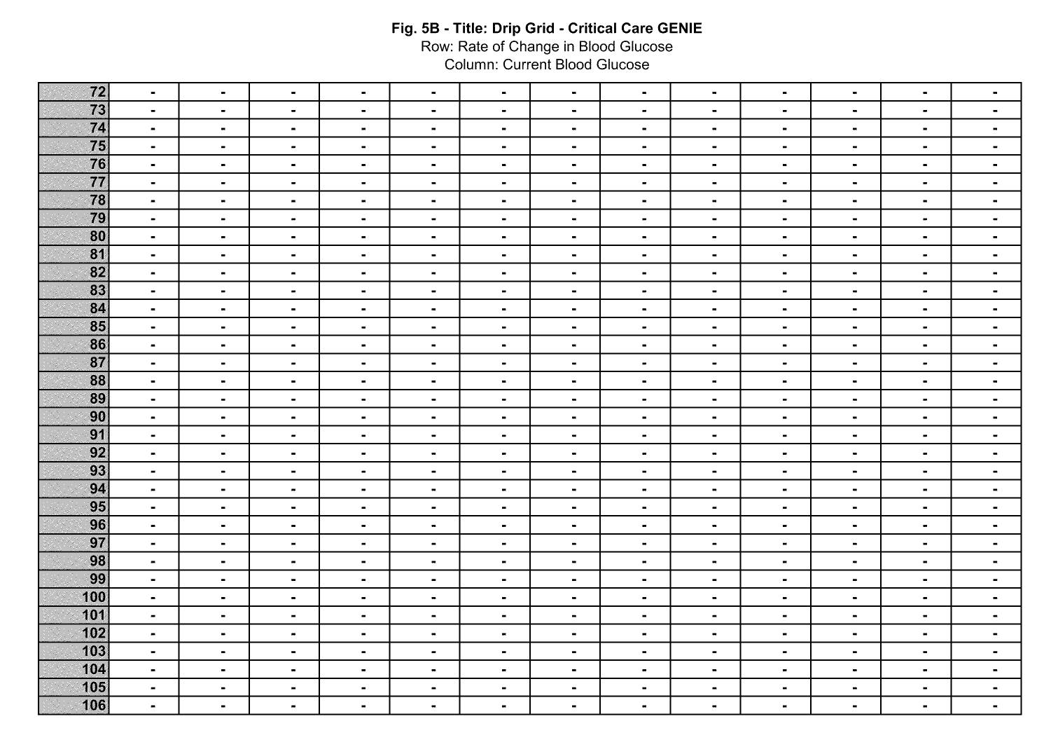

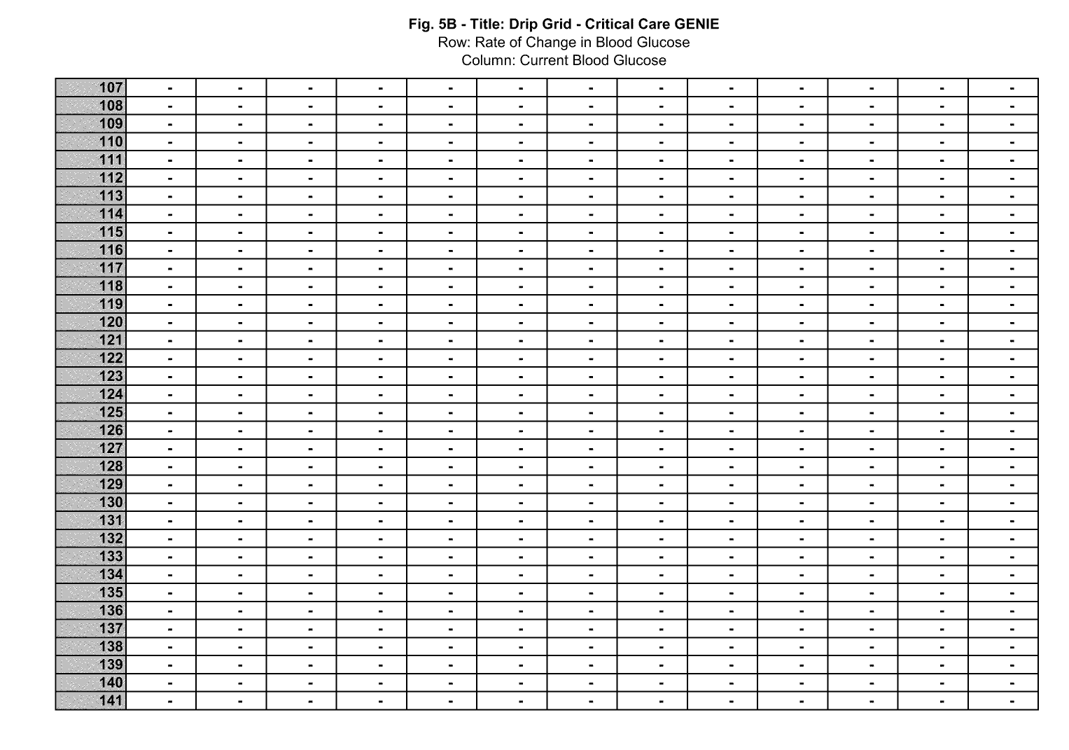

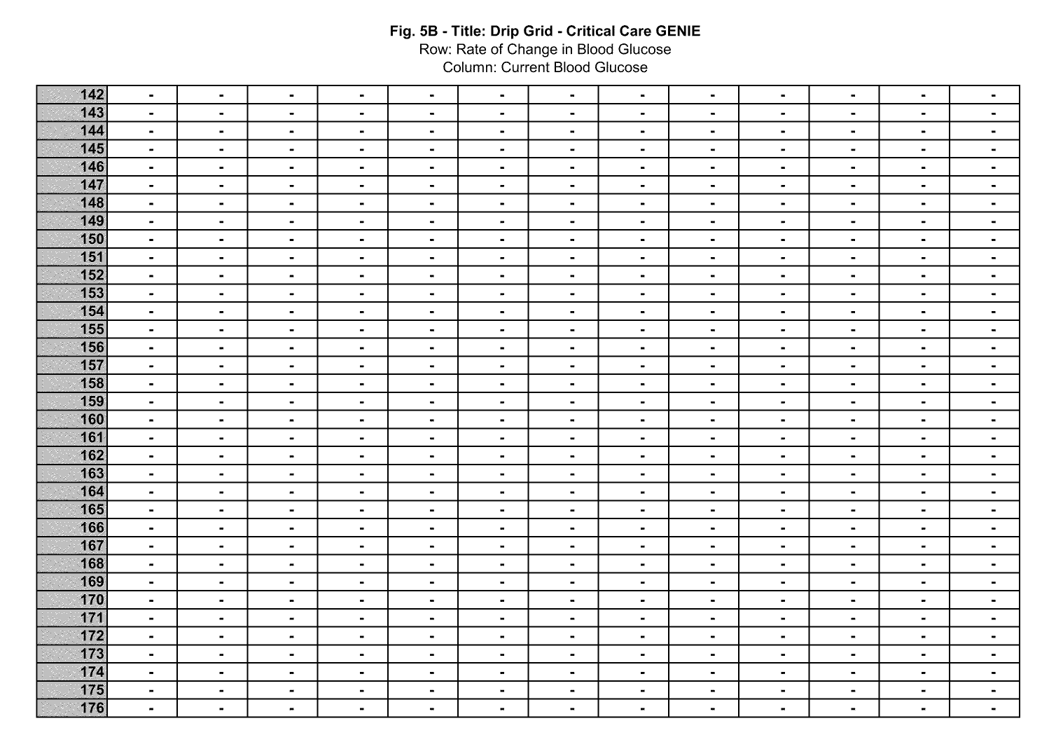

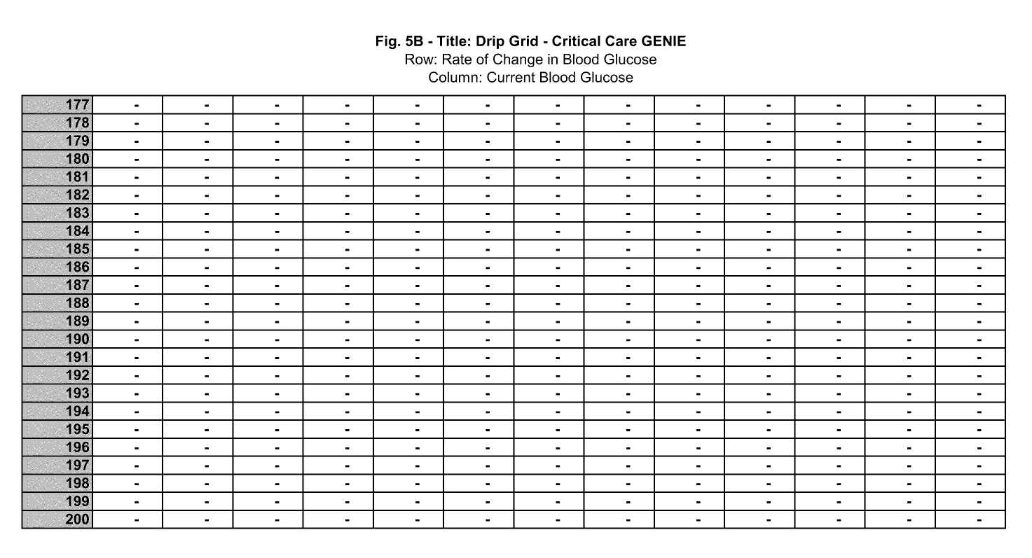

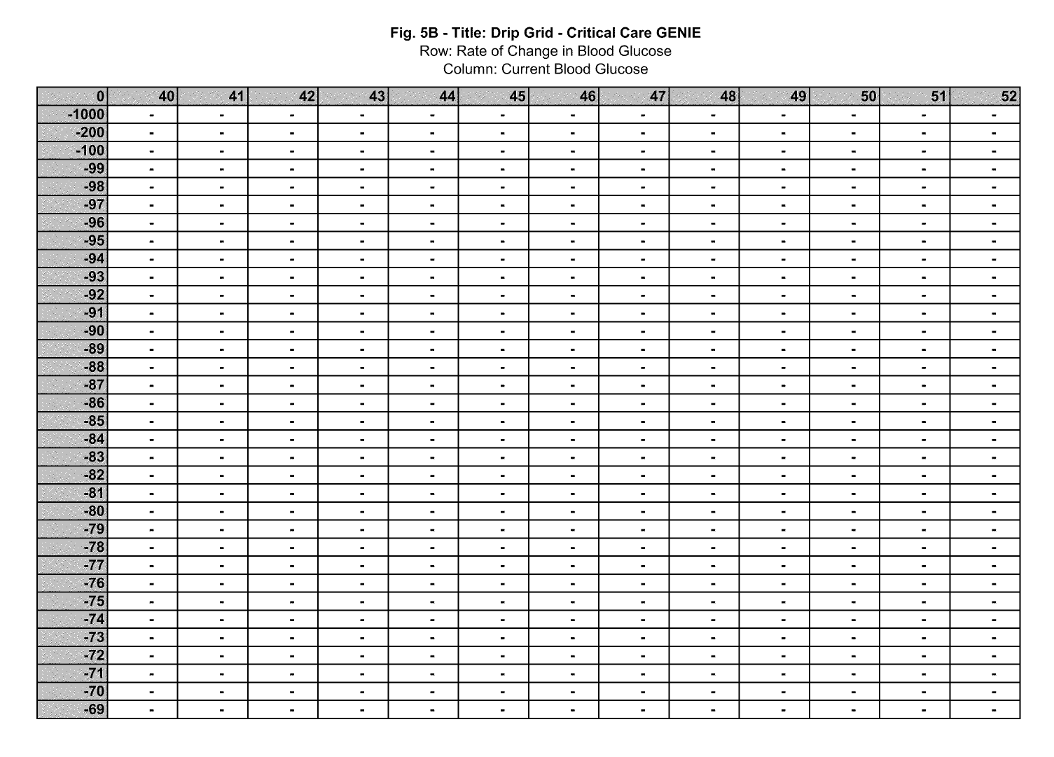

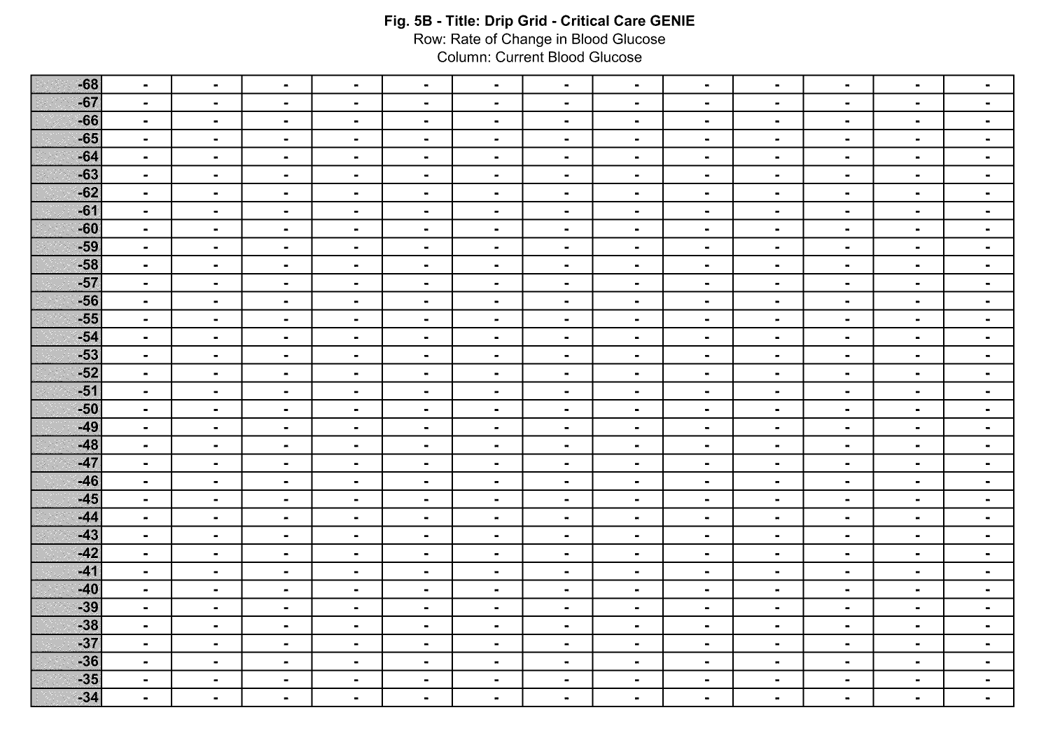

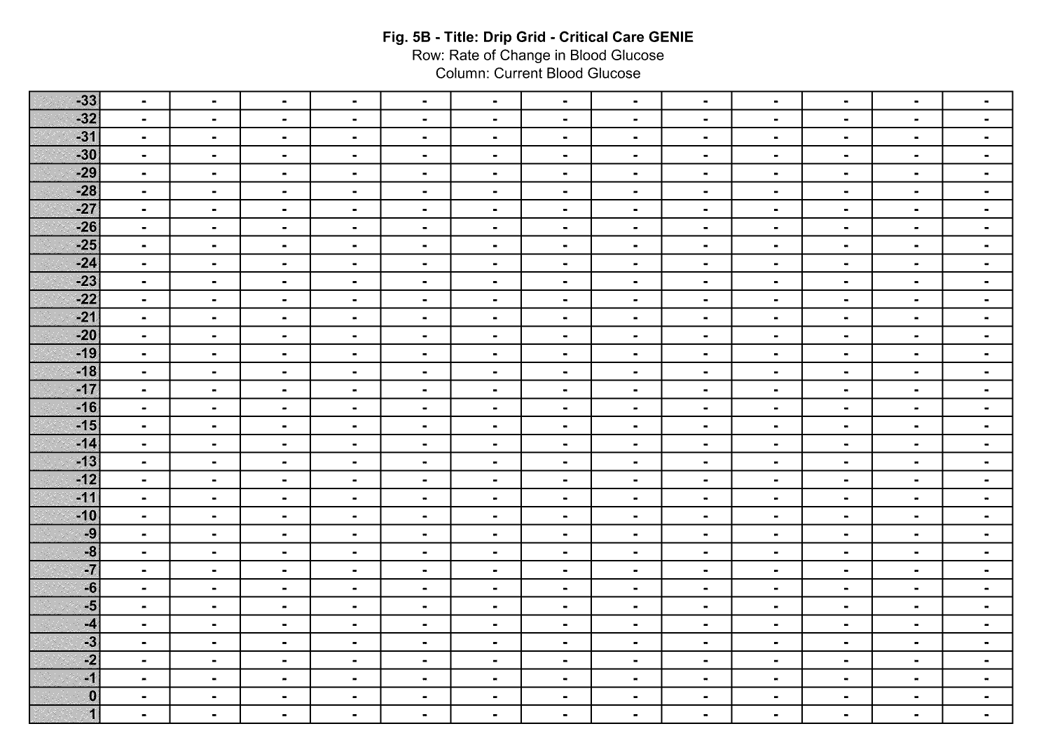

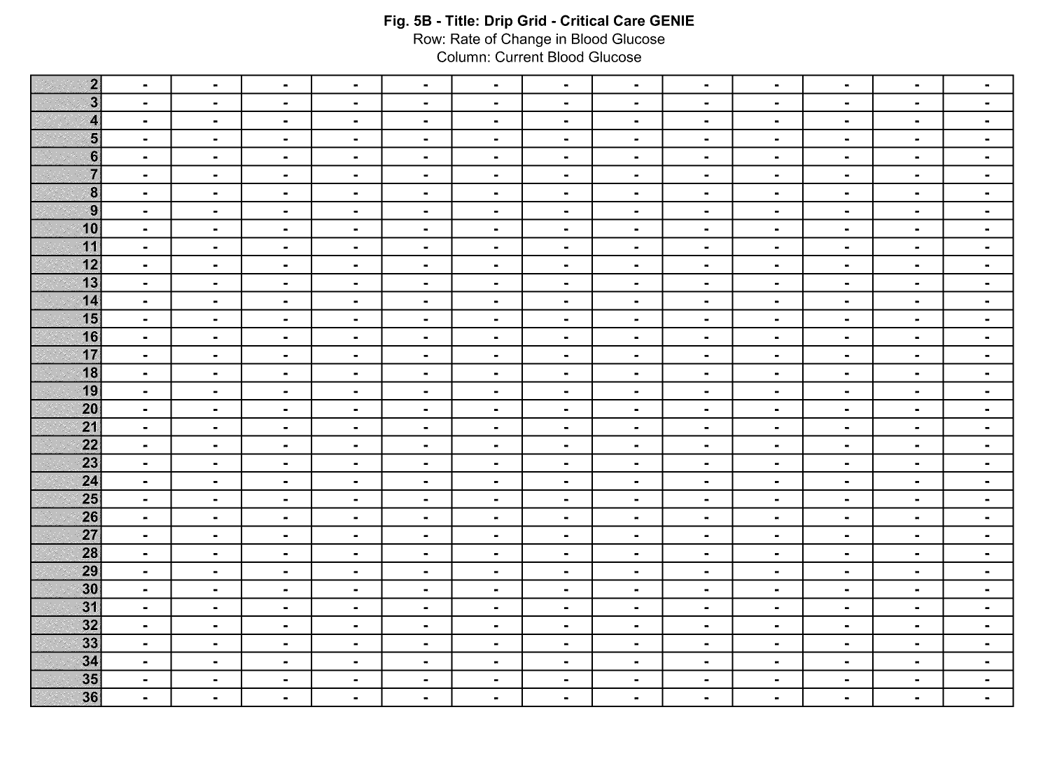

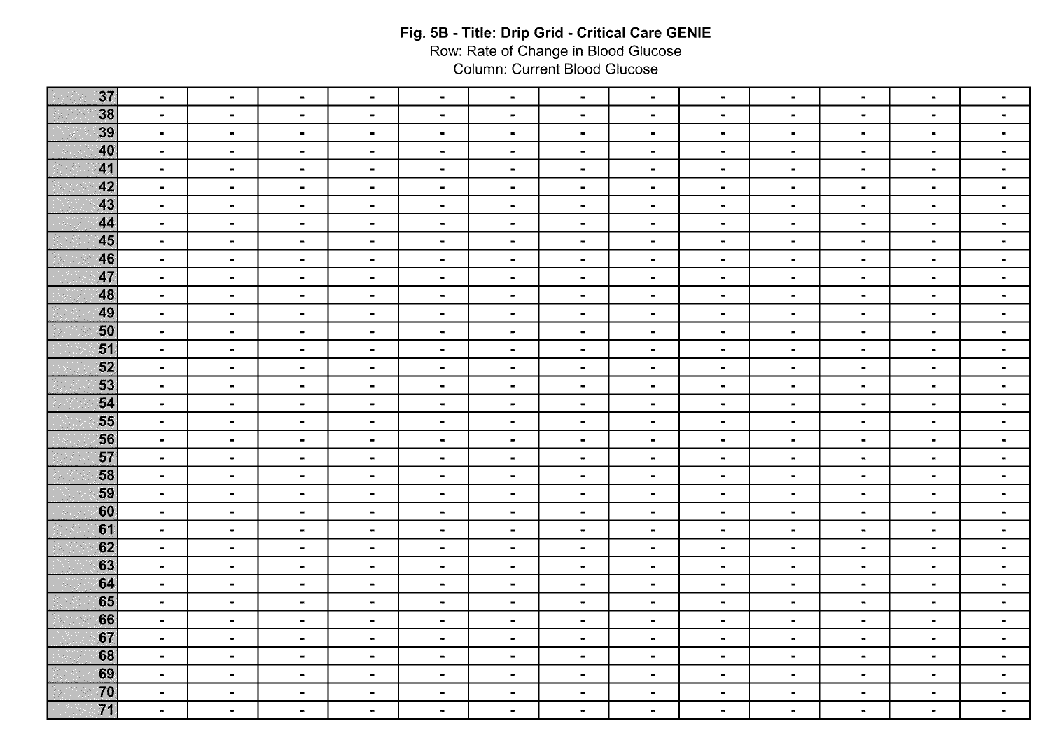

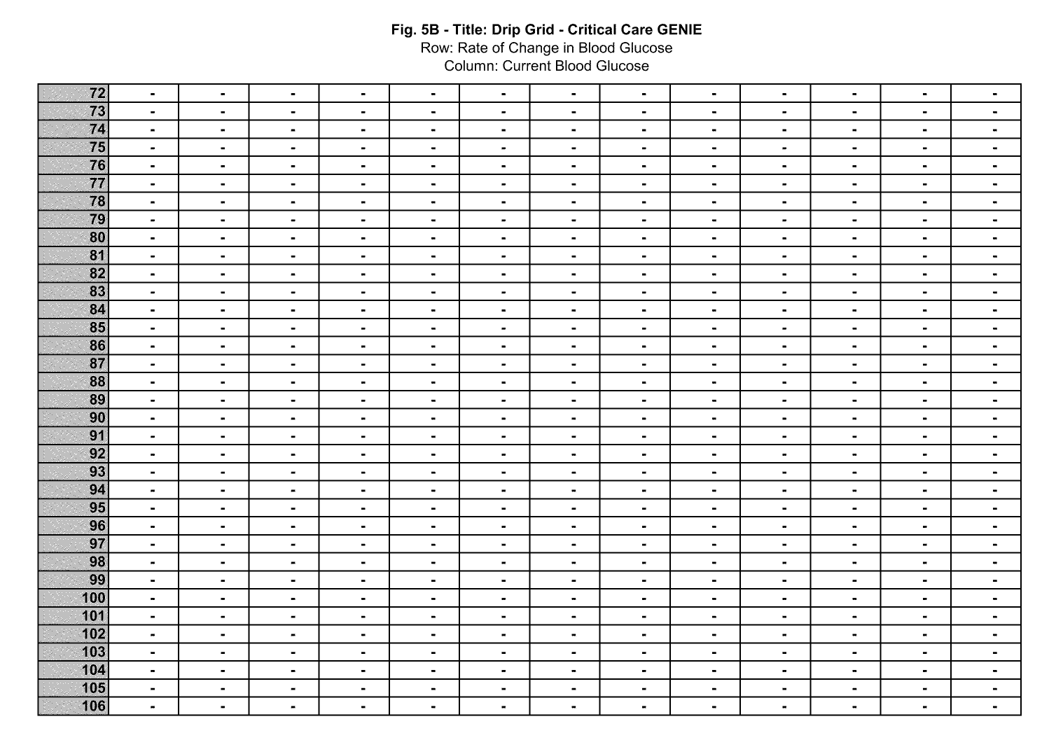

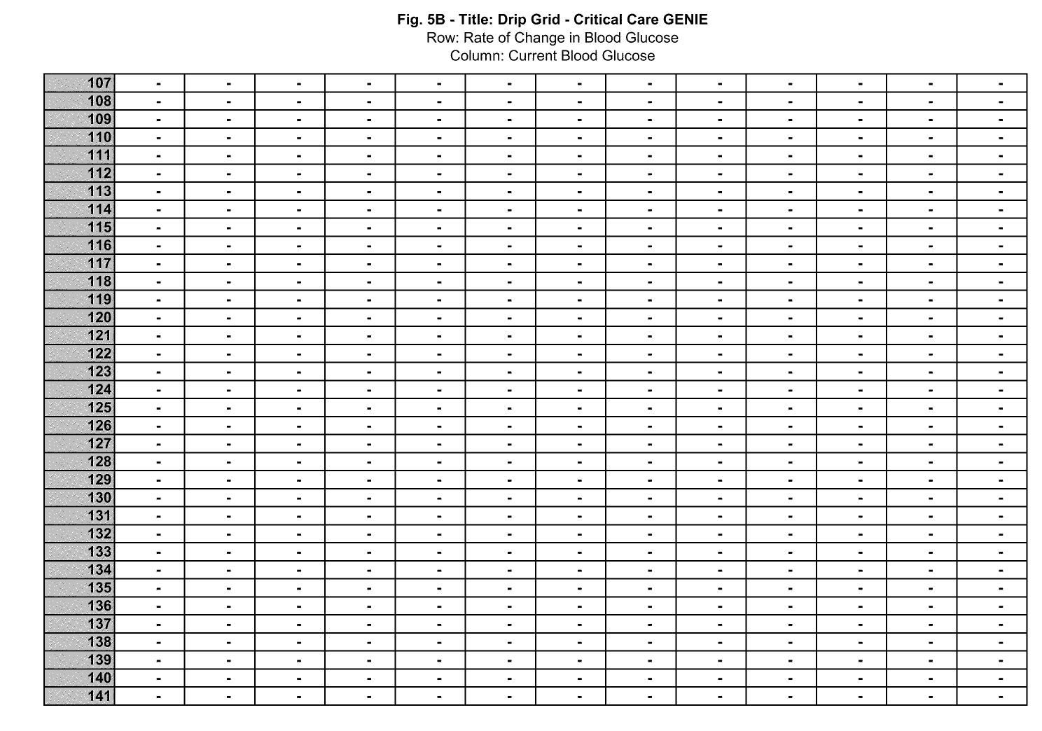

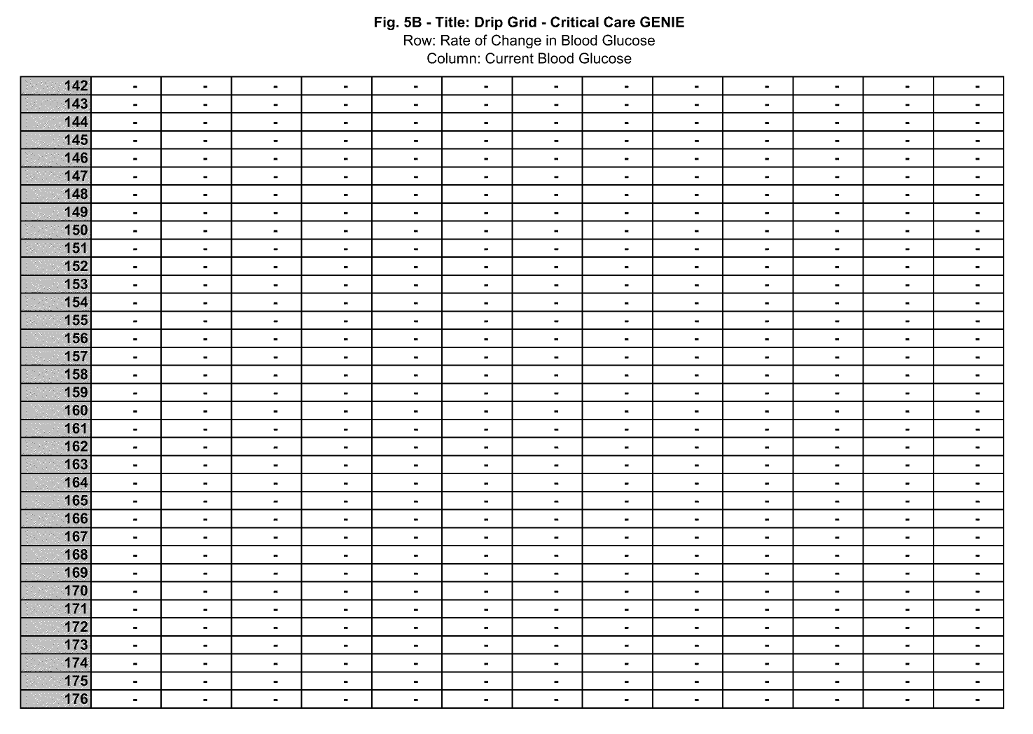

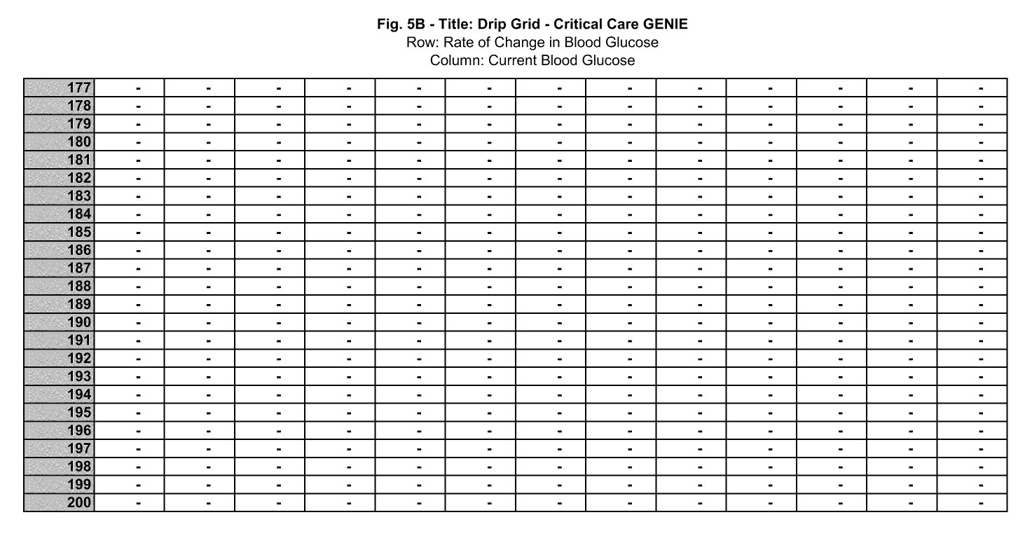

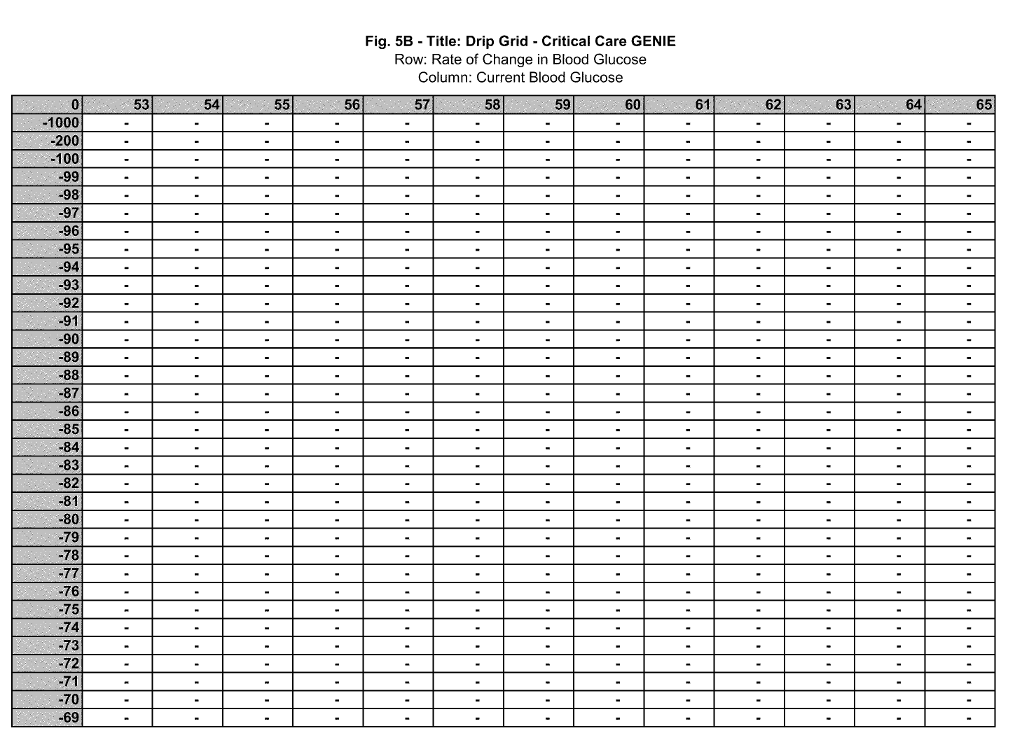

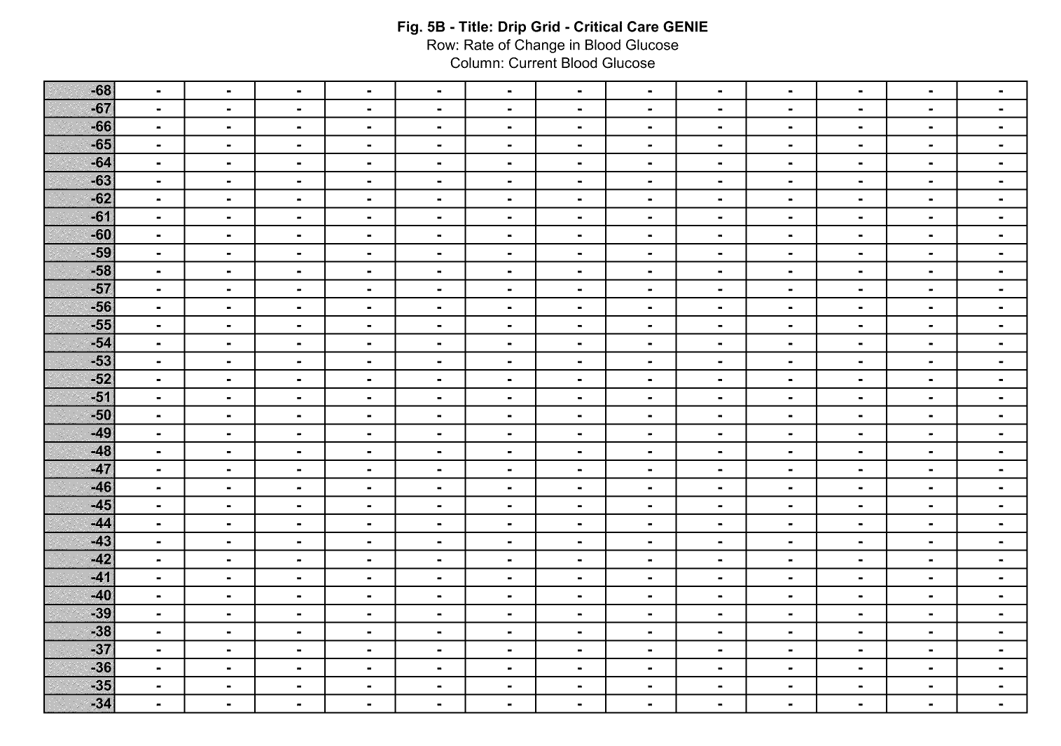

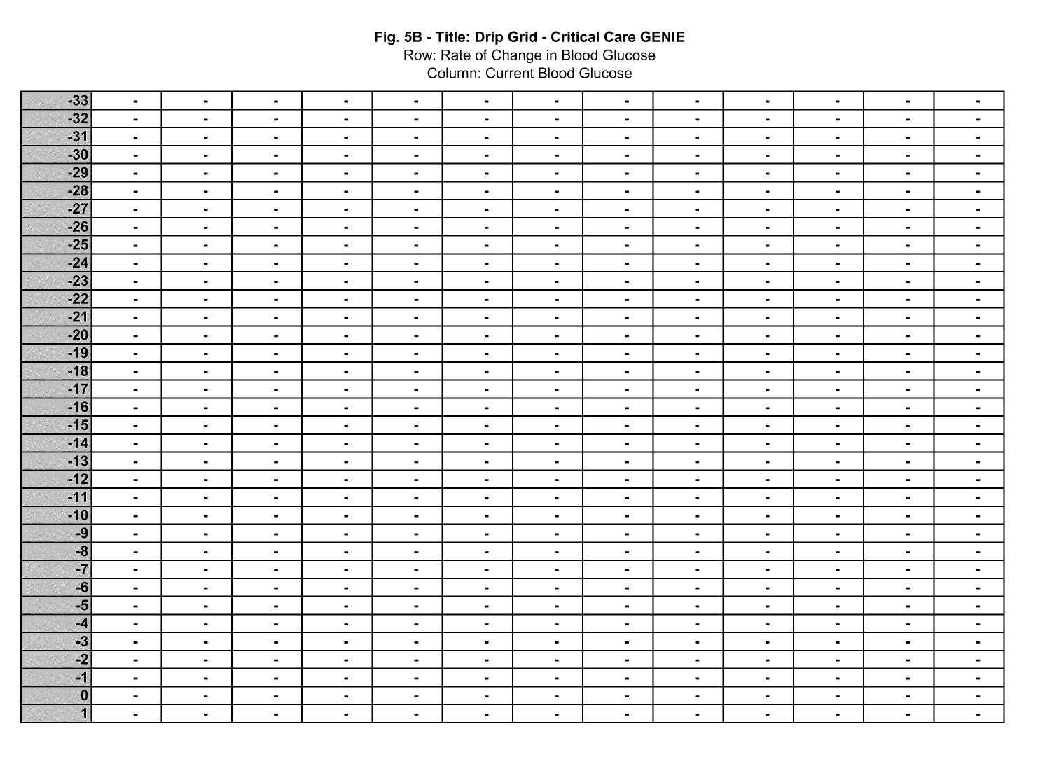

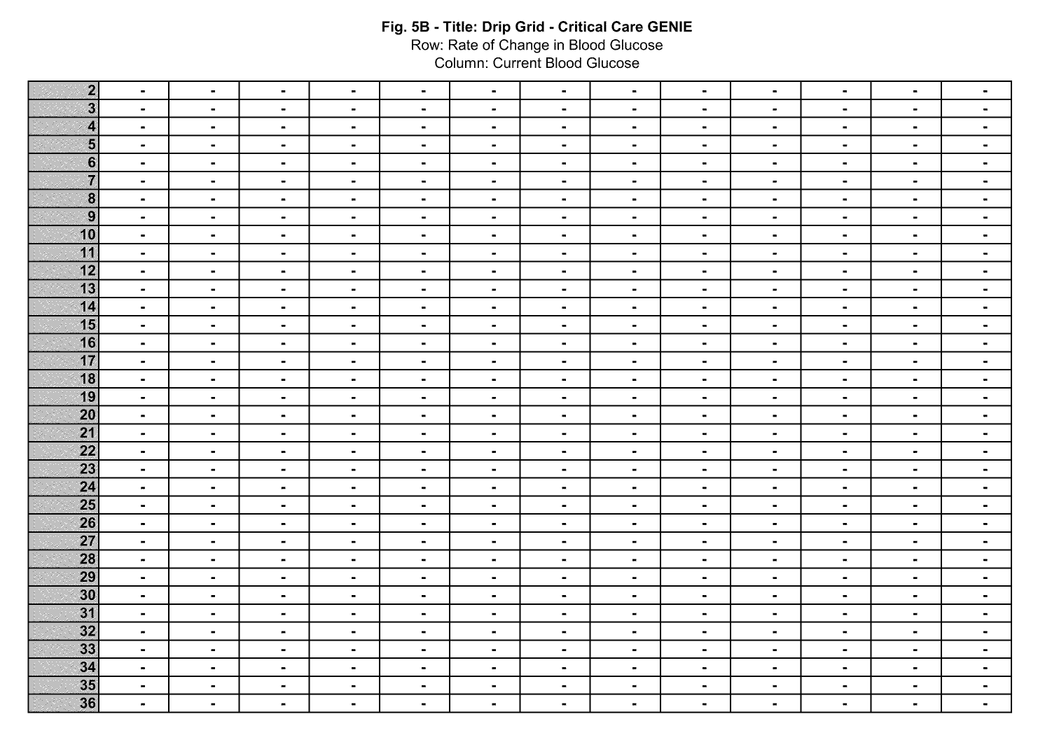

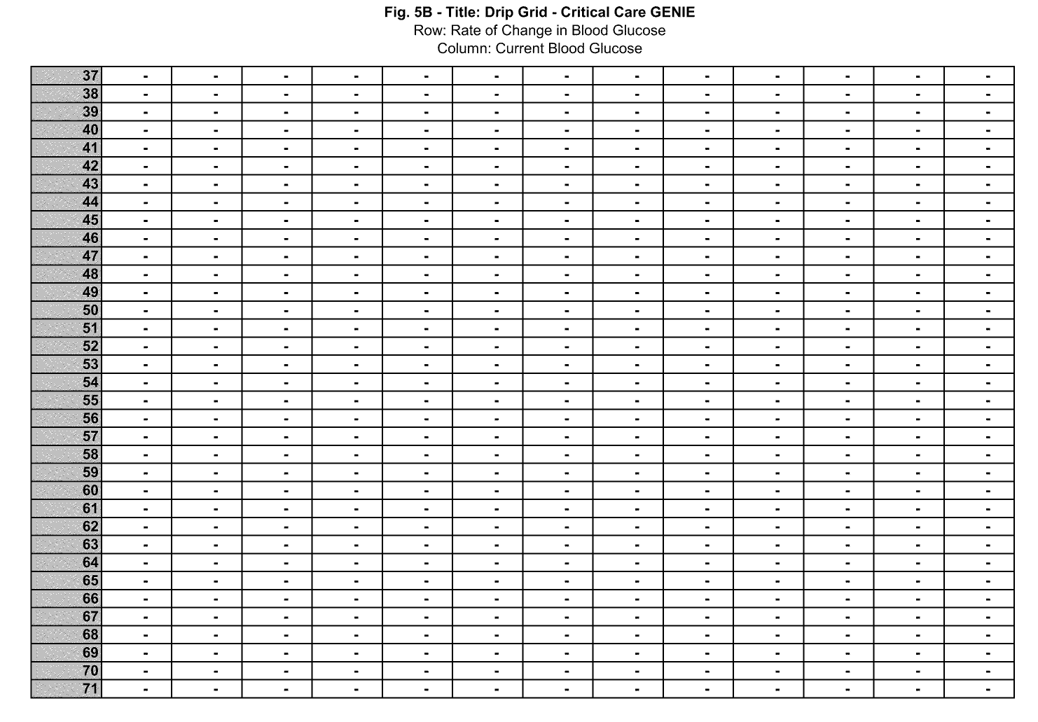

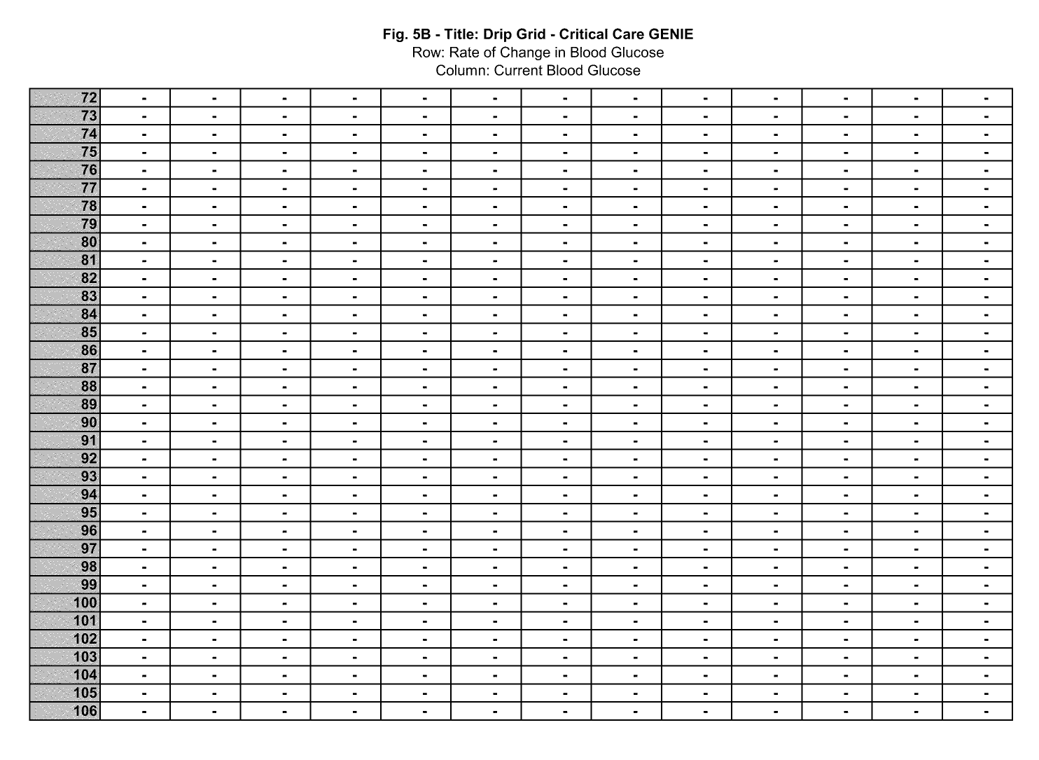

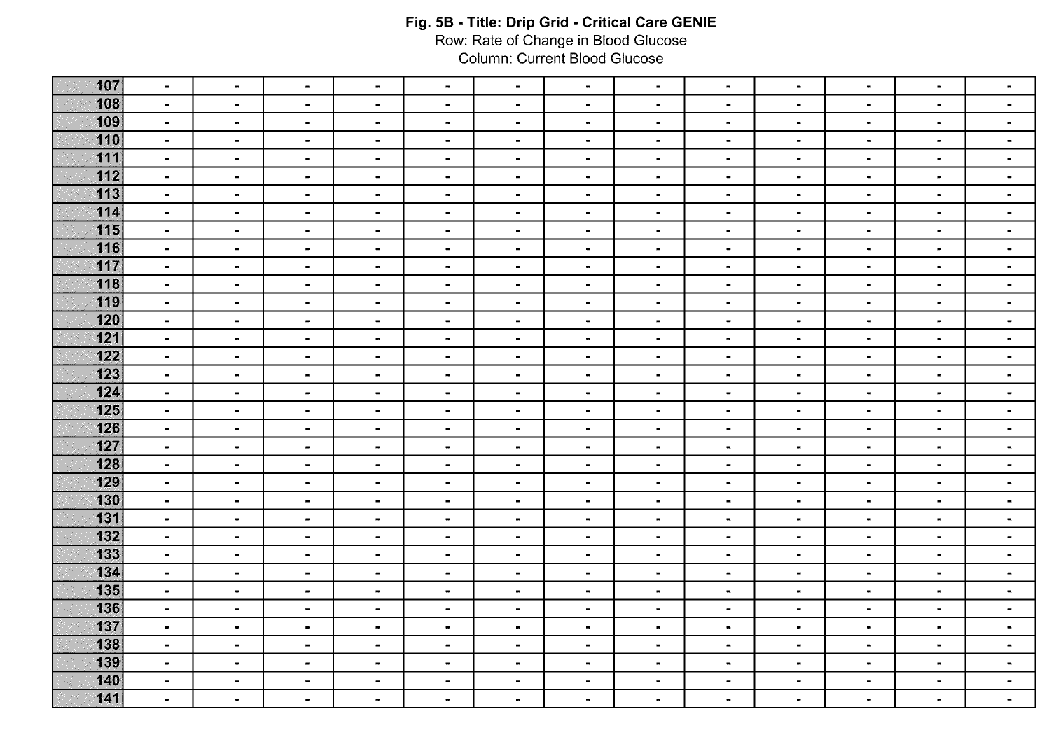

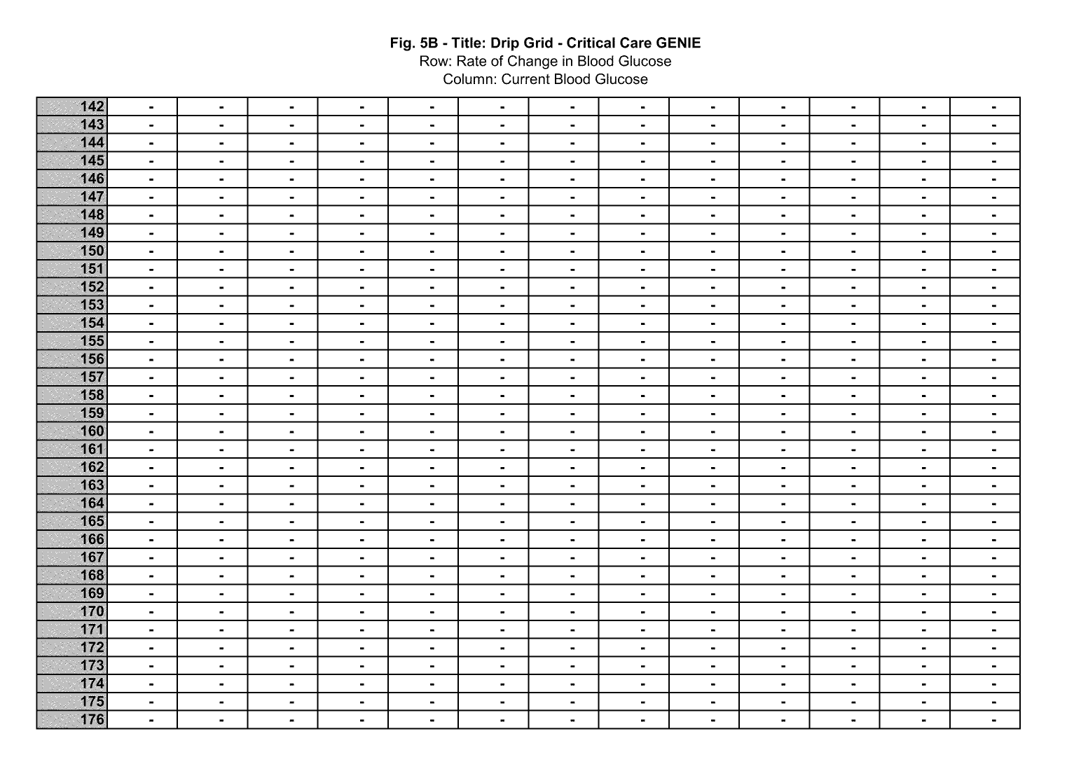

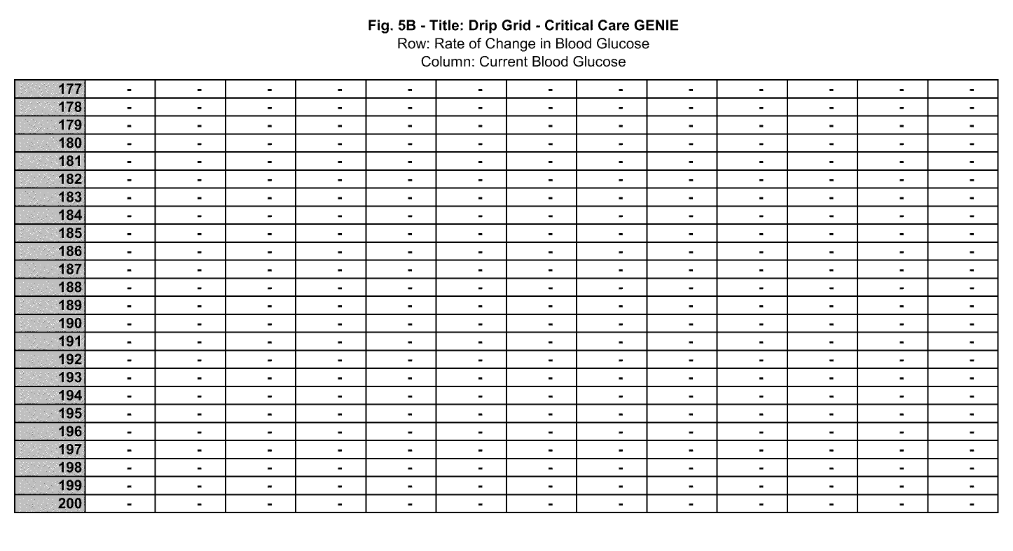

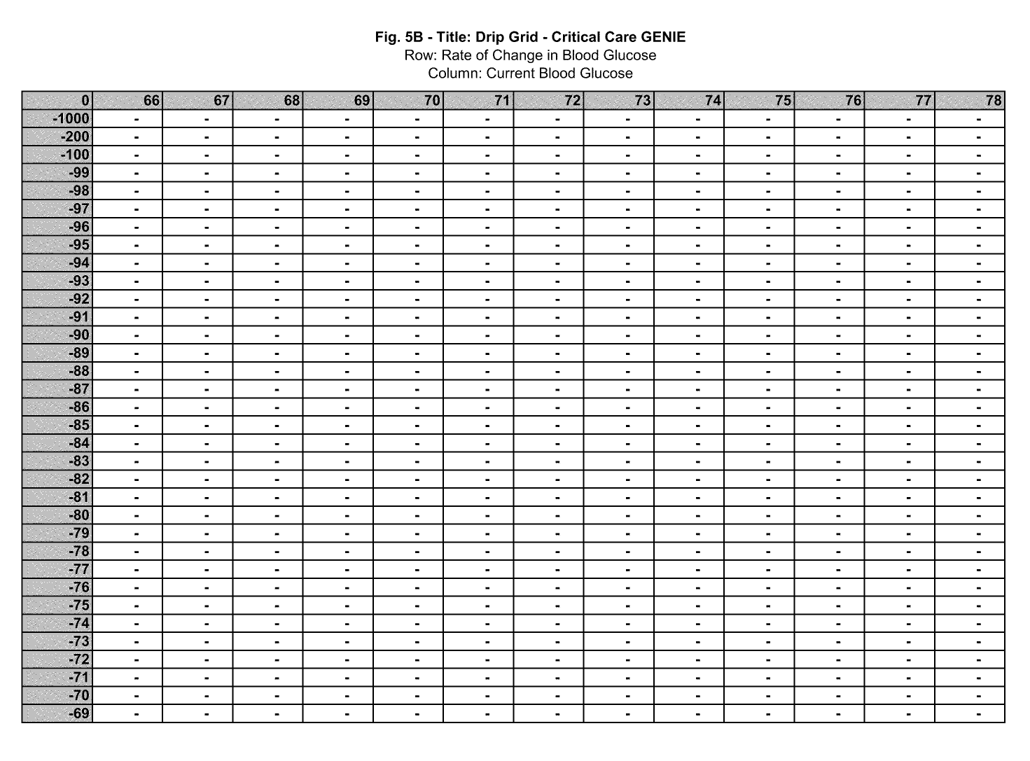

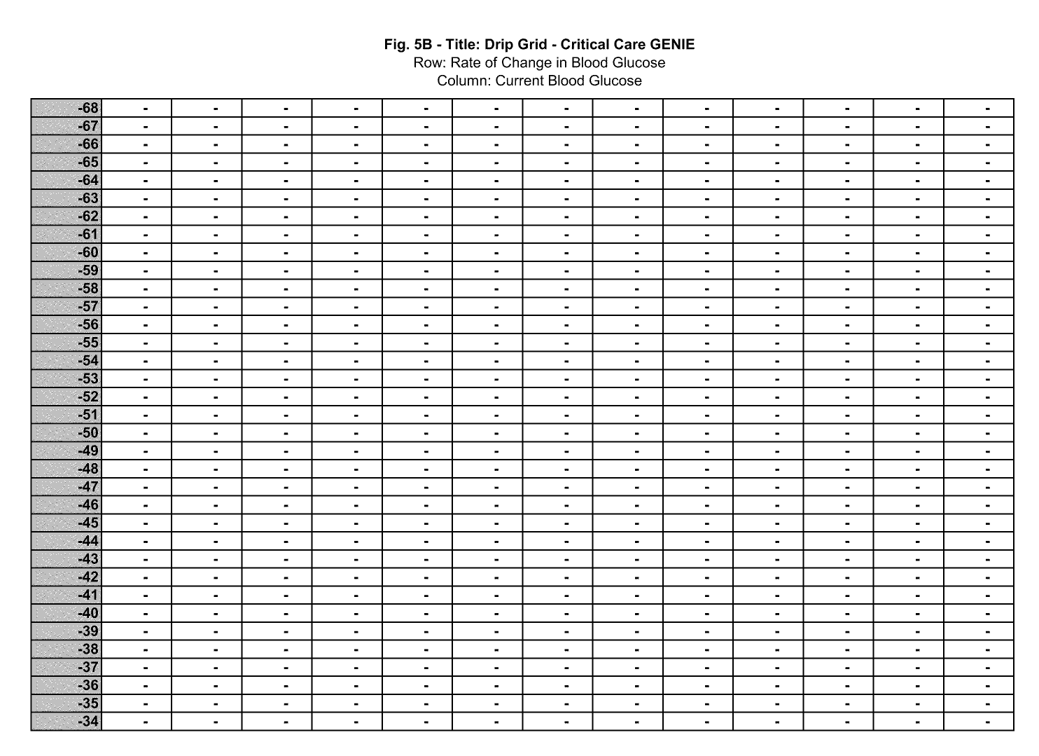

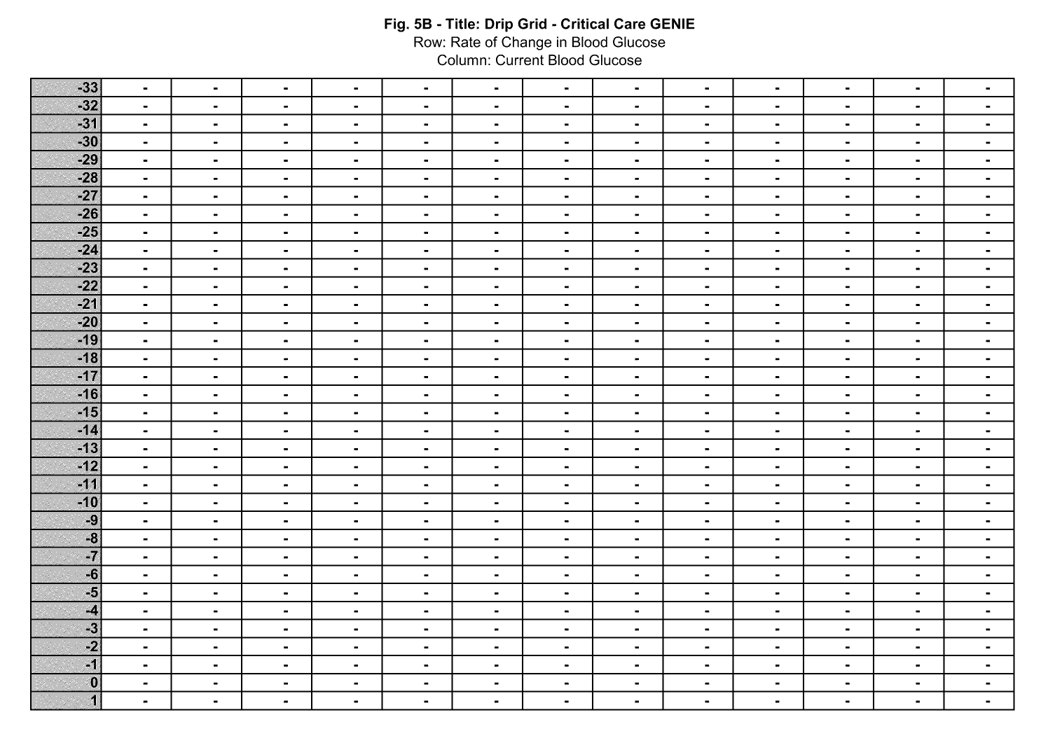

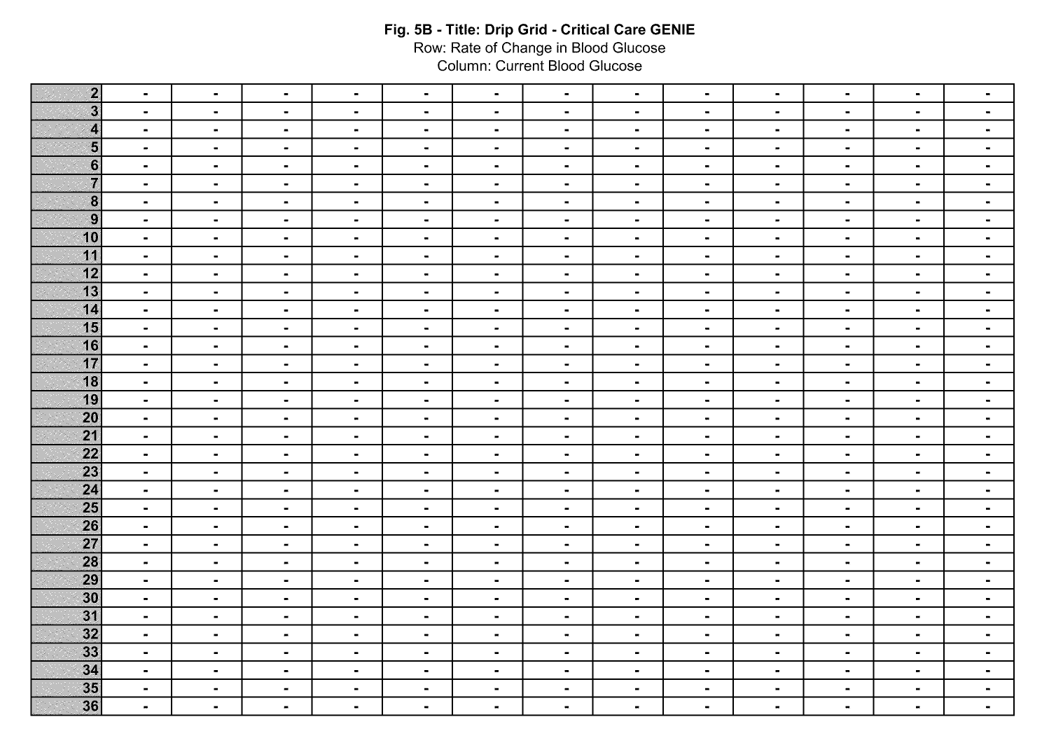

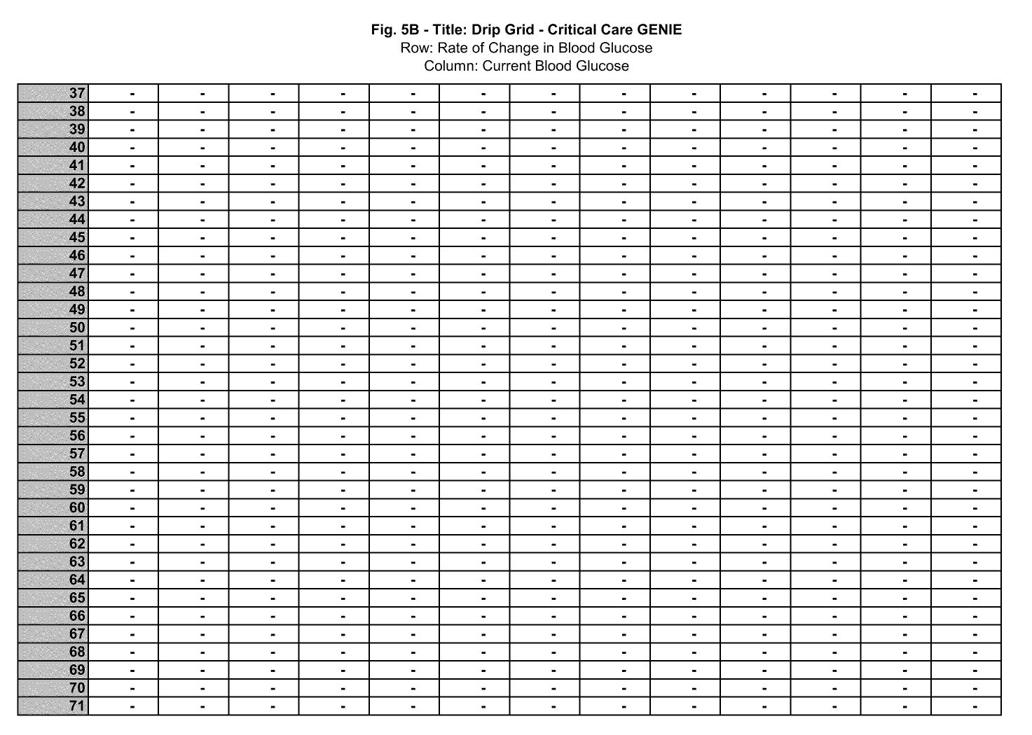

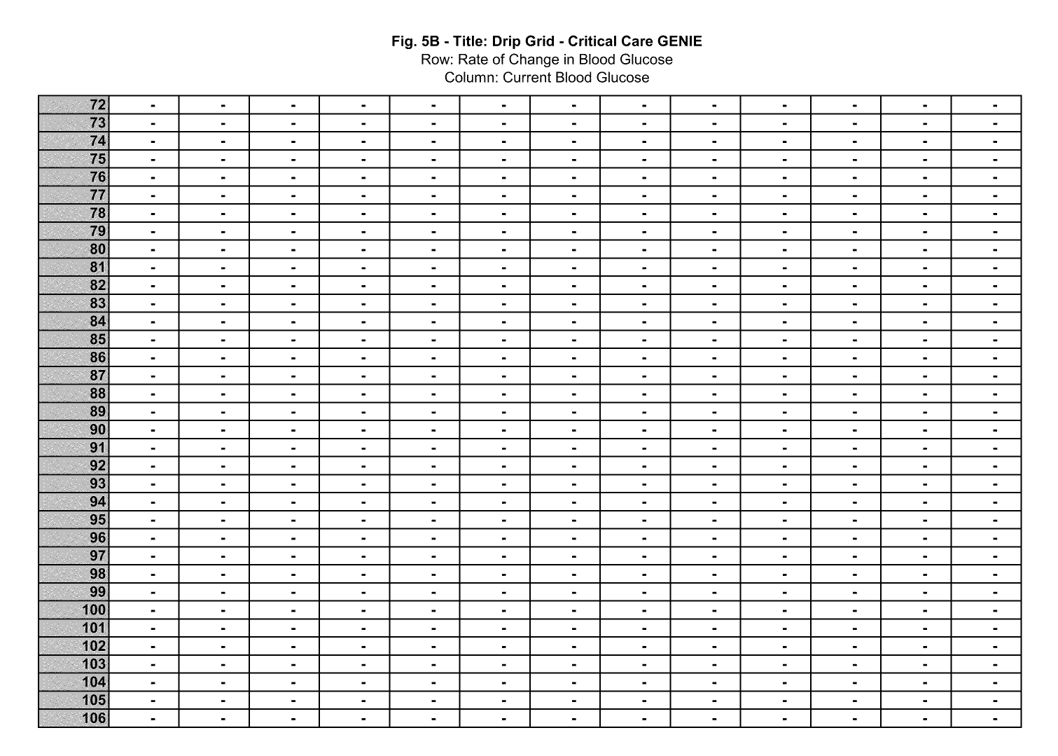

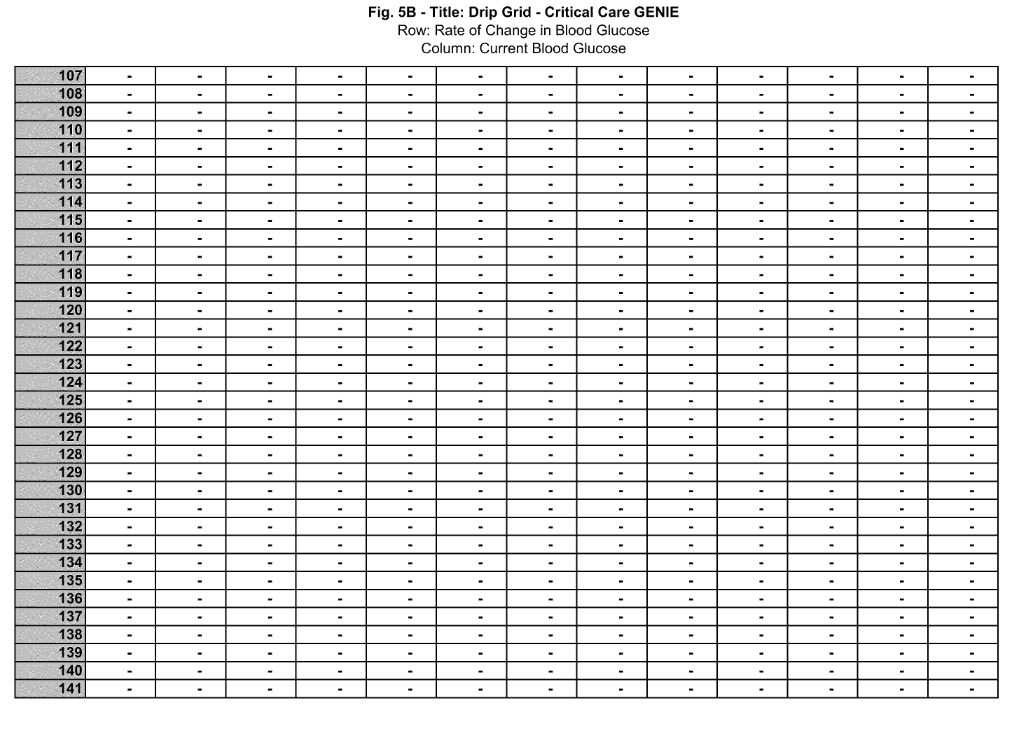

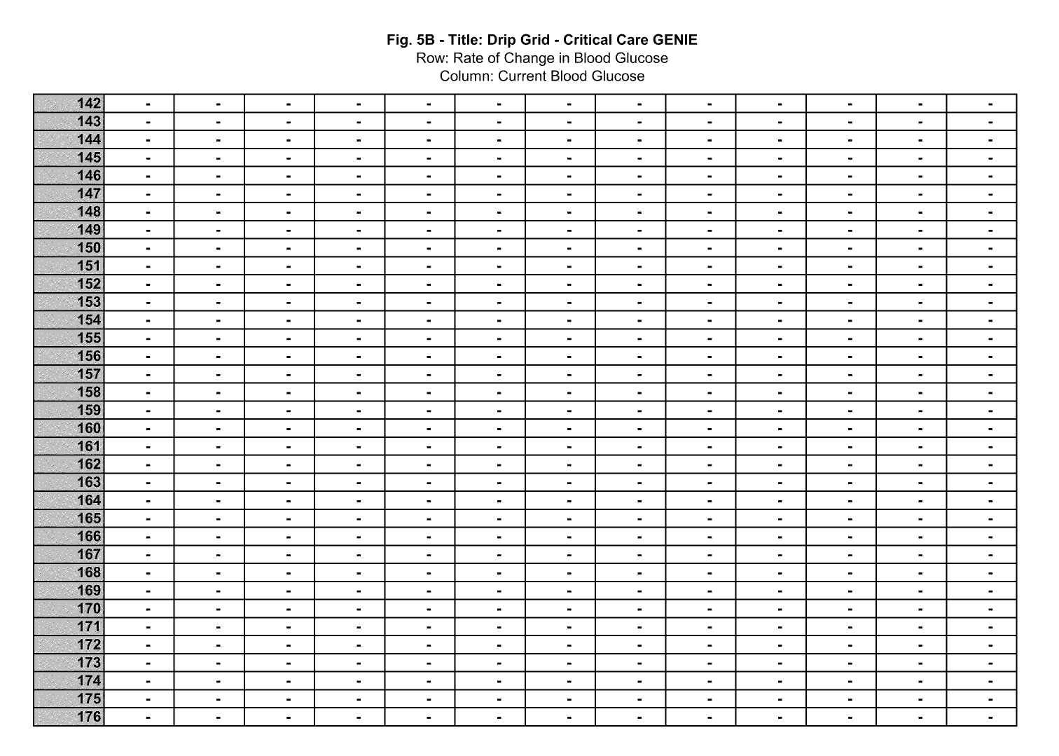

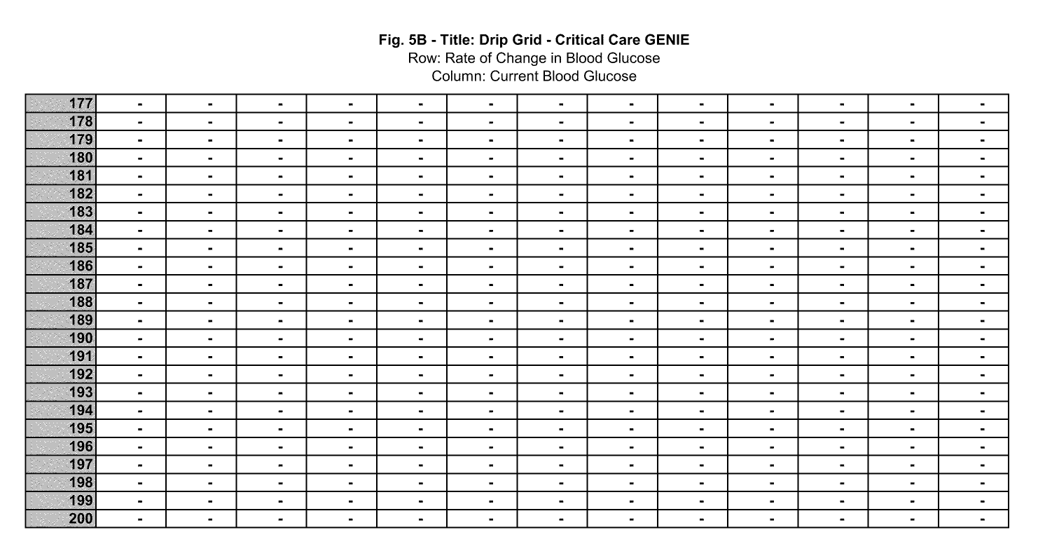

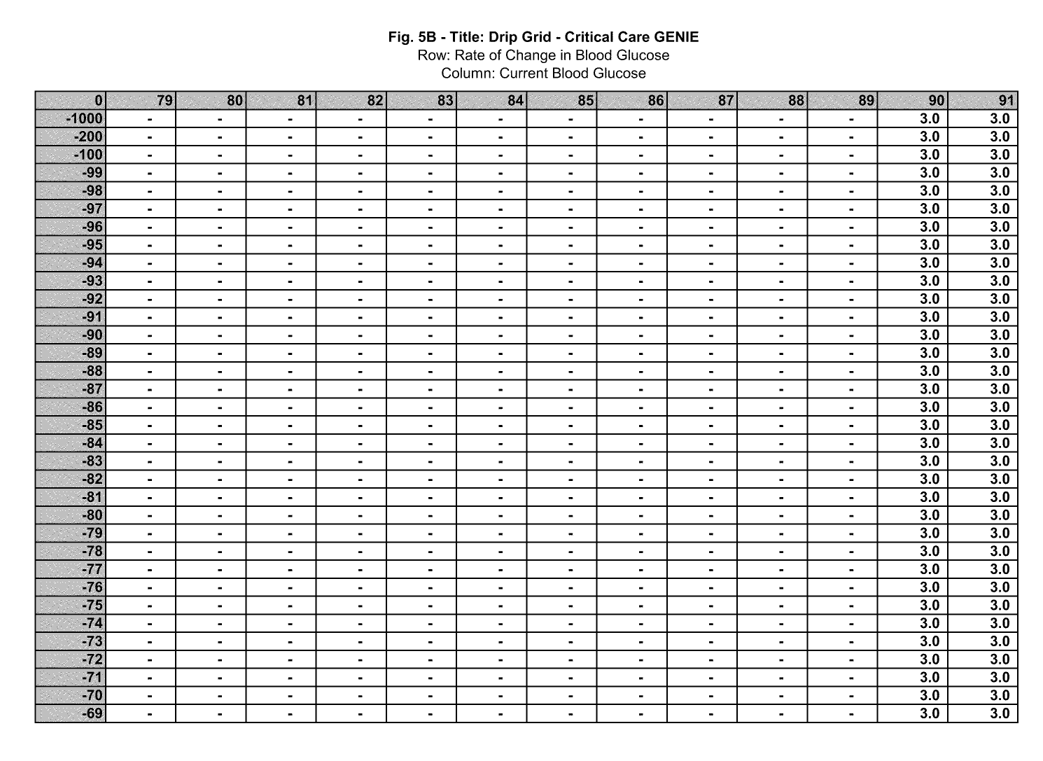

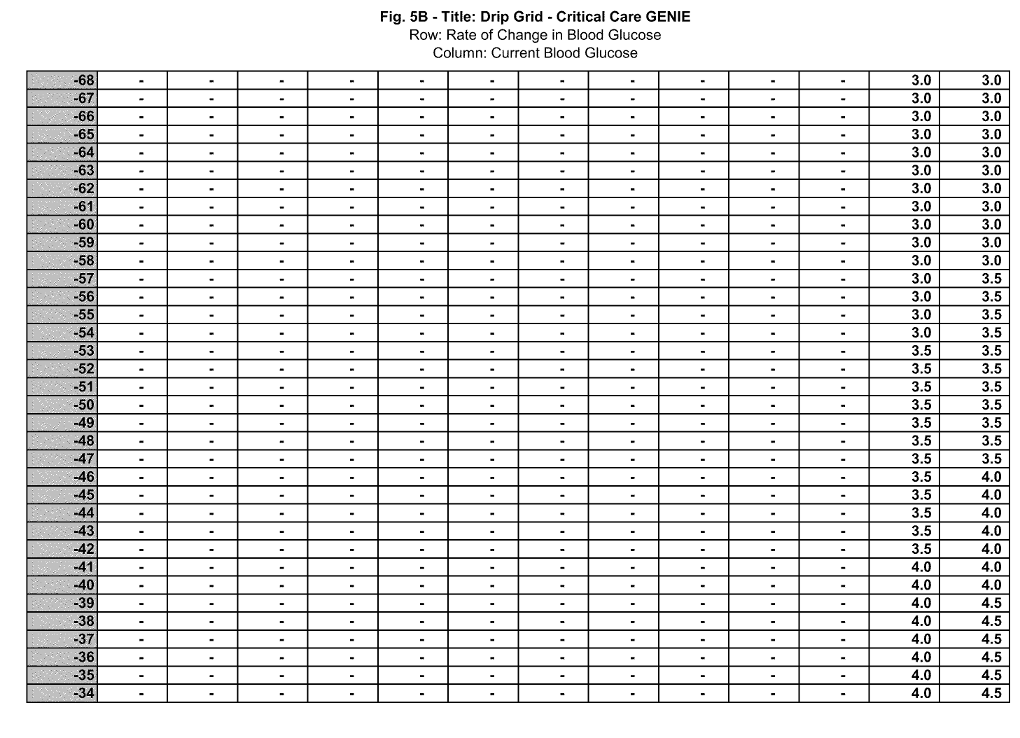

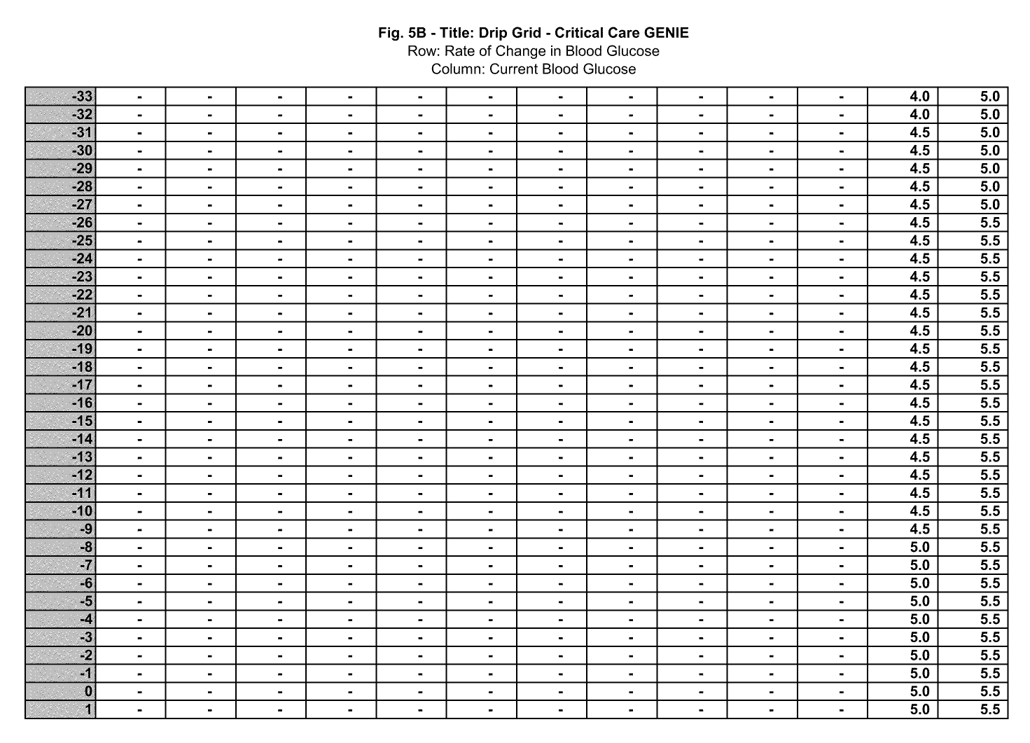

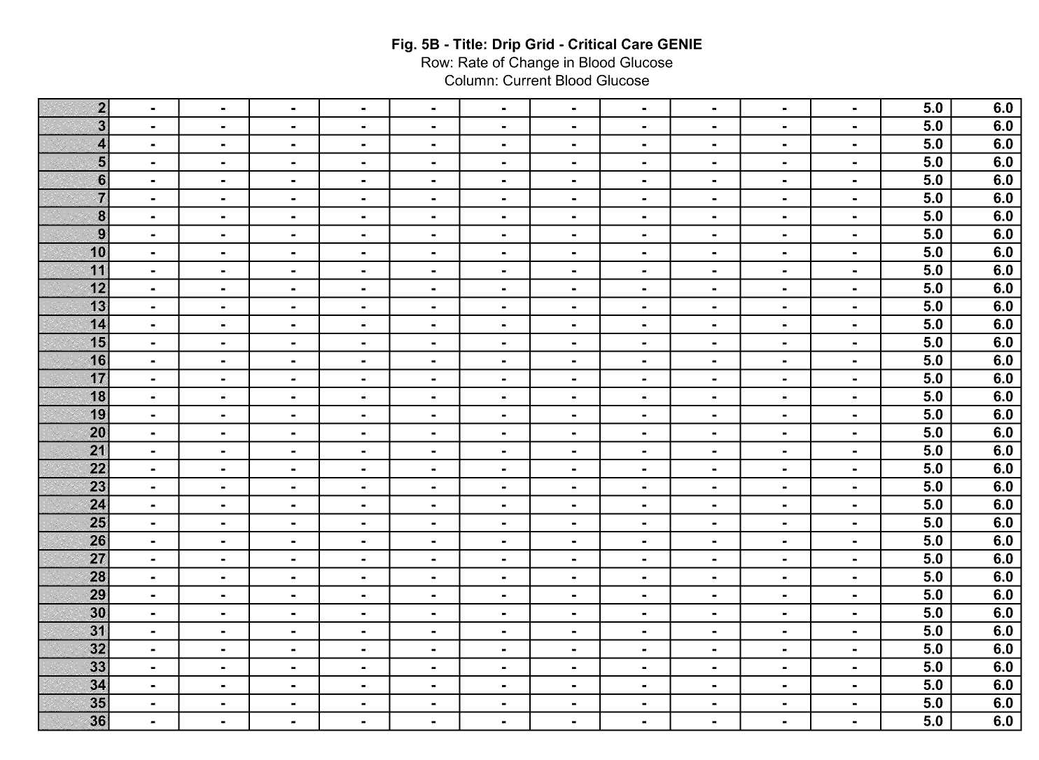

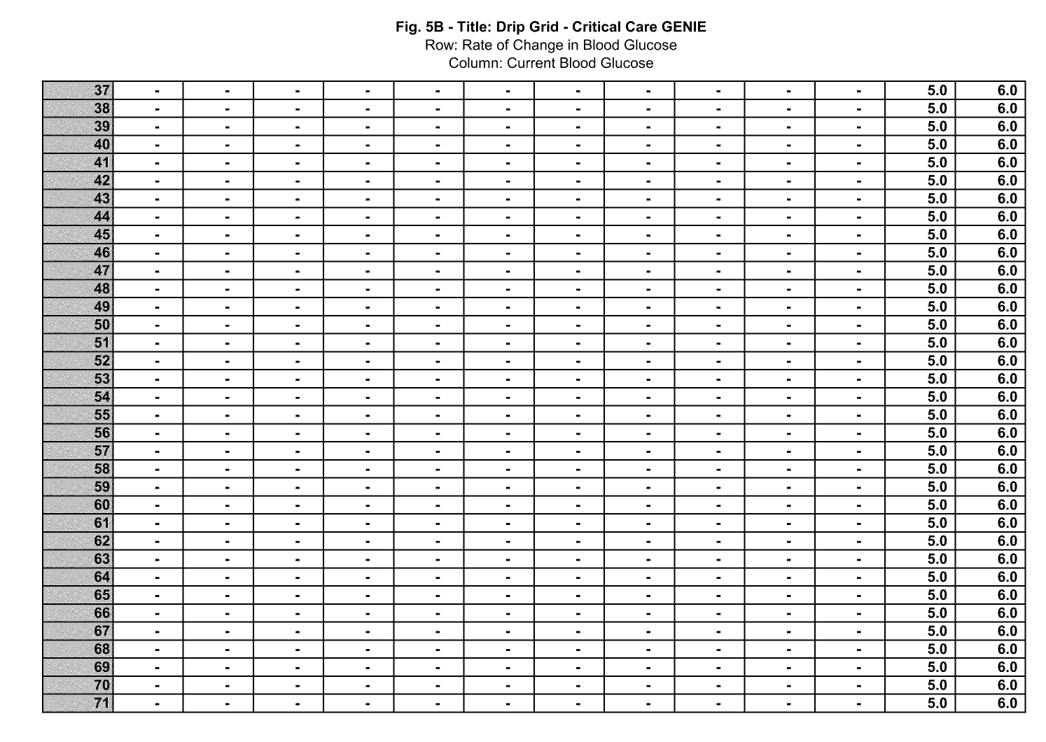

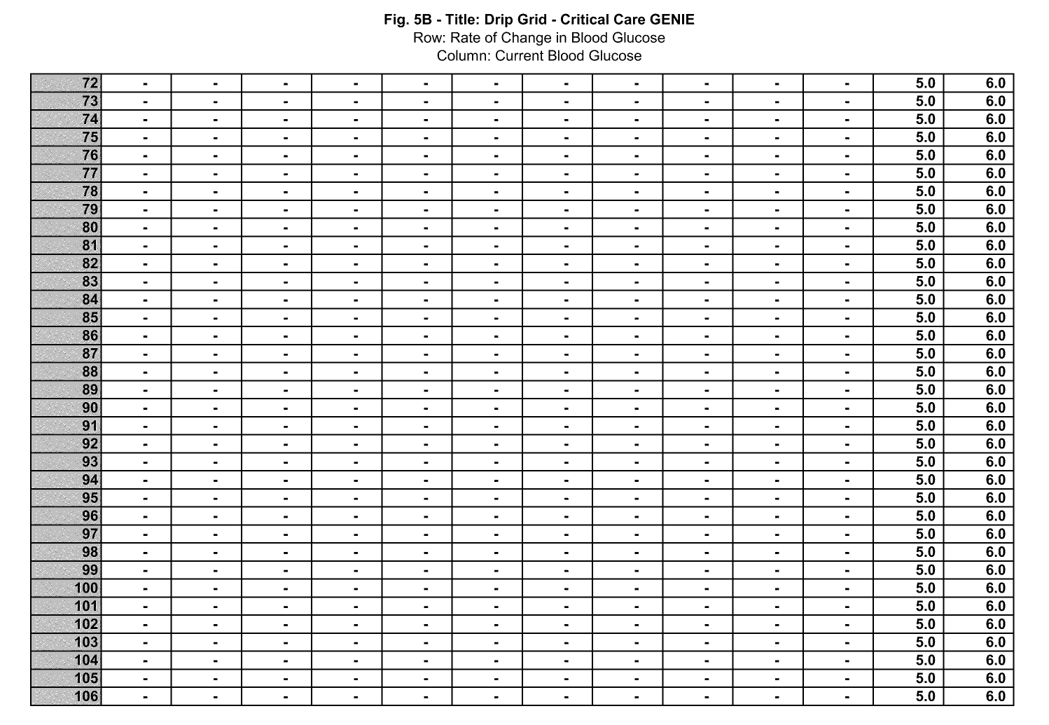

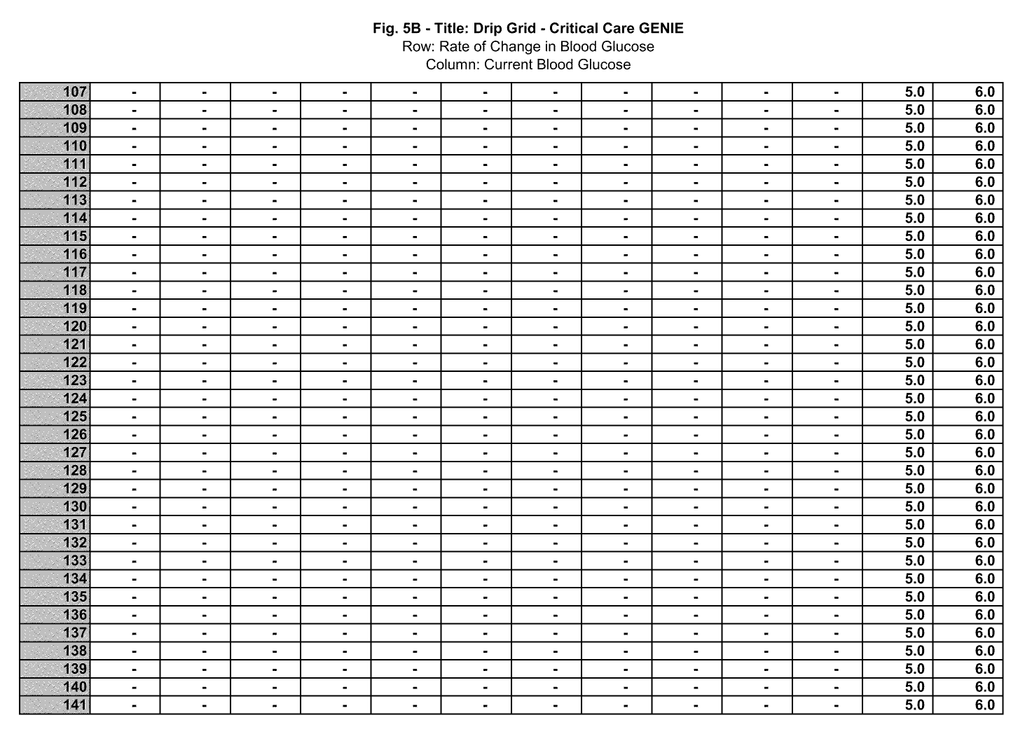

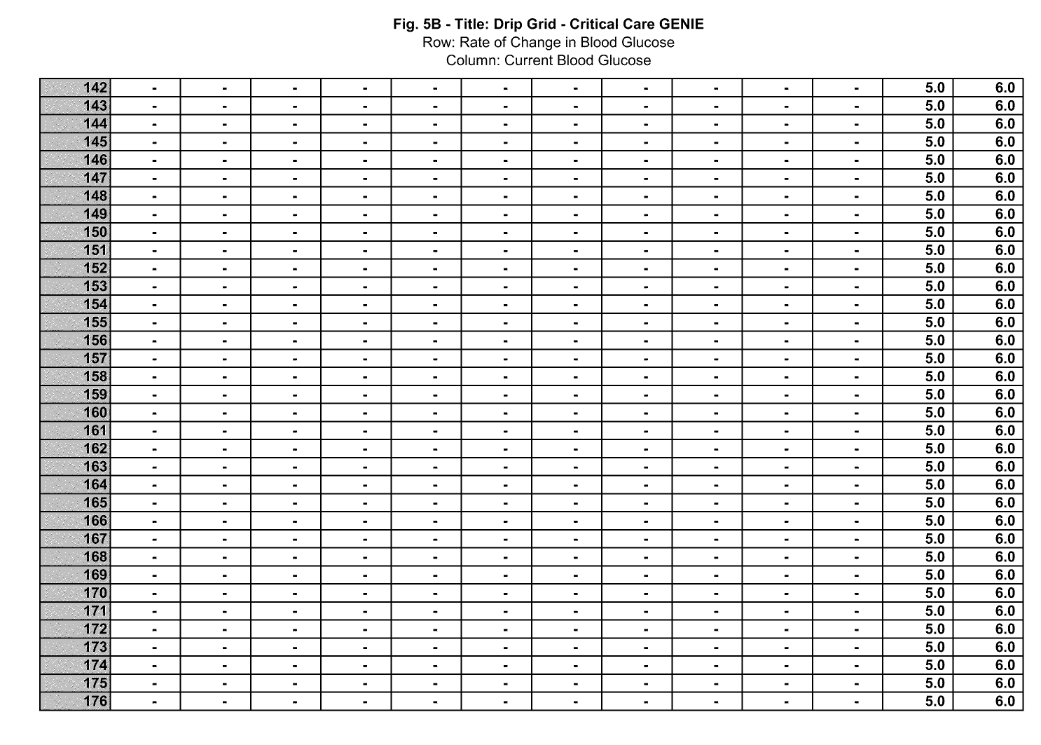

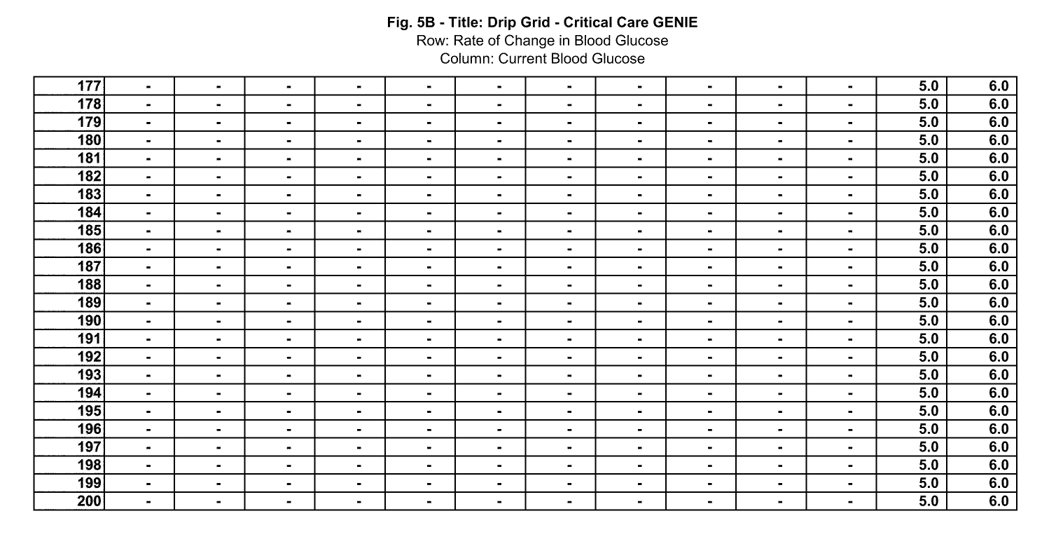

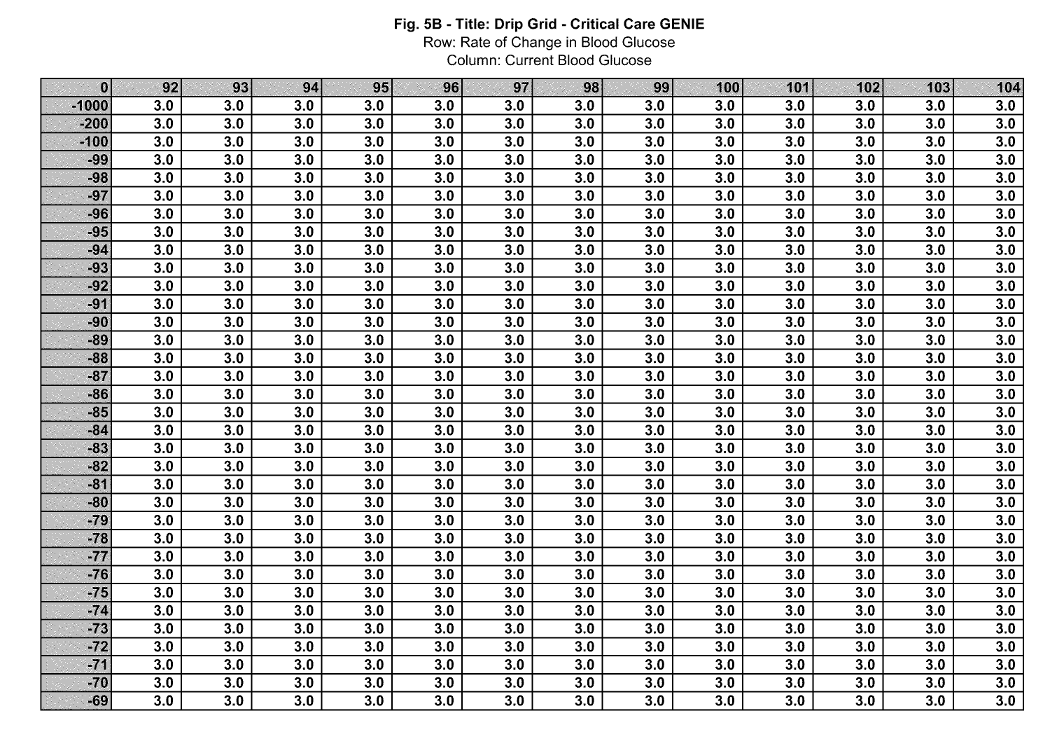

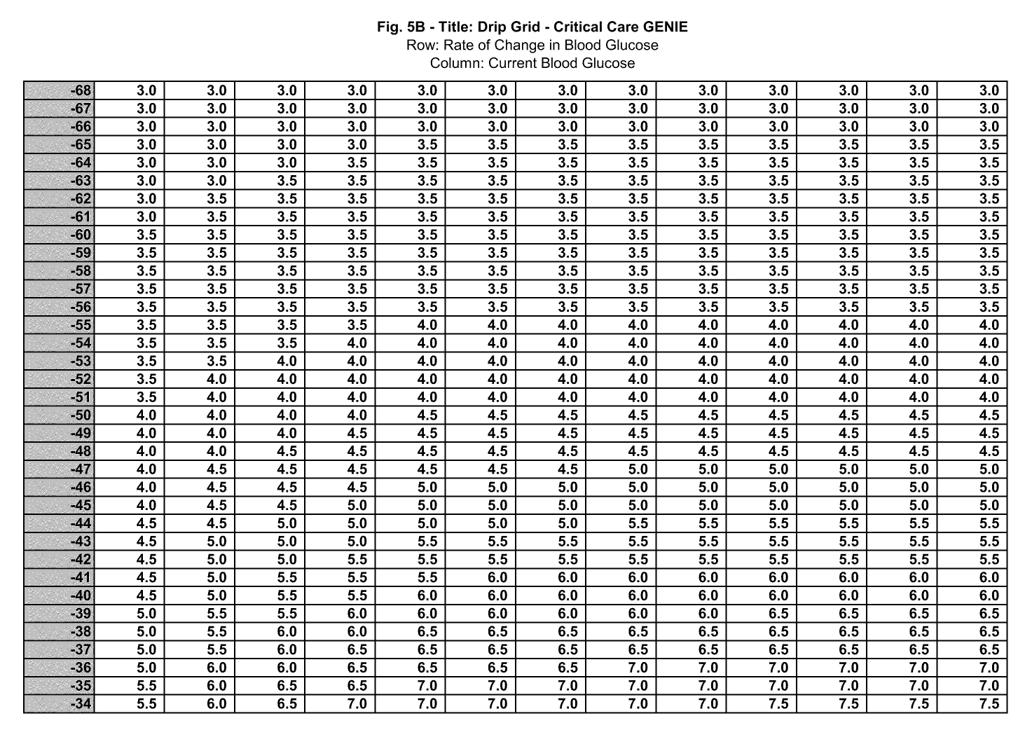

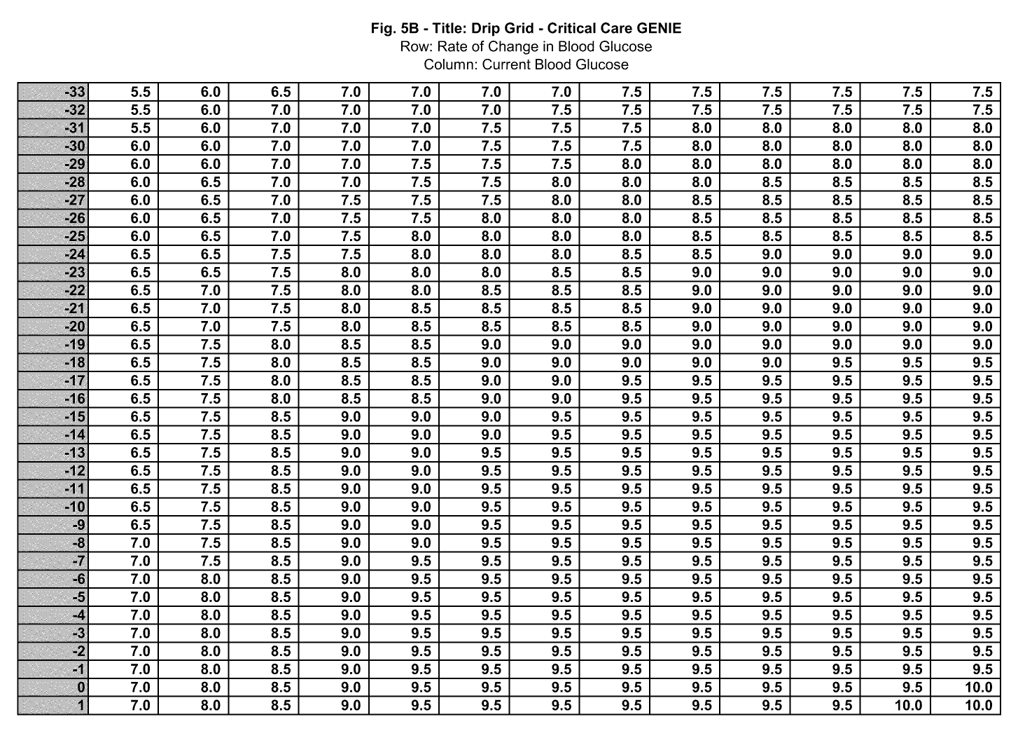

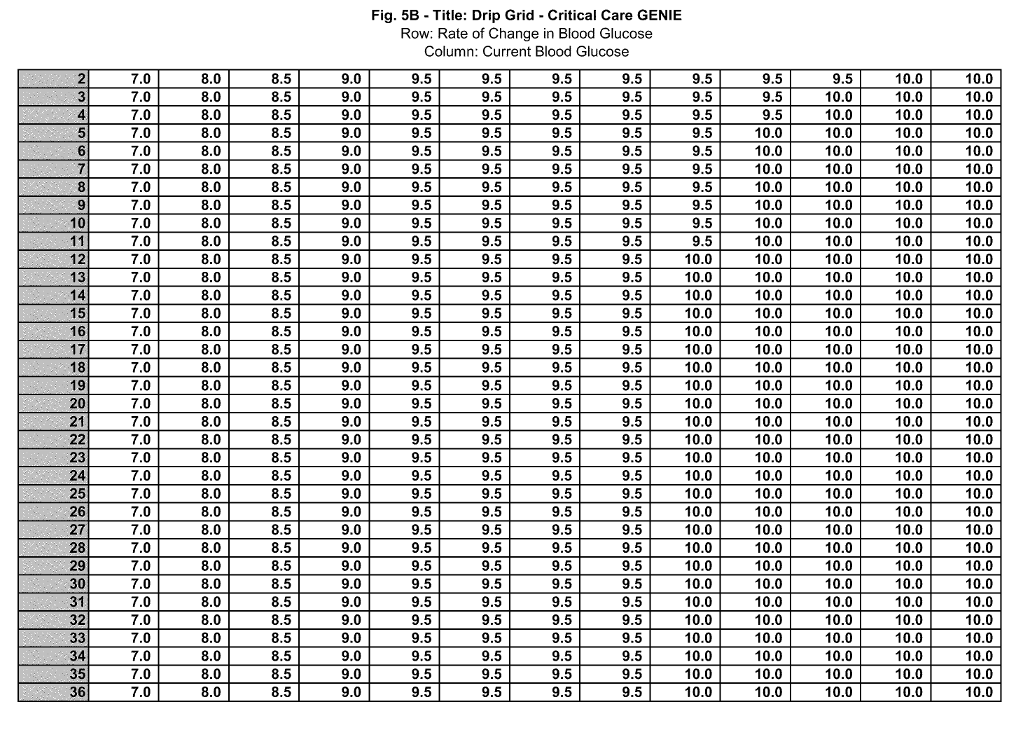

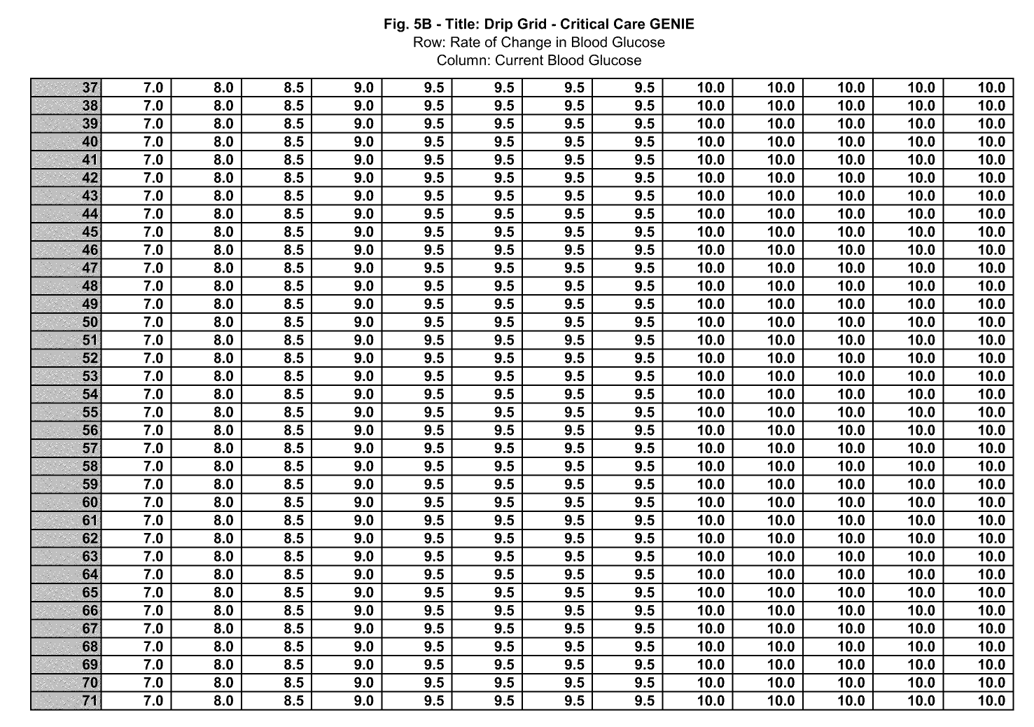

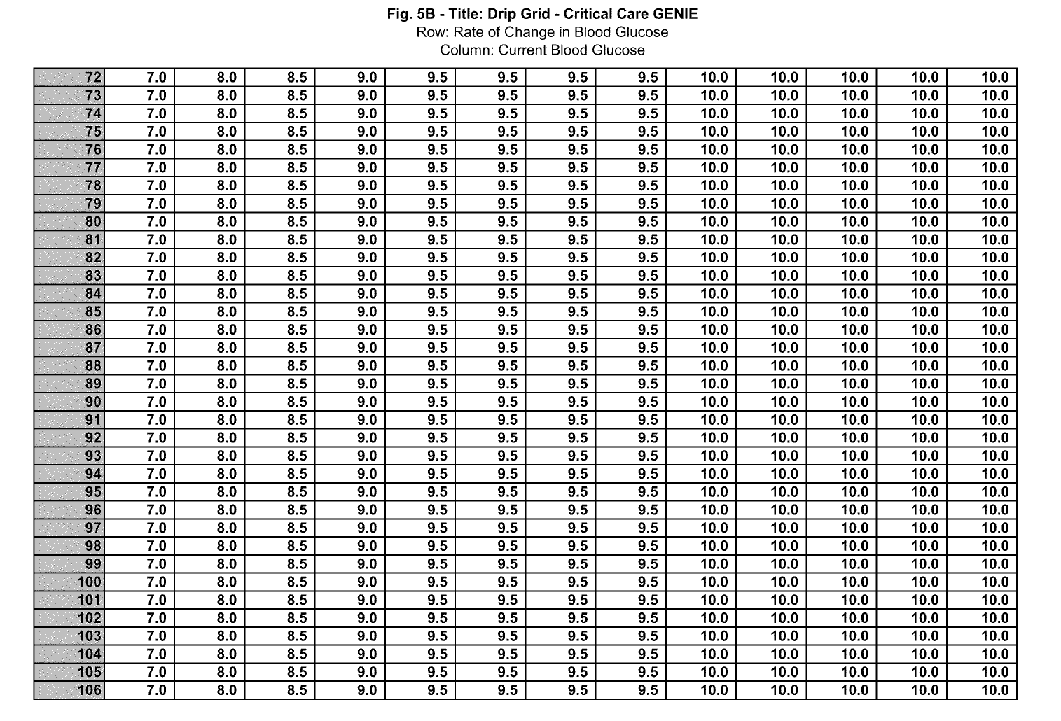

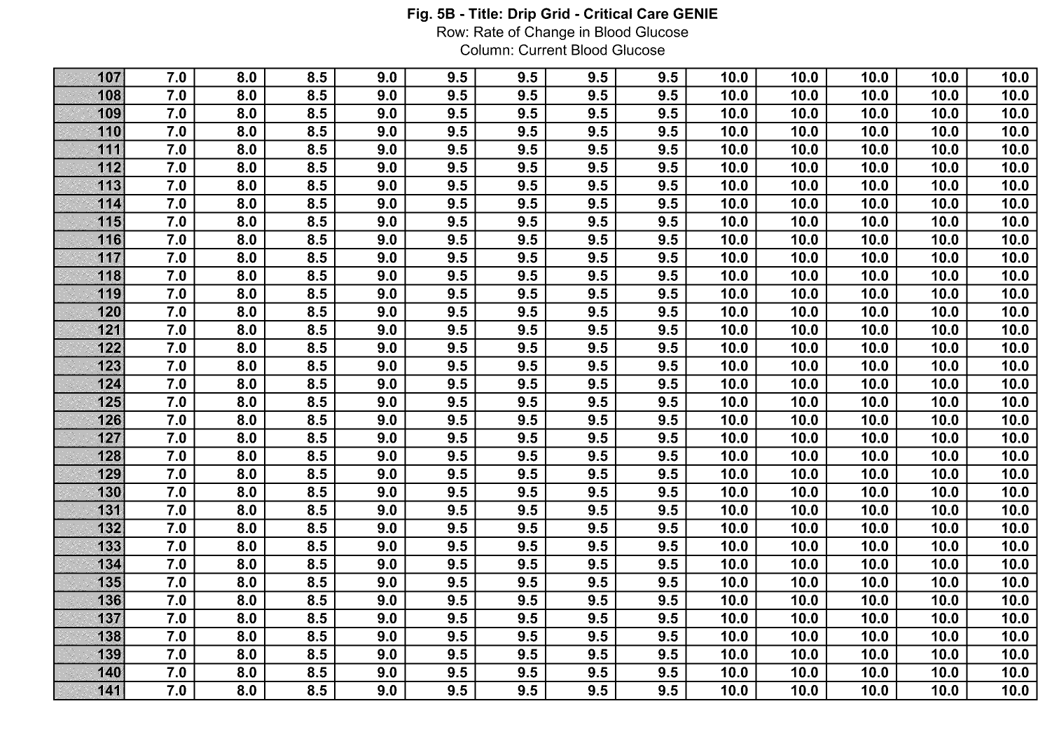

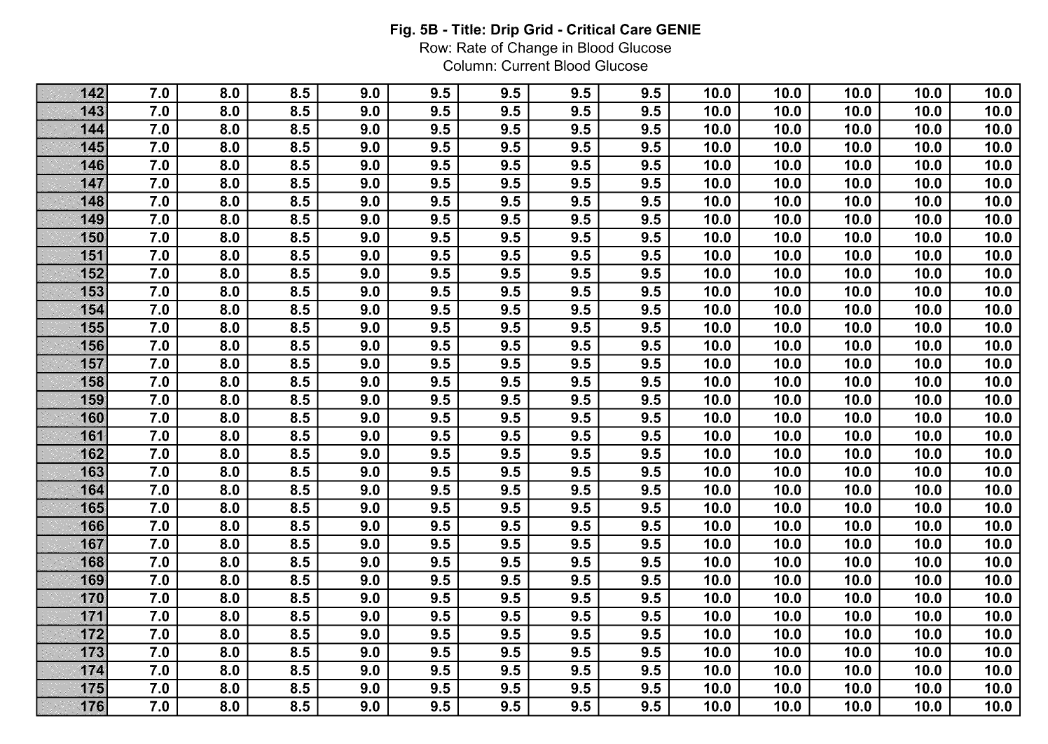

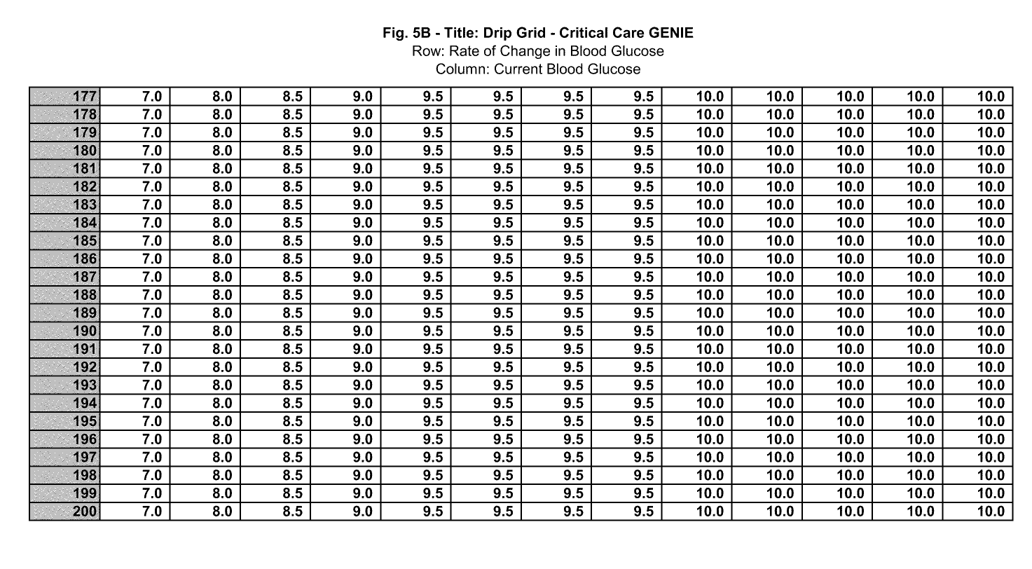

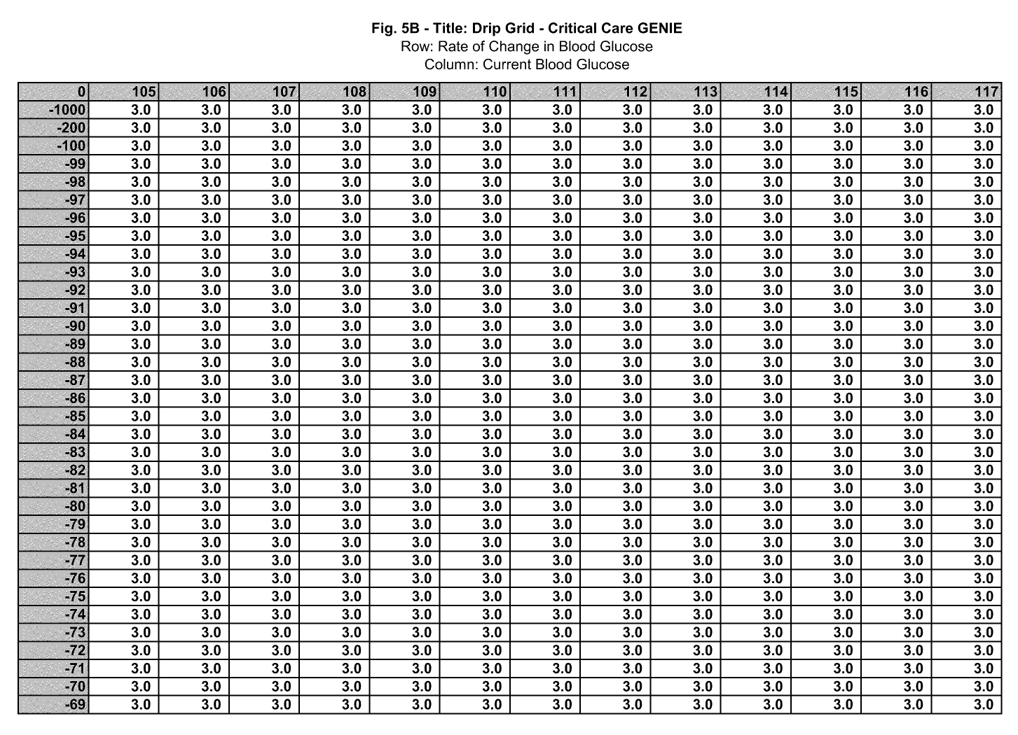

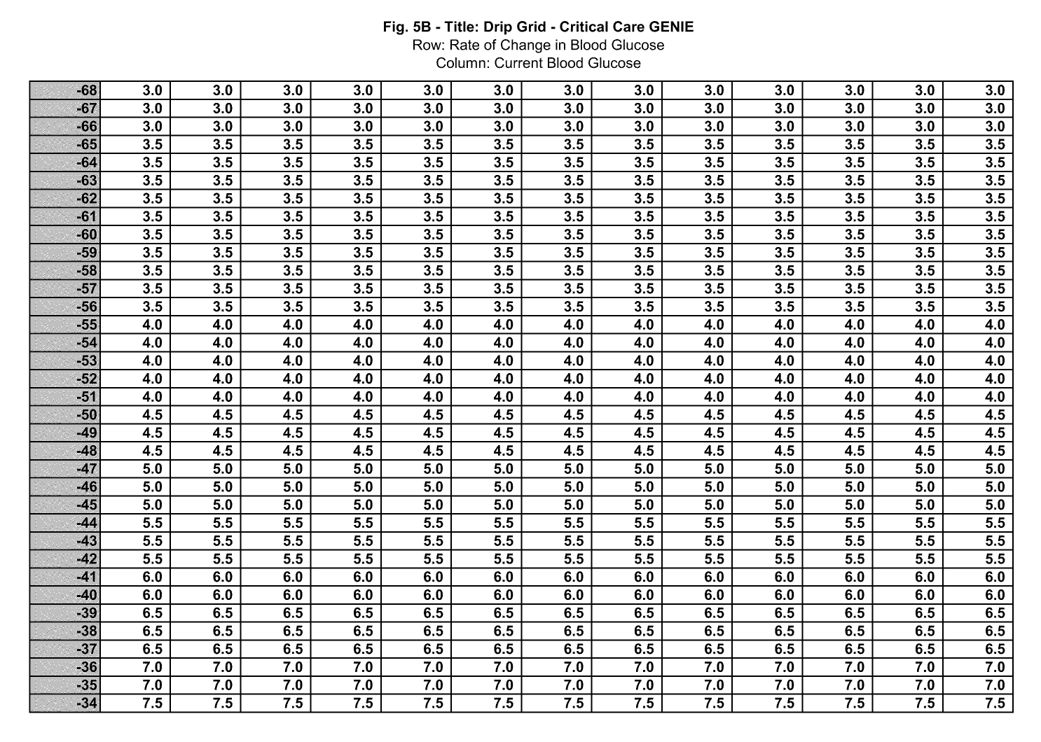

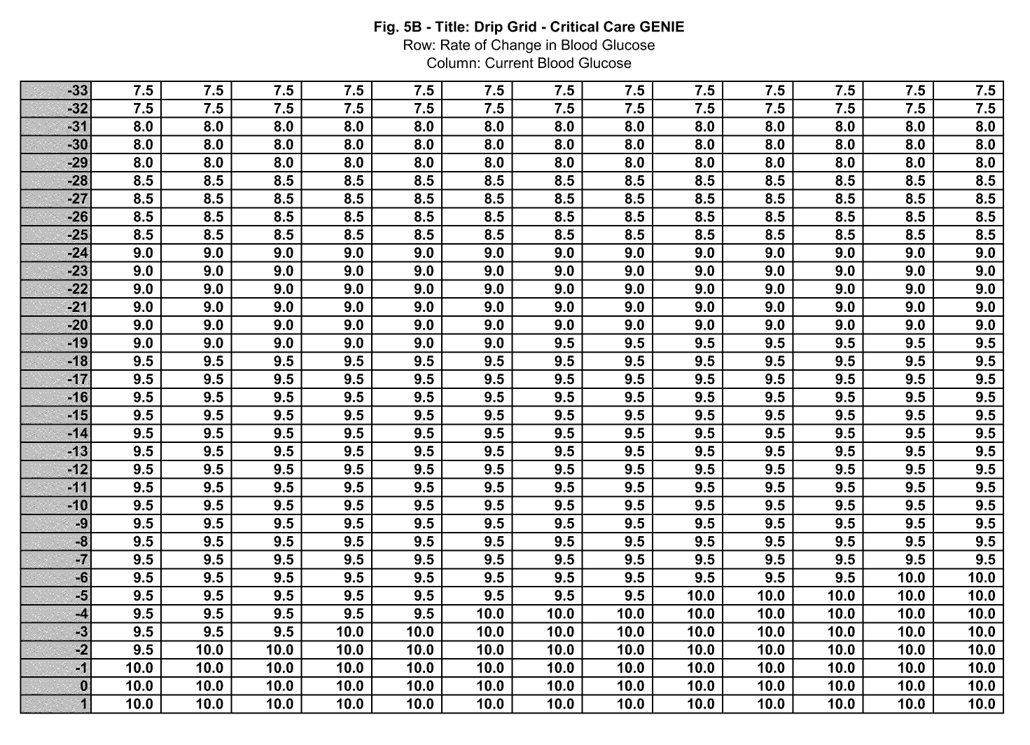

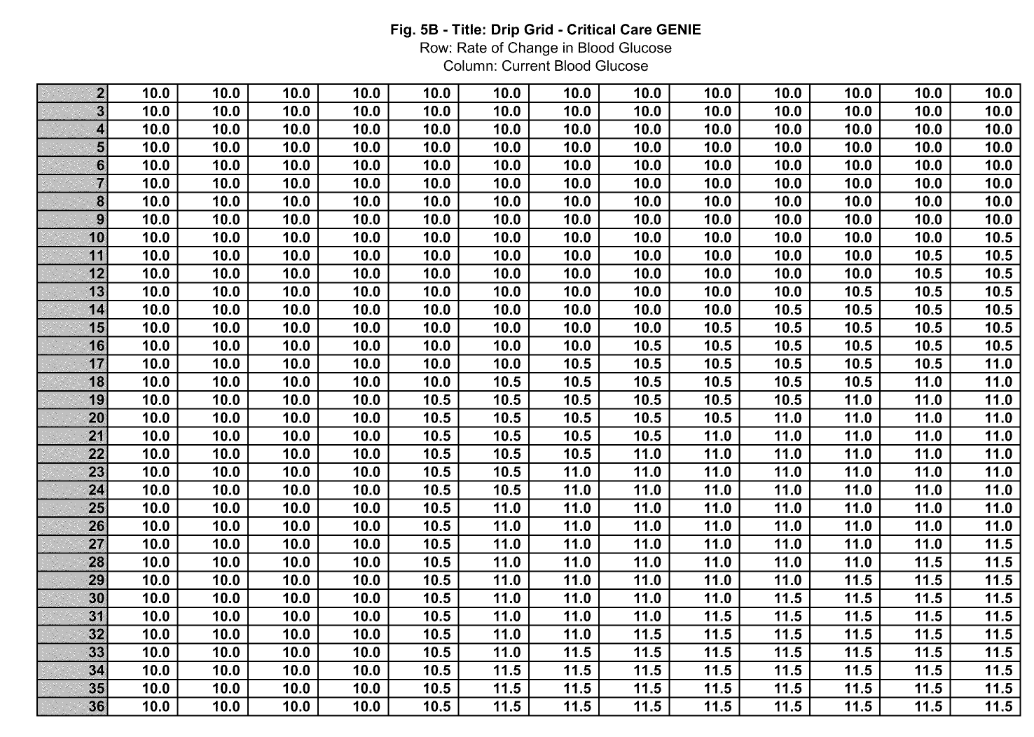

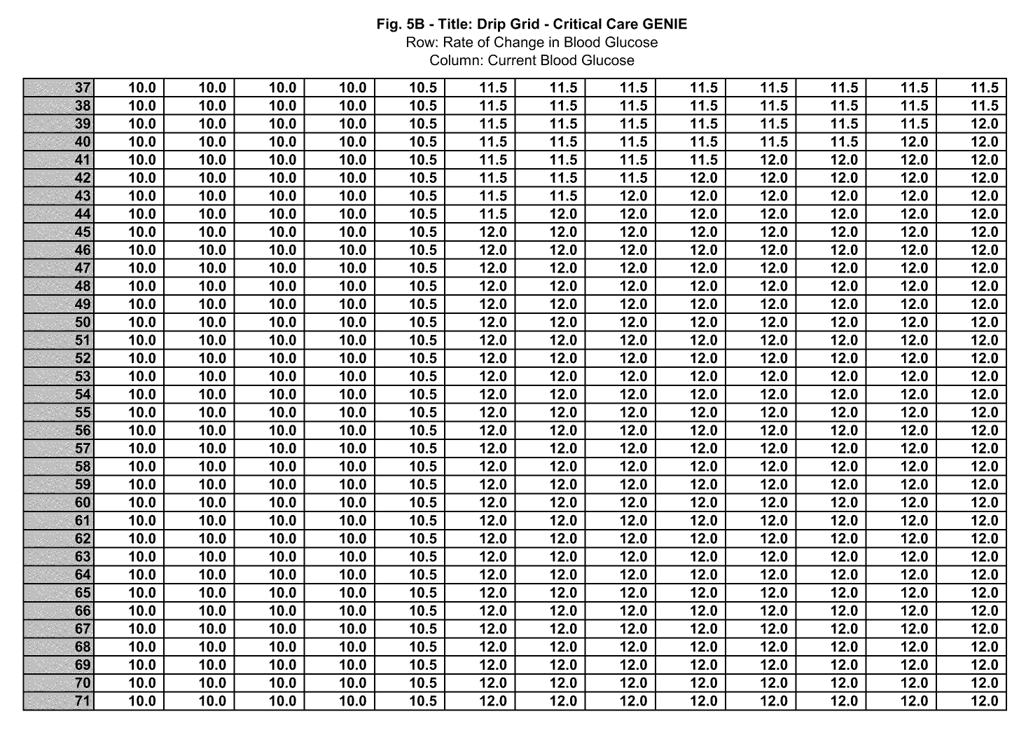

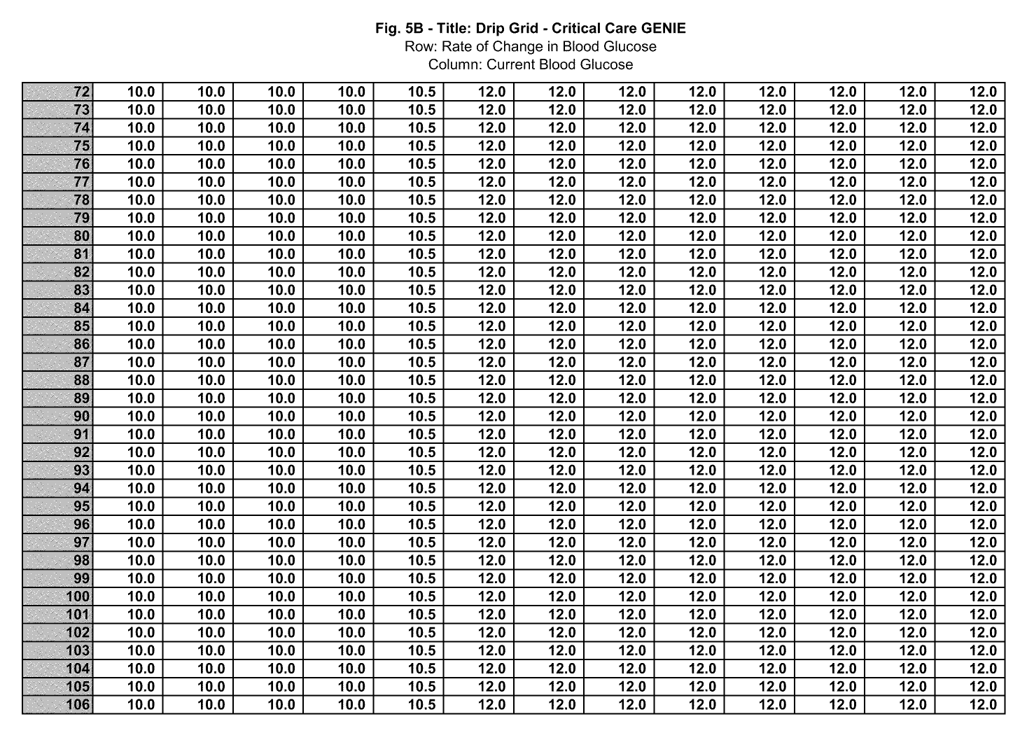

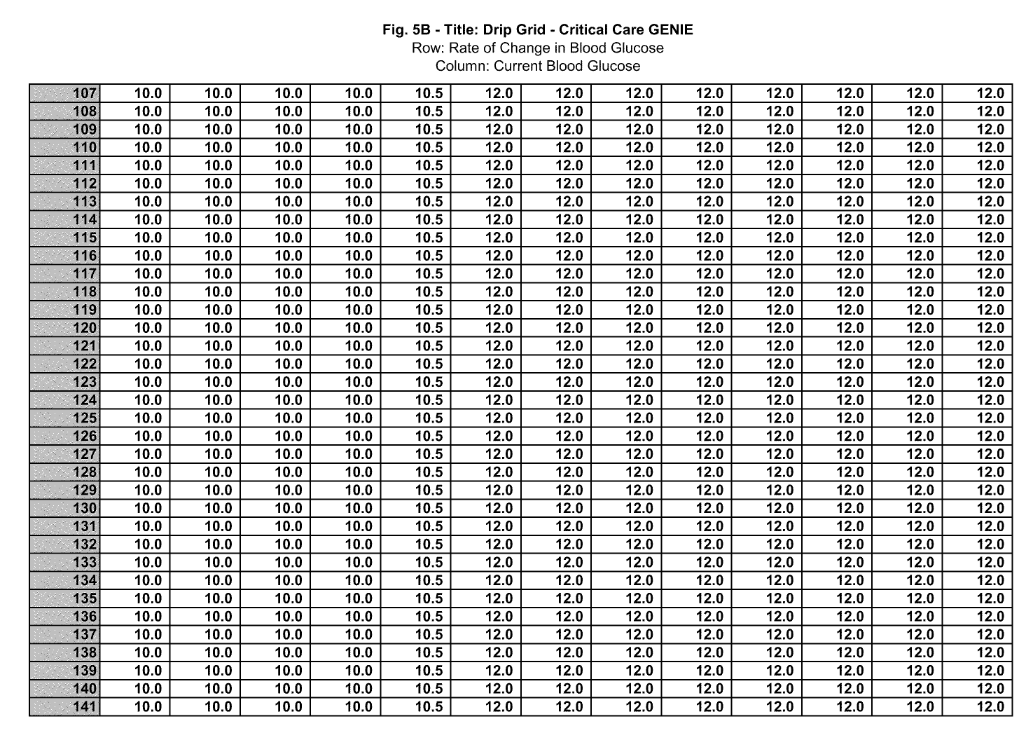

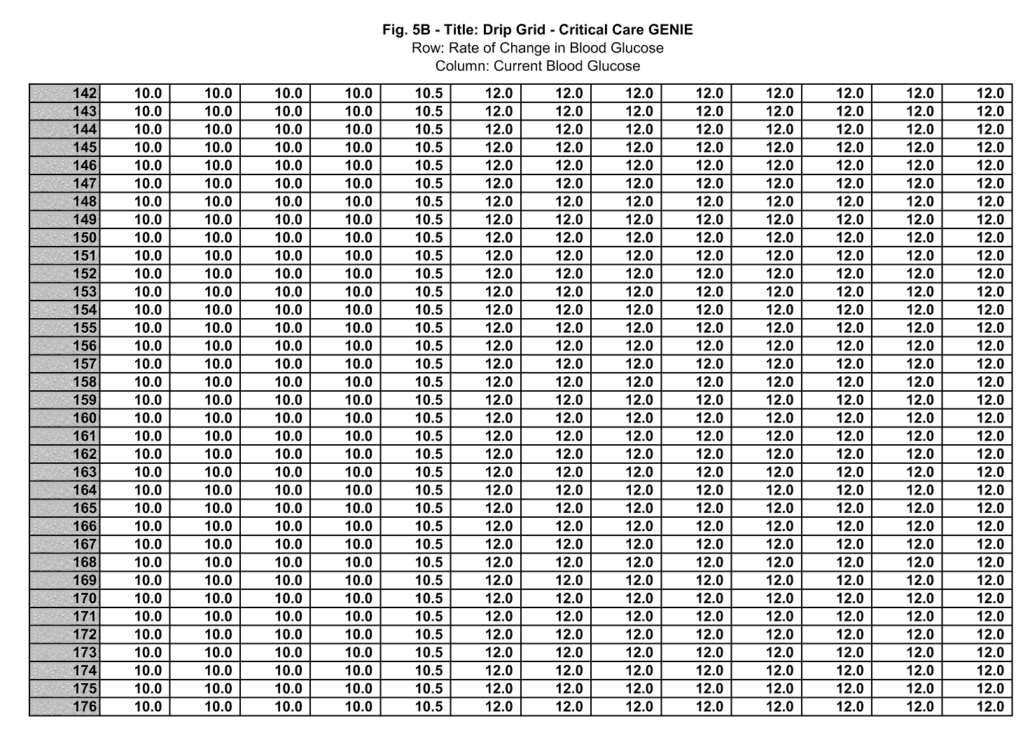

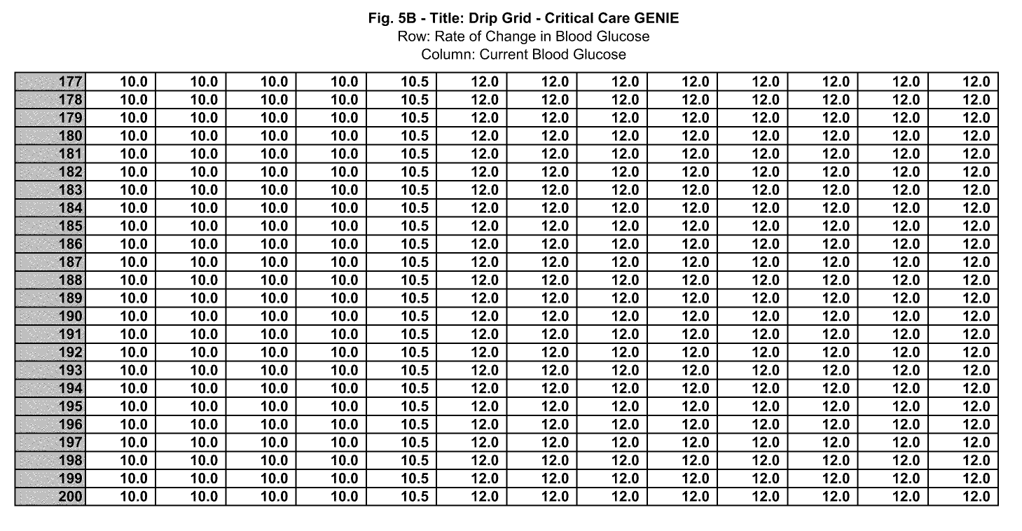

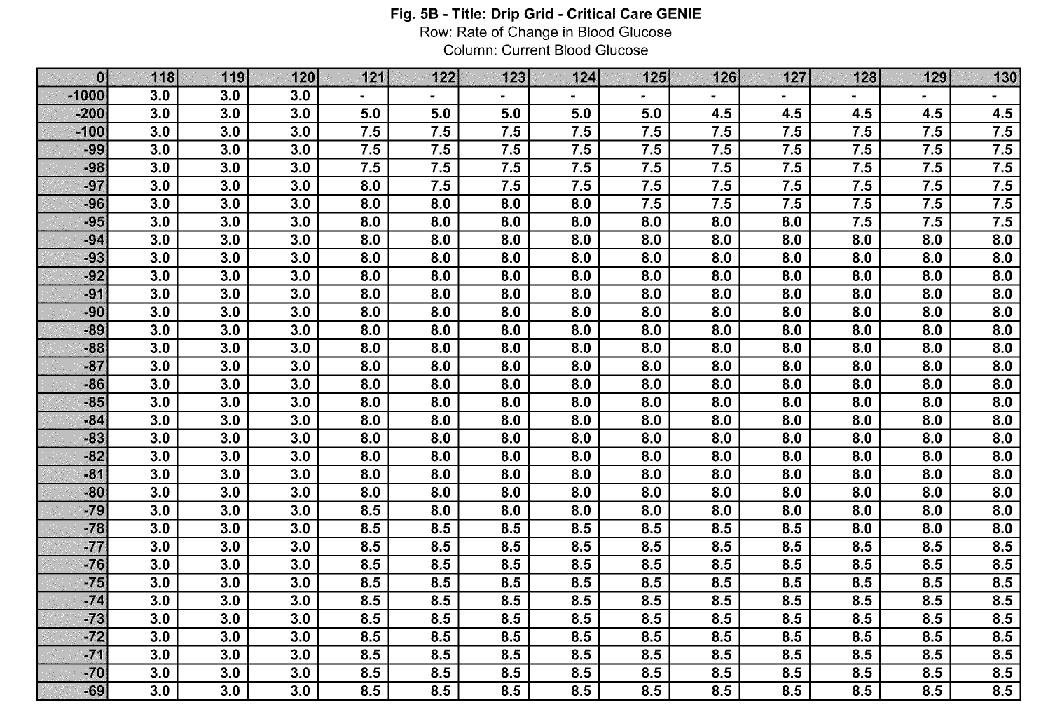

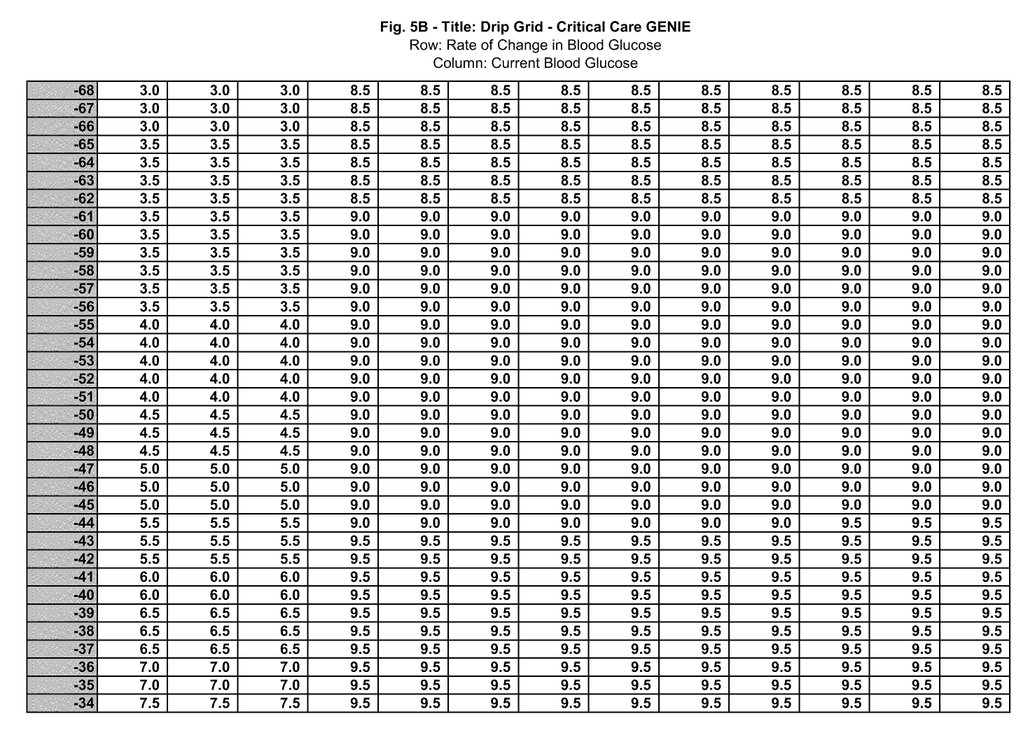

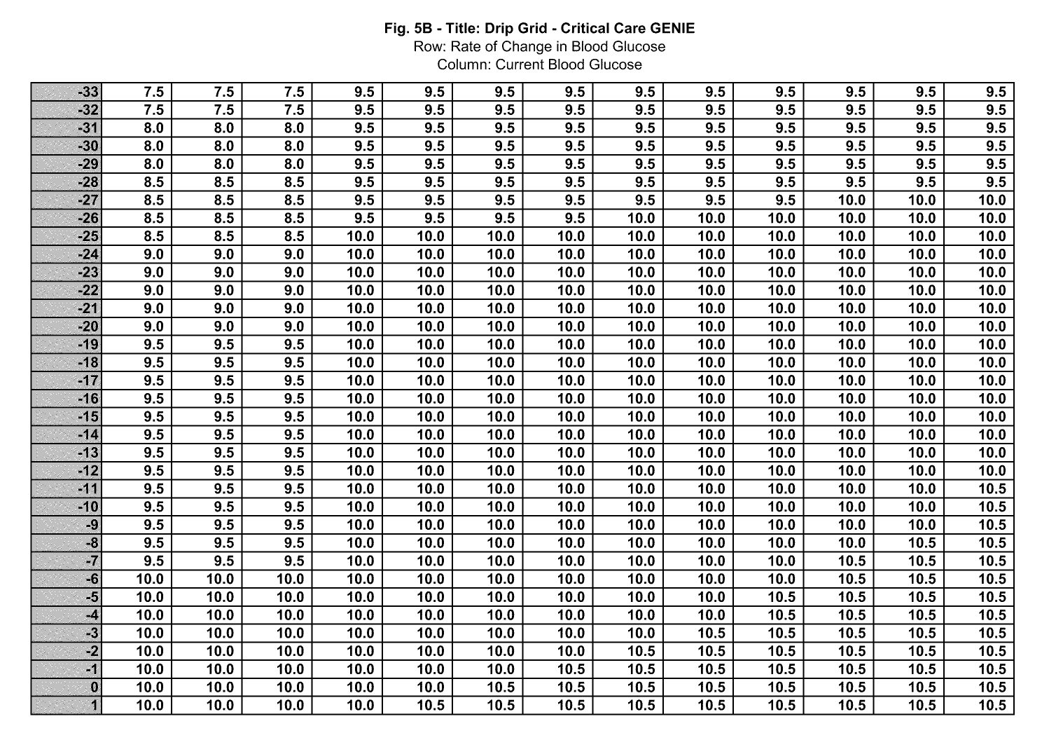

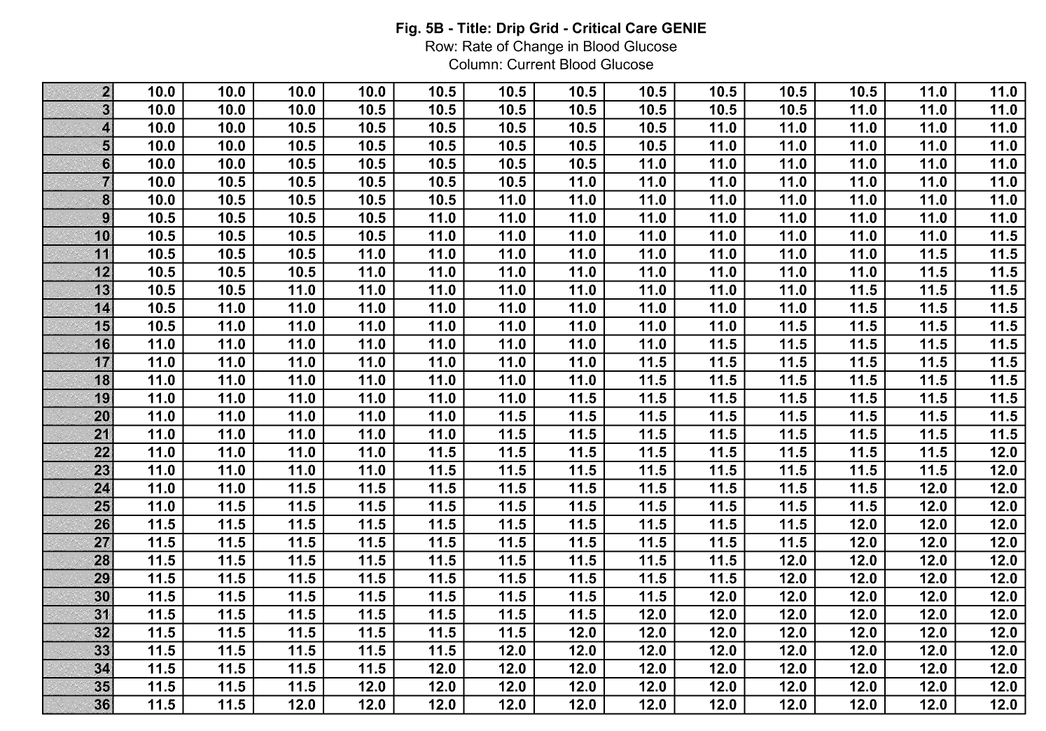

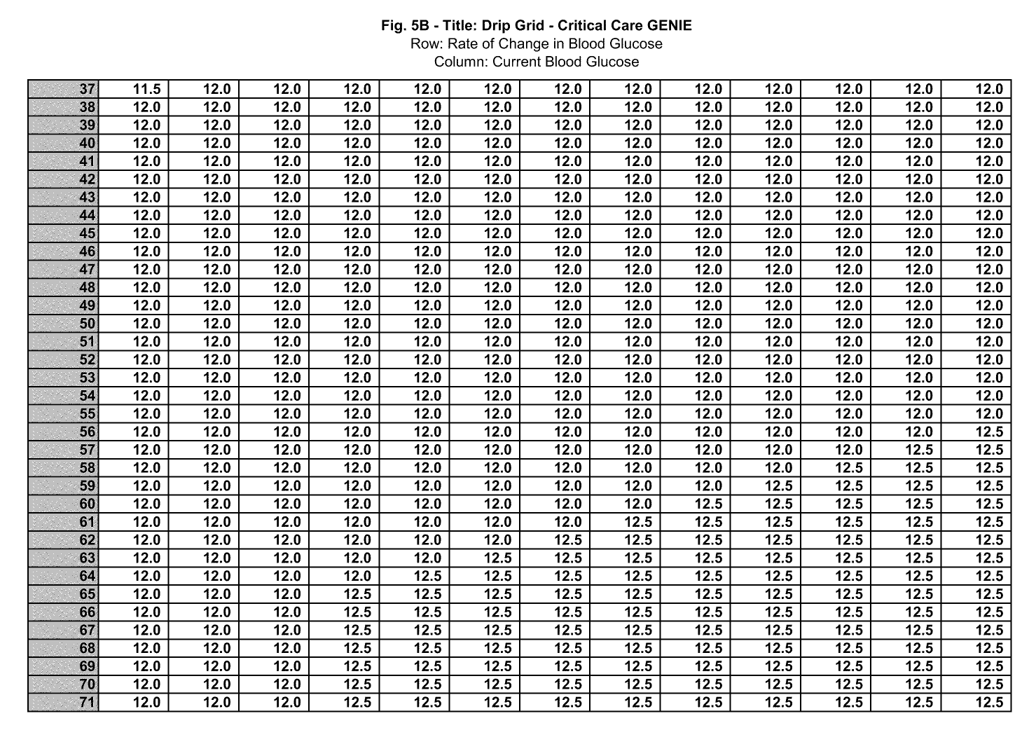

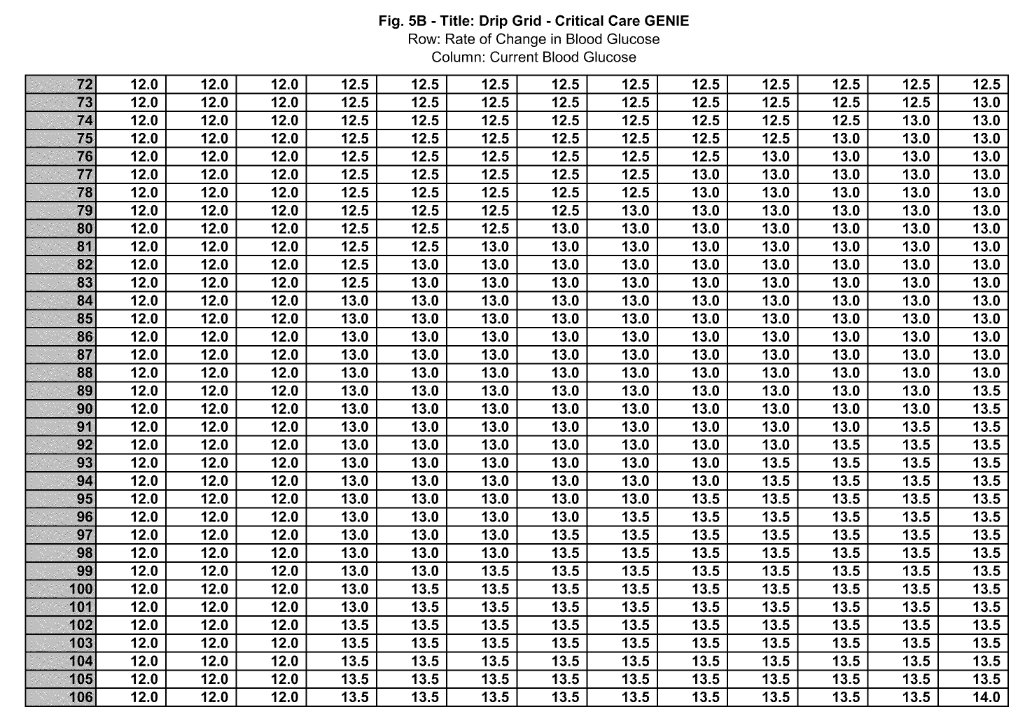

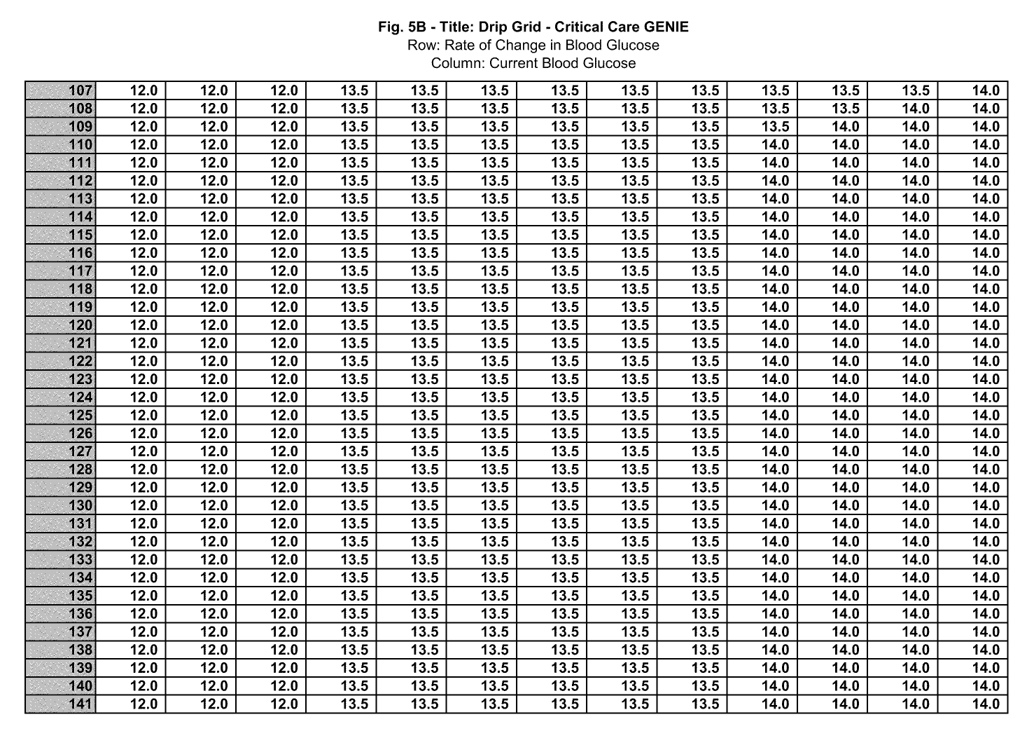

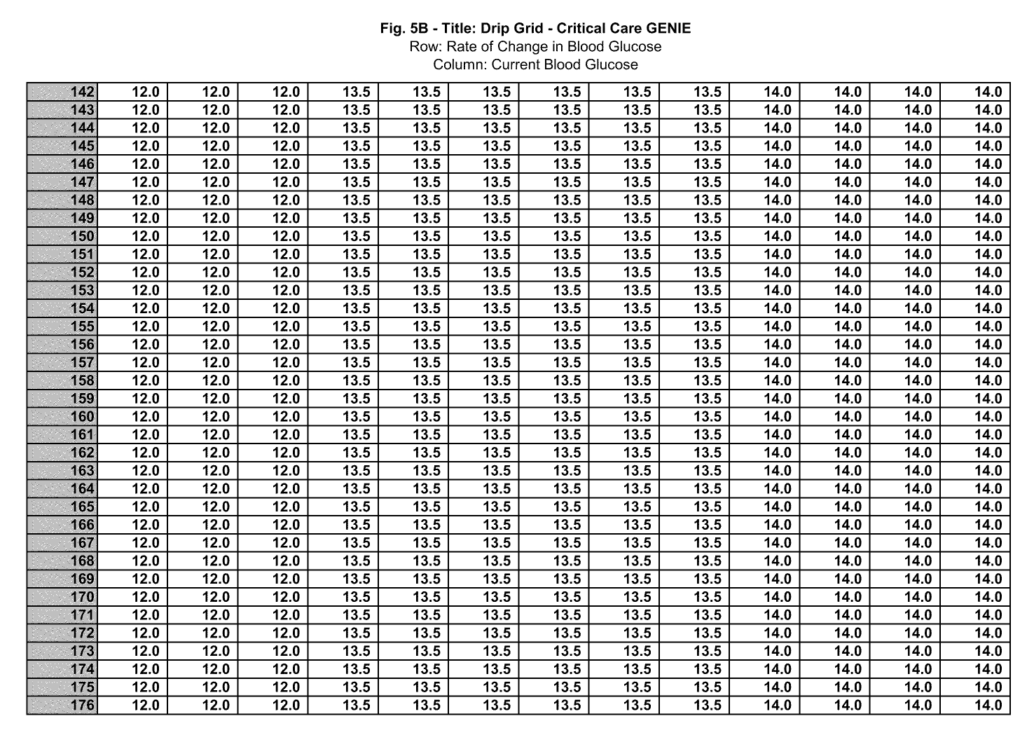

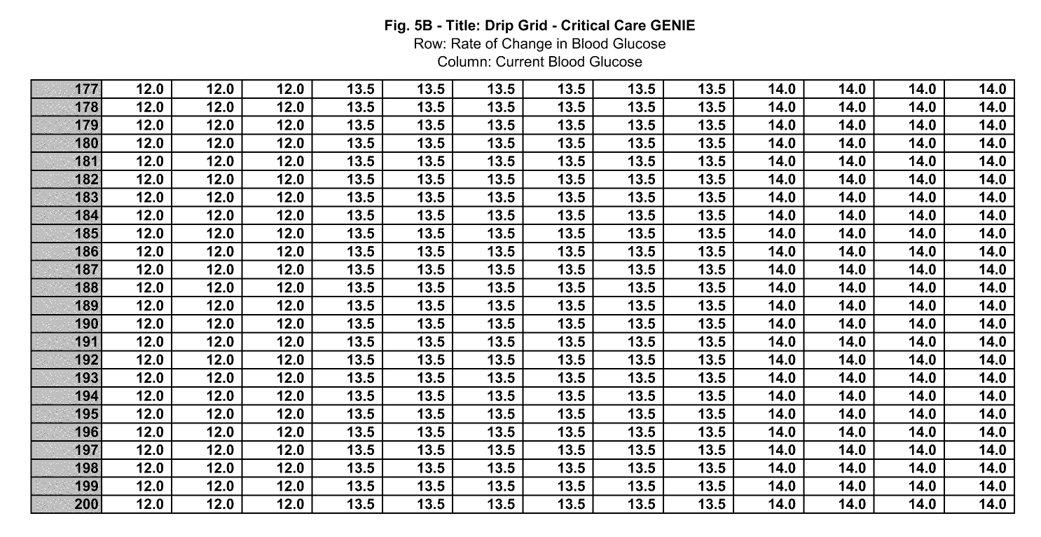

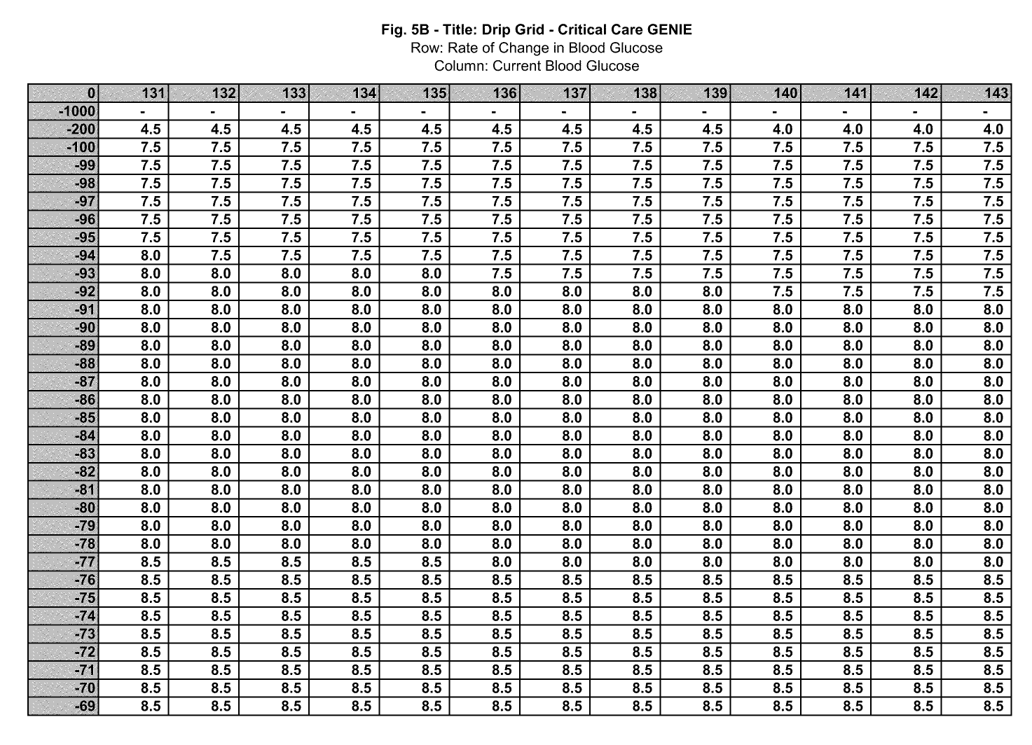

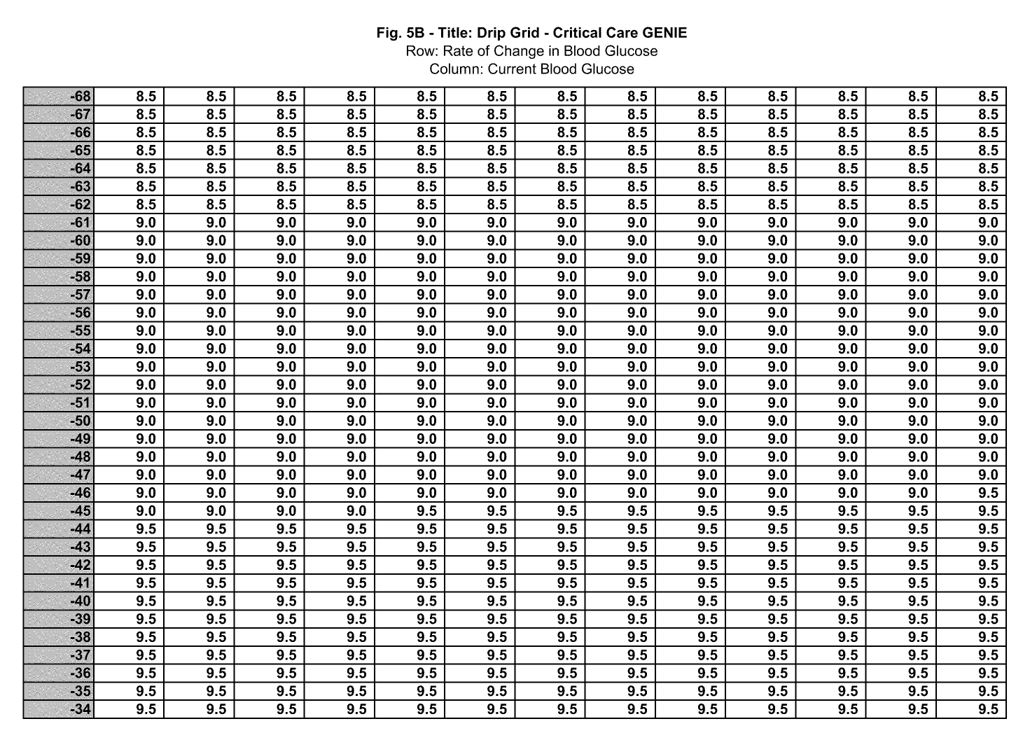

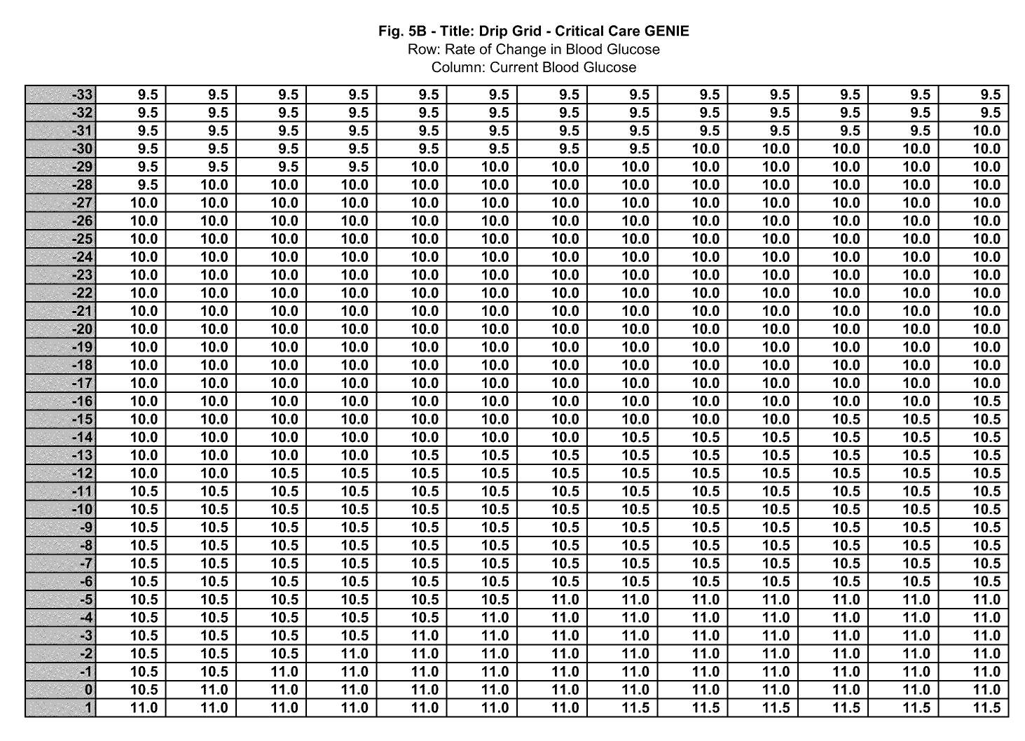

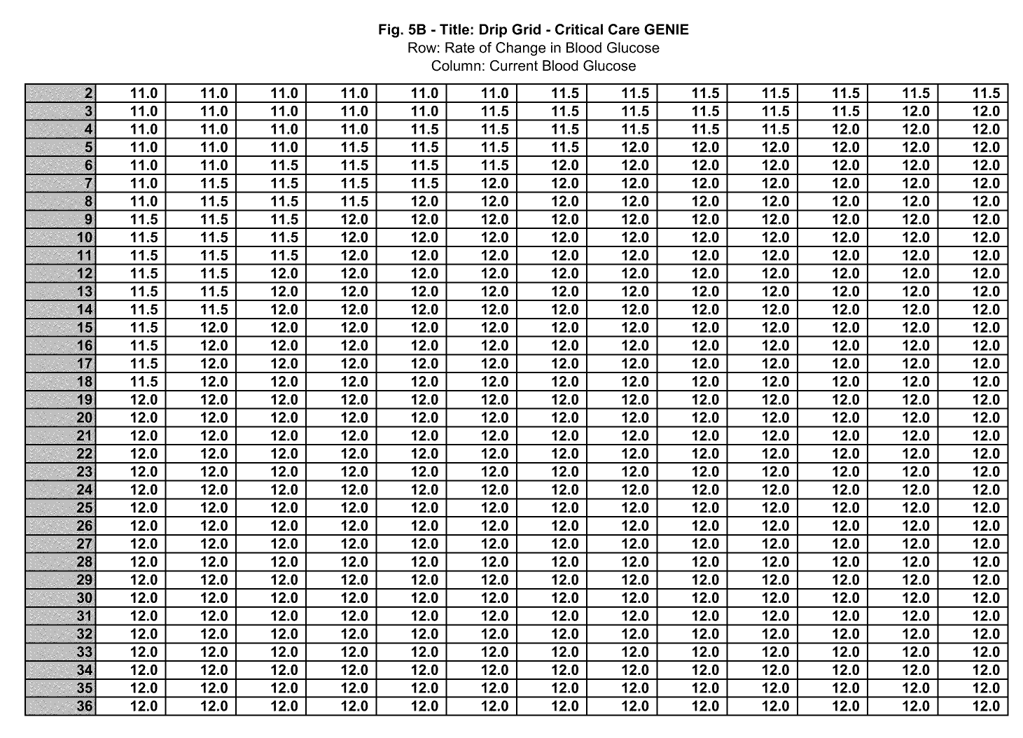

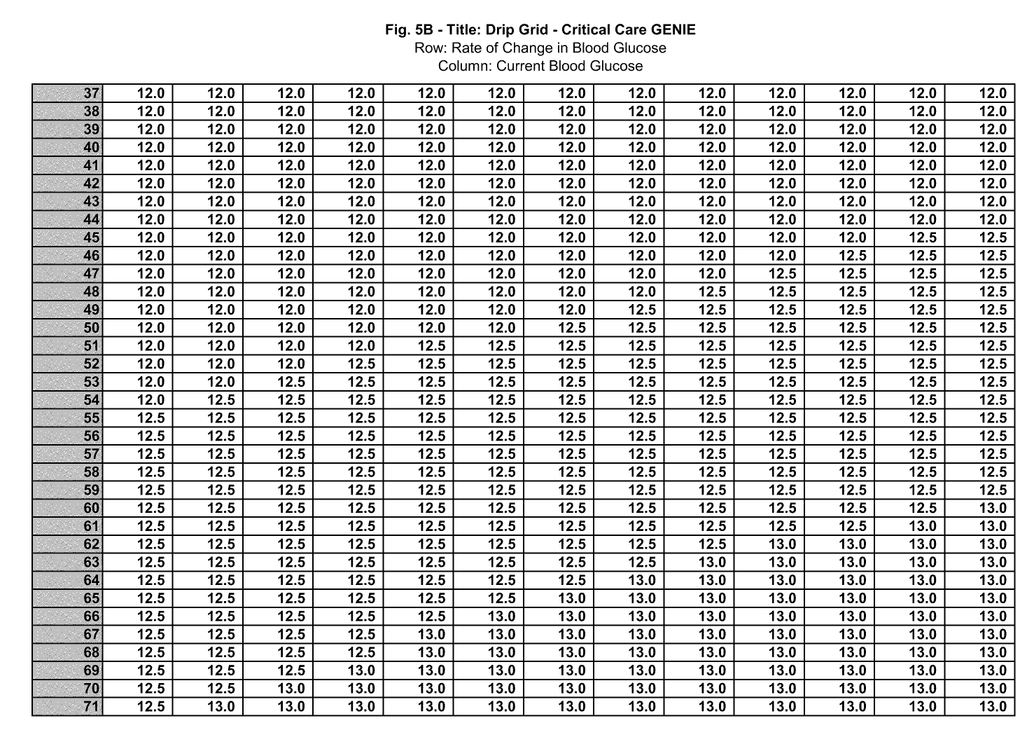

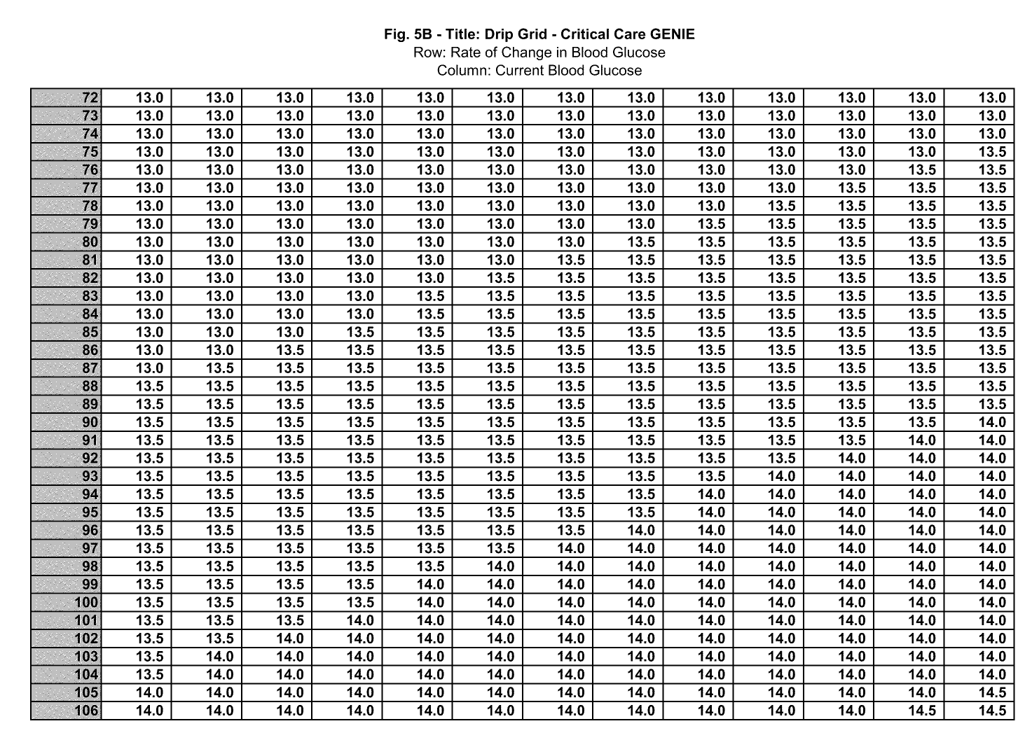

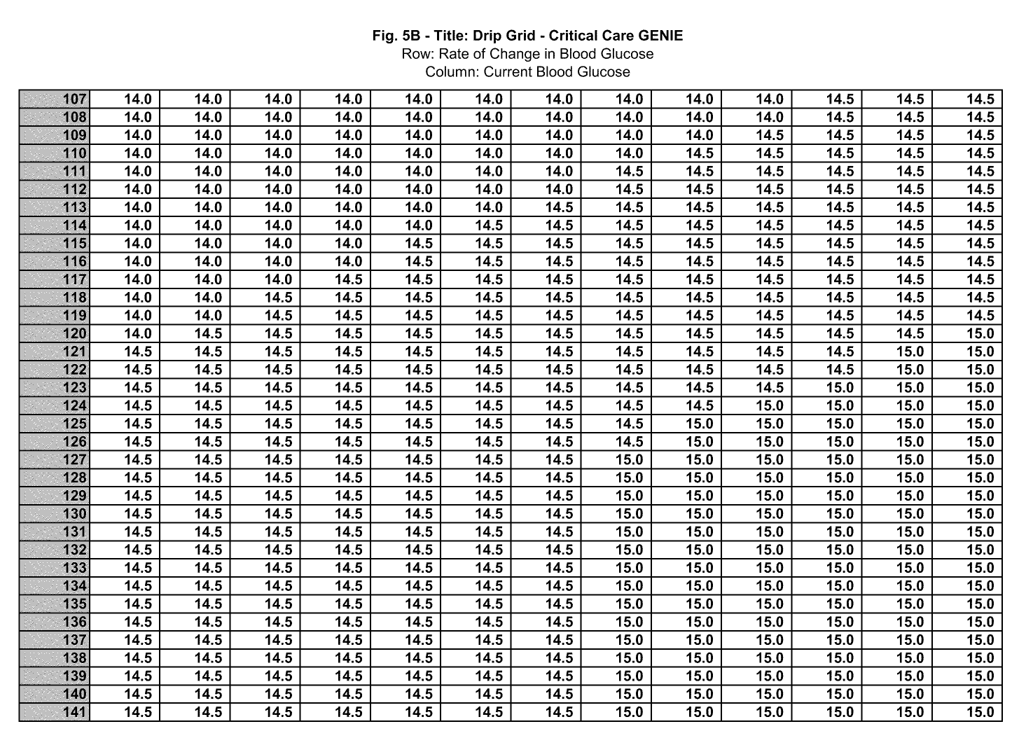

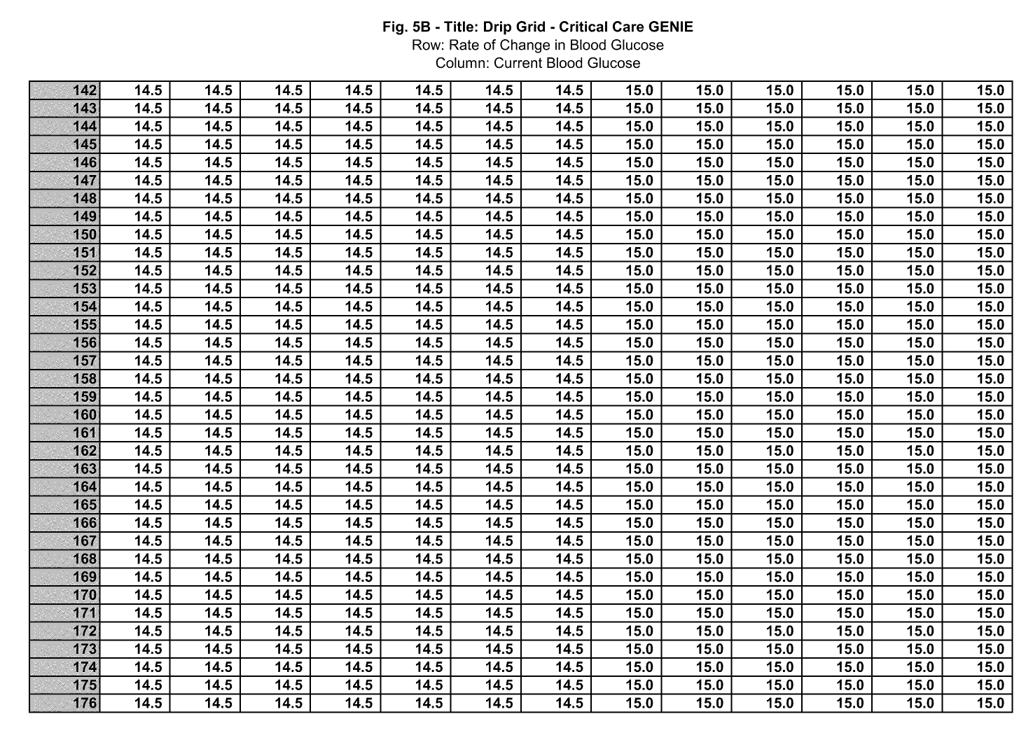

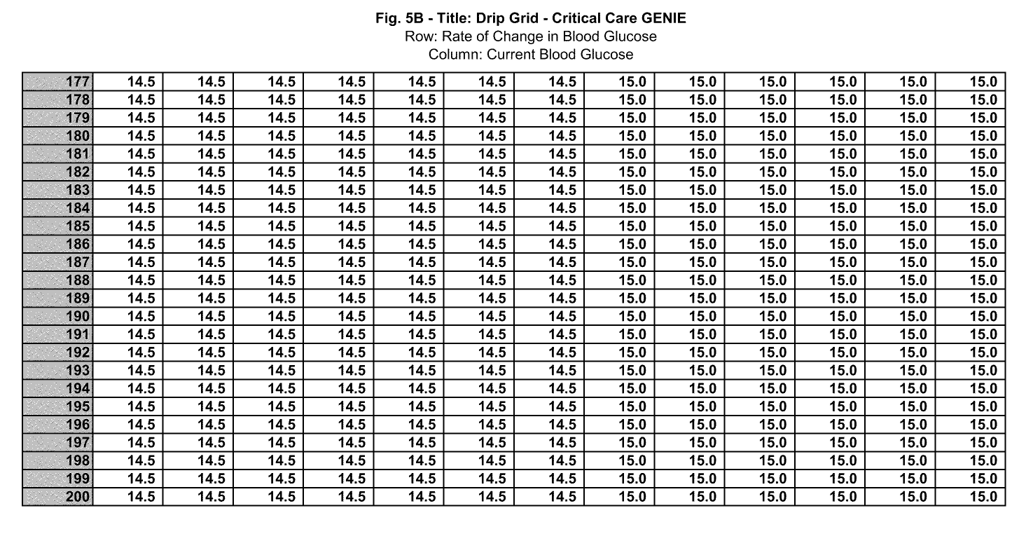

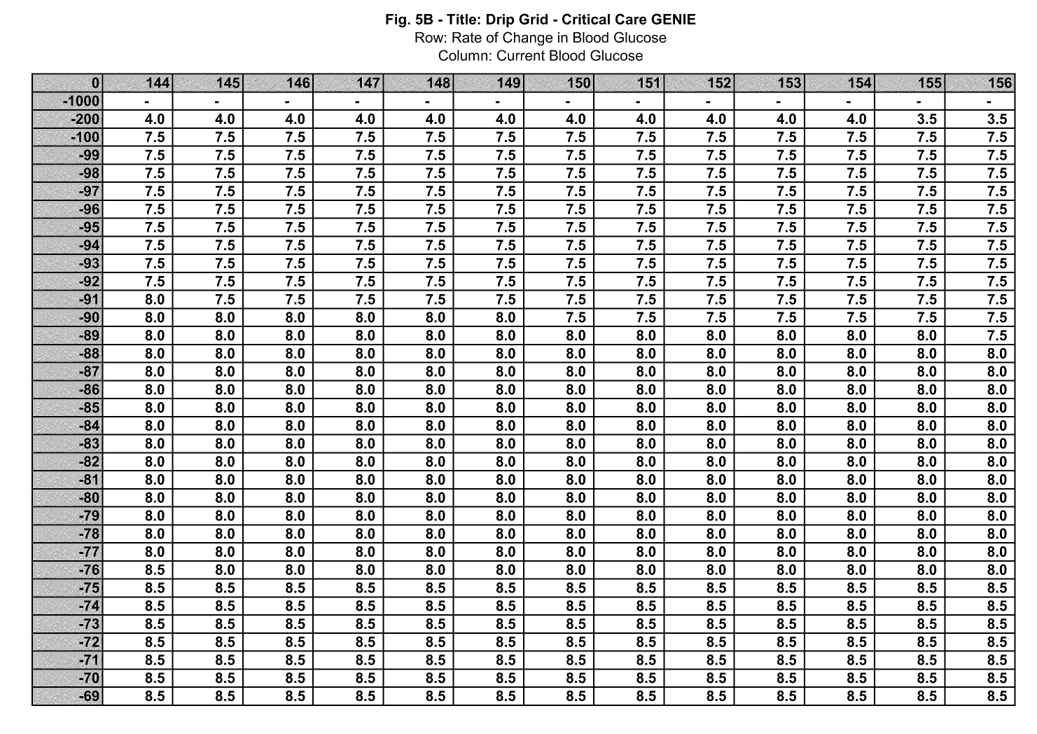

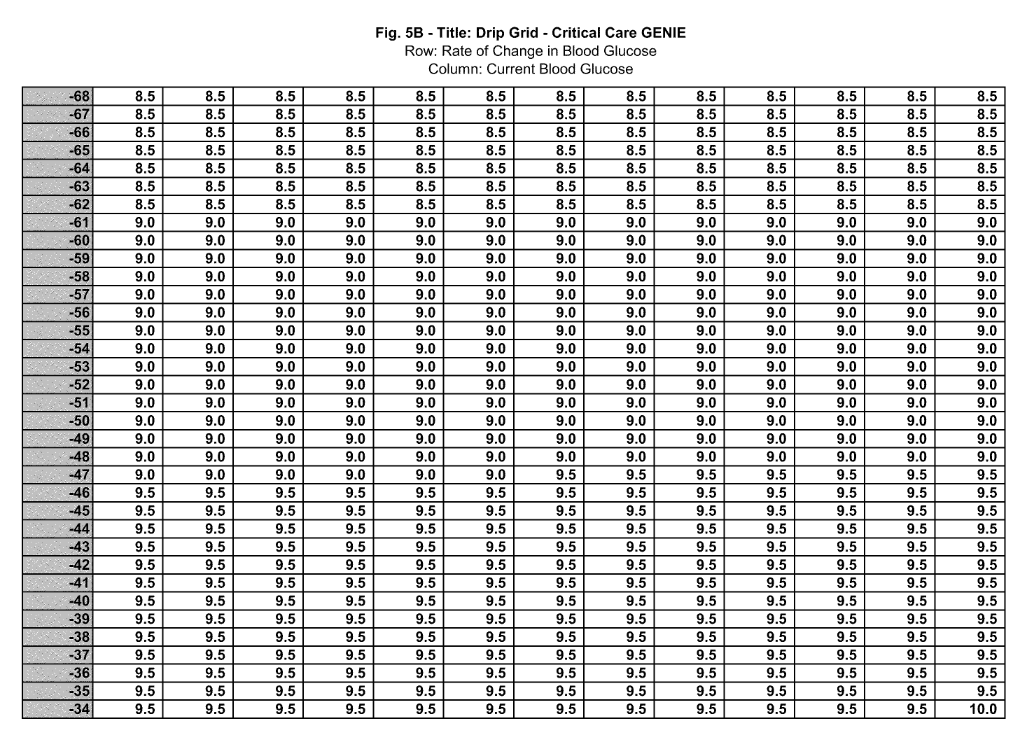

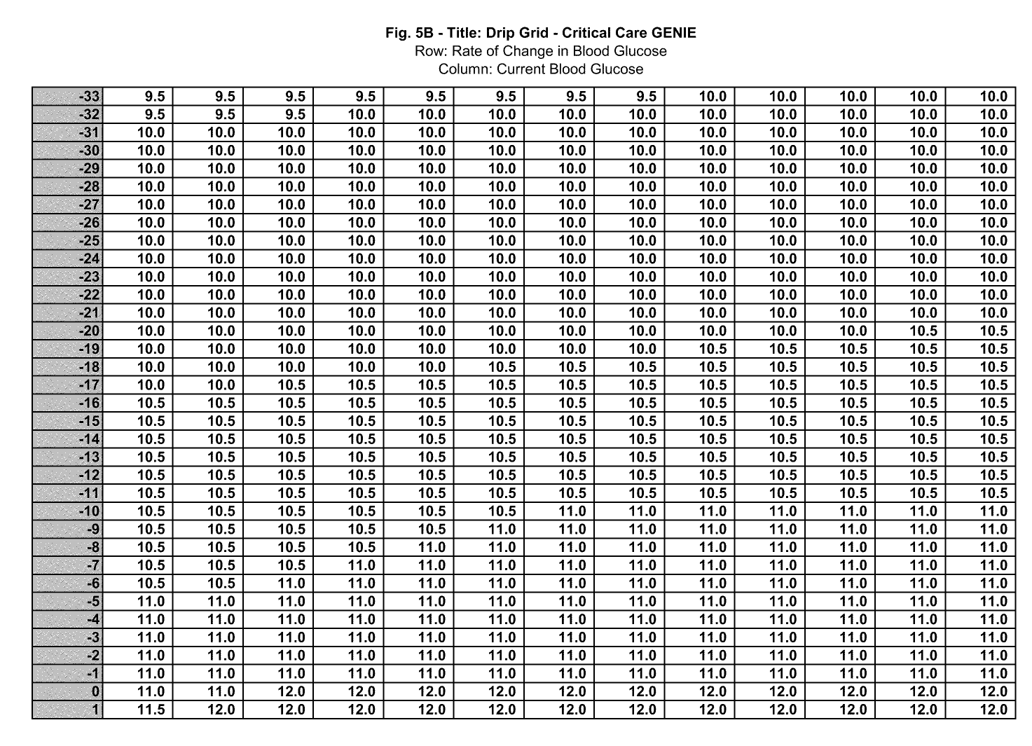

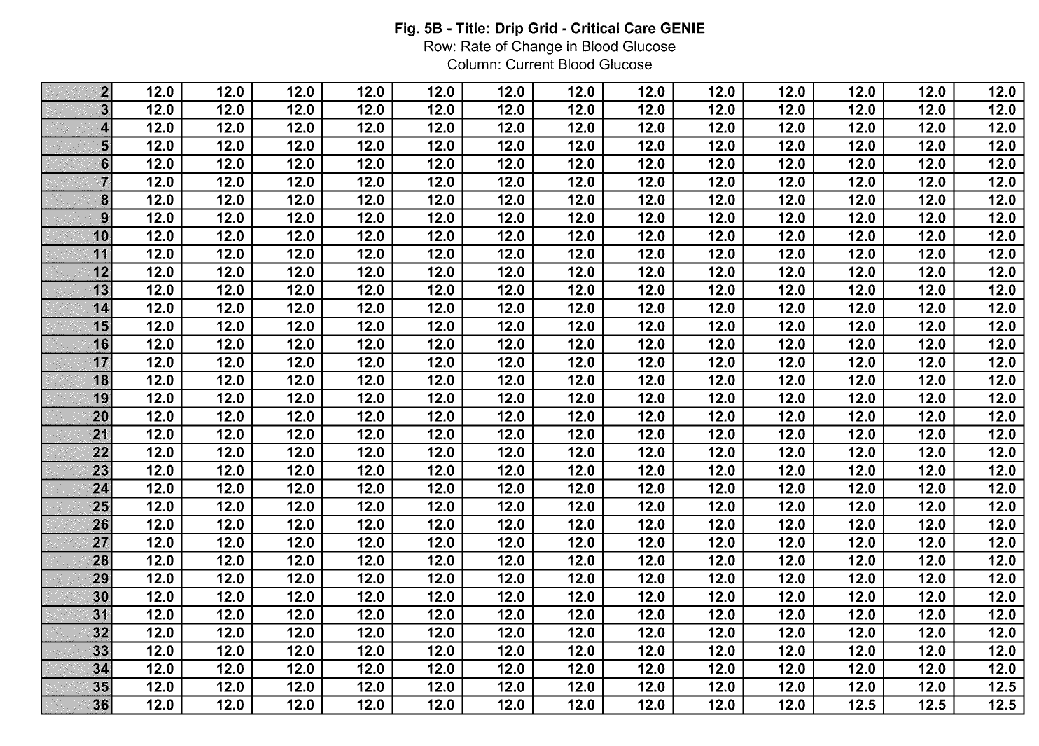

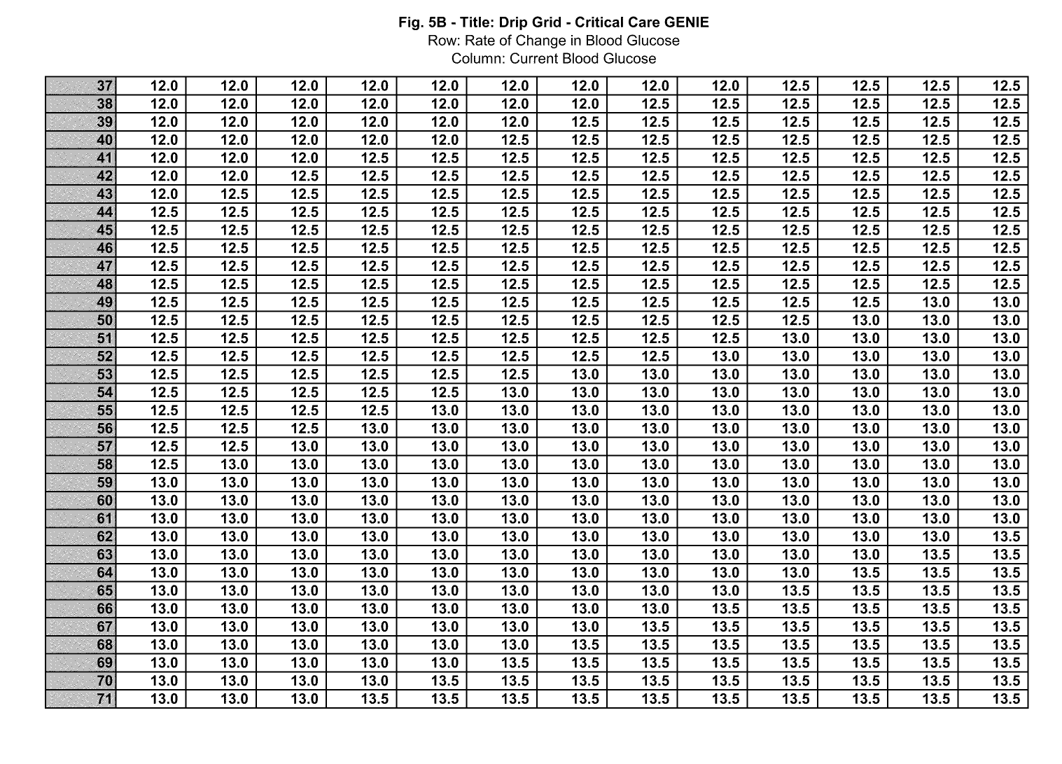

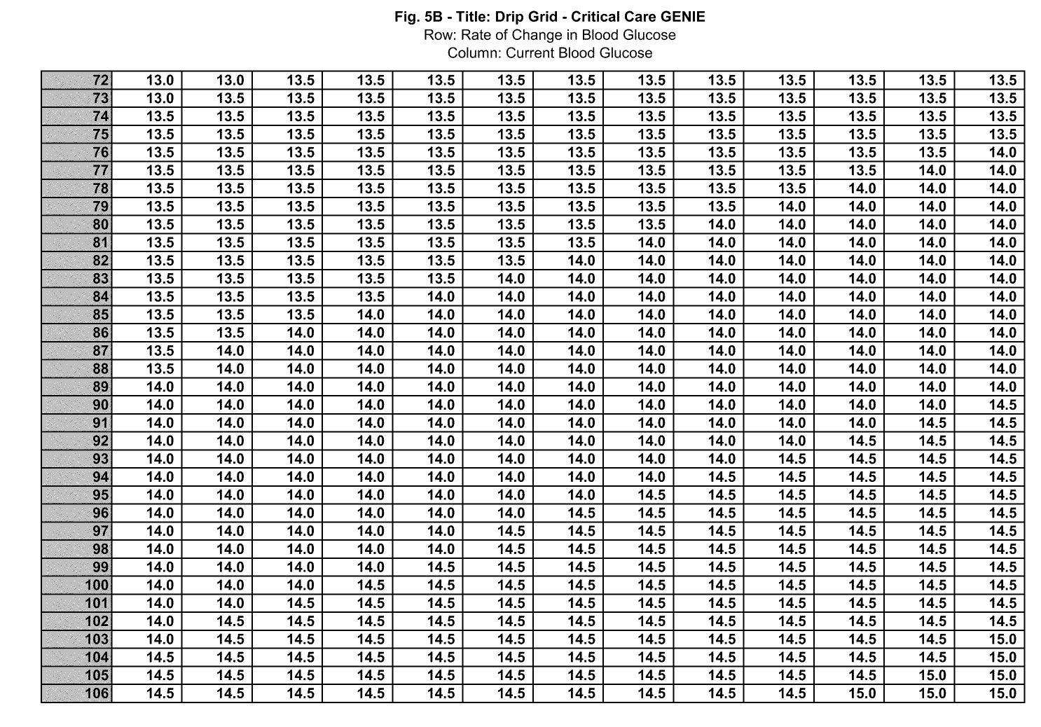

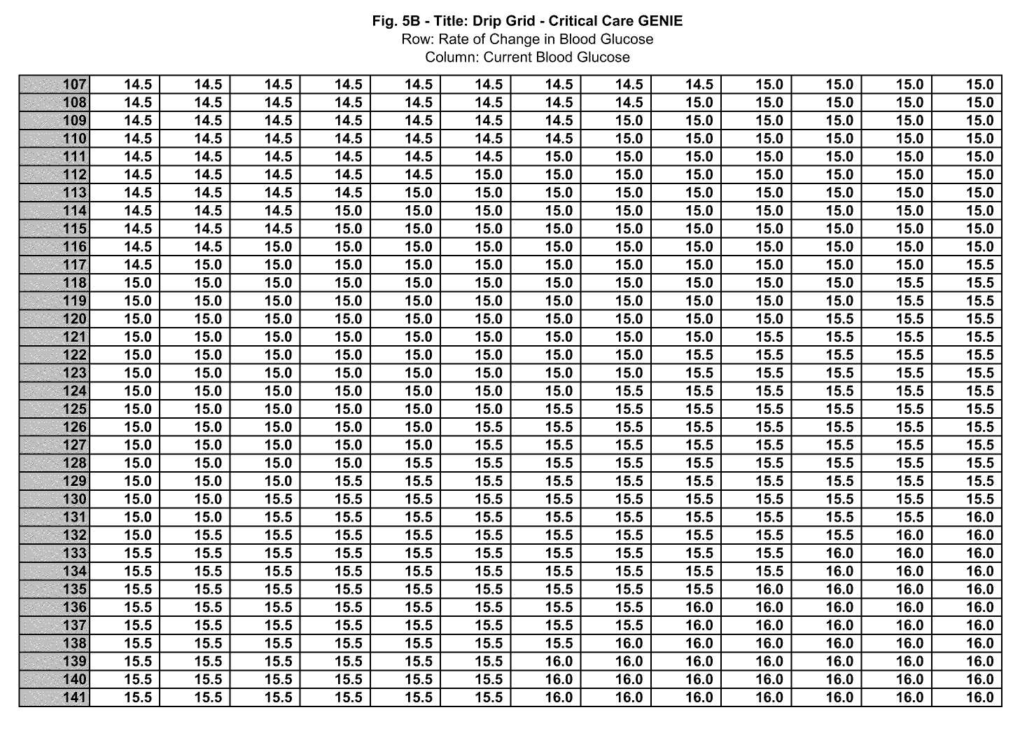

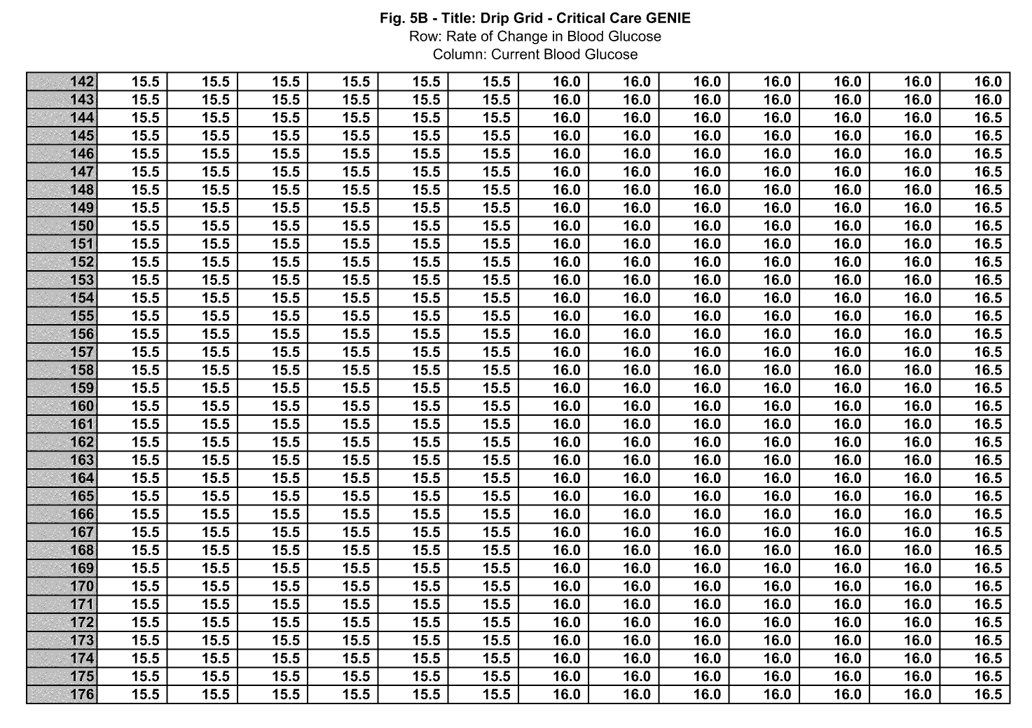

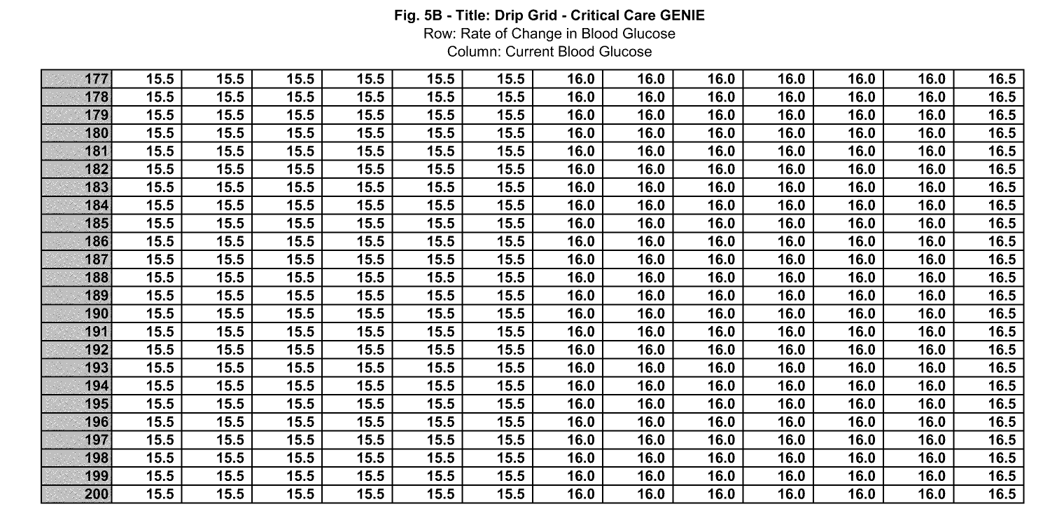

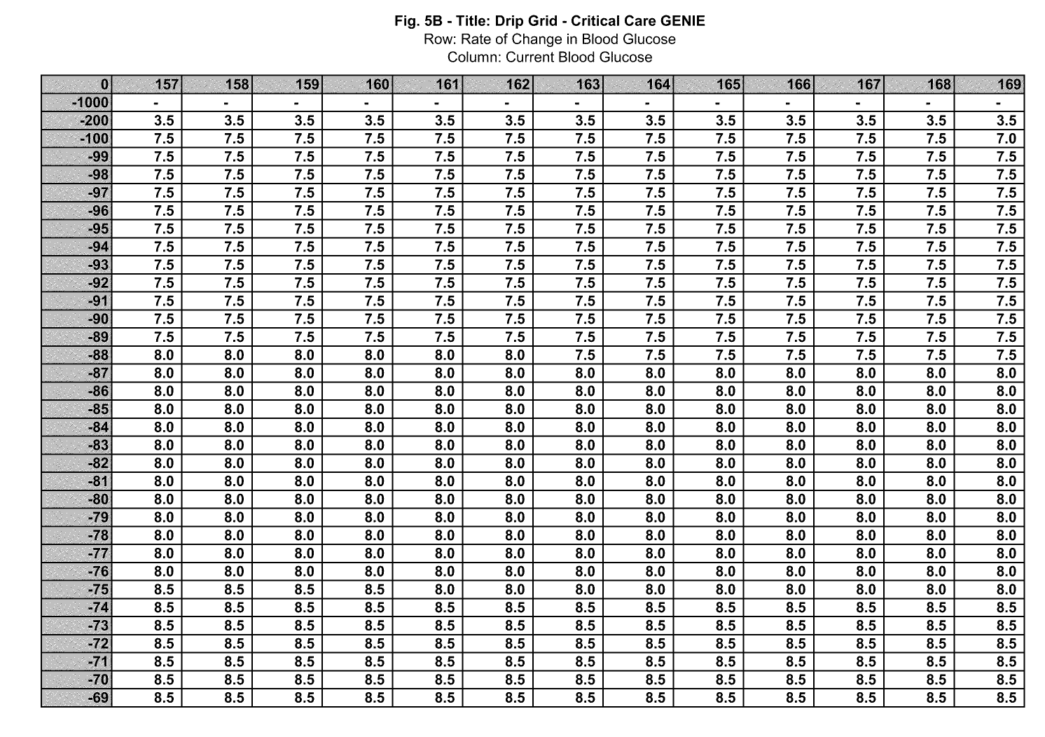

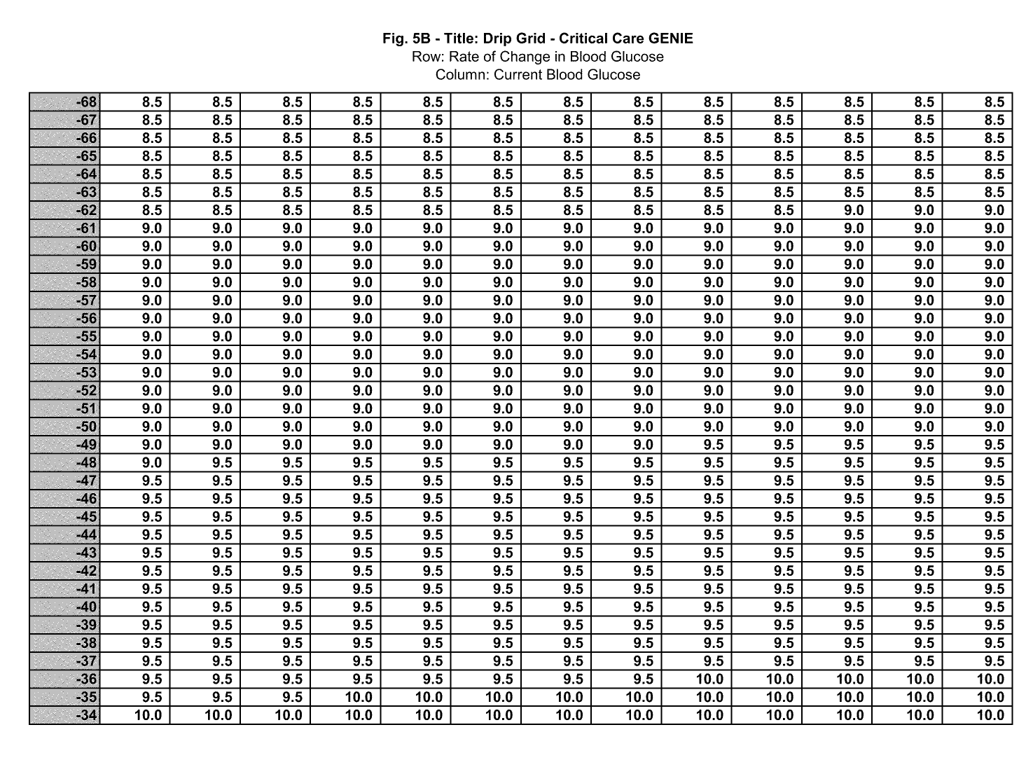

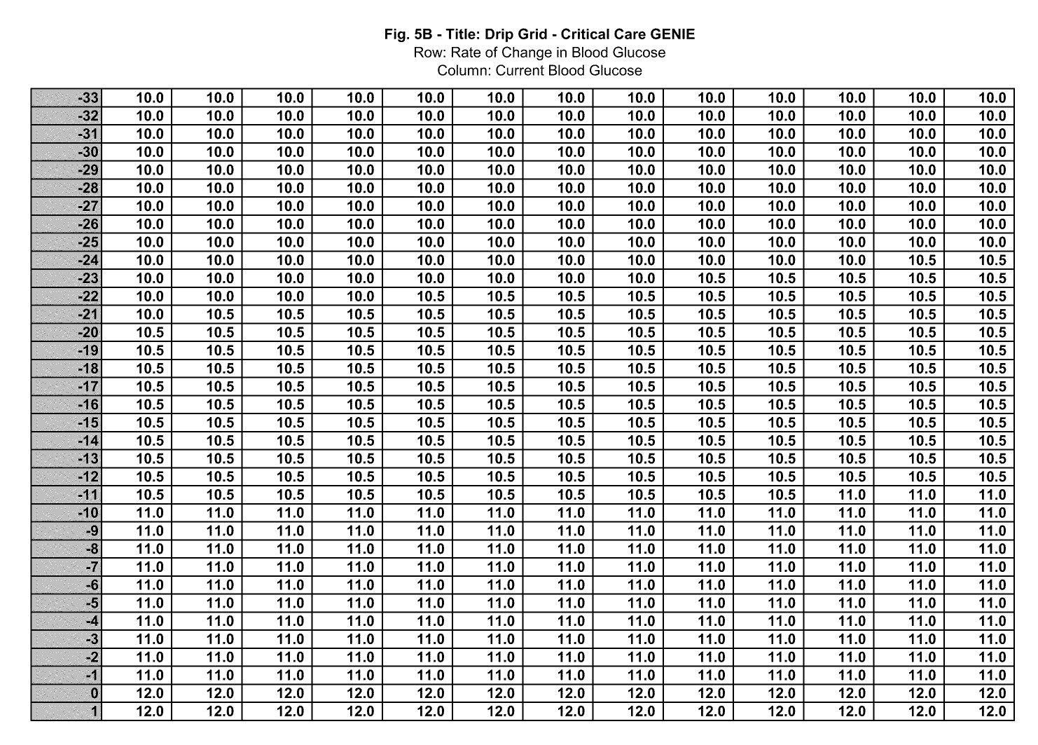

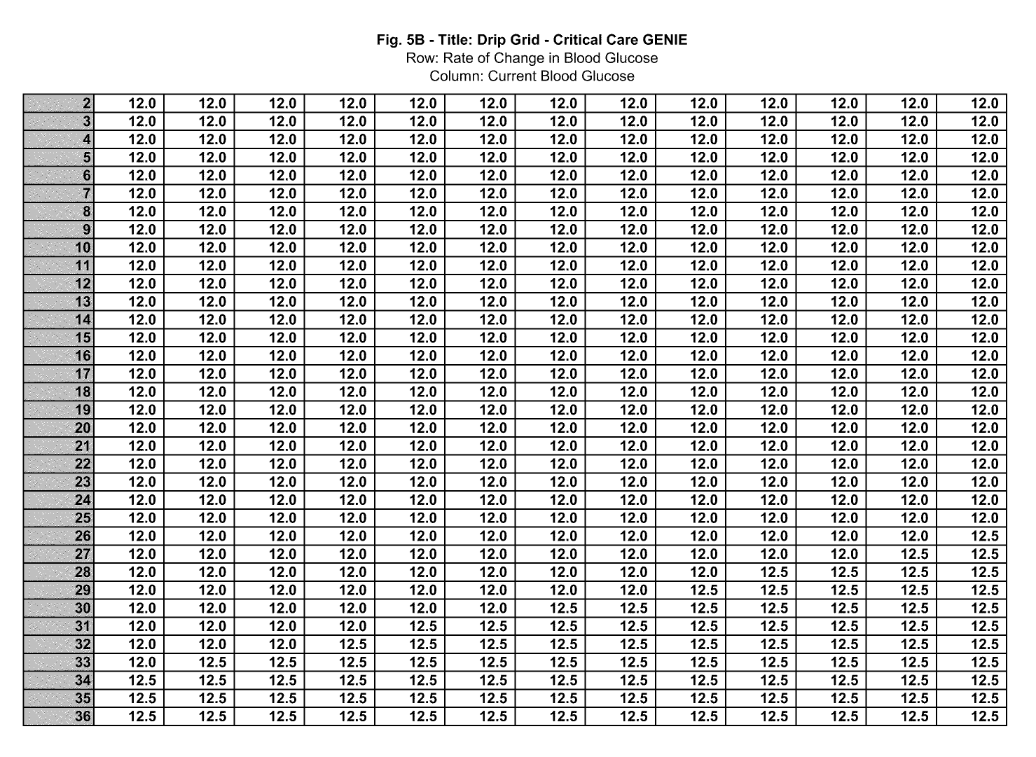

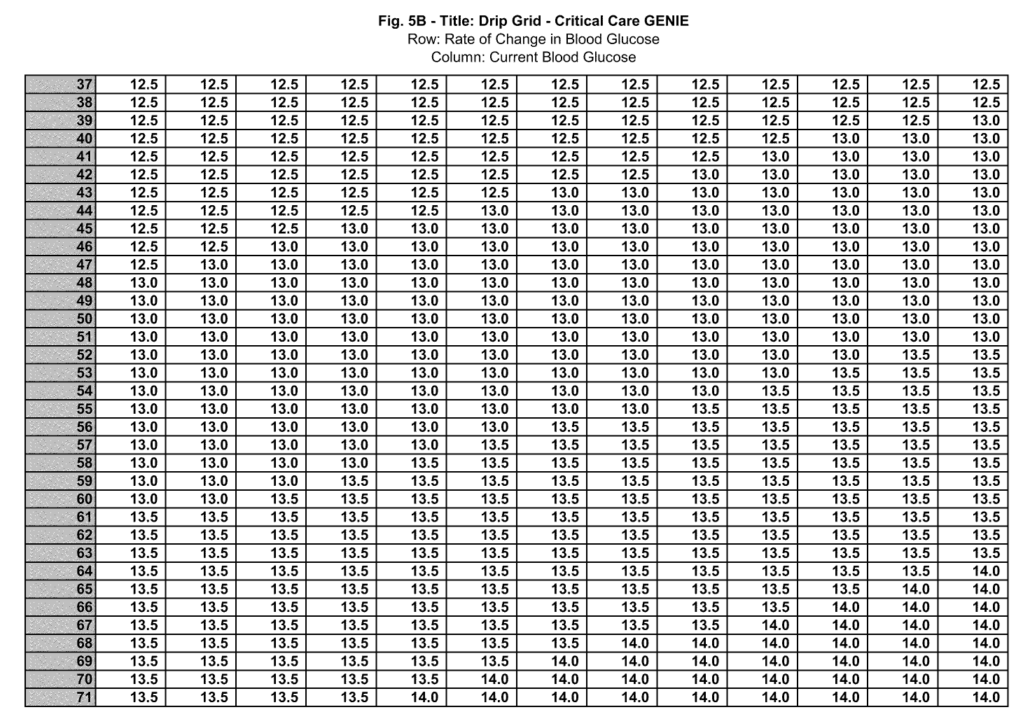

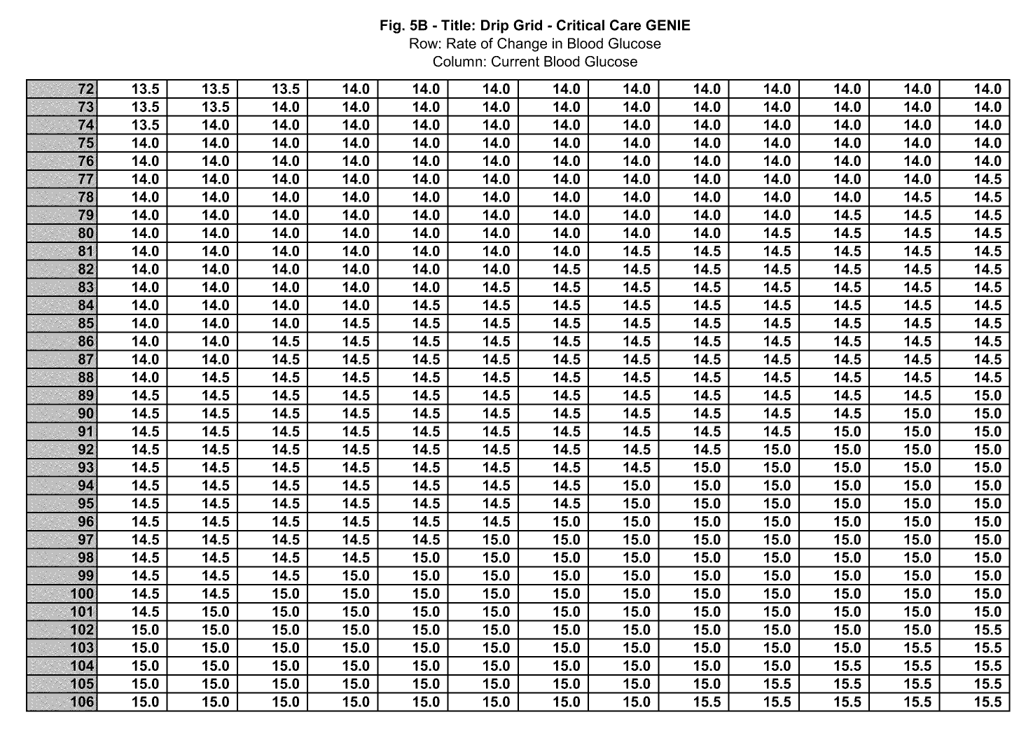

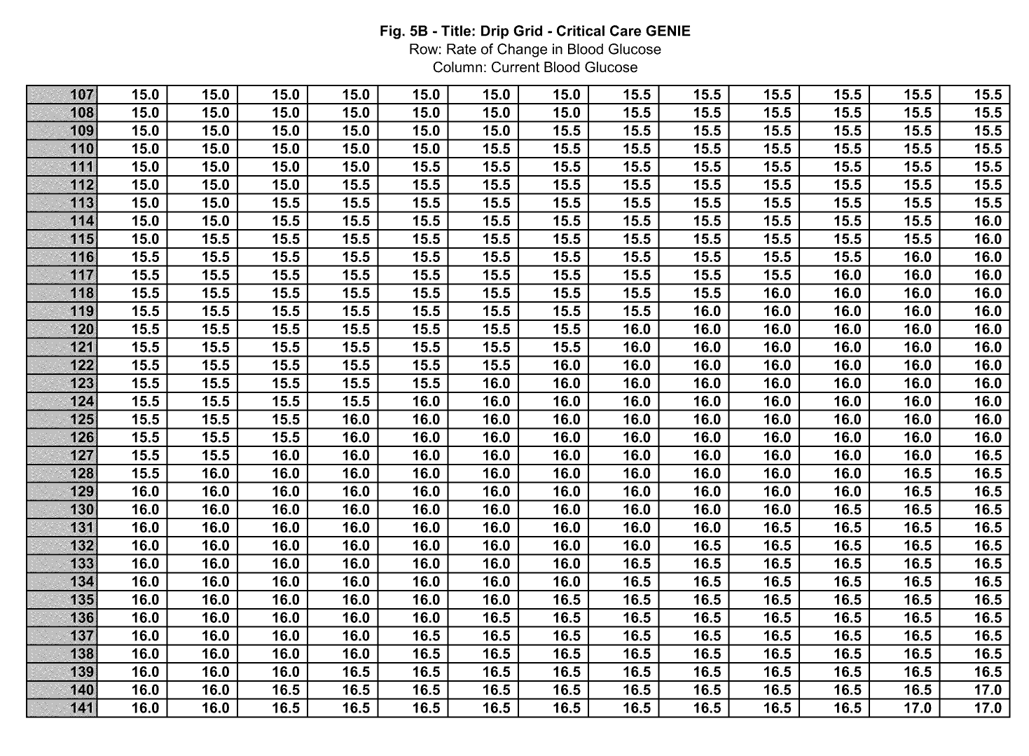

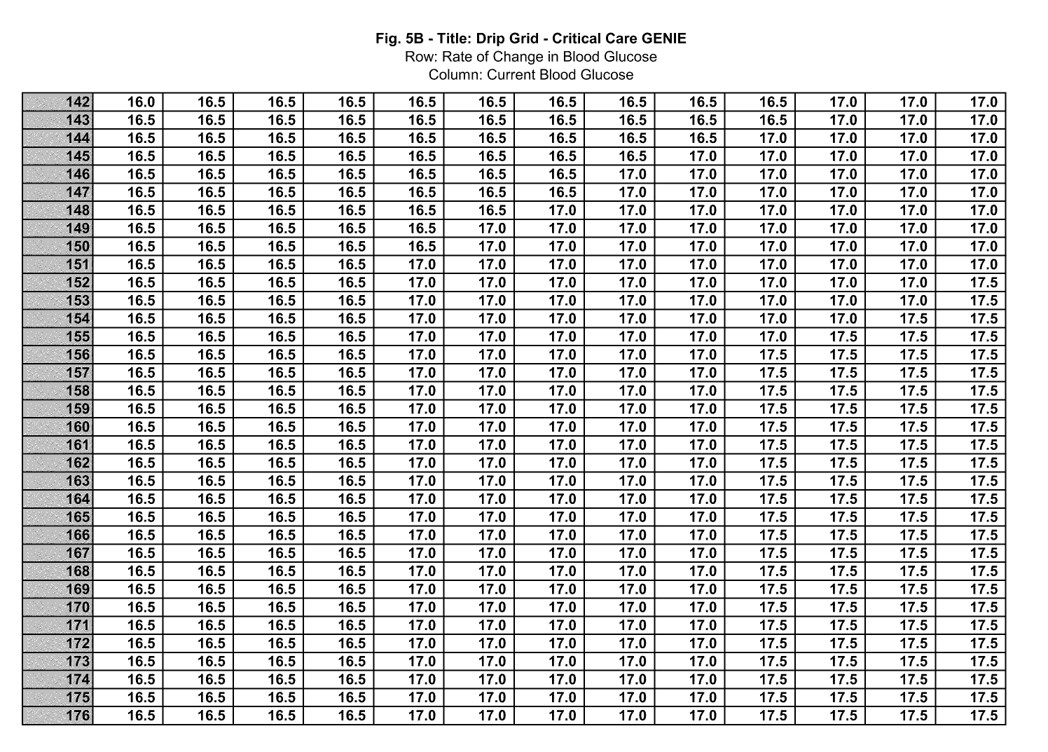

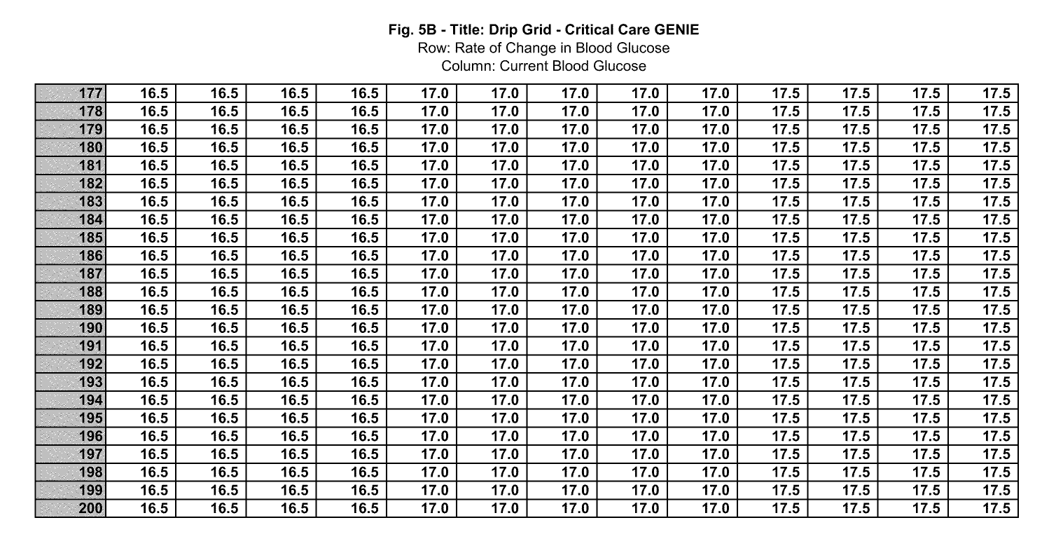

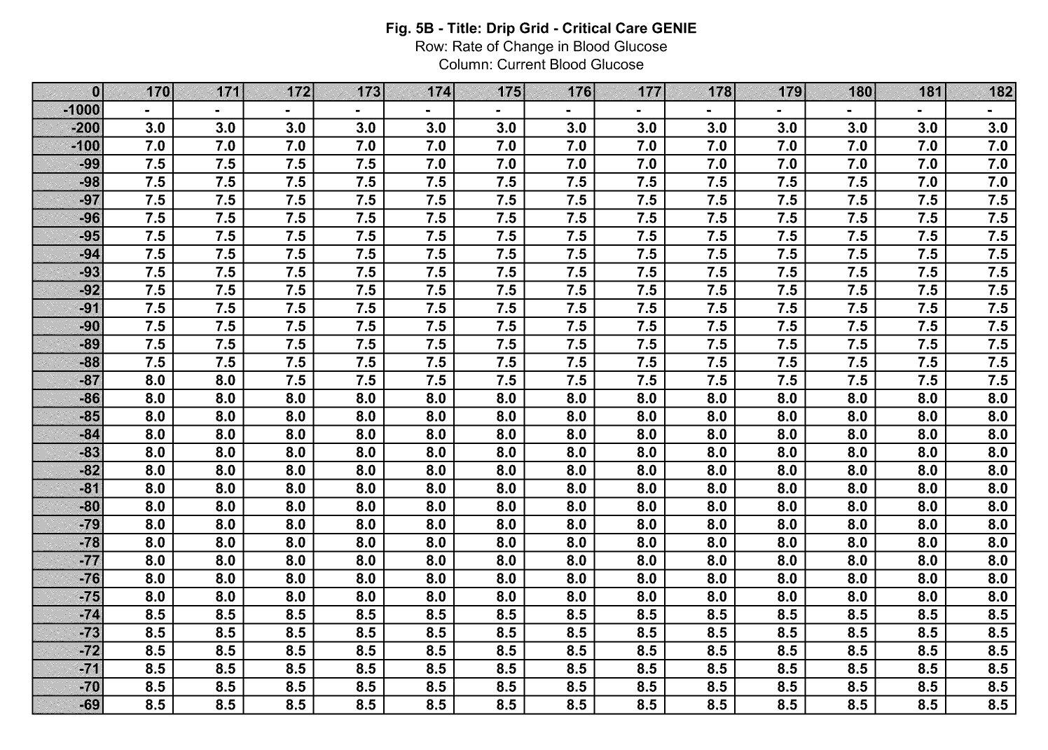

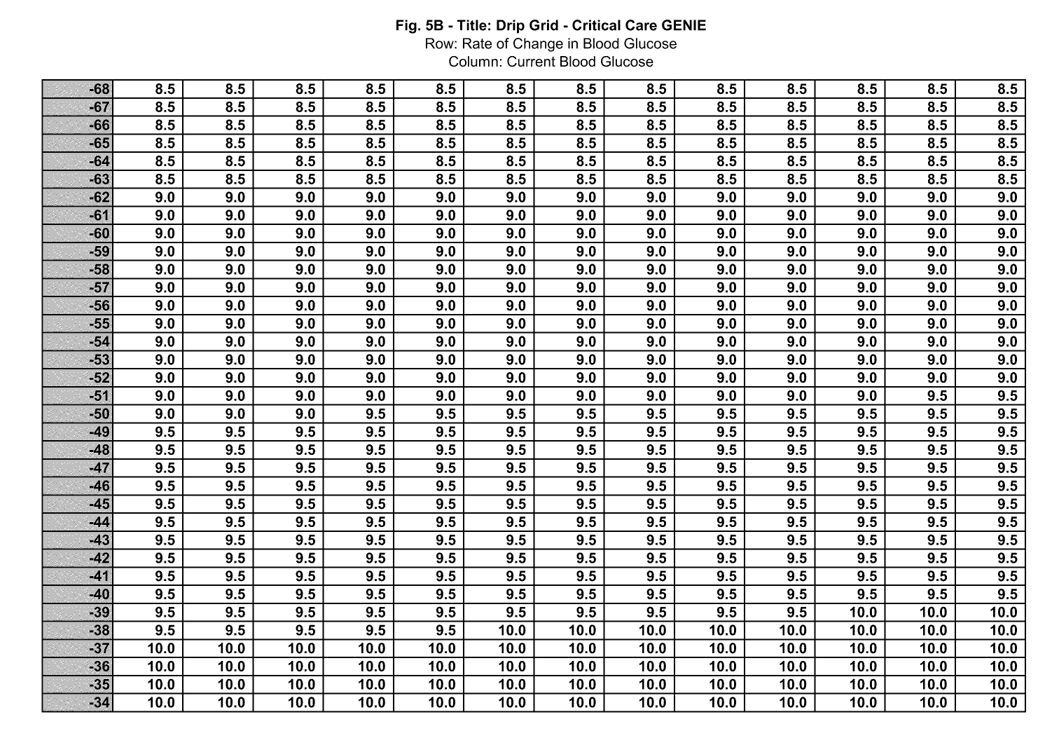

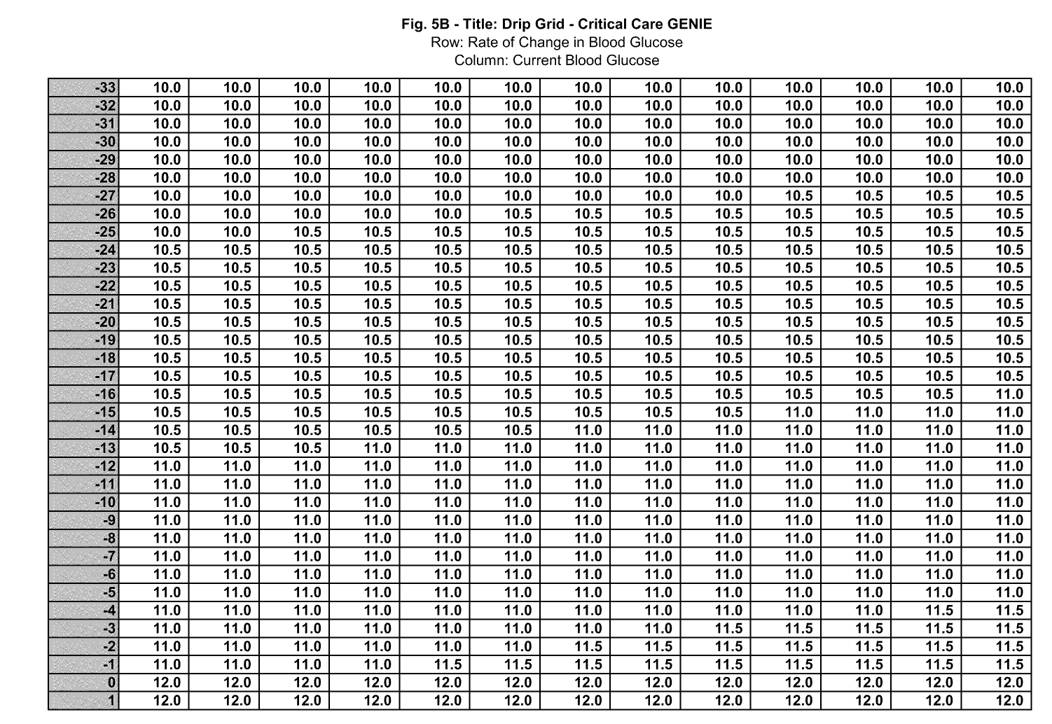

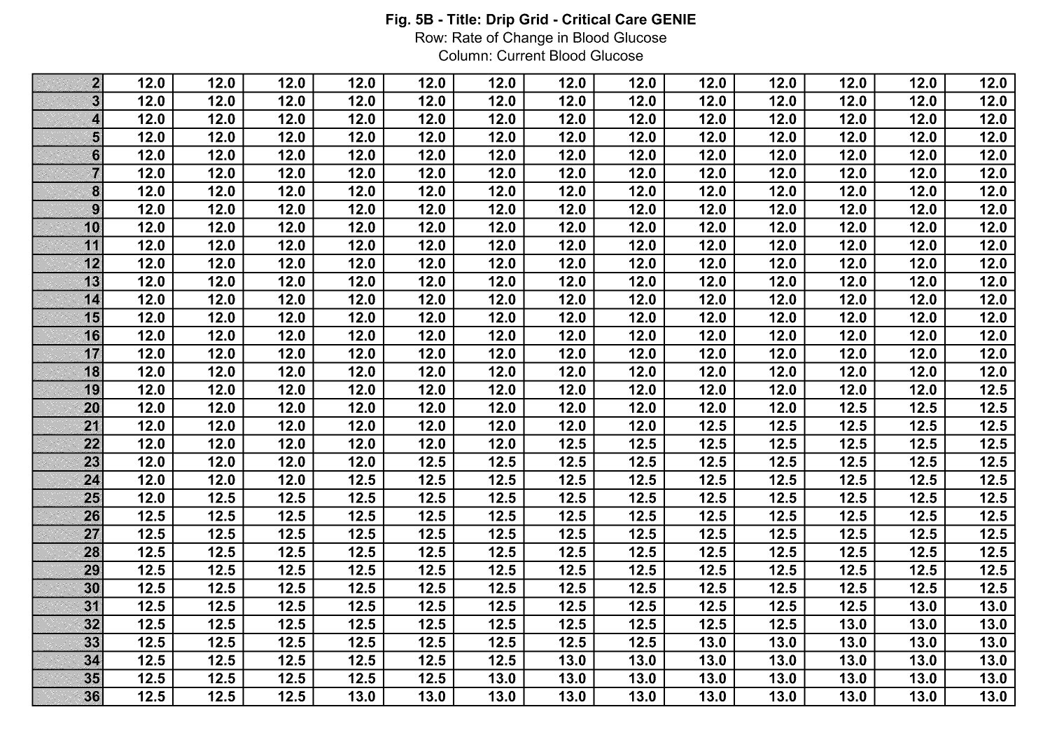

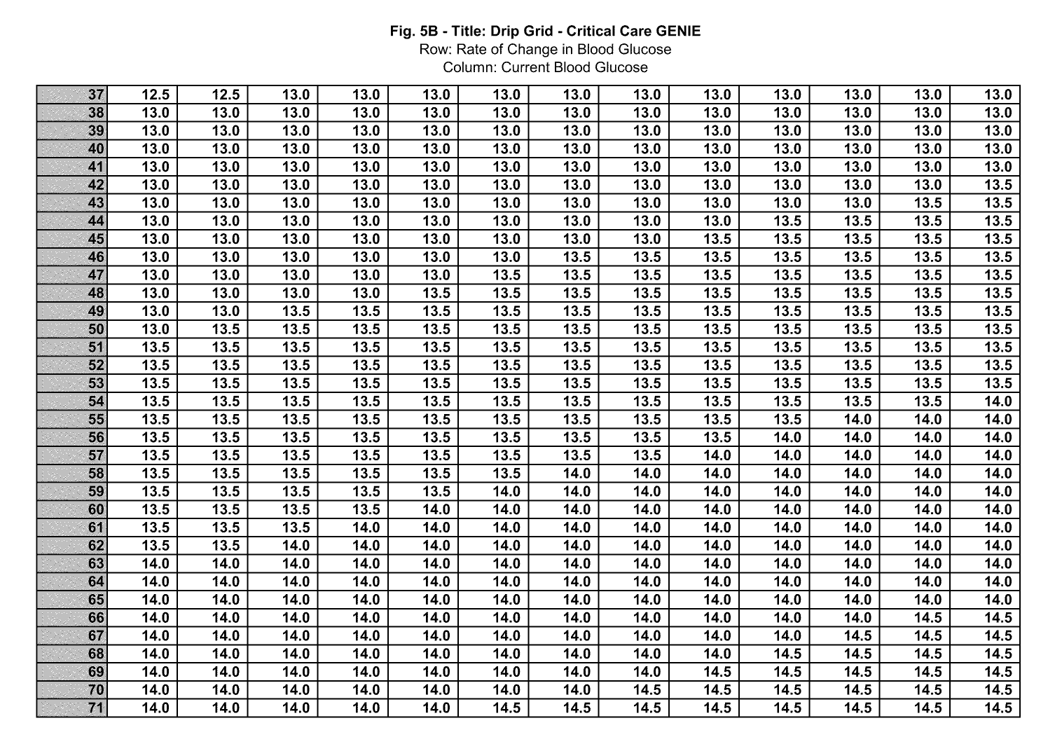

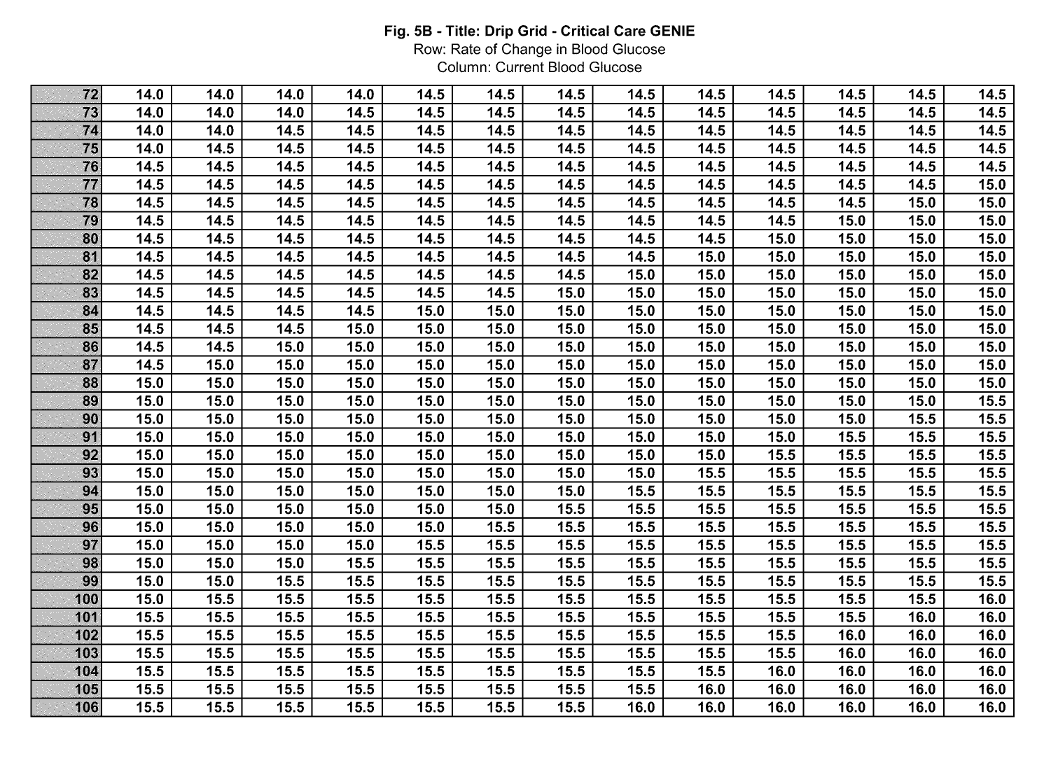

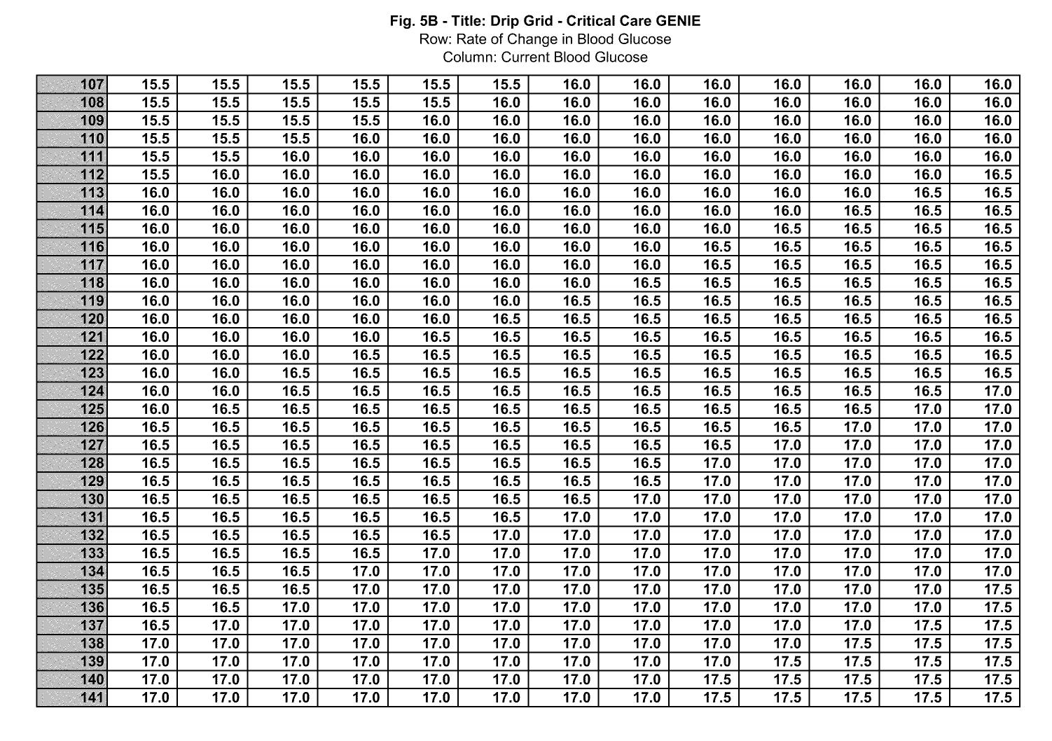

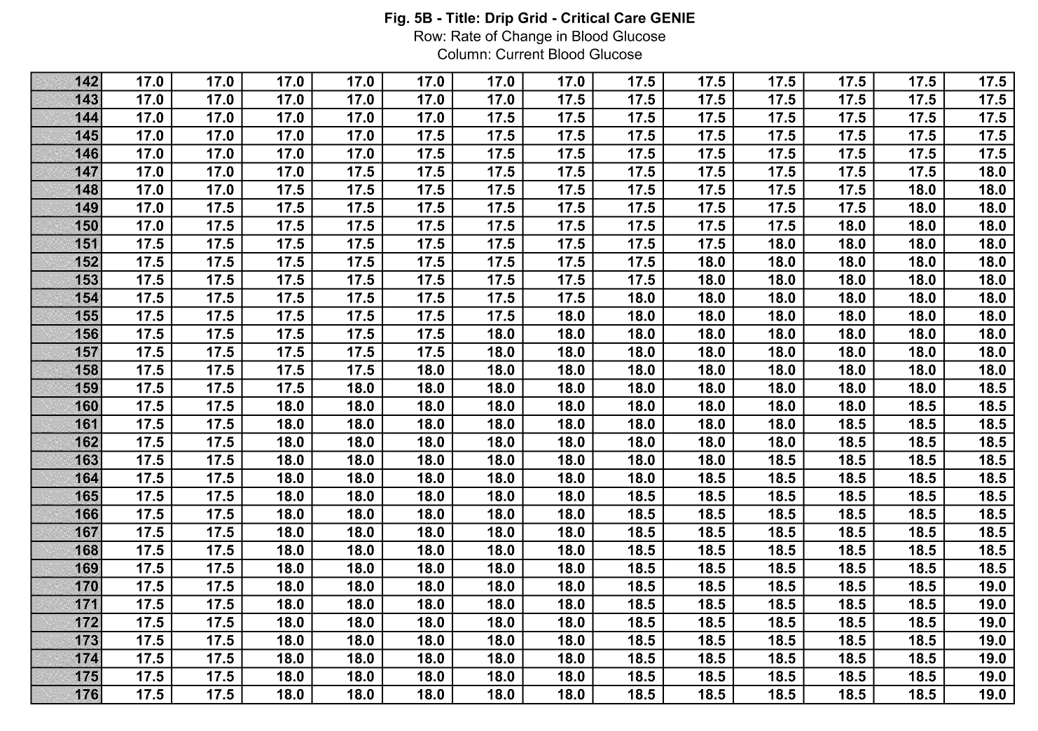

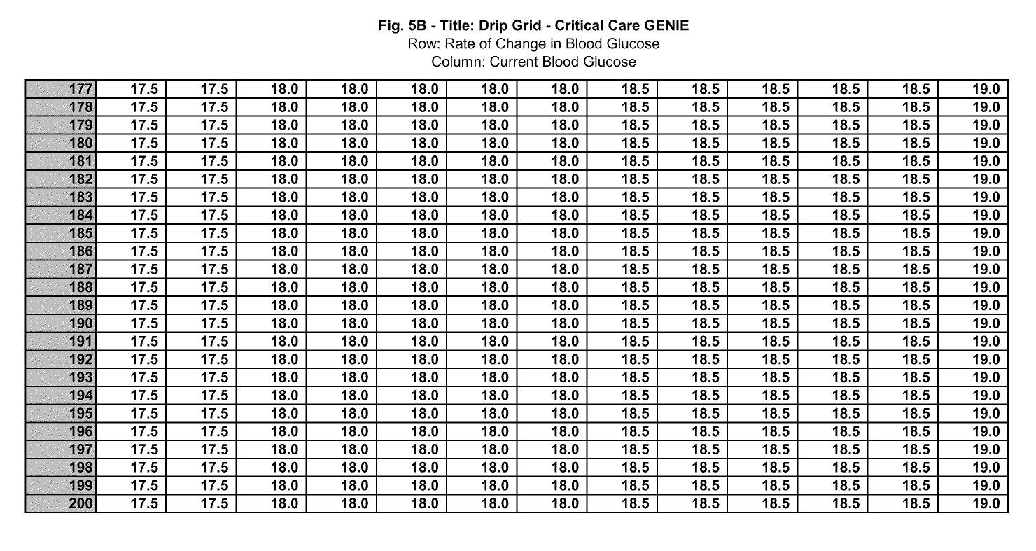

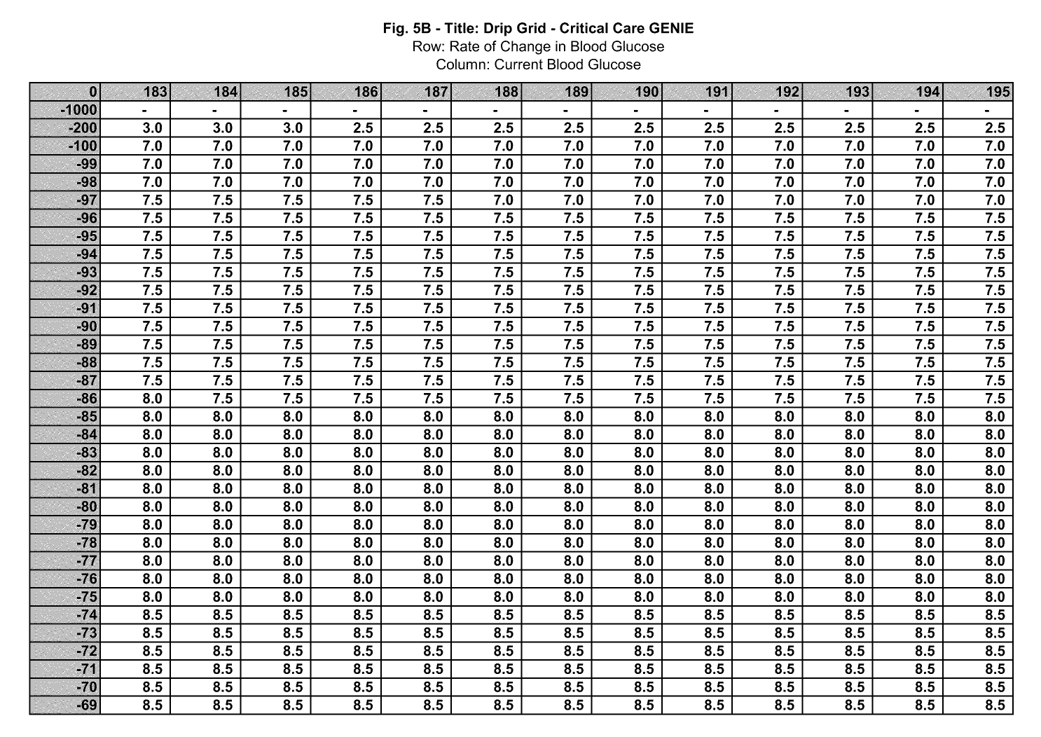

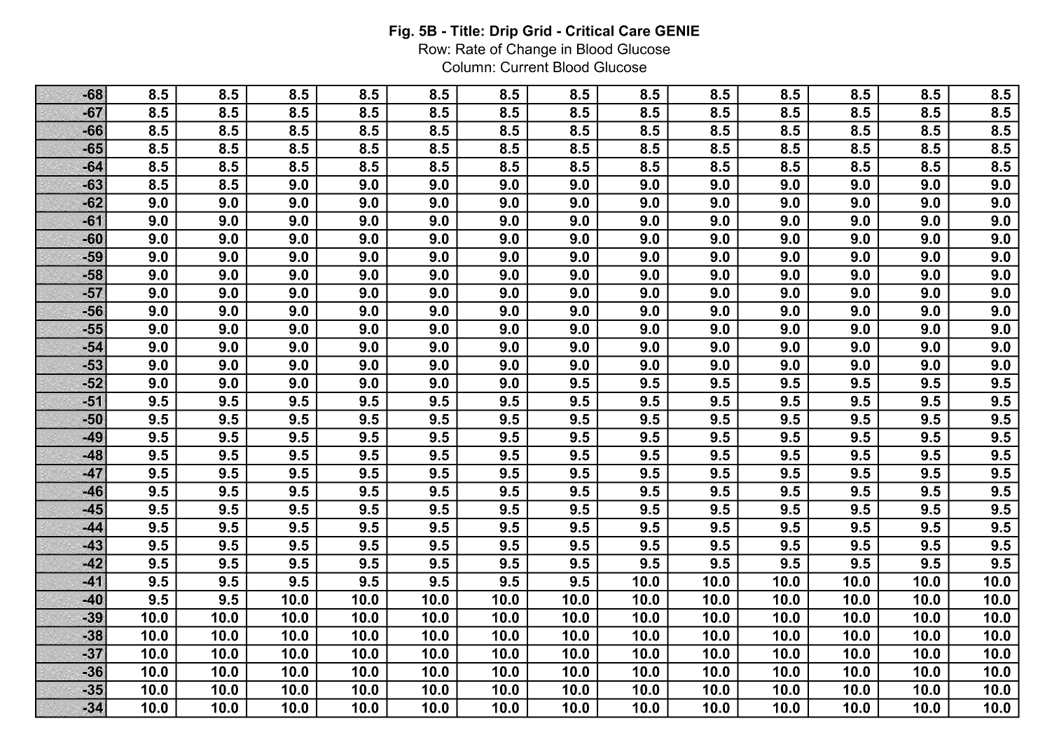

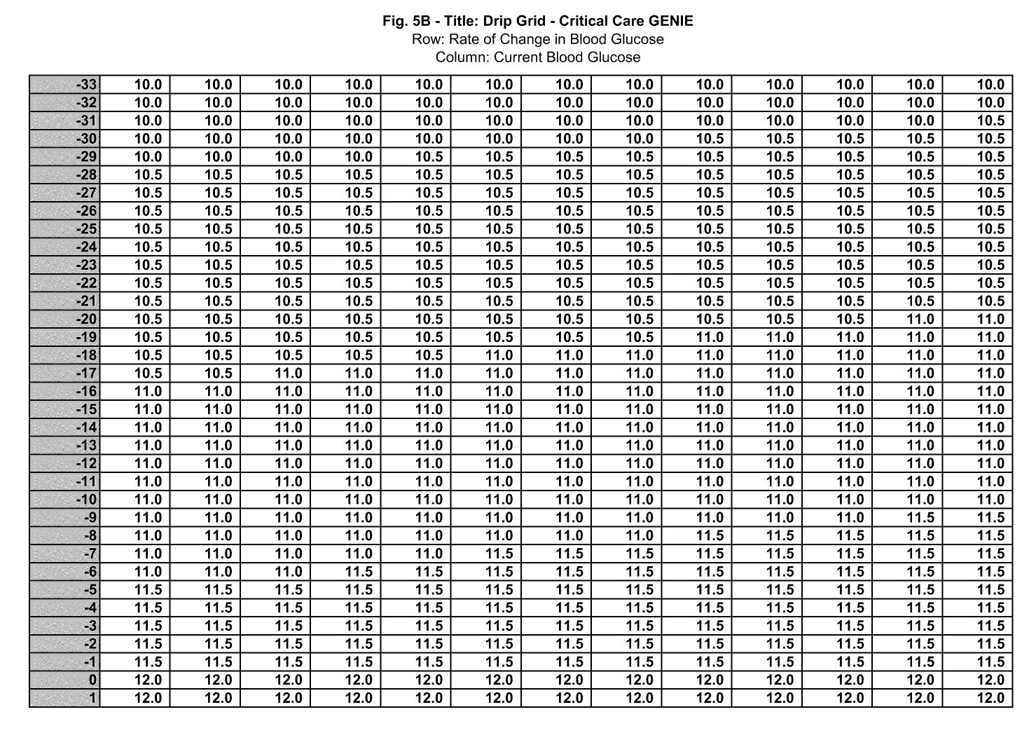

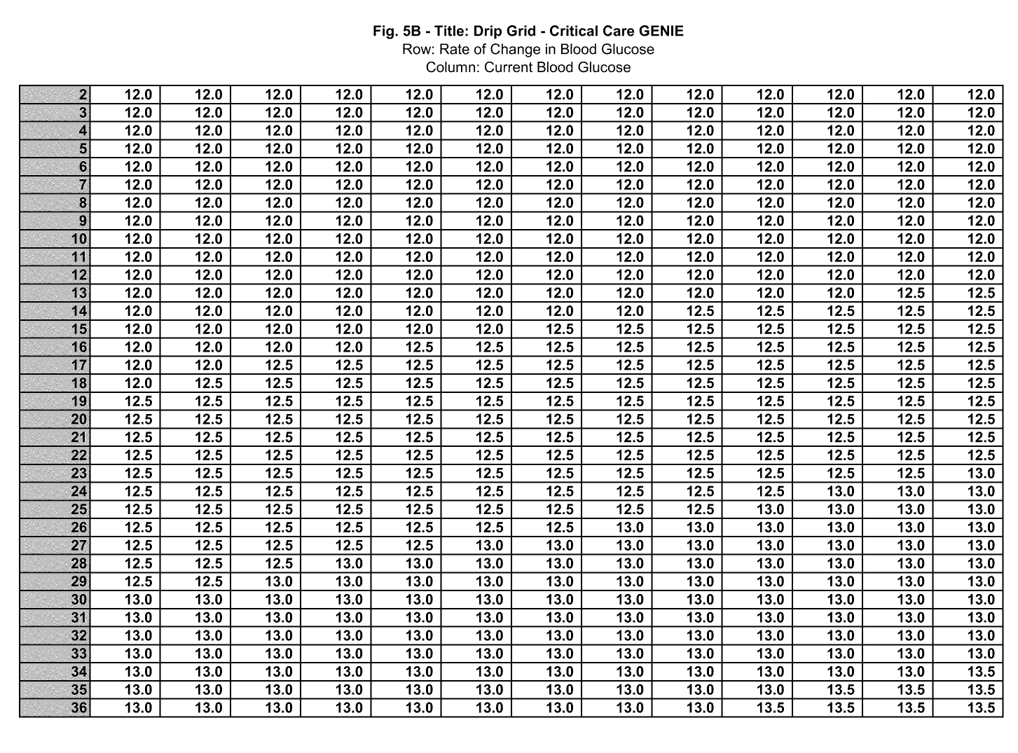

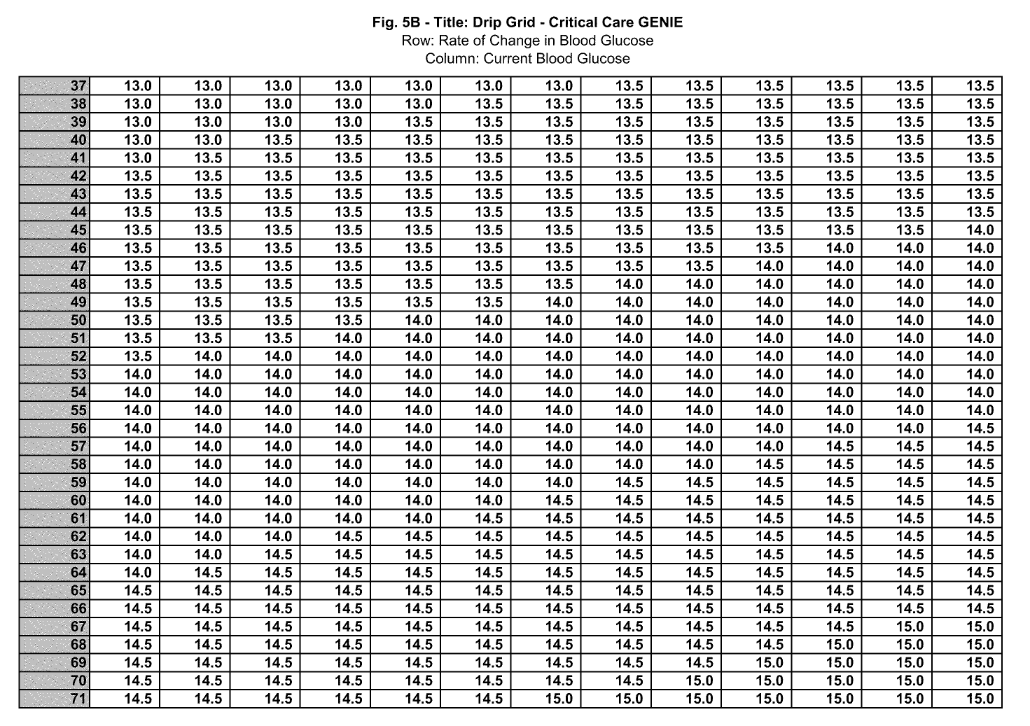

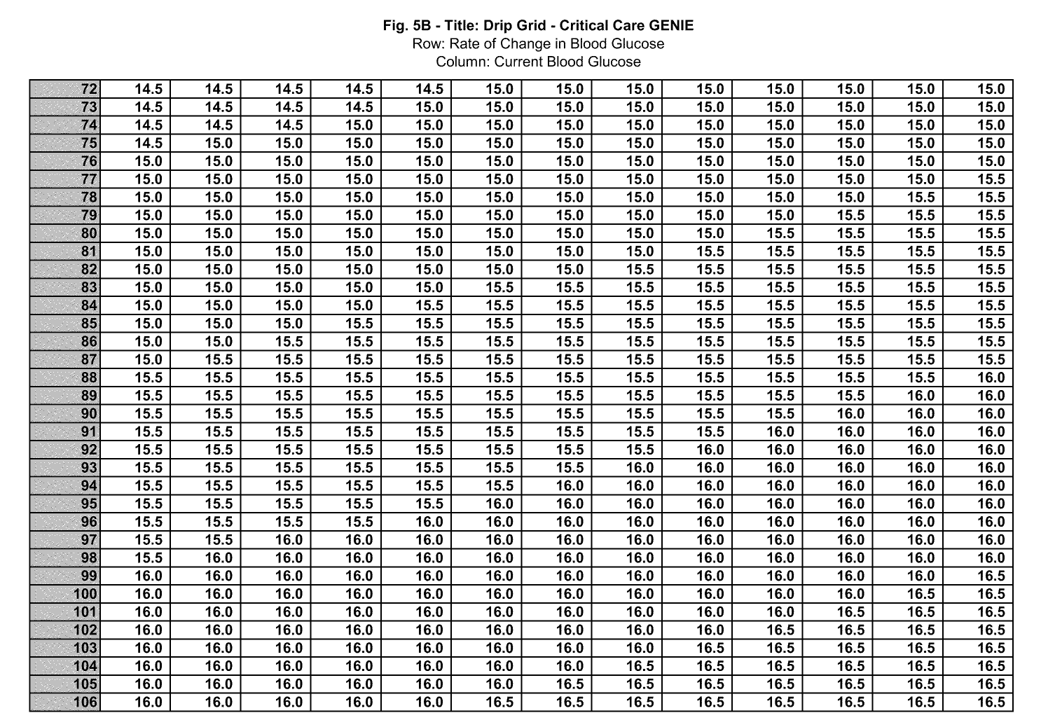

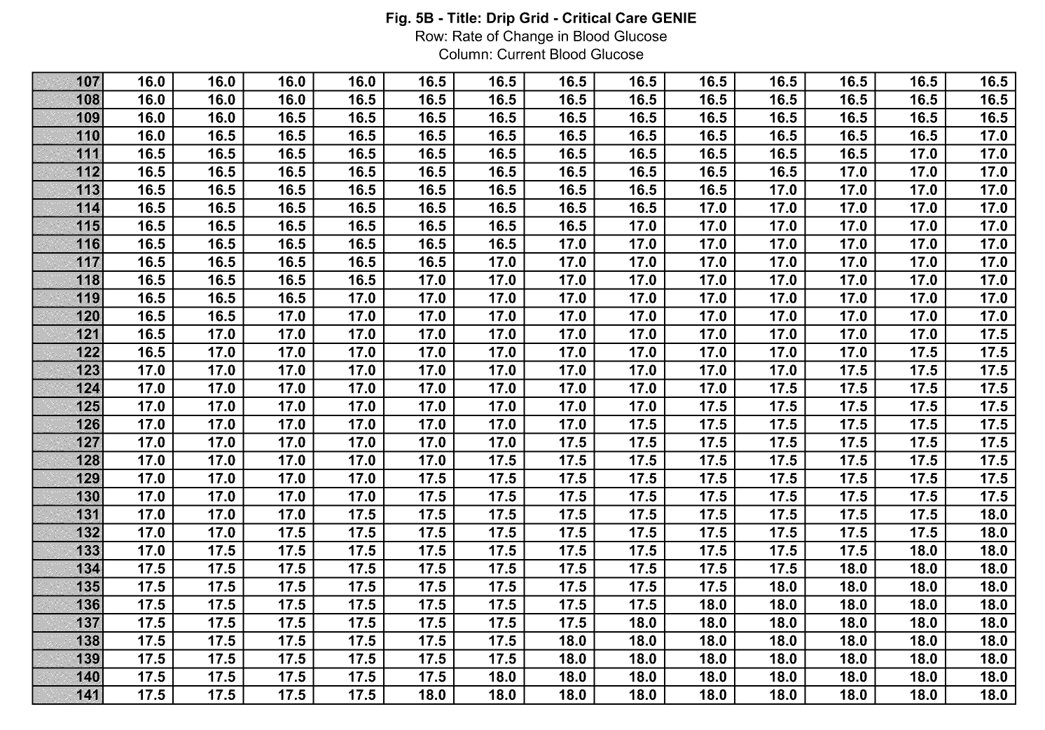

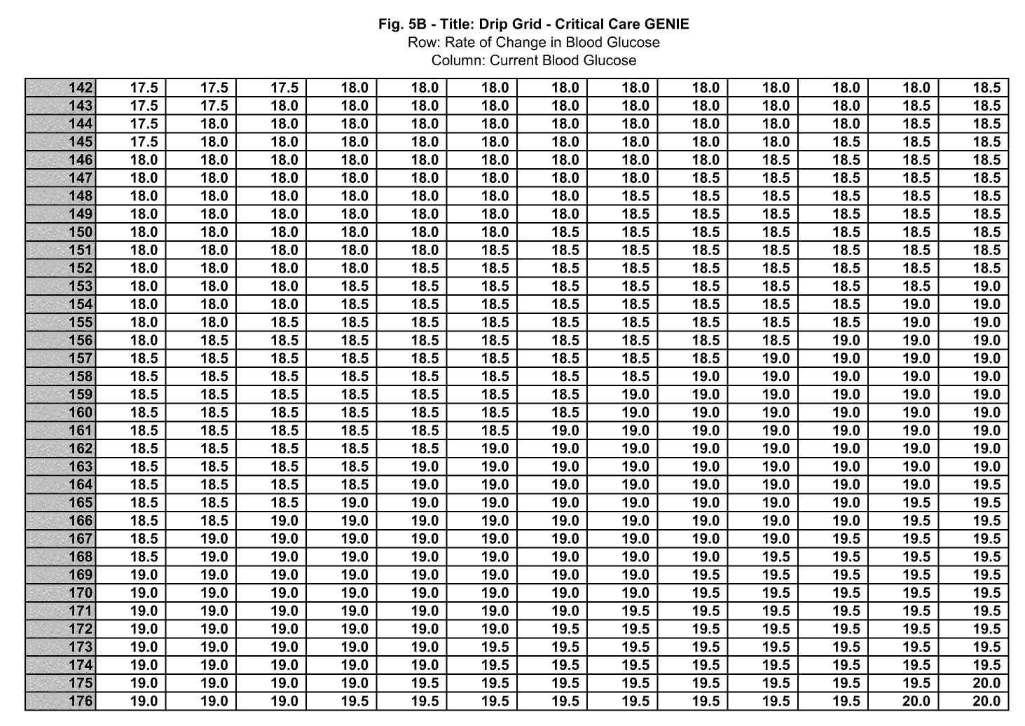

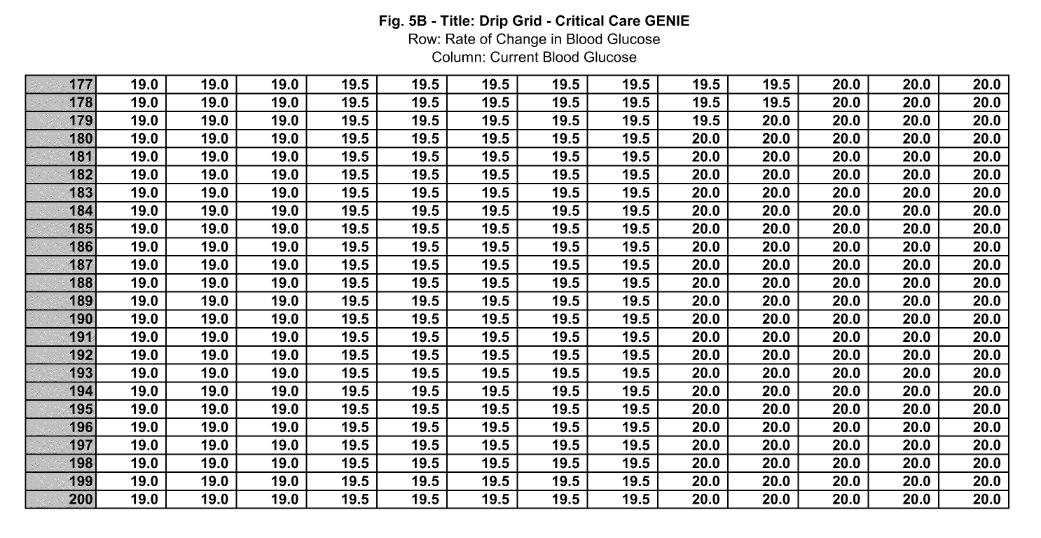

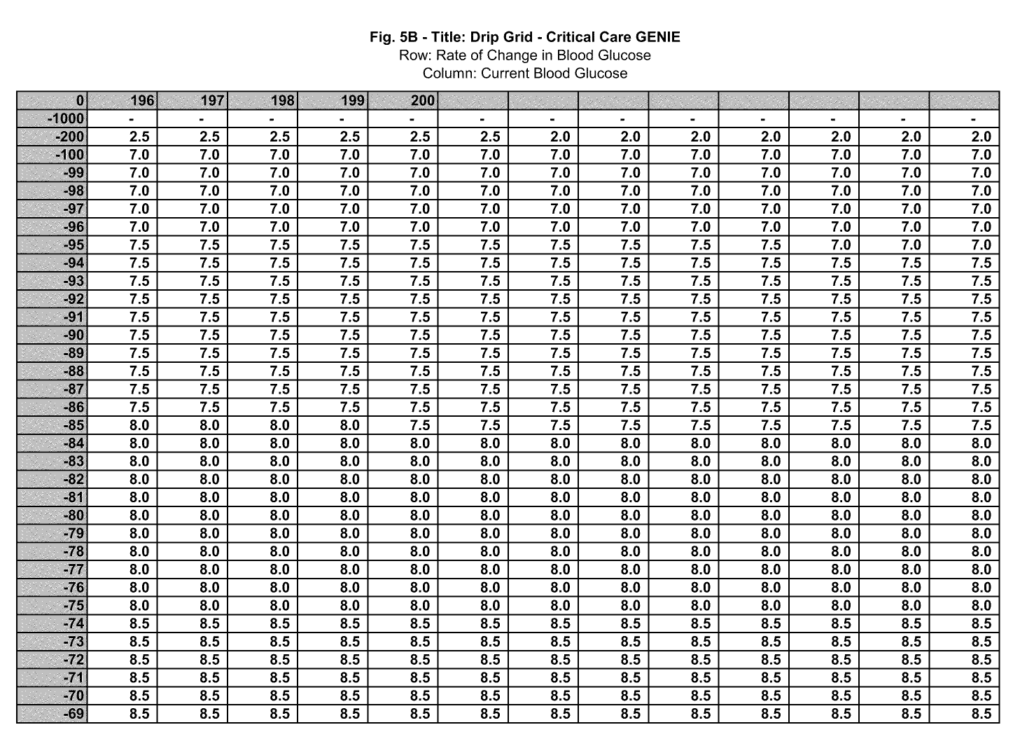

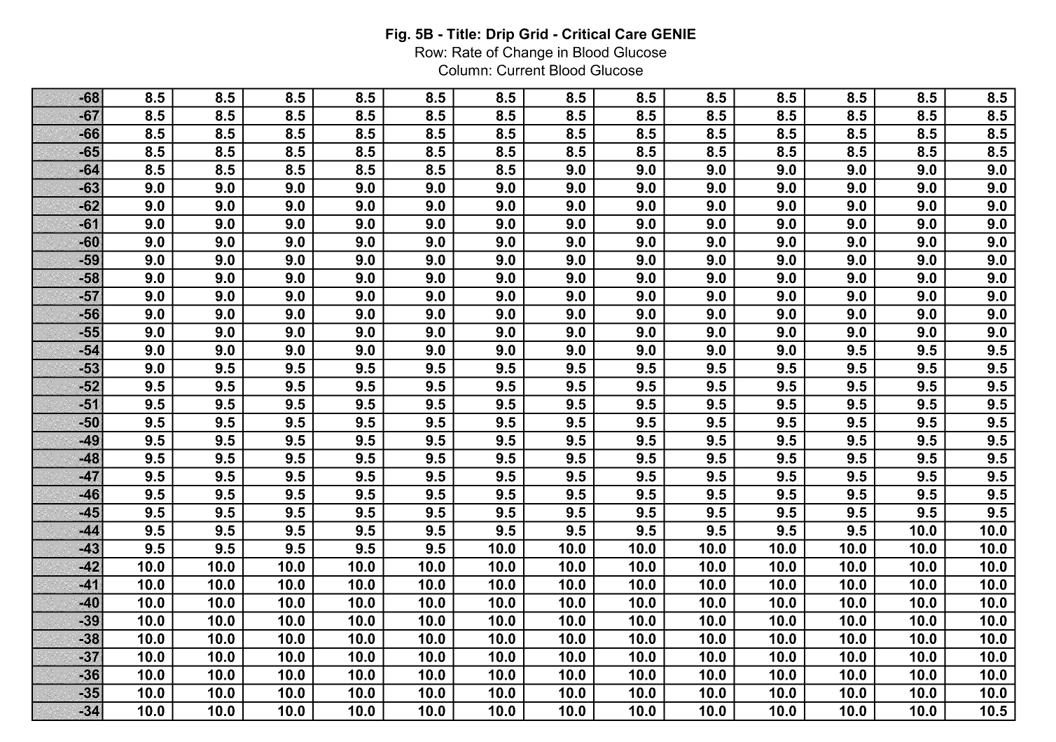

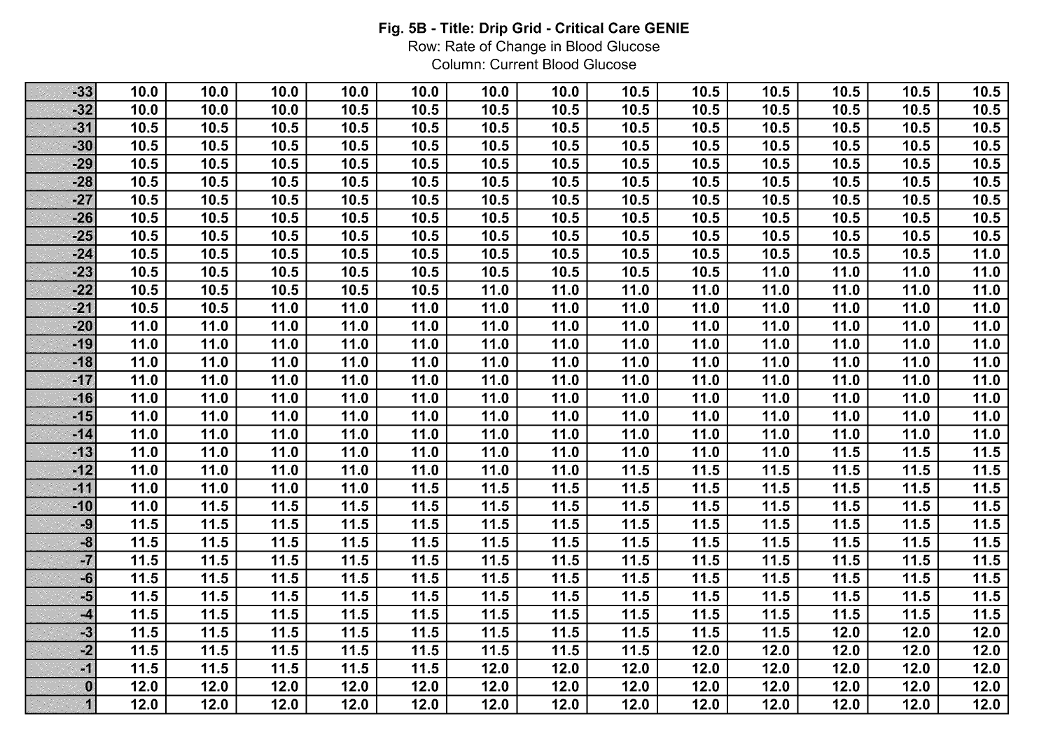

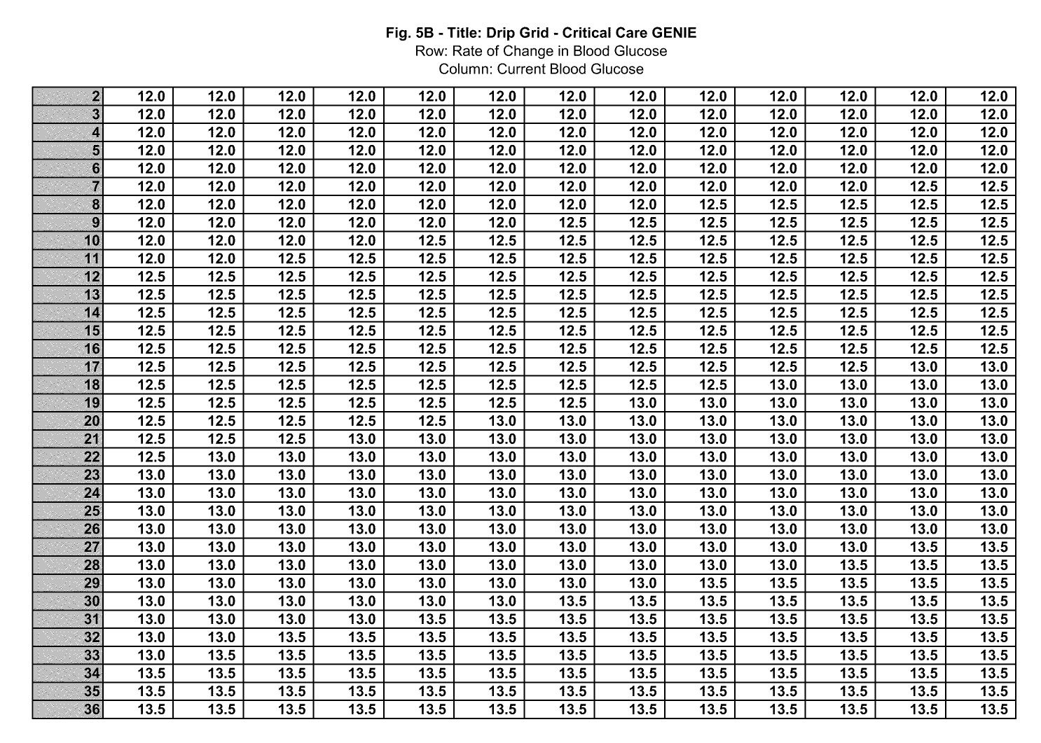

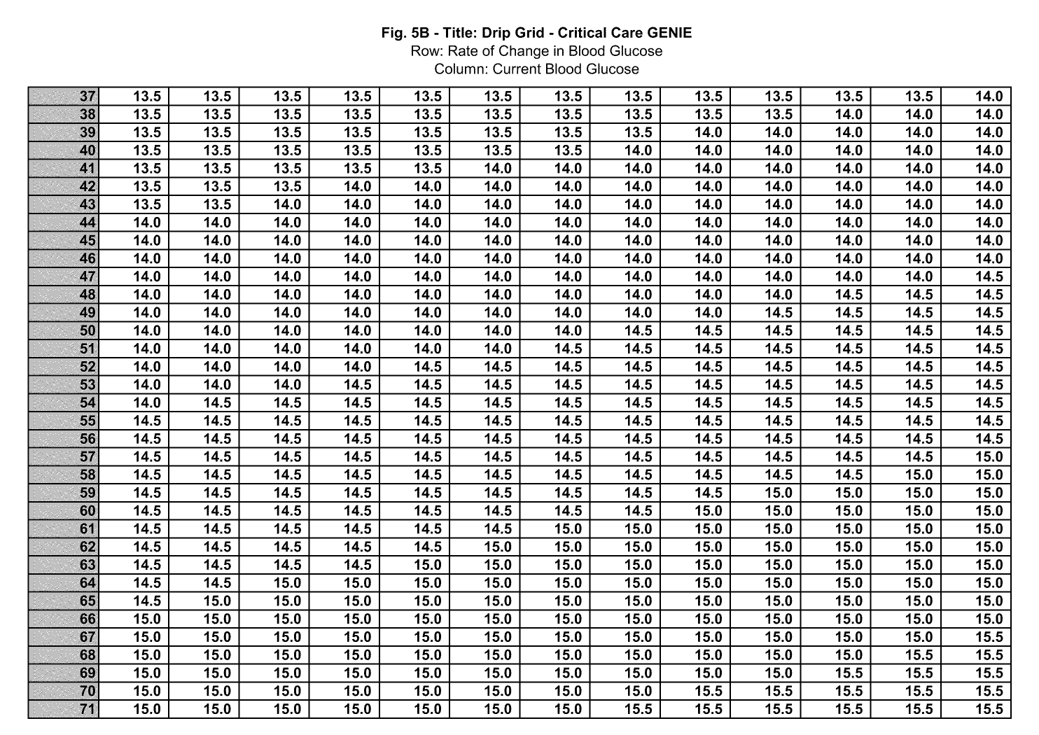

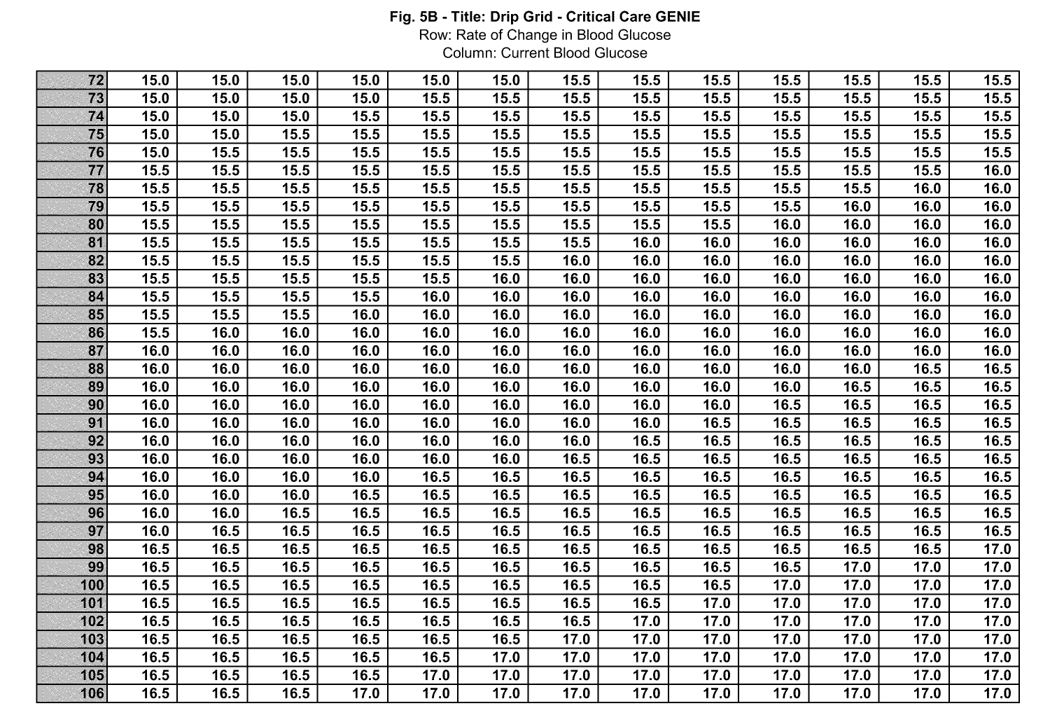

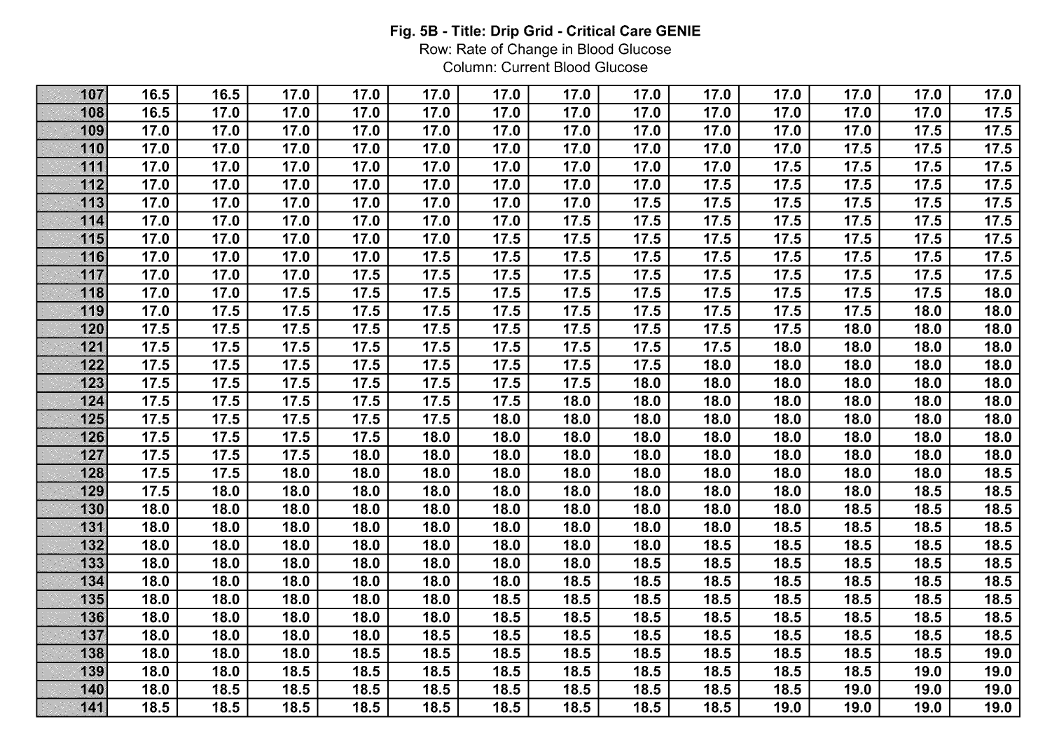

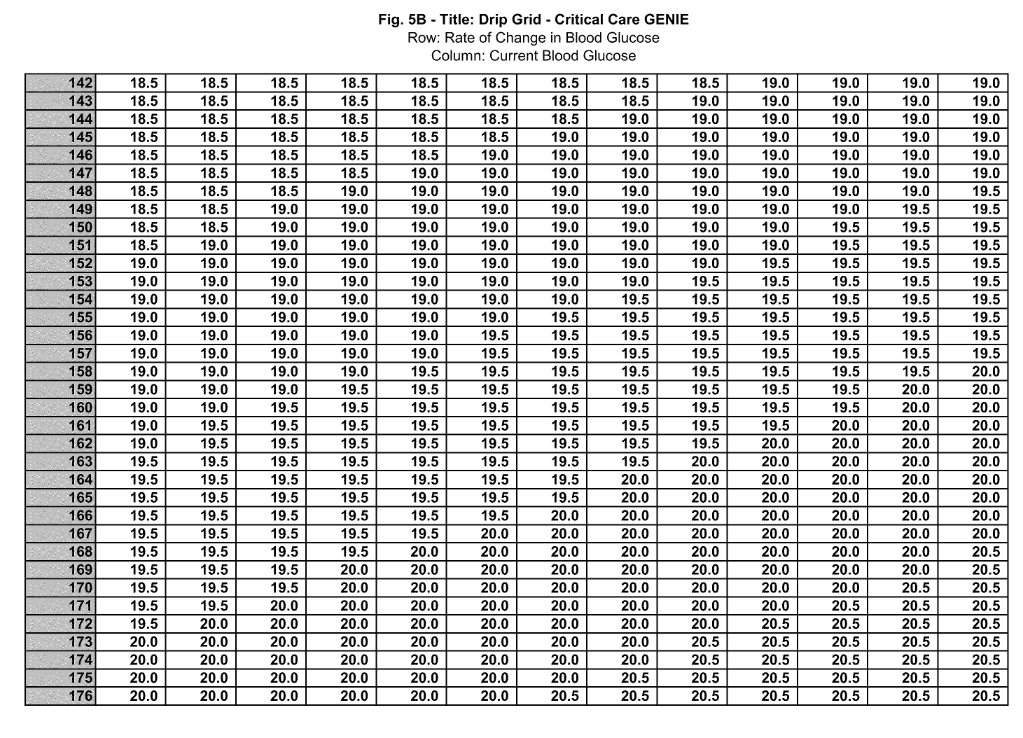

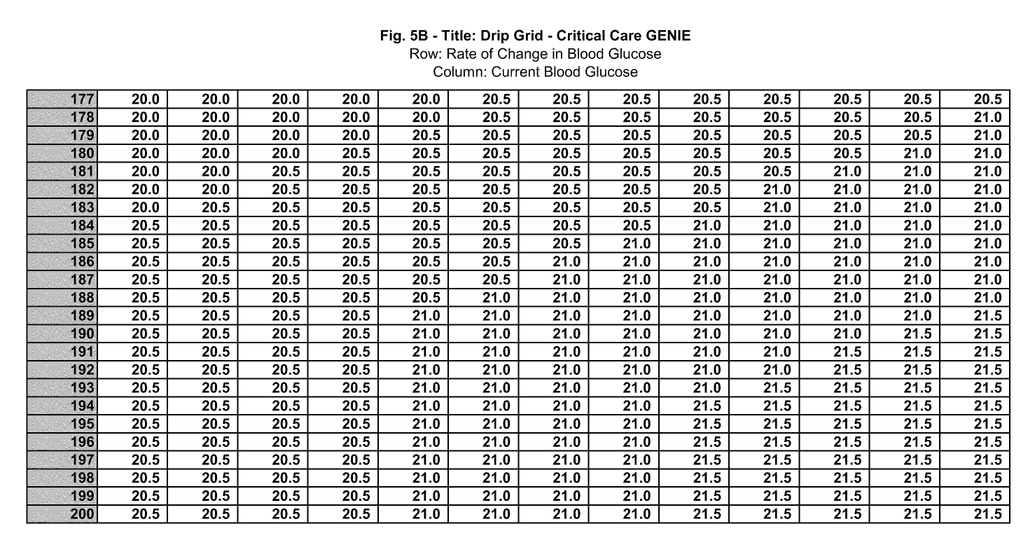

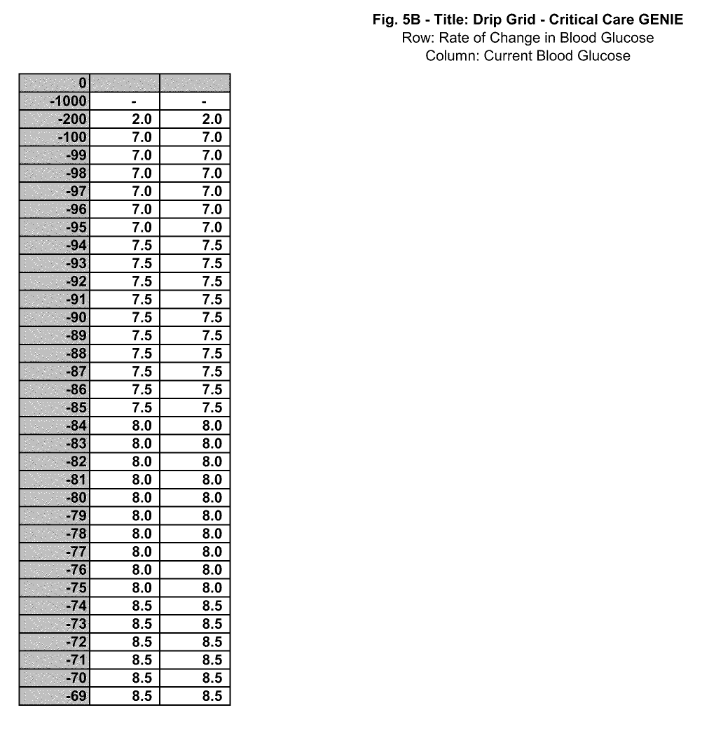

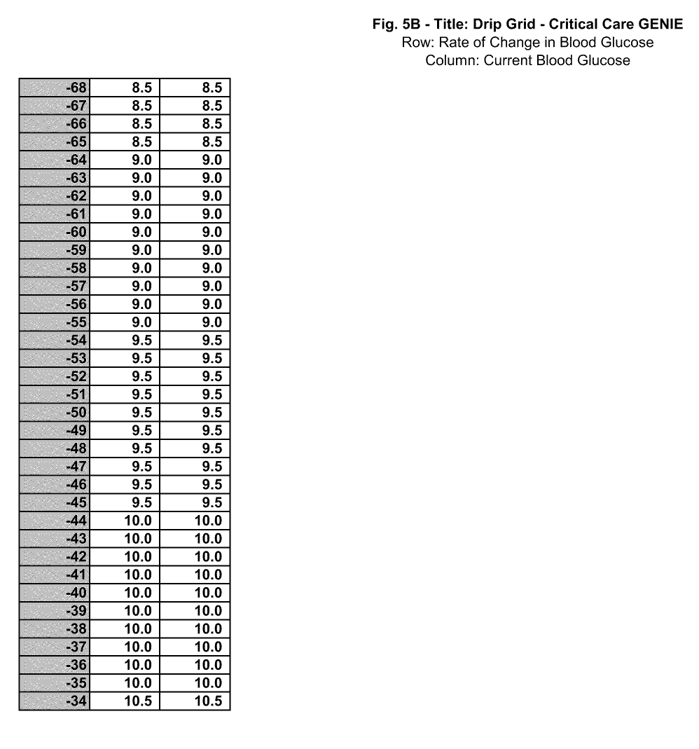

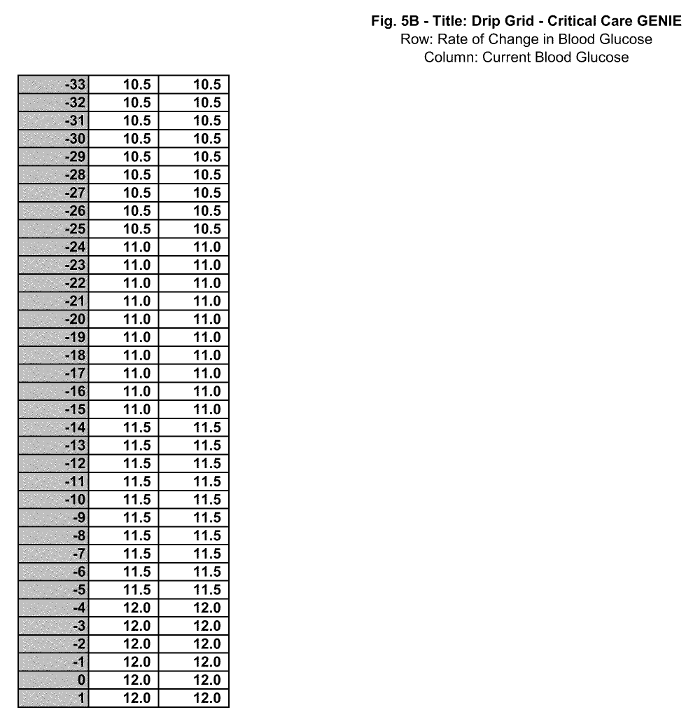

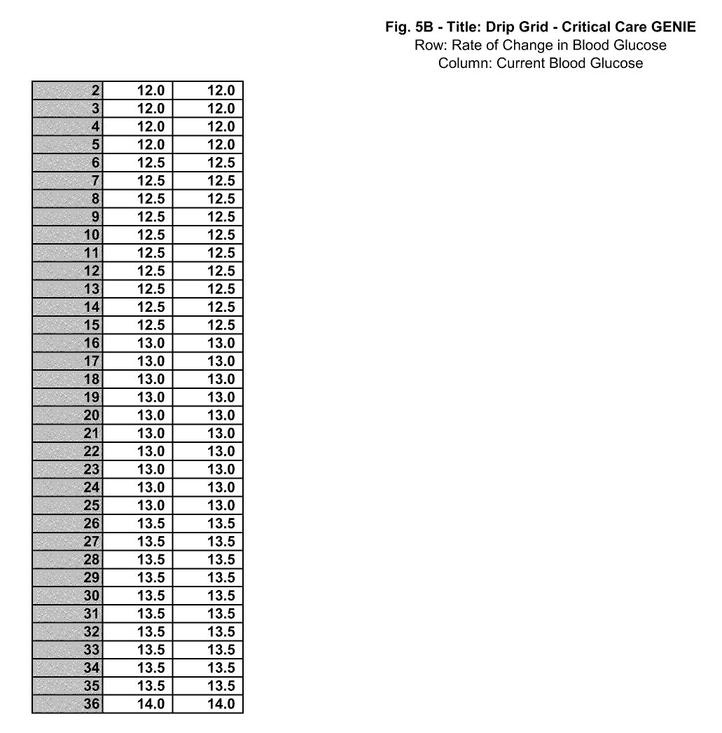

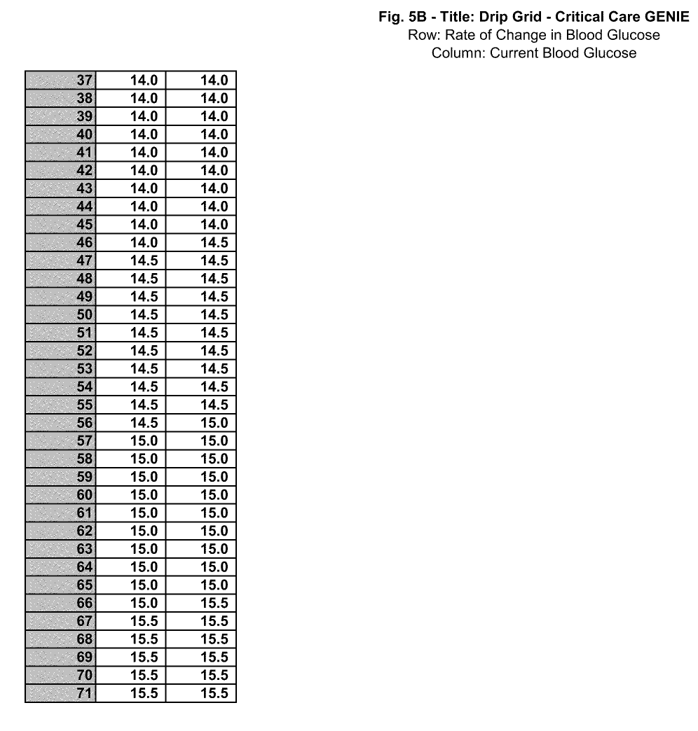

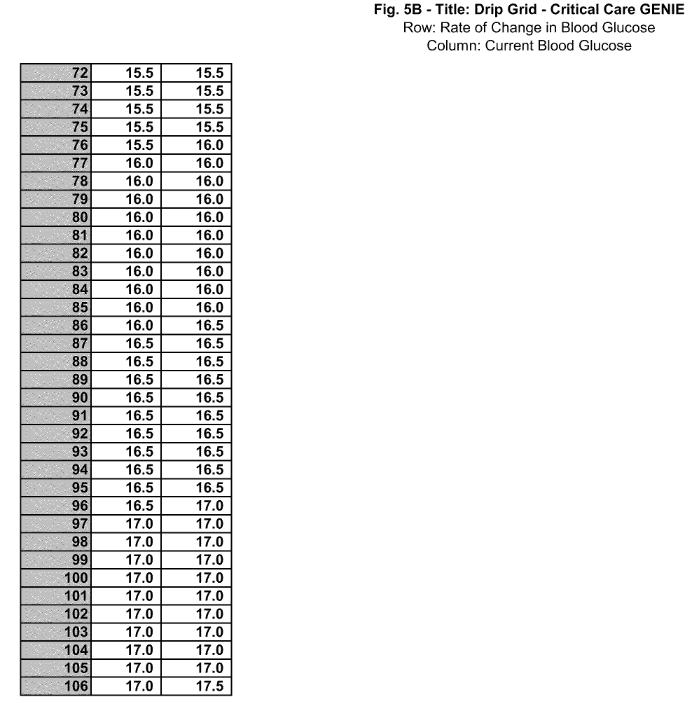

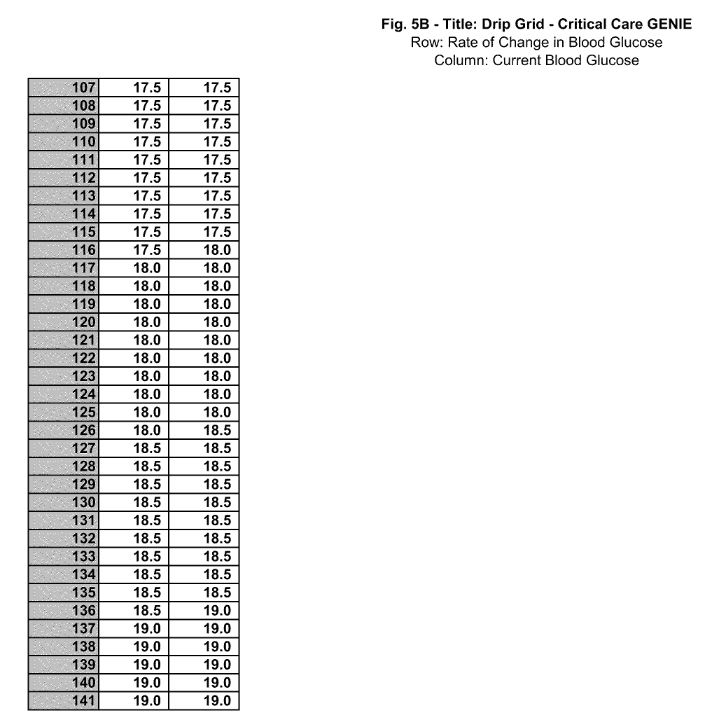

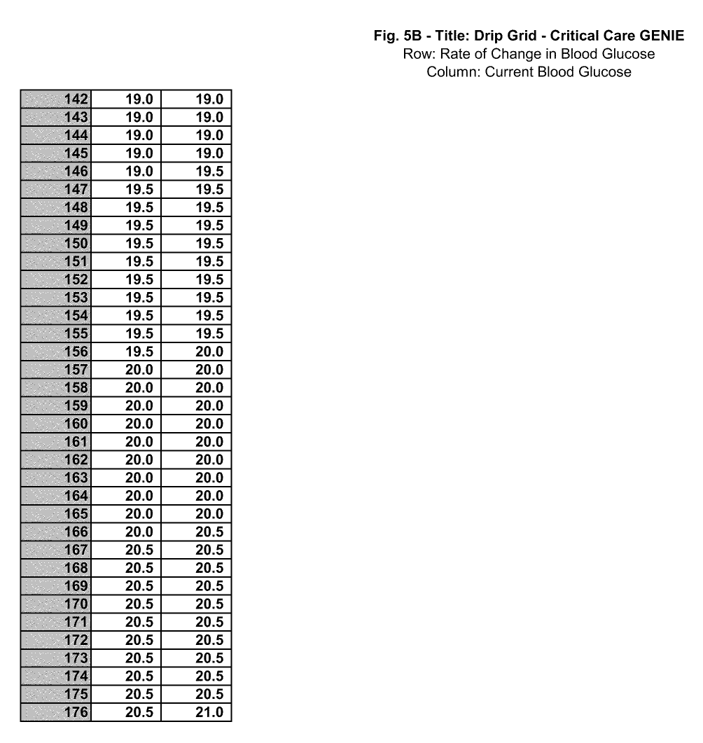

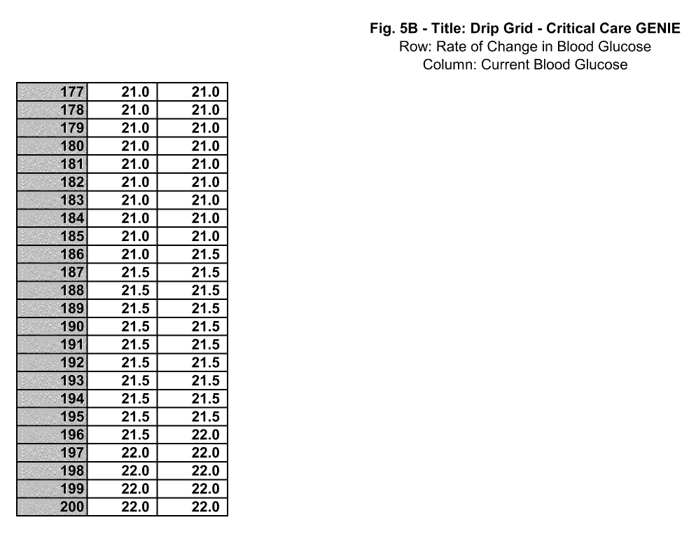

The Drip Grid referred to in the earlier discussion is an array with indices of rate of change in blood glucose (row) and current blood glucose level (column) that are specific to each of the GENIE's (FIGS. 5A and 5B). The indexed values held in the array are adjustment factors used to modify the current IV insulin drip rate.

The Drip Grid arrays for both ICU GENIEs (FIGS. 5A and 5B) show the “constants” that are used to make drip rate changes for subsequent dosing. These constants are the new drip rate, normalized for a prior drip rate of 10 units/hr. A “constant” of 5 indicates a 50% reduction from the preceding drip, whereas a constant of 15 indicates a 50% increase from the preceding drip rate. In other words, GENIE makes all changes relative to the preceding rate, thereby incorporating the patient's insulin sensitivity (referred to earlier as the “insulin response variable”) into the decision making strategy.

The constant values in the individual cells were experientially determined during development of GENIE, when it became evident that recommendations based on the formula that was used to develop the initial model did not provide the desired precision at blood glucose levels lower than 200 mg/dl.

The cell values in the table change according to two of the other three variables that are used to make decisions in GENIE (shown in FIG. 1B), namely blood glucose level (glycemic magnitude) and the rate of change in blood glucose (“glycemic response”) as follows:

1. The first row under the column headers in the uppermost row of the table, shows increasing blood glucose levels reading from left to right,

2. The first column after the row numbers in the extreme left column of the table shows the change in blood glucose from negative (a fall in blood glucose from earlier) to positive (an increase in blood glucose from earlier), reading from top to bottom.

The significance of the values of the constants in the table can be understood from the preceding description by keeping in mind the following:

(a) The section of the table in which there is a recommendation to administer no insulin via the drip shows cell values of represented by a hyphen or dash, (meaning 0 units/hr).

(b) The section of the table in which no change in drip rate is recommended shows cell values that are “10”.

(c) The section of the table in which there is a recommendation to decrease the drip rate, the cell values are numbers less than 10.

(d) The section of the table in which there is a recommendation to increase the drip rate, the cell values are numbers greater than 10.

The principles underlying the generation of these numbers become self-evident when it is, in turn, understood that:

(a) The higher the level of blood glucose the higher the cell value (constant)

(b) The greater the increase in blood glucose, the greater the cell value (constant)

Thus, for example, in the Drip Grid for Cardiac Surgery GENIE, the values of the constants in column “DG” for BG 110 range from a minimum of 3 at the top of the column to a maximum of 14. This means that the recommended change in drip rate in a patient with a BG of 110 can range from a decrease in drip rate to 30% of the previous rate to an increase by 40% of previous.

The factor that controls this gradation of constants is the rate of change in BG value. Thus, for instance, the values in row #214 (change in BG of 110) for the constants in the same table range from 0 (−) at the left extreme column and increase steadily starting from 3.0 in column “BS” (BG level of approximately 70) to a maximum of 17.5 at the right extreme column (BG 200). This means that a patient on Cardiac Surgery GENIE with a change in blood glucose of approximately 110 can have a drip change that ranges from a decrease to 30% of the preceding rate, to an increase by 75% of the previous.

A convenient way to understand the array is as a “decision surface” in which XY values are located on the surface with the user inputs of blood glucose change rate and level. A three-dimensional plot of the decision surface is shown in FIG. 5, which displays its irregular shape.

The elevation of the surface at any given location indirectly relates the percent change needed to be modified in the current drip rate. The chosen scale of the elevation at each point on this decision surface is set up to take advantage of the intuition of clinicians in treating patients. Each elevation value represents what a new IV insulin drip rate should be to treat a patient whose current drip rate is 10 units of IV insulin per hour. A value of 10.8, for example, would provide for an 8% increase in the IV insulin drip rate.

When a case in the Insulin Control Table accesses the Drip Grid for a new IV insulin drip setting, it then converts the value into the appropriate value for the new IV insulin drip rate. The Drip Grid value is divided by 10 to get a factor (1+the percent change in the drip rate) which is multiplied times the current case-specific drip rate. The result is then multiplied by 5, rounded, then divided by 5 to ensure all drip rates are rounded to the nearest 0.2 units/hour. Limits of a minimum of 0.6 units/hour and a maximum of “twice the current drip rate plus 10 units/hour” are then imposed on the result before it is returned to the user as a therapy recommendation.

B. Steps for Determining Insulin Dosing Recommendations that are Specific to Cardiac Surgery Genie

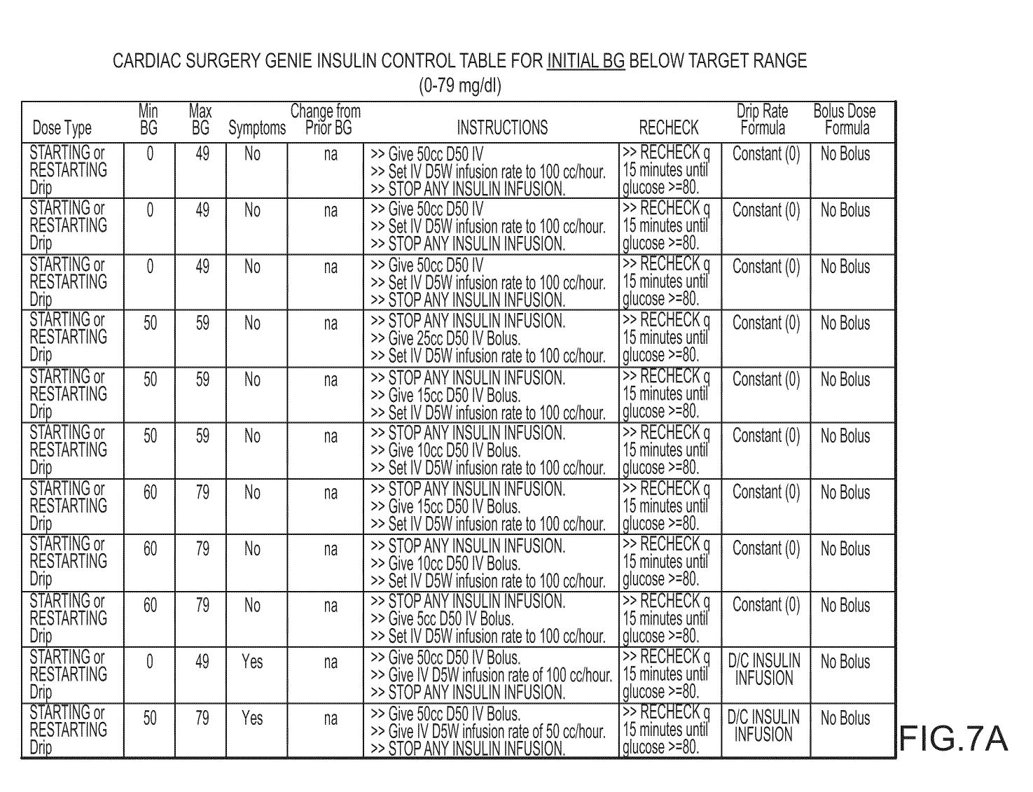

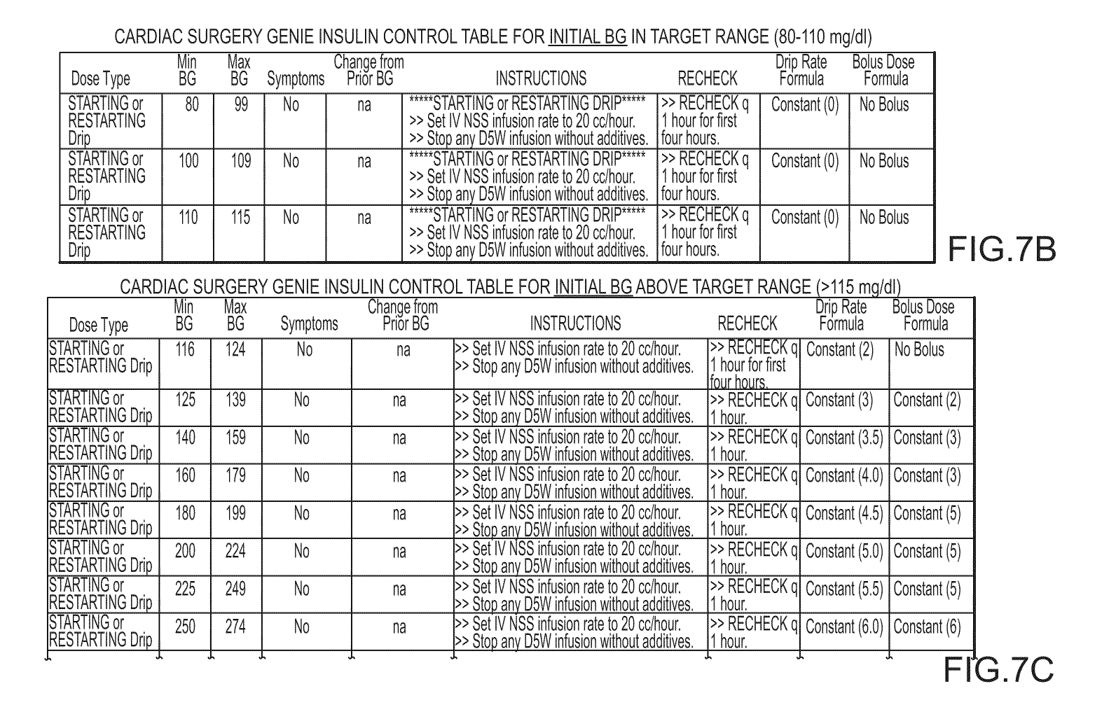

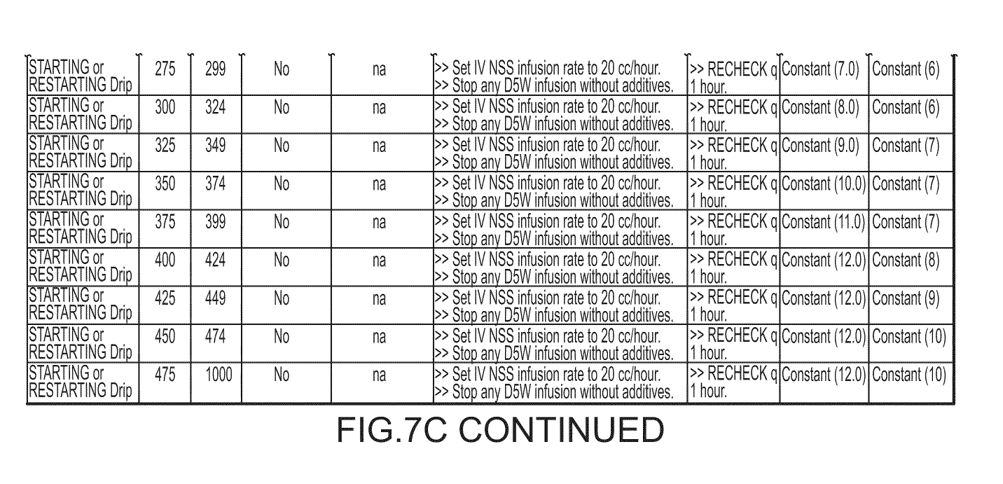

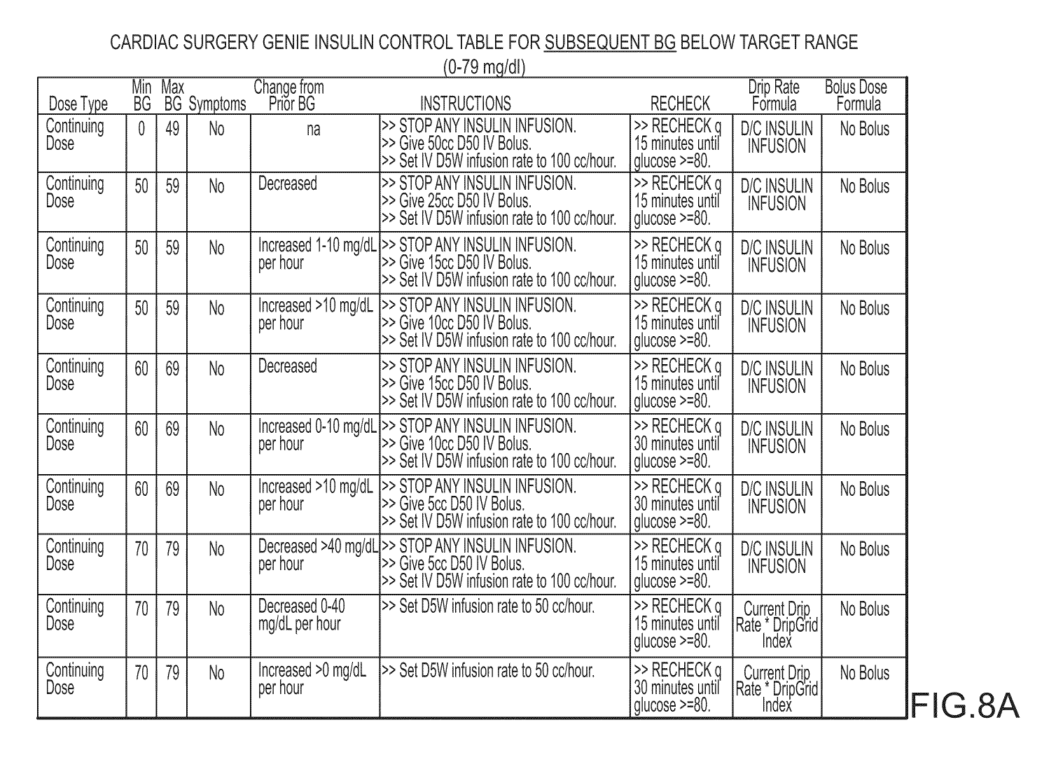

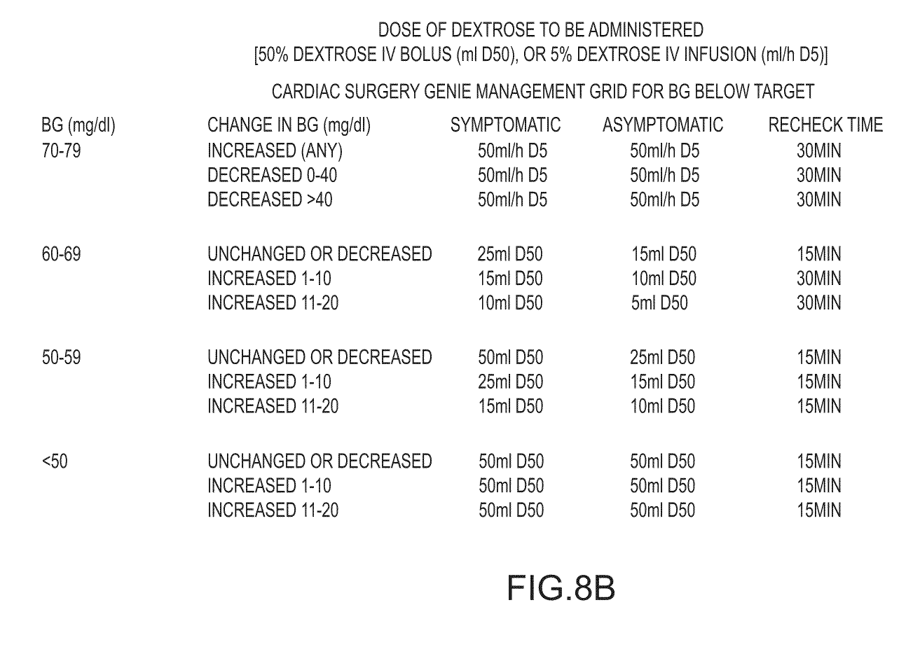

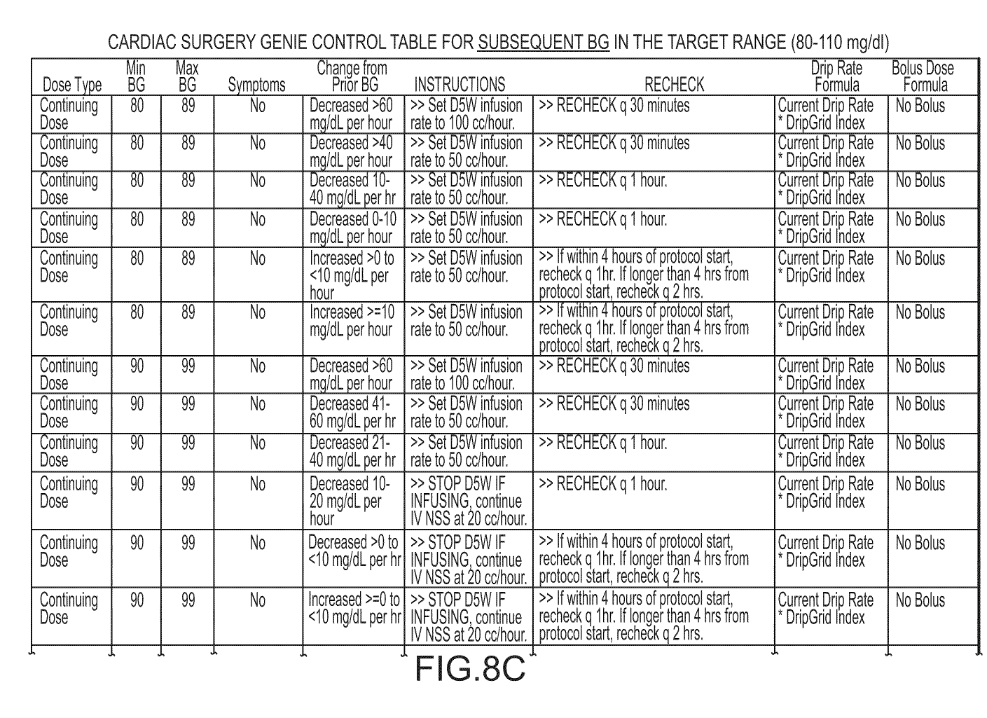

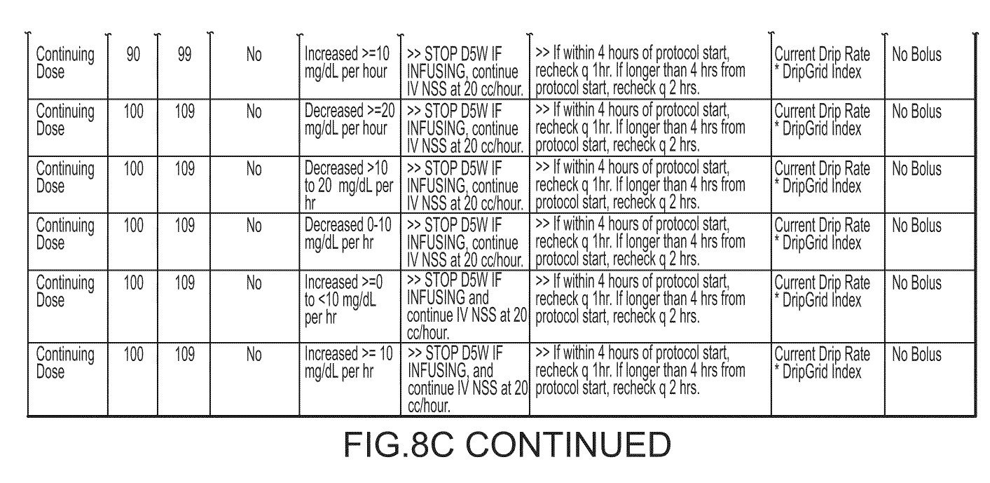

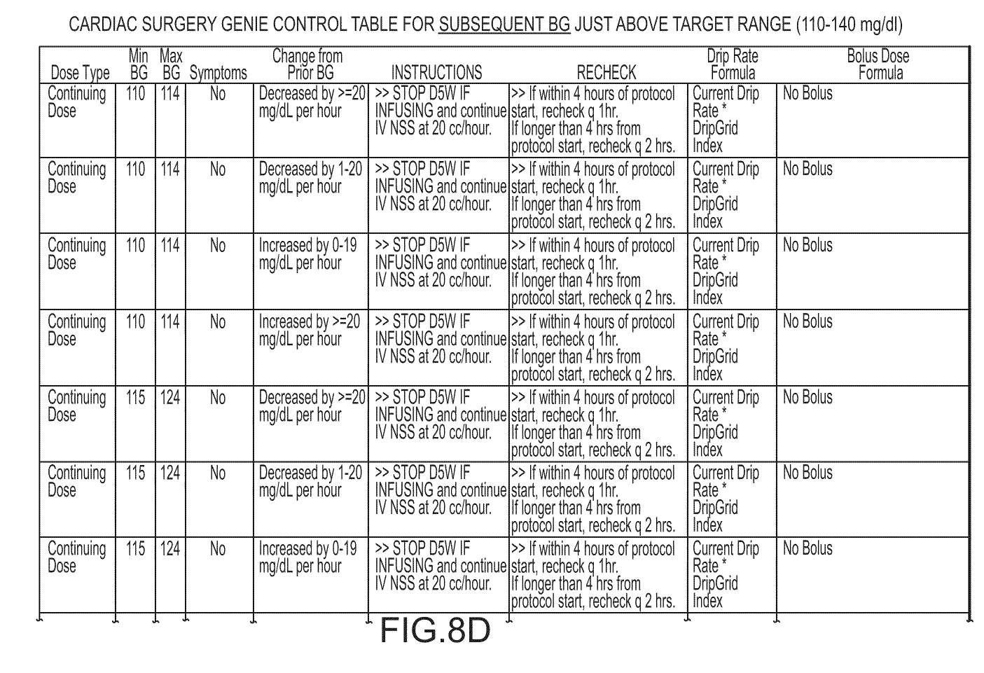

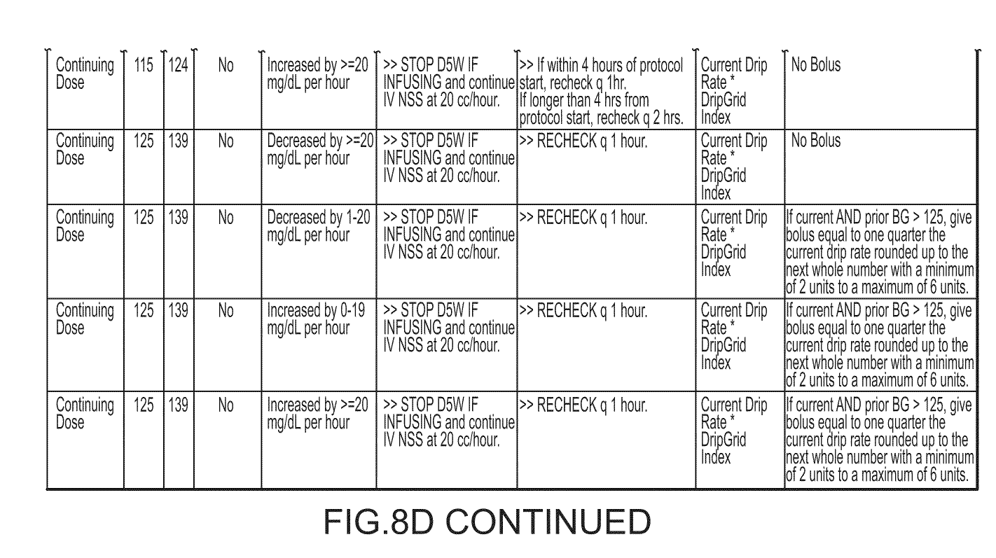

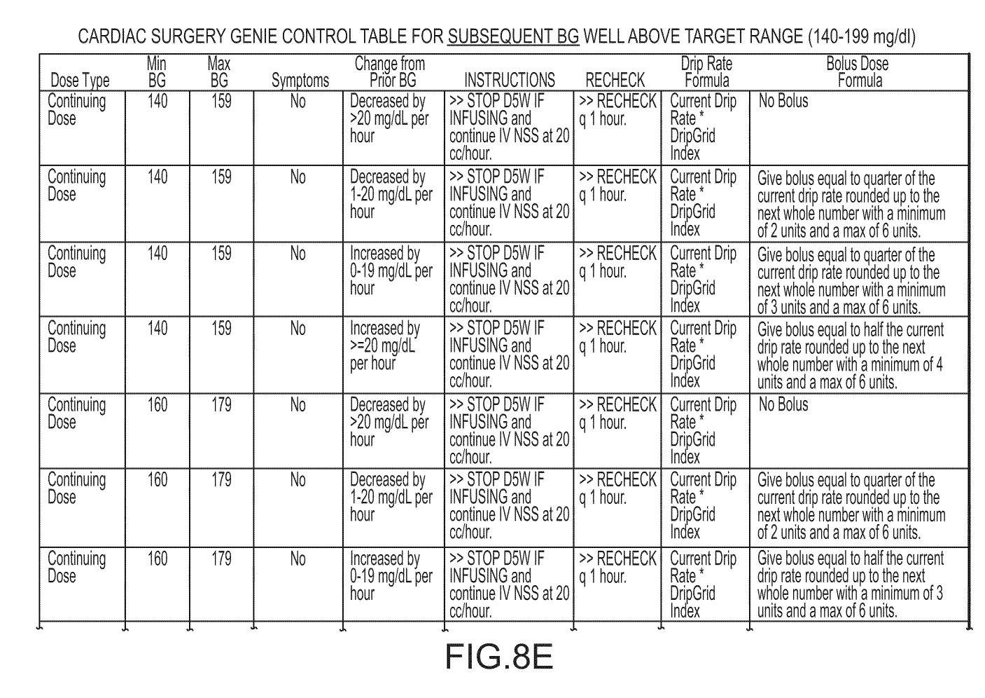

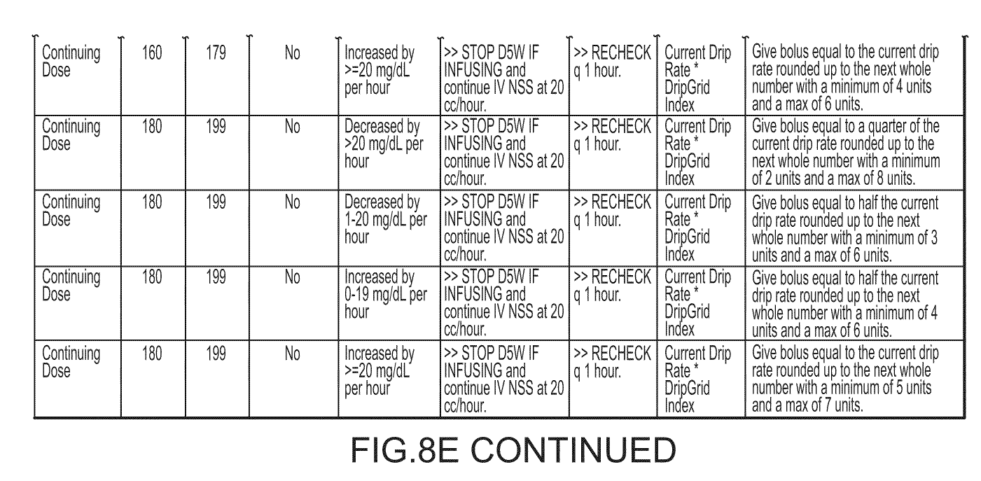

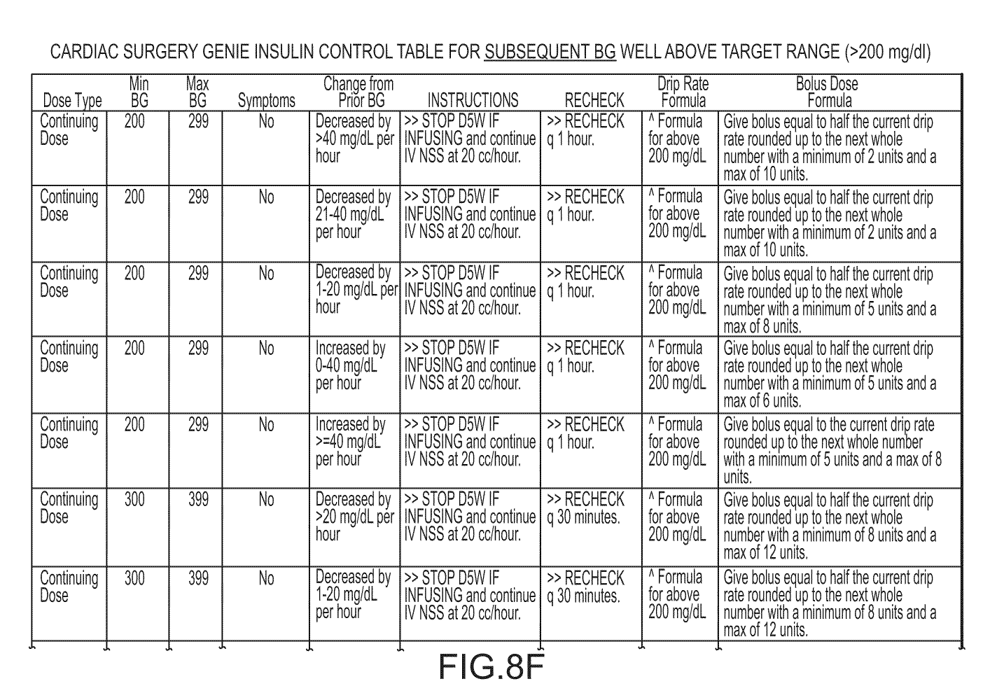

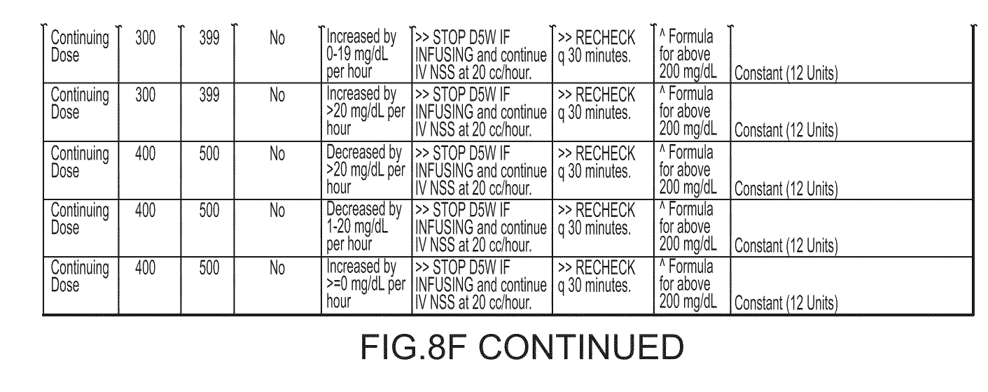

The Insulin Control Table for implementation of the common steps for Cardiac Surgery GENIE (Target Range approximately 80-110 mg/dl) is shown in FIGS. 7A-8F). These are divided according to the “Initial Dose” and “Subsequent Dose”, and further organized into sections that relate to specific Blood Glucose Ranges as follows:

1. Cardiac Surgery GENIE Initial Dosing recommendations

a. Cardiac Surgery GENIE Insulin Control Table for Initial BG
Below Target Range (approximately 0-79 mg/dl): FIG. 7A

The decision-making strategy in this range is included for the very rare circumstance when the patient has been on an alternative insulin management strategy, prior to starting GENIE, and is actually hypoglycemic as a result. The purpose is to provide instructions for management of hypoglycemia in a manner calibrated to the prevailing blood glucose without triggering a precipitous increase in BG. This strategy is implemented through the use of dextrose given either as a bolus of approximately 50% dextrose for symptomatic patients or significant hypoglycemia (<approximately 70 mg/dl) or as a continuous infusion of 5% dextrose for BG just below the target range (approximately 70-79 mg/dl).
b. Cardiac Surgery GENIE Insulin Control Table for Initial BG in Target Range (approximately 80-110 mg/dl): FIG. 7B

In this range, the initial management strategy is to not intervene with insulin, but to maintain support for blood glucose in the form of a 5% dextrose solution given at a low rate to avoid any precipitous drop in blood glucose. For patients within the range on protocol initiation, the GENIEs give NEITHER insulin nor dextrose. It is only other patients who enter the target range from higher BG's that could potentially receive dextrose. Instructions to recheck BG q hourly for the first 4 hours are included to make sure the undiagnosed diabetic patient with delayed onset of “stress hyperglycemia” is not overlooked.
c. Cardiac Surgery GENIE Insulin Control Table for Initial BG Above Target Range (>approximately 115 mg/dl): FIG. 7C

This is the range in which the full glycemic management strategy incorporated in GENIE is implemented.

Initial doses are looked up according to the patient's prevailing BG, and fractionated into bolus and drip doses, according to the Drip Rate Formula and Bolus dose Formula, as described above in Section A above.

2. Cardiac Surgery GENIE Subsequent Dosing Recommendations
a. Cardiac Surgery GENIE Insulin Control Table for SUBSEQUENT BG Below Target Range (approximately 0-79 mg/dl): FIG. 8A.

Treatment decisions for BGs below the target range (<approximately 80 mg/dl) that are attained after starting the GENIE (Continuing Doses) are based on the Below Target Management Grid” shown in FIG. 8B.

GENIE has several unique advantages as a result of the integration of this grid into the decision making strategy, which contribute directly to the virtual elimination of serious hypoglycemia with the use of this program:
(i) The first is the very fact of its incorporation into the main framework of the glycemic management program, which obviates the need for a separate protocol for management of hypoglycemia.
(ii) The second unique aspect is calibration of the aggressiveness of the intervention according to not only the prevailing blood glucose as a determinant of intervention (a common feature in all hypoglycemia management protocols), but also, uniquely in GENIE, the directional change from the preceding blood glucose level. This approach thus accounts for the substantial difference in the significance of a blood glucose level of approximately 50 mg/dl, depending on whether this level was attained from a preceding value of approximately 40 mg/dl, or approximately 60 mg/dl. In the former case (blood glucose rising from the previous value of approximately 40) the goal is to accelerate the increase in blood glucose. In the latter (blood glucose falling from the previous value of approximately 60) the goal is to both stop the decline as well as to reverse it. As consequence, the former situation calls for a smaller amount of intravenous glucose than the latter.
(iii) A third unique feature is to incorporate the presence or absence of symptoms of hypoglycemia into the decision, in recognition of the fact that symptomatic hypoglycemia calls for greater aggressiveness in intervention regardless of the actual level of blood glucose, than asymptomatic hypoglycemia.
(iv) A fourth is the recognition of the importance of providing a “safety net” in patients who are trending outside the target range, thereby providing a safety net against the onset of hypoglycemia when the BG falls below the target range, instead of waiting to intervene until BG reaches hypoglycemic levels of <approximately 70 mg/dl. In this circumstance, simply discontinuing the drip might result in a precipitous rise in BG. In the Cardiac Surgery GENIE program, when BG falls into the approximately 70-79 mg/dl range as a result of a decrease in BG that exceeds approximately 20 mg/dl, the “Instructions” dictate the use of a 5% dextrose infusion to support the blood glucose and bring it gently back to the target range, while simultaneously decreasing the insulin drip rate according to the “drip grid index”, but not simply discontinuing the drip. This constitutes a “twin dial strategy” that improves the precision of control of BG at the lower end of the target range, thereby protecting against abrupt swings in BG.
(v) A fifth unique feature is the fact that the required recheck times for the next blood glucose measurement are automatically adjusted according to both the actual BG level as well as the change in BG from previous value, once again reflecting the need to calibrate follow-up according to not just the severity of hypoglycemia but also the trend.
(vi) A sixth unique feature is the recognition of the importance of providing a “safety net” in patients who are trending outside the target range, which is an extension of the twin-dial strategy noted earlier.
b. Cardiac Surgery GENIE Insulin Control Table for SUBSEQUENT BG in the Target Range (approximately 80-110 mg/dl): FIG. 8C

The maintenance of BG in the target range of approximately 80-110 mg/dl is achieved through the exclusive use of the insulin drip, and no bolus doses. The drip rate calculations are derived from the “DRIP GRID INDEX” which is based on three of the four independent variables that comprise the unique combination of inputs used to determine decision-making strategy in GENIE, namely the “glycemic magnitude”, “glycemic response” and “insulin response” variable.

The rule base for drip rate adjustments is implemented in this range as follows:
(i) Maintaining insulin drip at a constant rate for a BG that remains unchanged (BG decreasing by less than approximately 10 mg/dl)
(ii) Reducing insulin drip rate when the BG enters the target range by a decline of greater than approximately 10 mg/dl (“glycemic magnitude”)
(iii) Uniquely calibrating the decrease in drip rate at any blood glucose level in the target range to the preceding decline that led to the BG reaching target, so that an increasing rate of change (approximately 10-19, 20-39, 40-59, or ≧60 mg/dl/hr) triggers a greater reduction in insulin drip (“glycemic response”)
(iv) Uniquely calibrating the decrease in drip rate to the existing insulin infusion rate, so that changes are relative to each individual patient's insulin sensitivity (“insulin response”), and not a fixed or absolute decrease in drip rate.
(v) Uniquely recognizing that a safety net, in the form of a 5% dextrose infusion is required for:
   a. BG levels in the lower end of the target range (approximately 80-89)
   b. BG levels in the approximately 90-99 range which occur as a result of a decline in BG>approximately 20 mg/dl
   c. Calibrating the rate of 5% dextrose infusion rate to the magnitude of the decline from the preceding BG
c. Cardiac Surgery GENIE Insulin Control Table for SUBSEQUENT BG Just Above Target Range (110-140 mg/dl): FIG. 8D

The rule bases in the approximately 110-140 range are set up to achieve the target range according this strategy by adjusting the insulin drip according to the insulin drip grid index, without using bolus insulin, according to the following respective goals:
(i) slow the descent into target range when BG is trending down too aggressively;
(ii) maintain the trajectory of the descent when BG is decreasing by a satisfactory amount;
(iii) re-establish downward momentum when BG is unchanged or not decreasing by a satisfactory amount; and
(iv) reverse the directional trend to a higher BG when BG is increasing.

For most circumstances that occur in this range, treatment decisions are implemented exclusively through the use of insulin drip adjustments, using the “Drip Grid Index”, which is based on a combination of three of the four independent variables that comprise the unique combination of inputs used to determine decision-making strategy in GENIE, namely the “glycemic magnitude”, “glycemic response” and “insulin response” variable.

In a single set of circumstances, when two successive BGs are approximately 125-139, a supplemental bolus dose of insulin is also given, which is calculated from the insulin drip rate, using the fourth independent variable in the unique combination used for decision making strategy, namely the fractional partition of insulin dose into drip and bolus, which is the “insulin dose variable”. This is the only exception in the range of approximately 125-140, and it constitutes a unique and specific strategy to prevent persistently sluggish responses resulting in successive readings staying above the target range without declining.

The Bolus and Drip Rate calculations applicable to this range are described above in Section A above.
d. Cardiac Surgery GENIE Insulin Control Table for SUBSEQUENT BG Above Target Range (approximately 140-199 mg/dl): FIG. 8E

The rule base in this BG range is similar to that in the mild hyperglycemic range (approximately 110-140 mg/dl), with one major conceptual difference, namely, that bolus insulin is an integral component of the strategy. This means that the “insulin dose variable” (i.e., fractional partition of the dose into bolus and drip insulin) is combined with the other three independent variables, which are “glycemic magnitude” (prevailing BG), “glycemic response” (rate of change in BG), and “insulin response” (the prevailing insulin responsiveness reflected in the existing drip rate). The drip rate calculations are based on a more aggressive strategy than in the mild hyperglycemia range, to calculate the insulin dose from the existing drip rate, thereby calibrating the dosing relative to the prevailing circumstances. This is unique to GENIE, in contrast to other programs which take into account at most the first two variables (prevailing BG and absolute change in BG). GENIE uniquely incorporates the patient's insulin responsiveness in the calculation of the insulin bolus.

The total insulin dose for fractional partitioning into drip and bolus doses is based on the Drip Grid Index and the Bolus Dose formula applicable to this range, as described in Section A above.
e. Cardiac Surgery GENIE Insulin Control Table for SUBSEQUENT BG Well Above Target Range (>approximately 200 mg/dl:) FIG. 8F.

Above a BG of approximately 200 mg/dl, GENIE uses a mathematical computation of insulin dose based on all four independent variables that comprise the decision-making strategy in GENIE, namely, “glycemic magnitude” (prevailing BG), “glycemic response” (rate of change in BG), “insulin response” (the prevailing insulin responsiveness reflected in the existing drip rate), and “insulin dose” for fractional partitioning of the dose into bolus and drip insulin. In this range, the philosophy is that errors in predicting response based on the use of a natural function are acceptable, since the BG is far enough above target range to allow for correctional action to be taken before the patient becomes hypoglycemic.

The total insulin dose for fractional partitioning into drip and bolus doses is based on the Drip Grid Index and the Bolus Dose formula applicable to this range, as described in Section A above.

C. Steps for Determining Insulin Dosing Recommendations that are Specific to Critical Care Genie

The Insulin Control Table for implementation of the common steps for Cardiac Surgery GENIE (Target Range 80-110 mg/dl) is shown in FIGS. 7 A-F). These are divided according to the “Initial Dose” and “Subsequent Dose”, and further organized into sections that relate to specific Blood Glucose Ranges as follows:

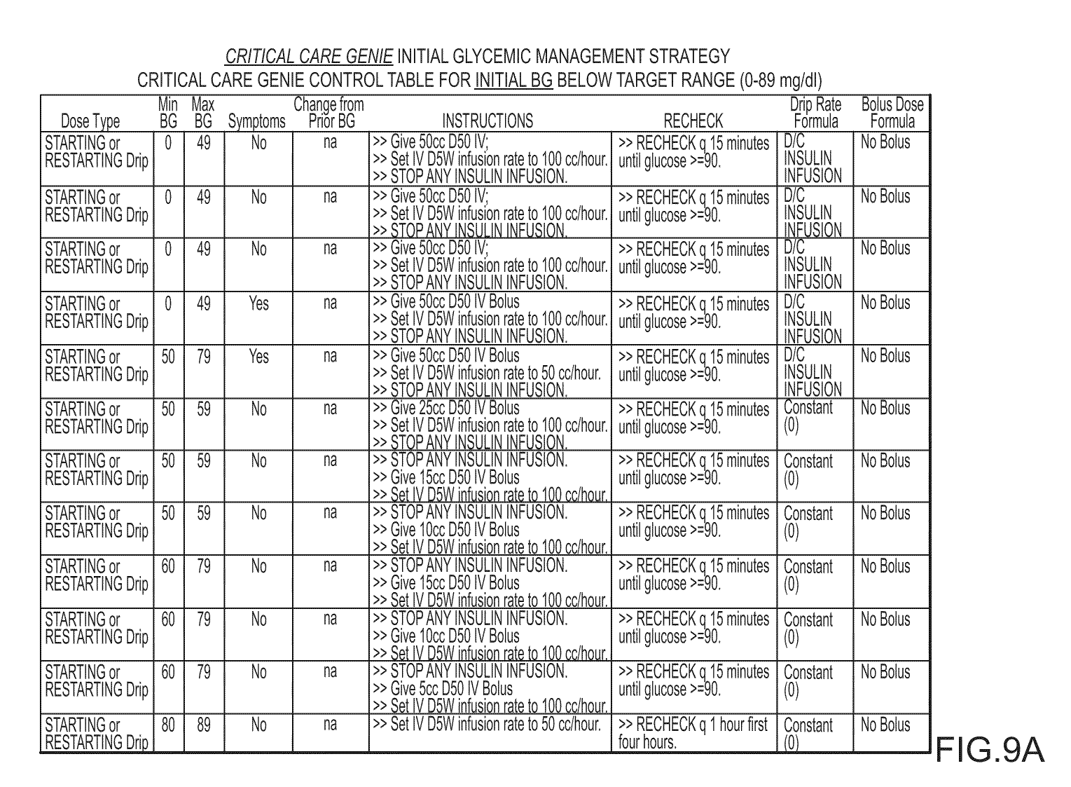

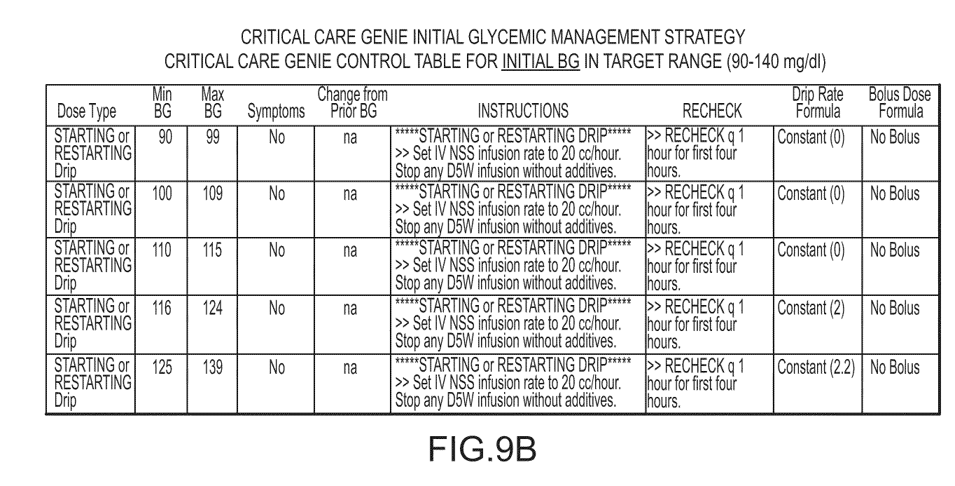

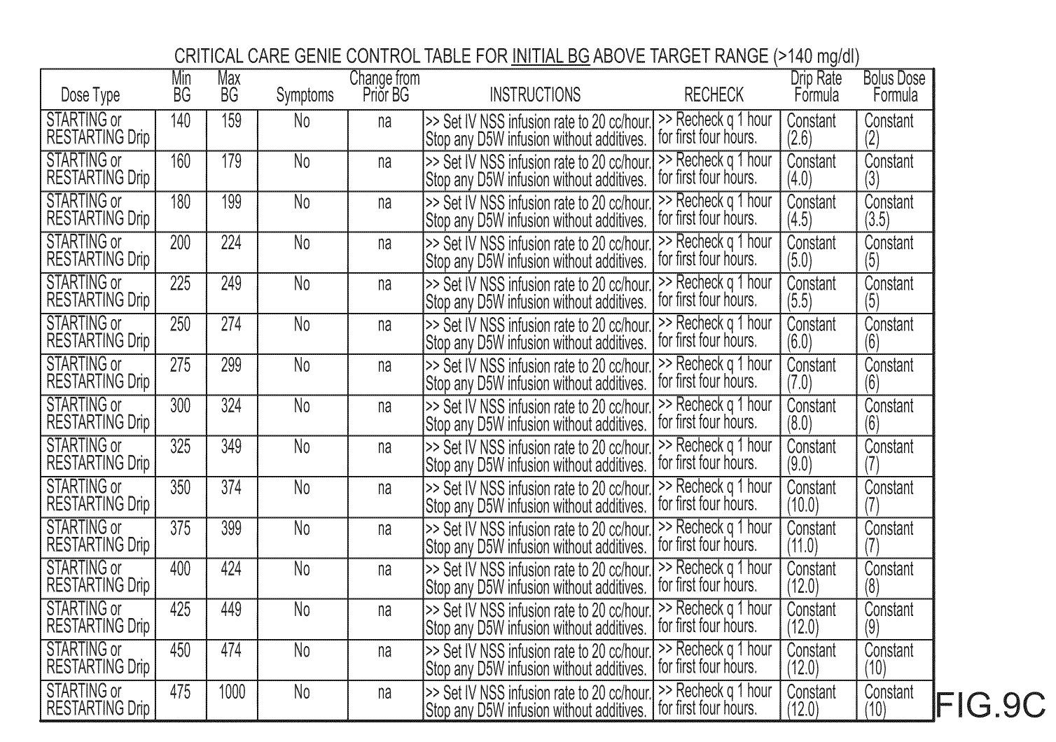

1. Initial Dosing Recommendations
a. Critical Care GENIE Insulin Control Table for Initial BG Below Target Range (0-89 mg/dl): FIG. 9A

The decision-making strategy in this range is included, as in the Cardiac Surgery GENIE, for the very rare circumstance when the patient has been on an alternative insulin management strategy prior to starting GENIE, and is actually hypoglycemic as a result. The purpose is to provide instructions for management of hypoglycemia in a manner calibrated to the prevailing blood glucose without triggering a precipitous increase in BG. This strategy is implemented, as in Cardiac Surgery GENIE, through the use of dextrose given either as a bolus of approximately 50% dextrose for symptomatic patients or significant hypoglycemia (<approximately 70 mg/dl) or as a continuous infusion of 5% dextrose for BG just below the target range (approximately 70-89 mg/dl for Critical Care GENIE, as opposed to approximately 70-79 mg/dl for Cardiac Surgery GENIE).
b. Critical Care GENIE Insulin Control Table for Initial BG in Target Range (approximately 90-140 mg/dl): FIG. 9B

The initial management strategy for BG in the target range (approximately 90-139 mg/dl) differs substantially from Cardiac Surgery GENIE (where the target range is approximately 80-115 mg/dl).

In the lower half of the target range of Critical Care GENIE (BG approximately 90-115 mg/dl), the initial management strategy is to not use any insulin, either as a drip or a bolus. Instructions to recheck BG q hourly for the first 4 hours are included to make sure the undiagnosed diabetic patient with delayed onset of “stress hyperglycemia” is not overlooked.

In the upper half of the target range (BG is above approximately 115-139 mg/dl), there continues to be no use of any bolus insulin, but a low dose insulin infusion is started according to the drip rate formula described in Section A above.
c. Critical Care GENIE Insulin Control Table for Initial BG Above Target Range (>approximately 140 mg/dl): FIG. 9C

This is the range in which the full glycemic management strategy incorporated in GENIE is implemented.

Initial doses are calculated according to the patient's prevailing BG, and fractionated into bolus and drip doses, according to the Drip Rate Formula and Bolus dose Formula described in Section A above.

2. Subsequent Dosing Recommendations
a. Critical Care GENIE Insulin Control Table for Subsequent BG

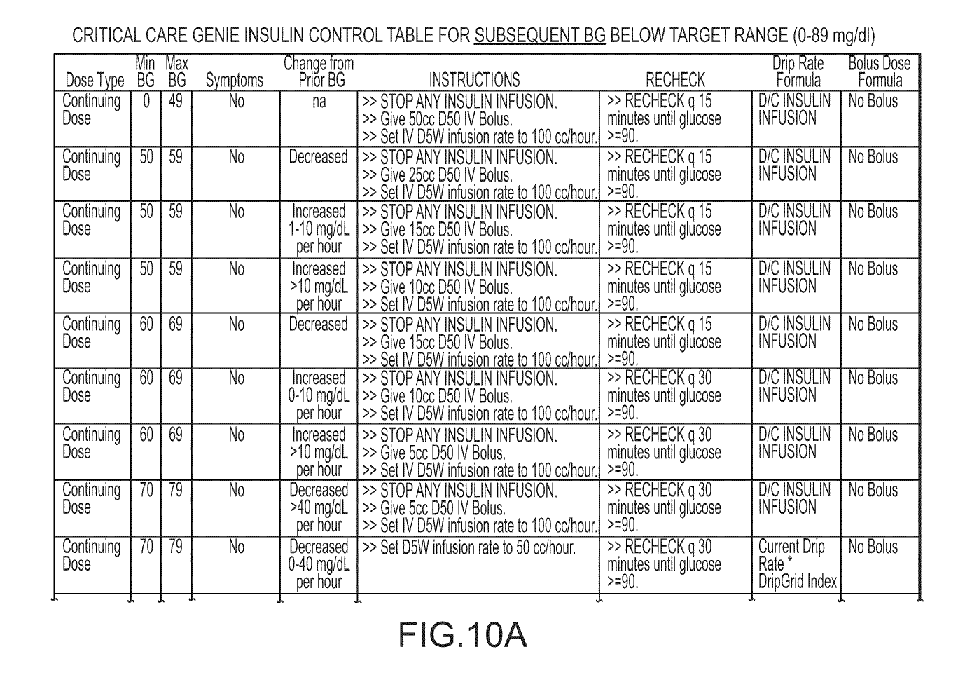

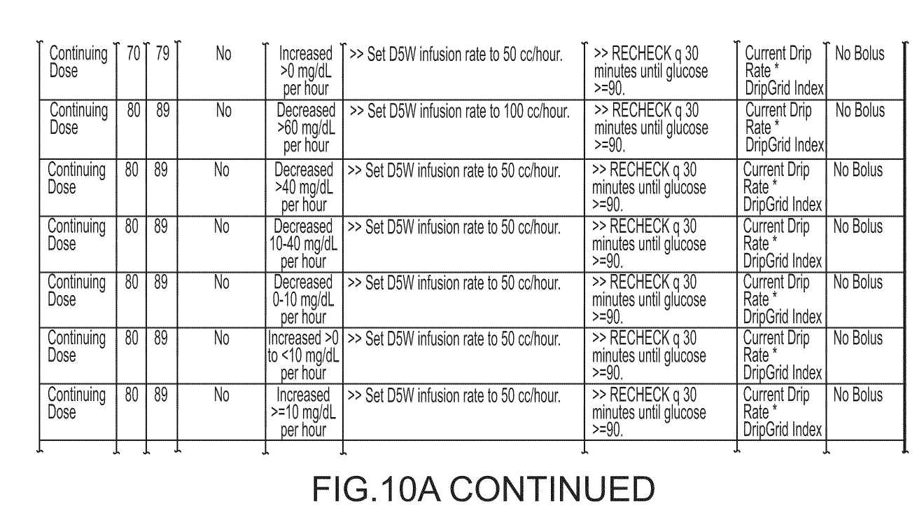

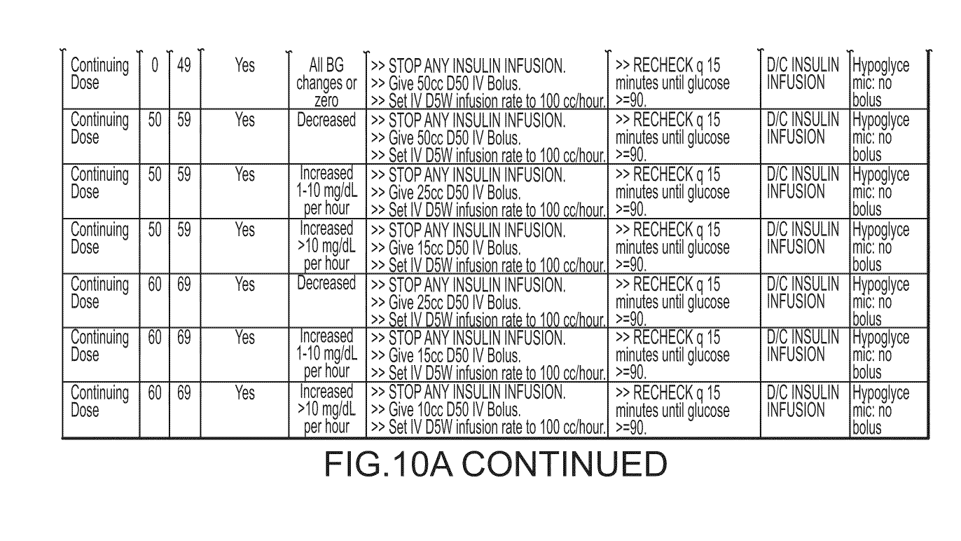

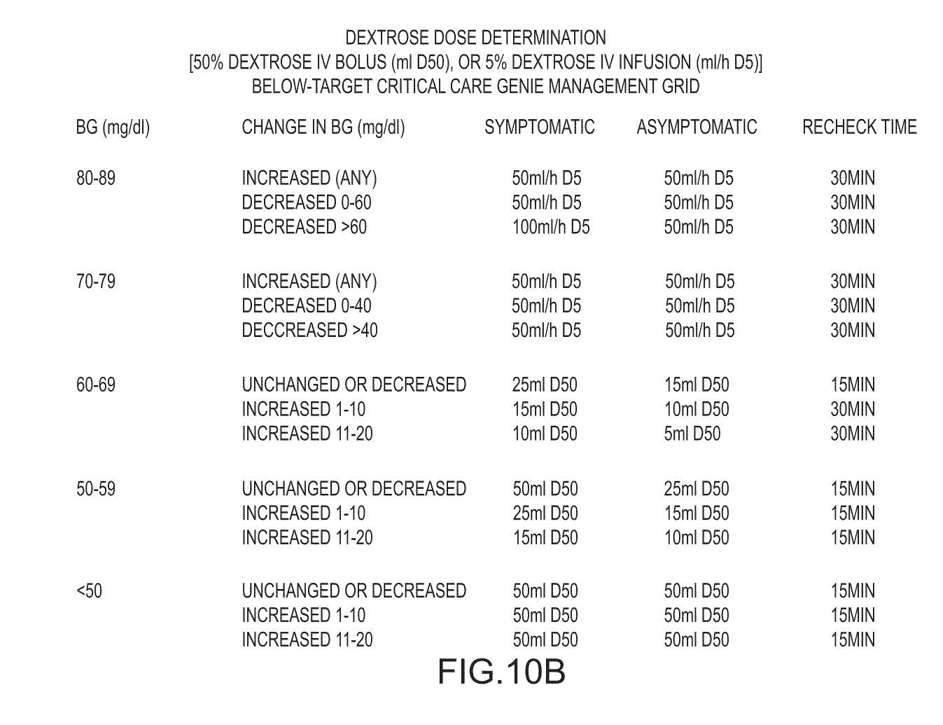

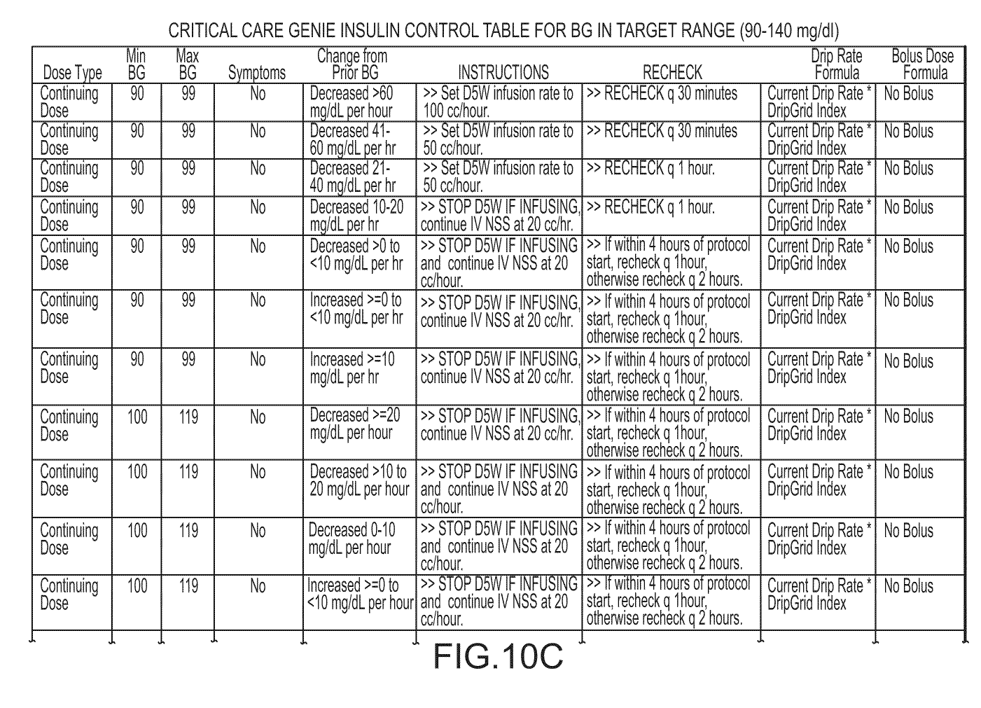

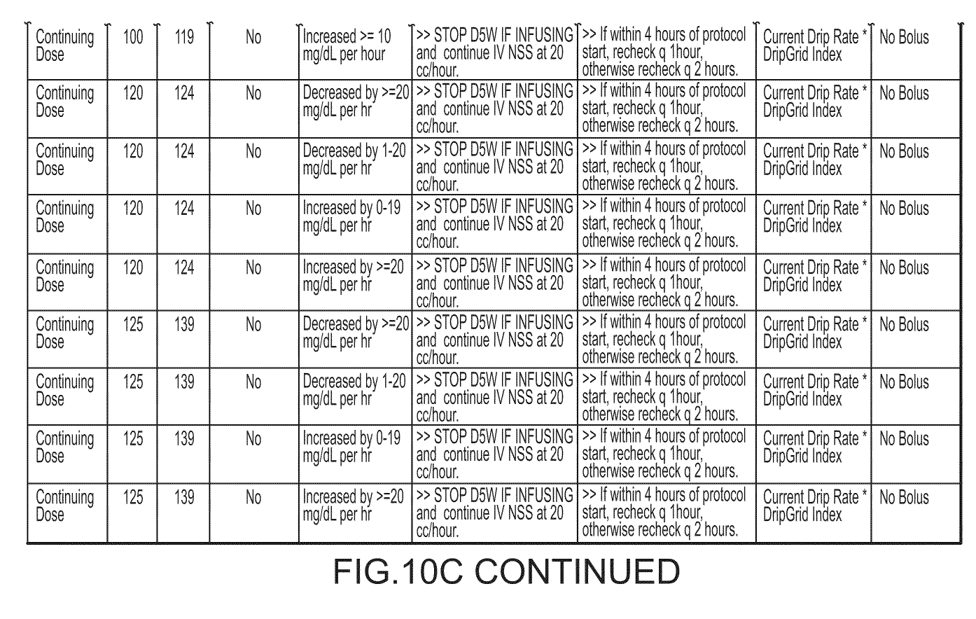

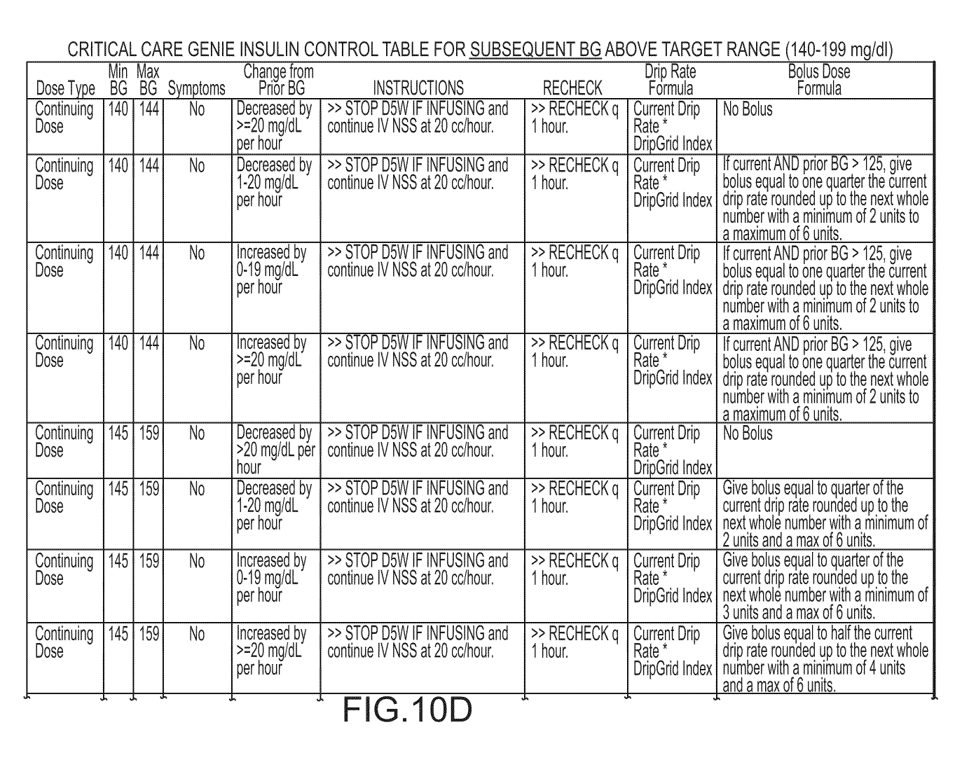

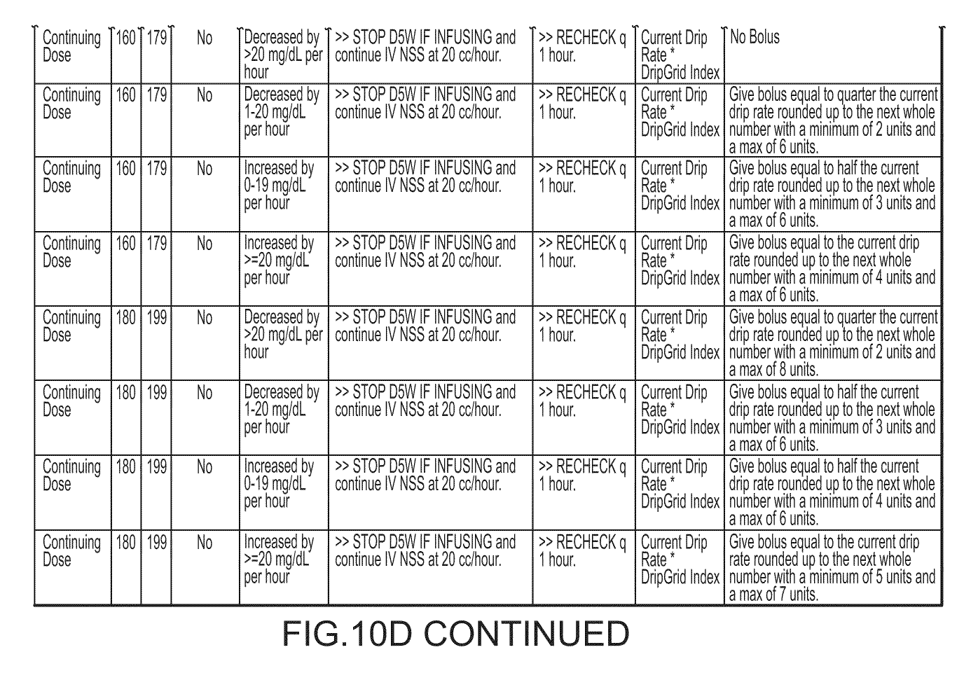

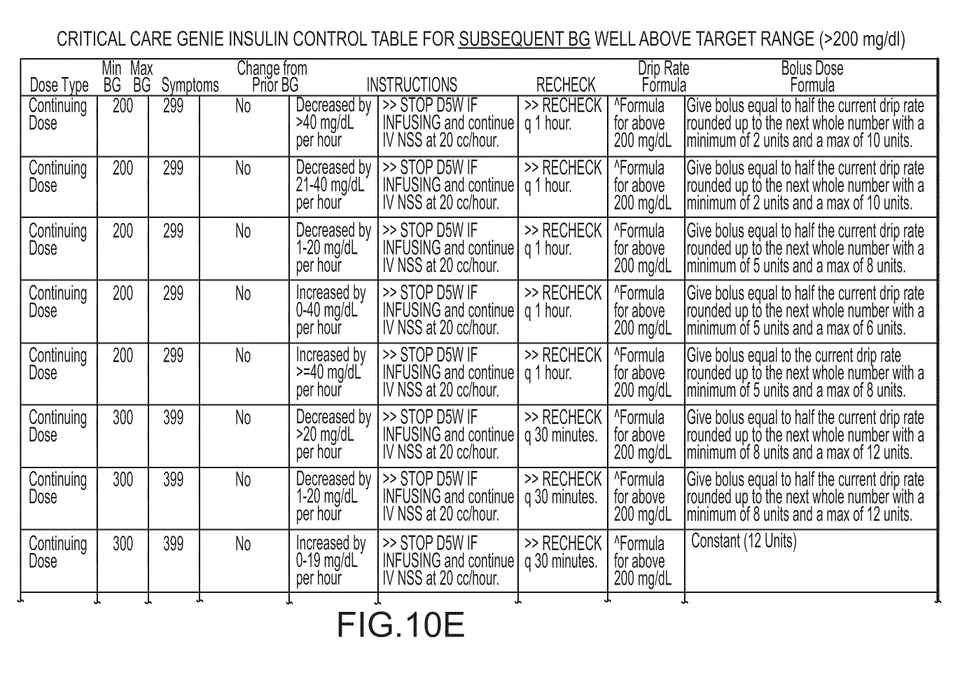

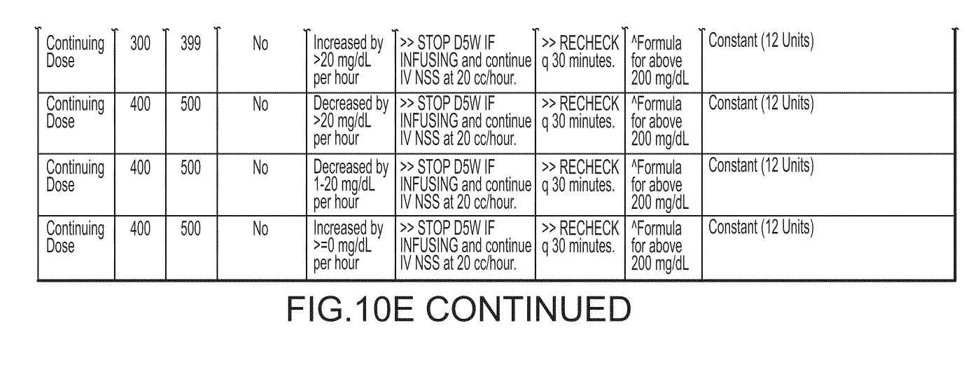

Below Target Range (approximately 0-89 mg/dl): FIG. 10A

Treatment decisions for BGs below the target range (<approximately 80 mg/dl) that are attained after starting the GENIE (Continuing Doses) are based on the “Below Target Management Grid” shown in FIG. 10B.

Critical Care GENIE has the same five unique advantages as those listed for the Cardiac Surgery GENIE. The seamless integration of this grid into the decision making strategy contributes directly to the virtual elimination of serious hypoglycemia with the use of this program. The principles are the same as those outlined for the Cardiac Surgery GENIE, with the only differences resulting from fact that the lower limit of the target range is approximately 90 mg/dl, as opposed to approximately 80 mg/dl. This means that the safety net (“twin-dial” strategy) provided against the onset of hypoglycemia through the simultaneous use of a 5% dextrose infusion while continuing the insulin infusion is implemented when the BG falls to approximately 89 mg/dl or less, instead of approximately 79 mg/dl.

The unique advantages (replicated from the Cardiac Surgery GENIE description) are:
(i) The very fact of the grid's incorporation into the main framework of the glycemic management program, which obviates the need for a separate protocol for management of hypoglycemia.
(ii) The calibration of the aggressiveness of the intervention according to not only the prevailing blood glucose as a determinant of intervention (a common feature in all hypoglycemia management protocols), but also, uniquely in GENIE, the directional change from the preceding blood glucose level. This approach thus accounts for the substantial difference in the significance of a blood glucose level of approximately 50 mg/dl, depending on whether this level was attained from a preceding value of approximately 40 mg/dl, or approximately 60 mg/dl. In the former case (blood glucose rising from the previous value of approximately 40) the goal is to accelerate the increase in blood glucose. In the latter (blood glucose falling from the previous value of approximately 60) the goal is to both stop the decline as well as to reverse it. As consequence, the former situation calls for a smaller amount of intravenous glucose than the latter.
(iii) Incorporating the presence or absence of symptoms of hypoglycemia into the decision, in recognition of the fact that symptomatic hypoglycemia calls for greater aggressiveness in intervention regardless of the actual level of blood glucose, than asymptomatic hypoglycemia.
(iv) The recognition of the importance of providing a “safety net” in patients who are trending outside the target range, thereby providing a safety net against the onset of hypoglycemia when the BG falls below the target range, instead of waiting to intervene until BG reaches hypoglycemic levels of <approximately 70 mg/dl. In this circumstance, simply discontinuing the drip might result in a precipitous rise in BG. In the Critical Care GENIE program, when BG falls into the approximately 90-99 mg/dl range as a result of a decrease in BG that exceeds approximately 20 mg/dl, the “Instructions” dictate the use of a 5% dextrose infusion to support the blood glucose and bring it gently back to the target range, while simultaneously decreasing the insulin drip rate according to the “drip grid index”, but not simply discontinuing the drip. This constitutes a “twin dial strategy” that improves the precision of control of BG at the lower end of the target range, thereby protecting against abrupt swings in BG.
(v) The fact that the required recheck times for the next blood glucose measurement are automatically adjusted according to both the actual BG level as well as the change in BG from previous value, once again reflecting the need to calibrate follow-up according to not just the severity of hypoglycemia but also the trend.
b. Critical Care GENIE Insulin Control Table for Subsequent BG in Target Range (approximately 80-110 mg/dl): FIG. 10C

Treatment decisions for BGs in the target range (approximately 90-140 mg/dl) that are attained after starting the GENIE (Continuing Doses) are similar in principle to those used in Cardiac Surgery GENIE in the target range for that program of approximately 80-110 mg/dl. The strategy similarly makes adjustments in the target range to the insulin drip exclusively, without the use of bolus doses. The drip rate calculations are derived from the “DRIP GRID INDEX”, which is described in the section of “Therapy Recommendations”. Computations in this range are based on three of the four independent variables that comprise the unique combination of inputs used to determine decision-making strategy in GENIE, namely the “glycemic magnitude”, “glycemic response” and “insulin response” variable. The rule base for drip rate adjustments is implemented in this range as described in Section A above.

The principles can be summarized as follows:
(i) Maintaining insulin drip at a constant rate for a BG that remains unchanged (BG decreasing by less than approximately 10 mg/dl)
(ii) Reducing insulin drip rate when the BG enters the target range by a decline of greater than approximately 10 mg/dl (“glycemic magnitude”)
(iii) Uniquely calibrating the decrease in drip rate at any blood glucose level in the target range to the preceding decline that led to the BG reaching target, so that an increasing rate of change (approximately 10-19, 20-39, 40-59, or >60 mg/dl/hr) triggers a greater reduction in insulin drip (“glycemic response”)
(iv) Uniquely calibrating the decrease in drip rate to the existing insulin infusion rate, so that changes are relative to each individual patient's insulin sensitivity (“insulin response”), and not a fixed or absolute decrease in drip rate.
(v) Uniquely recognizing that a safety net, in the form of a 5% dextrose infusion is required for:
   a. BG levels in the lower end of the target range (approximately 90-99)
   b. BG levels in the approximately 90-99 range which occur as a result of a decline in BG>approximately 20 mg/dl
   c. Calibrating the rate of 5% dextrose infusion rate to the magnitude of the decline from the preceding BG
c. Critical Care GENIE Insulin Control Table for Subsequent BG

Above Target Range (approximately 140-199 mg/dl): FIG. 10D

The strategy for maintaining glycemic control for BGs outside the target range of Critical Care GENIE, up to approximately 199 mg/dl, is similar to Cardiac Surgery GENIE, utilizing insulin drip rate adjustments and insulin bolus recommendations that are calculated from the identical four independent variables (glycemic magnitude, glycemic response, insulin responsiveness and insulin dose). The two programs, however, differ in the aggressiveness of intervention in this range, because of differing proximity of the approximately 140-199 range to the target ranges of the two programs (“Just Above” the target range of approximately 90-140 mg/dl for Critical Care GENIE, compared to “Well Above” the target range of approximately 80-115 mg/dl in Cardiac Surgery GENIE.

The Drip rate adjustments and bolus calculations are calculated according to the Drip Grid Index and Formula, and the Bolus Doses according to the Bolus dose formula described in Section A above.
d. Critical Care GENIE Insulin Control Table for Subsequent BG Well Above Target Range (≧approximately 200 mg/dl): FIG. 10E

The principles and approach to BGs in the severely hyperglycemic range (>approximately 200) is the same in both Cardiac Surgery and Critical Care GENIE, using a mathematical computation of insulin dose based on all four independent variables that comprise the decision-making strategy in GENIE, namely, “glycemic magnitude” (prevailing BG), “glycemic response” (rate of change in BG), “insulin response” (the prevailing insulin responsiveness reflected in the existing drip rate), and “insulin dose” for fractional partitioning of the dose into bolus and drip insulin. In this range, the philosophy is that errors in predicting response based on the use of a natural function are acceptable, since the BG is far enough above target range to allow for correctional action to be taken before the patient becomes hypoglycemic.

Two formulae are used, one for the Drip Rate, and the other for the Bolus dose, which reads off the drip rate, as described in Section A above.

D. Steps for Determining Insulin Dosing Recommendations for Glycemic Management under Exceptional Circumstances that are Common to Both ICU GENIEs

1. Steroid Administration: In this circumstance, the increase in BG occurs some 8-10 hours after the dose of steroid is administered. Both ICU GENIEs account for the hyperglycemia caused by steroid administration by recommending the administration of approximately 10 units of NPH insulin in anticipation of the expected increase in BG 6-10 hours later. The expectation is that this will damp down the acute hyperglycemic excursion, so that additional management can follow the usual GENIE management strategy without requiring insulin administration via large bolus doses

2. Hespan Administration: In this circumstance, the increase in BG is attributable to the continuous entry into the blood stream of an exogenous glucose load that is found in Hespan. The specific strategy here is to supersede all recommendations for bolus insulin administration, as would occur in the event of a spontaneous increase in BG, because these might precipitate an acute drop in blood glucose. The only recommendation would thus be to increase the insulin drip rate to the extent required for the glycemic excursion, lasting for the duration of Hespan administration.

3. Food Intake: The logic applied is the exact opposite of that used for continuous glucose entry from IV Hespan, because glucose entry into the system from food intake is short-lived and ideally suited to management through bolus insulin administration, rather than the insulin drip. Since the glucose excursion depends on the amount of carbohydrate ingested, so that the dose is calibrated to both the amount of food ingested (calculated as a nurse-estimated qualitative fraction of a standardized 75 gm carbohydrate meal) and the prevailing blood glucose at the time. No drip rate adjustments are made in this circumstance.

DKA Genie

The DKA GENIE program uses a straightforward algorithm for management of the insulin drip to determine dosing recommendations, using three of the four independent variables described above: (a) blood glucose level (glycemic magnitude) (b) change in blood glucose (“glycemic response”) and (c) the current insulin drip rate (“insulin response”).

The DKA GENIE program provides an “initial management strategy” for “Starting dose of insulin” that is “weight-based”, using a ratio of approximately 0.1 units/kg body weight, rounded up to the next closest even number, which is both the initial dose of IV bolus insulin, as well as the starting drip rate for an insulin infusion.

The DKA GENIE makes recommendations for subsequent insulin dosing by calibrating the changes in insulin drip (with any further bolus insulin dosing) to both glycemic magnitude and glycemic response. Recommendations are then made for q 1 hourly rechecks of blood glucose with each subsequent adjustment of insulin drip rate until termination. Additional prompts to the treating physician may be provided both with Initial Dose recommendations and with Subsequent Dosing changes.

The DKA GENIE uniquely provides a generic alert for fluid therapy along with the Initial Dosing recommendations. A generic alert may be provided for potassium replacement along with all dosing recommendations (both Initial and Subsequent, regardless of BG level). A generic alert may be provided for adjusting fluid type and rate of administration along with subsequent dosing recommendations. Several alerts may be provided along with subsequent dosing recommendations when two successive BGs are <150 mg/dl for “Drip Termination”. An option to calculate the dose of basal insulin to be administered prior to drip termination may be provided through a prompt, which calculates a recommended glargine dose from the last insulin infusion rate.

The DKA GENIE Start-up screen provides a unique safety feature against inappropriate use of DKA GENIE by requiring inputs that meet criteria for DKA, as follows: (a) Significant Hyperglycemia defined as a Blood Glucose level >approximately 300, AND (b) EITHER (i) Significant ketosis, defined as the presence of ketones either in Urine ≧2+, or in Plasma ≧1 in 8 dilution, OR (ii) Significant Acidosis, defined as either a Blood pH <approximately 7.30 or Serum bicarbonate <approximately 20 mmol/l.

The DKA GENIE alerts the ordering physician when the patient does not meet criteria for DKA and provides an option for a “Manual Over-ride” of DKA criteria in a patient with Type 1 Diabetes who is admitted to the ICU for a reason other than DKA.

The DKA GENIE's unique “Manual Over-ride” feature for the patient with Type 1 diabetes who does not have DKA provides an additional safety feature against inappropriate use by recommending the use of Critical Care GENIE as the appropriate alternative choice because the patient is likely to be insulin resistant, based on the home insulin requirement

Detailed Description of Rules for Determining DKA GENIE Recommendations

Initial Dose Recommendations:

Formula for Initial Dose:

Initial Bolus dose in units=(Pt wt in kg×0.01)

Starting Drip Rate in units/hour=(Pt wt in kg×0.01)

Subsequent Dose Recommendations:

DKA GENIE Insulin Control Rule Base is as follows:

(i) BG≧approximately 300 mg/dl, AND BG level decreased by approximately 40 to 99 mg/dl/hr: “Maintain current drip rate”

(ii) BG≧approximately 300 mg/dl AND BG level decreased >approximately 100 mg/dl/hr, “Decrease current drip rate by 50%”;

(iii) BG≧approximately 300 mg/dl AND BG level changed by +19 to −39 mg/dl/hr: “Increase current drip rate by 50%”;

(iv) BG≧approximately 300 mg/dl, AND BG level increased by ≧approximately 20 mg/dl/hr: “Increase drip rate by 100%”;

(v) BG approximately 200-299 mg/dl, AND BG level increased by ≧approximately 20 mg/dl/hr: “Increase current drip rate by 50%”

(vi) BG<approximately 200 mg/dl AND BG level decreased approximately 20-100 mg/dl: “Decrease current drip rate by 50%”

(vii) BG<approximately 200 mg/dl AND BG level decreased approximately 0-20 mg/dl: “Maintain current drip rate”.

(viii) BG approximately 150-200 mg/dl AND BG increased by approximately 20-50 mg/dl: “Increase current drip rate by 25%”

(ix) BG<approximately 150 mg/dl AND BG decreased by approximately 20-100 mg/dl: “Decrease current drip rate by 50%”.

(x) BG<approximately 150 mg/dl AND BG changed −20 to +19 mg/dl: “Maintain current drip rate”.

(xi) BG<approximately 150 mg/dl AND BG decreased by >100 mg/dl: “Decrease current drip rate by 75%”.

(xii) BG<approximately 150 mg/dl on two successive occasions AND BG changed by approximately ±10 mg/dl: “Maintain current drip rate. Consider discontinuing IV insulin”.

Drip Termination Dose Recommendations:

(i) Inputs: 2 successive BG<approximately 150 mg/dl, ±10 mg/dl difference

(ii) Dose of Lantus in units (to be administered 2 hours prior to drip termination)=Current Drip rate×24.

### DKA GENIE Treatment Alerts

(i) The DKA GENIE alert for fluid therapy along with the Initial Dosing recommendations is: “Ensure that the patient is adequately rehydrated with normal saline to replace intravascular volume, provided cardiac and renal function are adequate.

(ii) The DKA GENIE alert for potassium replacement along with all dosing recommendations (both Initial and Subsequent, regardless of BG level), is: “Follow potassium levels closely to determine need for replacement and monitor response to therapy”.

(iii) The DKA GENIE alert for adjusting fluid type and rate of administration along with subsequent dosing recommendations when BG is still above approximately 300 mg/dl is: (a) “If BP and pulse are normal, consider reducing rate of normal saline administration to approximately 250 ml/hr”. “(b) If urine output is >180 ml/hr (3 ml/min), consider changing IV fluid to 0.45% (half-normal) saline”.

(iv) The DKA GENIE alert for adjusting fluid type and rate of administration along with subsequent dosing recommendations when BG level falls below approximately 300 mg/dl to adjust fluid type and rate of administration is: “If BP and pulse are normal, and urine output is >approximately 180 ml/hr (3 ml/min), consider changing IV fluid to Quarter-normal) saline”.

(v) The DKA GENIE alert to adjust fluid type and rate of administration along with subsequent dosing recommendations when BG level falls below approximately 200 mg/dl is: “If BP and pulse are normal, and urine output is >180 ml/hr, consider changing fluid type to 5% dextrose at approximately 125 ml/hr”

(vi) The DKA GENIE alerts for “Drip Termination” when two successive BGs are <approximately 150 mg/dl are: (1) “Measure serum electrolytes to calculate anion gap (AG). If AG≦2, consider terminating DKA GENIE.” (2) If considering drip termination, administer basal insulin (NPH or glargine) TWO hours prior to terminating the insulin drip, according to home insulin regimen”.

Safety Features

(i) The DKA GENIE Safety feature against inappropriate use of DKA GENIE: The Start-up screen uniquely requires the following inputs to make sure that criteria for DKA are met:
a. Type 1 Diabetes WITH
b. Significant Hyperglycemia defined as a Blood Glucose>approximately 300 mg/dl, AND
c. EITHER
Significant ketosis, defined as the presence of ketones either in Urine ≦2+, or in Plasma ≦1 in 8 dilution, OR
Significant Acidosis, defined as either a Blood pH <approximately 7.30 or Serum bicarbonate <20 mmol/l.

(ii) The DKA GENIE Safety feature when the treating physician does not affirm that the patient has Type 1 diabetes: DKA GENIE directs the treating physician to consider using Critical Care GENIE.

(iii) The DKA GENIE option for a “Manual Over-ride” of DKA criteria for the patient with Type 1 diabetes who does not have DKA provides several additional safety features against inappropriate use.
Requiring two inputs: (a) home insulin doses and (b) body weight, from which the patient's home insulin requirement is calculated in units/kg. The “Manual Over-ride” feature is activated only if it is <approximately 0.2 units/kg body weight.
If the whole insulin requirement exceeds approximately 0.2 units/kg body weight, the DKA GENIE recommends the use of Critical Care GENIE as the appropriate alternative choice because the patient is likely to be insulin resistant, despite an apparent diagnosis of Type 1 diabetes.
When the “Manual Over-ride” feature is activated, DKA GENIE bypasses the usual initial dose recommendation for bolus insulin and recommends only the calculated starting insulin drip rate.
When the “Manual Over-ride” feature is activated, DKA GENIE includes a system for bypassing the “Drip Termination” recommendation, thereby allowing for the patient with Type 1 diabetes who is not in DKA to be maintained on an insulin drip despite successive blood glucose levels in the target range (90-150 mg/dl).

Subcutaneous Genie

The Subcutaneous Insulin GENIE is intended for use outside the intensive care unit setting, in contrast to other three GENIE programs (Cardiac Surgery, Critical Care and DKA GENIEs), and is based on a different model from any of the three GENIE programs, all of which provide recommendations for intravenous insulin administration by drip and bolus.

The Subcutaneous Insulin GENIE provides recommendations for basal-bolus insulin therapy by the subcutaneous route, consisting of bolus doses of an ultra fast-acting analog insulin, such as aspart, lispro or glulisine insulin, and basal doses of a 24-hour insulin (glargine). The Subcutaneous Insulin GENIE is used to either transition patients from an intravenous insulin treatment regimen in the ICU (Cardiac Surgery and Critical Care GENIE) to subcutaneous insulin therapy outside the ICU, or to initiate a tight glycemic control program (approximately 90-180 mg/dl) with subcutaneous insulin in patients who are admitted to a non-intensive care hospital setting.

The Subcutaneous Insulin GENIE program uses a unique concept of administering a bolus of short-acting analog insulin (aspart/Novolog) immediately after the patient eats, rather than before meals, as in other traditional subcutaneous basal-bolus insulin regimens. The analog insulin recommendations can be applied equally to other short-acting analog insulin, such as lispro/Humalog or glulisine/Apidra. Bolus doses are calculated from both the blood glucose level and the grams of carbohydrate consumed by the patient, rather than blood glucose alone. An essential and unique component is the requirement for a “consistent carbohydrate diet” containing approximately 75 gms of carbohydrate at each meal. The “consistent carbohydrate diet” enables the nurse to make a reasonably accurate approximation of the amount of carbohydrate consumed at each meal, drawing on their already existing skill of assessing that a patient has eaten “most”, “half”, “little”, or “none” of the offered meal. It is, therefore, unique in adjusting insulin dose to meal intake, obviating the need to account separately for situations when the patient fails to eat or is kept fasting in anticipation of some medical procedure, which would necessitate that bolus insulin be withheld in other protocols, for fear of hypoglycemia, and incurring the risk of hyperglycemia as “the lesser of the two evils”.

The Subcutaneous Insulin GENIE program recalibrates the dose of bolus insulin to be administered at each meal according to insulin sensitivity, based on the glycemic response to the previously administered bolus dose in relation to the amount of carbohydrate consumed.

The Subcutaneous Insulin GENIE program requires the routine administration of glargine, a type of basal insulin with a 24 hour duration of action and no peak, in two divided doses 12 hours apart, rather than once a day, to allow for more precise calculation of the basal insulin requirement and a more rapid escalation of basal insulin doses. Splitting the basal insulin doses into two doses provides additional safety if low glycemic levels indicate termination of basal insulin dosing.

The dose of glargine is recalculated every day, based on the fasting blood glucose level and the previously administered dose of glargine. An automated hypoglycemia prevention and intervention protocol is incorporated for patients with blood glucose <approximately 70 mg/dl, obviating the need for separate orders for management of hypoglycemia.

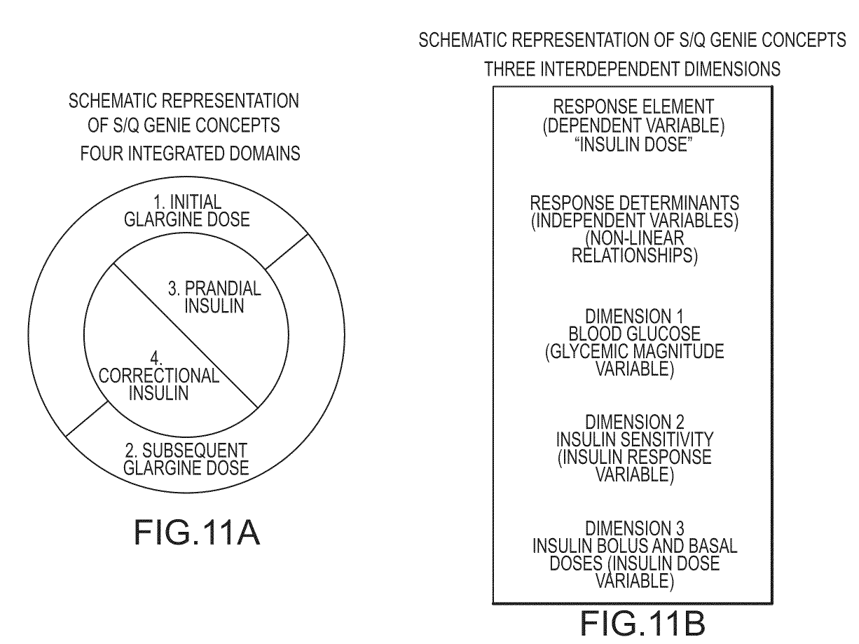

Detailed Description of Rules for Determining Subcutaneous GENIE Recommendations (FIGS. 11A-13B)

Concepts Underlying Decision Making Strategy

The Subcutaneous Insulin GENIE (Sub/Q GENIE) is based on similar concepts as the two ICU GENIEs, with four integrated domains and the incorporation of three of the four independent variables used in the ICU GENIEs, as shown in FIGS. 11A and 11B. However, there are significant differences between the platforms of the Sub/Q and the ICU GENIEs:

1. In the first place, the target range of approximately 90-180 in Sub/Q GENIE calls for a less aggressive strategy for implementing the concepts.

2. Second, the route of administration of insulin results in markedly different insulin pharmacokinetics both for bolus therapy (subcutaneous aspart vs. intravenous regular), as well as for basal therapy (subcutaneous glargine vs. continuous intravenous infusion).

3. Finally, the far less frequent blood glucose monitoring requirements (four times a day vs. every 1-2 hours, respectively) dictates a more measured intervention strategy measured in several hours rather than in minutes.

The Sub/Q GENIE's starting User Interface (shown in FIG. 11A and 11B) requires a distinction to be made between:

A. Initiation of a Basal-Bolus Insulin regimen (Initial Dosing Recommendations),

B. Maintenance of the Basal-Bolus Insulin regimen (Subsequent Dosing Recommendations), and

C. Termination of the Basal-Bolus Insulin regimen at the time of discharge (Discharge Instructions).

The decision-making strategy and rule base thus differ under these 3 different scenarios, and differs for the basal component (glargine) and the bolus component (aspart/Novolog insulin).

A. Initiation of Basal-Bolus Insulin Regimen (Initial Dosing Strategy)

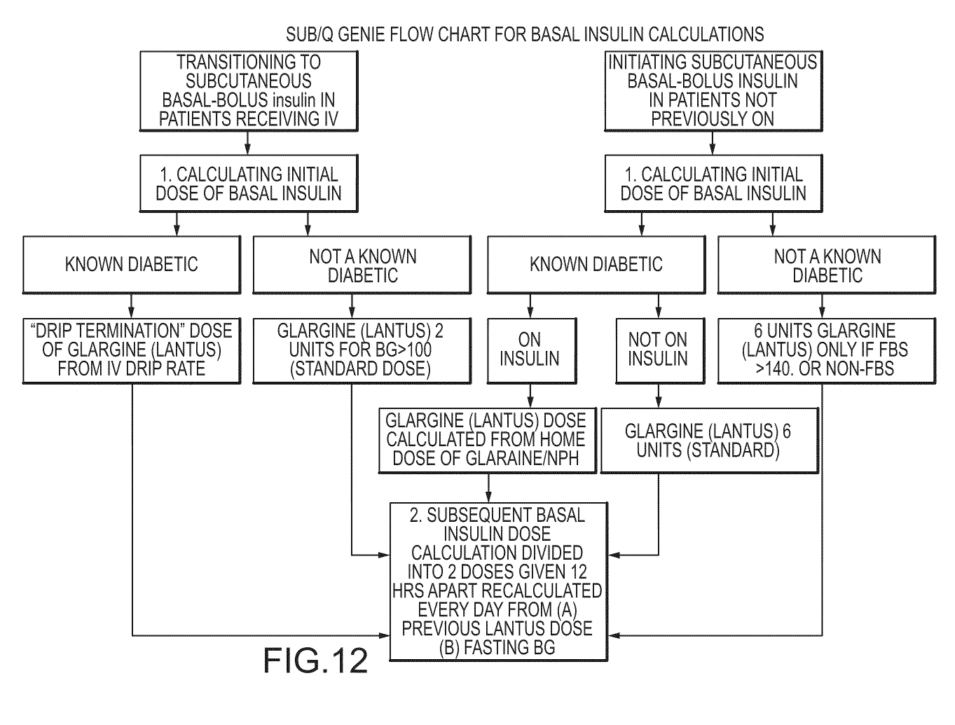

The dosing strategy for glargine is shown schematically in FIG. 12F, which outlines the approach to both the initial dose (depending on whether the patient is being transitioned off an IV insulin drip or not) and the subsequent dose, which is discussed in the next section. As noted earlier, at initiation, basal insulin (glargine) is the only insulin given, so that the strategy focuses solely on basal insulin dosing at initiation. On the other hand, subsequent insulin dosing comprises both basal and bolus insulin, where bolus insulin administration and dosing strategy are tied to meals, as will be described later.

Concepts underlying Initial Glargine Dose Determination:

Sub/Q GENIE takes the following factors into consideration in determining dosing recommendations:

1. Circumstance (i.e. Transition from IV insulin drip vs. Initiation in a patient not previously on a drip)

2. Diabetic vs. Non diabetic status.

3. Anti-diabetic treatment (diet, orals or insulin) in the diabetic patient

4. Severity of hyperglycemia in the non-diabetic patient

Rule Base for Initial Glargine Dose Calculation:

1. When transitioning from an IV insulin drip, the initial basal insulin dose (“Drip Termination Glargine dose) is calculated from the current insulin drip rate at the time the decision is taken to terminate IV insulin therapy, with a minimum of 10 units and a maximum of 34 units being administered 2 hours prior to drip termination.

2. When initiating therapy without a prior insulin drip, the initial basal dose for a diabetic patient on insulin is calculated from the patient's home insulin regimen, depending on whether the patient is taking NPH insulin, 70/30 insulin, or glargine at home:
a. For a patient on NPH at home: GENIE calculates the Initial glargine dose using a standard formula, whereby the total 24 hour basal glargine requirement is assessed as being 80% of the total daily NPH insulin dose. This is then divided in half (for q 12 hour administration) and administered as the Initial dose.
b. For a patient on 70/30 insulin at home, GENIE takes 70% of the total daily dose as being NPH, and then makes the same computation (i.e. takes 80% of the NPH dose, and recommends that half that amount be administered as the Initial dose.
c. For a patient on glargine at home, GENIE recommends that 50% of the total daily Glargine dose be administered as the initial dose.

3. When initiating therapy without a prior insulin drip for a diabetic patient not on insulin (i.e. controlled on diet or oral agents), GENIE recommends a standard initial basal dose of 6 units of glargine.

4. When initiating therapy for a non-diabetic patient with hyperglycemia (defined as a fasting BG>140, or a diurnal BG>180) in hospital, without a prior insulin drip, GENIE recommends 2 units of Glargine as a starting dose.

B. Maintenance of Basal-Bolus Insulin Regimen (Subsequent Dosing

### Strategy)

As outlined earlier, subsequent dosing involves both basal (Glargine) and Bolus (aspart) insulin at mealtimes, which involve different strategies and rule bases.

1. Subsequent Basal Insulin (Glargine) Dosing Strategy

Concepts underlying Subsequent Glargine Dose Determination:

The dosing strategy for glargine is shown schematically in FIG. 12, which outlines the approach to both the initial dose (discussed in the previous section) and the subsequent dose. The common concept variables that are incorporated into the decision making strategy for Subsequent Glargine dosing are:

1. Glycemic magnitude as reflected in Blood Glucose adjusting the subsequent basal insulin dose depending on the patient's glucose level in response to the previous glargine dose.

2. Insulin response, based on the prior Basal Insulin Dose (Last Glargine dose given)

Rule Base for Subsequent Glargine Dose Calculation:

GENIE uniquely calculates the basal Insulin dosing based on a q 12 hour administration regimen, so that doses of “AM” glargine (to be given at 7 AM), and PM glargine (to be given at 9 PM) are calculated separately.

Calculation of AM Glargine Dose

GENIE makes recommendations as follows, depending on Fasting Blood Glucose (FBG) with a target of approximately 90-150:

1. If FBG is <60, no basal insulin is recommended

2. If FBG is 60-79, GENIE recommends a 4 unit decrease from the last glargine dose

3. If FBG is 80-99, GENIE recommends a 2 unit decrease from the last glargine dose

4. If FBG is 100-119, GENIE recommends the same dose of glargine as the last glargine dose

5. If FBG is 120-149, GENIE recommends a 2 unit increase from the last glargine dose

6. If FBG is ≧150, GENIE recommends a 2 unit increase from the last glargine dose, PLUS an additional unit for every 40 mg/dl that the FBG exceeds 149 mg/dl, rounded to the nearest whole number

Calculation of PM Glargine Dose

GENIE makes recommendations as follows, depending on 9 PM BG:

1. If 9 PM BG is <80, no PM basal insulin is recommended

2. If 9 PM BG is ≧80, GENIE recommends the same dose of glargine as the last (AM) glargine dose

2. Subsequent Bolus (Mealtime) Insulin (aspart/Novolog) Strategy

GENIE takes into account two components that make up the total mealtime bolus insulin dose: the component related to meal carbohydrate consumption, called Prandial Insulin, and the component related to the blood glucose level at the time the meal is consumed, called Correctional Insulin. The variables that are used in the computation are the glycemic magnitude (premeal BG level) and insulin response (previous glargine dose as a reflection of insulin sensitivity).

Prandial Insulin Dose Calculations
a. GENIE recommends Prandial bolus Insulin doses only if pre-meal BG is >150.
b. The calculation is founded on a well-established assumption that the amplitude of the excursion in BG after a meal is directly related to the amount of carbohydrate consumed.
c. The relationship between insulin requirement and carbohydrate consumed for an average patient can be approximated by a standard insulin-to-carbohydrate ratio of 1 unit/15 gm of carbohydrate consumed.
d. GENIE corrects this “average” computation for insulin sensitivity by factoring in the patients last glargine dose as follows.
   i. A dose of glargine of 20 units or less (remembering that GENIE requires q 12 hour dosing, so this represents only 50% of total glargine dose) is assumed to represent “average” insulin sensitivity, whereby the computation of prandial bolus is based on ratio of 1 unit/15 gm carbohydrate consumed.
   ii. A glargine dose >20 units is assumed to represent a decrease in insulin sensitivity, so computation is based on a ratio of 1 unit/10 gm carbohydrate consumed.
e. GENIE uniquely requires that the patient be given a standardized meal tray with a 75 gm carbohydrate content in each meal in order to make the response more predictable.
f. The Nurse is required to make a qualitative assessment of the fraction of food the patient has consumed (“none”, ““little”, “about half”, or “most”)
g. The dose of insulin is then calculated from quantitatively equivalent fractions of the 75 gm load, (computed as 0, 25%, 50% 100% of the load, respectively)

Correctional Insulin Dose Calculations
h. Correctional insulin is added to the mealtime coverage based on the premeal blood glucose level
i. This coverage is based on a ratio of 1 unit/50 mg/dl above 150 mg/dl, rounded up to the nearest whole number.

The Total Mealtime Bolus (Prandial+Correctional) is administered uniquely in GENIE as post-meal coverage within half an hour of meal consumption.

C. Termination of Basal-Bolus Insulin Regimen (Discharge Instructions

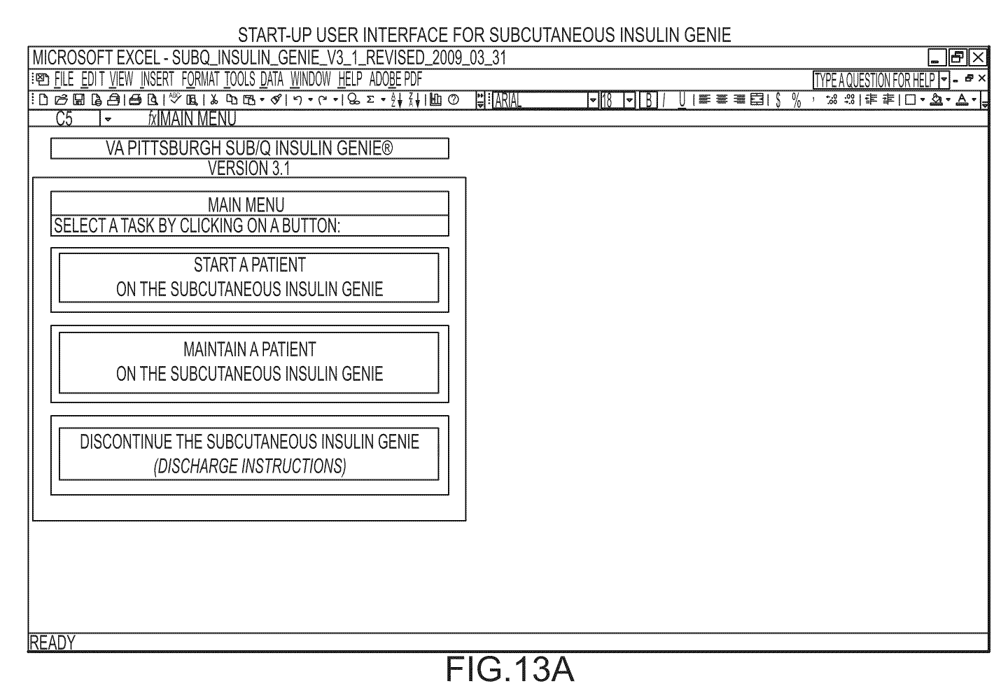

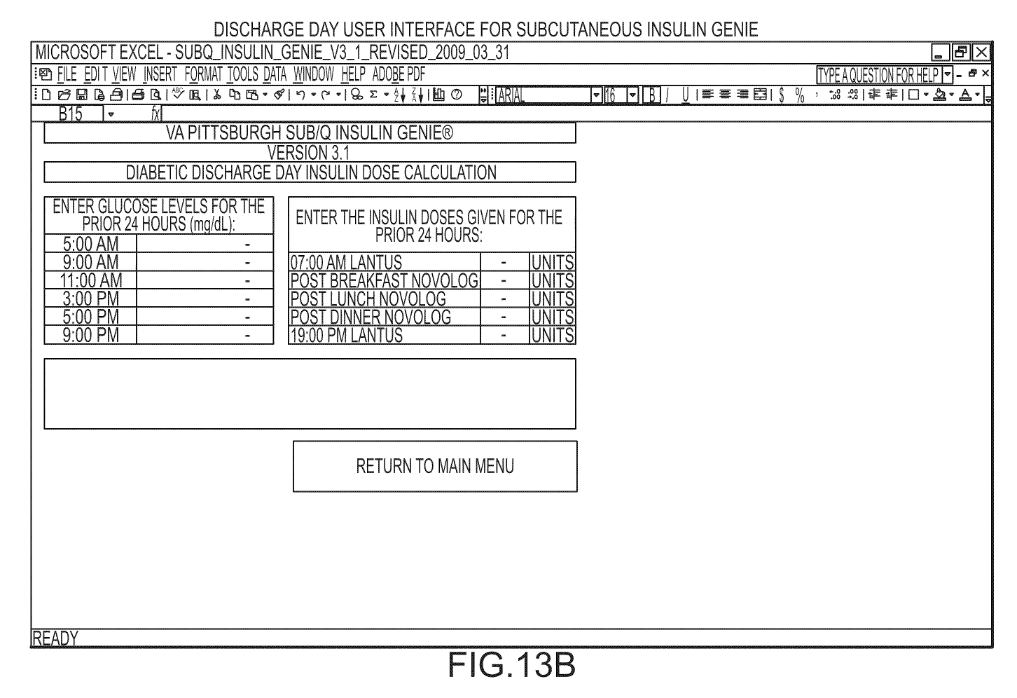

Sub/Q GENIE makes recommendations for appropriate insulin dosing upon discharge from hospital using a User Interface (FIGS. 13A and 13B) selected by clicking on the appropriate box in the Main Menu shown in FIG. 12.

The rule base for Insulin Dose Determination at Discharge takes the same factors into consideration to make recommendations consistent with prior strategy, including:
a. Glycemic magnitude variable: GENIE uses the percentage of blood glucose values in the last 24 hours above or below 150 to assess:
   i. The need for Insulin or Oral agents
   ii. The need change (increase) the current insulin doses
b. Insulin response variable: GENIE uses the total glargine (Lantus) dose as a measure of insulin sensitivity to assess whether the patient can be discharged on oral agents or insulin, depending on whether glargine .a of 0-20,), insulin dose.
c. Insulin dose variable: The dose of insulin is calculated from the total insulin dose.

The table shown below shows the manner in which the discharge day decision strategy is implemented for both diabetic and non-diabetic patients.

| | BG >150 in | 24 hr Total | GENIE |
|---|---|---|---|
| Diabetic | last 24 h | insulin use | Recommendations |
| Yes | <=50% | <=20 | Discharge on Glyburide (5 mg BID) and |
| | | | DO NOT give Metformin. |
| Yes | <=50% | 21-40 | Discharge on Glyburide 5 mg at 24 units |
| | | | before breakfast qAM and 12 units qPM. |
| Yes | <=50% | >40 | Discharge on 70/30 Humalog at 24 units |
| | | | qAM before breakfast and 12 units qPM. |
| Yes | >50% | <=20 | Discharge on Glyburide (5 mg BID) and |
| | | | 12 units NPH at bedtime. |
| Yes | >50% | 21-40 | Discharge on 70/30 Humalog at 24 units |
| | | | before breakfast qAM and 12 units qPM. |
| Yes | >50% | >40 | Discharge on 70/30 Humalog at 30 units |
| | | | before breakfast qAM and 16 units qPM. |
| No | <=50% | <=20 | Discharge on no anti-hyperglycemia |
| | | | therapy. |
| No | <=50% | 21-40 | Discharge on 5 mg Glyburide qAM and |
| | | | 5 mg Glyburide qPM. |
| No | <=50% | >40 | Discharge on 5 mg Glyburide BID AND |
| | | | 10 units NPH at bedtime. |
| No | >50% | <=20 | Discharge on 5 mg Glyburide qAM. |
| No | >50% | 21-40 | Discharge on 5 mg Glyburide BID AND |
| | | | 10 units NPH at bedtime. |
| No | >50% | >40 | Discharge on 70/30 insulin at 20 units |
| | | | qAM and 10 units NPH at bedtime. |

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. An automated method for diabetes control comprising the steps of:
establishing a blood glucose target for an insulin user;
measuring an existing blood glucose level for the insulin user;
inputting the existing blood glucose level into at least one computer processor, the at least one computer processor being formed to execute at least one algorithm designed to calculate a corrective amount of insulin to be administered intravenously in an integrated basal-bolus manner to the insulin user if the existing blood glucose level exceeds the blood glucose target, wherein the at least one algorithm is based on a plurality of factors that contribute to a non-linear glucose response;
automatically delivering the corrective amount of insulin to the insulin user; and,
repeating the measuring, inputting, and delivering steps one or more times to maintain the insulin user within the blood glucose target range.

2. The method of claim 1, wherein the blood glucose target is 80-110 mg/dl (milligrams per deciliter).

3. The method of claim 1, wherein the blood glucose target is 90-140 mg/dl (milligrams per deciliter).

4. The method of claim 1, wherein the plurality of factors comprise a magnitude of the existing blood glucose level, the preceding blood glucose level, a time between the existing blood glucose level and a preceding blood glucose level, a calculated rate of change in the blood glucose, a total insulin dose, and a fractional change in total insulin dose.

5. The method of claim 1, further comprising the step of progressively reducing the corrective amount of insulin to the insulin user as insulin sensitivity improves as the existing blood glucose level reaches the blood glucose target.

6. The method of claim 1, further comprising the step of providing a safety warning in an integrated user interface to recheck the existing blood glucose level at a variable predetermined time.

7. The method of claim 1, wherein the method is designed for use in a hospital intensive care unit.

8. An automated method for diabetes control comprising the steps of:
establishing a blood glucose target for an insulin user;
establishing a carbohydrate target for the insulin user;
measuring an existing blood glucose level for the insulin user;
measuring an existing amount of carbohydrate consumed by the insulin user;
inputting the existing blood glucose level into at least one computer processor, the at least one computer processor being formed to execute at least one algorithm designed to calculate a corrective amount of insulin to be administered subcutaneously in an integrated basal-bolus manner to the insulin user if the existing blood glucose level exceeds the blood glucose target in relation to the existing amount of carbohydrate consumed;
automatically delivering the corrective amount of insulin to the insulin user after the insulin user eats a meal; and,
repeating the measuring, inputting, and delivering steps one or more times to maintain the insulin user within the blood glucose target range.

9. The method of claim 8, wherein the blood glucose target is 90-140 mg/dl (milligrams per deciliter).

10. The method of claim 8, wherein the carbohydrate target is 75 gm (grams) of carbohydrate per meal.

11. The method of claim 8, wherein the corrective amount of insulin to be administered subcutaneously comprises a bolus dose of a fast-acting analog insulin and a basal dose of a 24-hour insulin.

12. The method of claim 11, wherein the bolus dose of the fast-acting analog insulin is selected from the group comprising aspart insulin, lispro insulin and glulisine insulin.

13. The method of claim 11, wherein the basal dose of the 24-hour insulin comprises glargine.

14. The method of claim 8, wherein the method is designed for use in a hospital non-intensive care unit setting.

15. An automated system for diabetes control comprising:
a database for obtaining a blood glucose target for an insulin user;
a glucose measuring device for measuring an existing blood glucose level for the insulin user;
an insulin delivery unit; and,
a computer having a computer processor that receives blood glucose level measurements from the glucose measuring device, executes a software program that calculates a corrective amount of insulin to be administered intravenously in an integrated basal-bolus manner to the insulin user if the existing blood glucose level exceeds the blood glucose target, wherein the software program is based on a plurality of factors that contribute to a non-linear glucose response, and further wherein the software program has a communication element that communicates to the insulin delivery unit the corrective amount of insulin to be administered to the insulin user.

16. The system of claim 15, wherein the blood glucose target is 80-110 mg/dl (milligrams per deciliter).

17. The system of claim 15, wherein the blood glucose target is 90-140 mg/dl (milligrams per deciliter).

18. The system of claim 15, wherein the plurality of factors comprise a magnitude of the existing blood glucose level, a magnitude of the preceding blood glucose level, a calculated rate of change in the blood glucose, a time between the existing blood glucose level and a preceding blood glucose level, a total insulin dose, and a fractional change in total insulin dose.

19. An automated system for diabetes control comprising:
a database for obtaining a blood glucose target for an insulin user;
a database for obtaining a carbohydrate target for the insulin user;
a glucose measuring device for measuring an existing blood glucose level for the insulin user;
a carbohydrate measuring device for measuring an existing amount of carbohydrate consumed by the insulin user;
an insulin delivery unit; and,
a computer having a computer processor that receives blood glucose level measurements from the glucose measuring device and receives carbohydrate consumed measurements from the carbohydrate measuring device, and executes a software program that calculates a corrective amount of insulin to be administered subcutaneously in an integrated basal-bolus manner to the insulin user if the existing blood glucose level exceeds the blood glucose target in relation to the existing amount of carbohydrate consumed, wherein the software program has a communication element that communicates to the insulin delivery unit the corrective amount of insulin to be administered to the insulin user after the insulin user eats a meal.

20. The system of claim 19, wherein the blood glucose target is 90-140 mg/dl (milligrams per deciliter).
